SEQUENCE LISTING (304 sequences)

SEQ ID NO: 1 - protein, 6 aa, Artificial Sequence
  Synthesized
  Misc_feature (1)..(6): X = any amino acid
XXXXRF

SEQ ID NO: 2 - protein, 6 aa, Artificial Sequence
  Synthesized
  Misc_feature (1)..(6): X = any amino acid
XXXXFH

SEQ ID NO: 3 - protein, 6 aa, Artificial Sequence
  Synthesized
  Misc_feature (1)..(6): X = any amino acid
XXXXKF

SEQ ID NO: 4 - protein, 6 aa, Artificial Sequence
  Synthesized
  Misc_feature (1)..(6): X = any amino acid
XXXXQR

SEQ ID NO: 5 - protein, 6 aa, Artificial Sequence
  Synthesized
  Misc_feature (1)..(6): X = any amino acid
XXXXRM

SEQ ID NO: 6 - protein, 6 aa, Artificial Sequence
  Synthesized
  Misc_feature (1)..(6): X = any amino acid
XXXXHM

SEQ ID NO: 7 - protein, 6 aa, Artificial Sequence
  Synthesized
  Misc_feature (1)..(6): X = any amino acid
XXXXKL

SEQ ID NO: 8 - protein, 6 aa, Artificial Sequence
  Synthesized
  Misc_feature (1)..(6): X = any amino acid
XXXXRL

SEQ ID NO: 9 - protein, 6 aa, Artificial Sequence
  Synthesized
  Misc_feature (1)..(6): X = any amino acid
XXXLRF

SEQ ID NO: 10 - protein, 6 aa, Artificial Sequence
  Synthesized
  Misc_feature (1)..(6): X = any amino acid
XXXIRF

SEQ ID NO: 11 - protein, 6 aa, Artificial Sequence
  Synthesized
  Misc_feature (1)..(6): X = any amino acid
XXXFRF

SEQ ID NO: 12 - protein, 6 aa, Artificial Sequence
  Synthesized
  Misc_feature (1)..(6): X = any amino acid
XXXWRF

SEQ ID NO: 13 - protein, 6 aa, Artificial Sequence
  Synthesized
  Misc_feature (1)..(6): X = any amino acid
XXXMRF

SEQ ID NO: 14 - protein, 6 aa, Artificial Sequence
  Synthesized
  Misc_feature (1)..(6): X = any amino acid
XXKLRF

SEQ ID NO: 15 - protein, 6 aa, Artificial Sequence
  Synthesized
  Misc_feature (1)..(6): X = any amino acid
XXRLRF

SEQ ID NO: 16 - protein, 6 aa, Artificial Sequence
  Synthesized
  Misc_feature (1)..(6): X = any amino acid
XXHLRF

SEQ ID NO: 17 - protein, 6 aa, Artificial Sequence
  Synthesized
  Misc_feature (1)..(6): X = any amino acid
XXTLRF

SEQ ID NO: 18 - protein, 6 aa, Artificial Sequence
  Synthesized
  Misc_feature (1)..(6): X = any amino acid
XXFLRF

SEQ ID NO: 19 - protein, 6 aa, Artificial Sequence
  Synthesized
  Misc_feature (1)..(6): X = any amino acid
XXSLRF

SEQ ID NO: 20 - protein, 6 aa, Artificial Sequence
  Synthesized
  Misc_feature (1)..(6): X = any amino acid
XXILRF

SEQ ID NO: 21 - protein, 6 aa, Artificial Sequence
  Synthesized
  Misc_feature (1)..(6): X = any amino acid
XXLLRF

SEQ ID NO: 22 - protein, 6 aa, Artificial Sequence
  Synthesized
  Misc_feature (1)..(6): X = any amino acid
XXALRF

SEQ ID NO: 23 - protein, 6 aa, Artificial Sequence
  Synthesized
  Misc_feature (1)..(6): X = any amino acid
XXWLRF

SEQ ID NO: 24 - protein, 6 aa, Artificial Sequence
  Synthesized
  Misc_feature (1)..(6): X = any amino acid
XXMLRF

SEQ ID NO: 25 - protein, 3 aa, Artificial Sequence
  Synthesized
FRF

SEQ ID NO: 26 - protein, 3 aa, Artificial Sequence
  Synthesized
LRF

SEQ ID NO: 27 - protein, 3 aa, Artificial Sequence
  Synthesized
WRF

SEQ ID NO: 28 - protein, 3 aa, Artificial Sequence
  Synthesized
HRF

SEQ ID NO: 29 - protein, 4 aa, Artificial Sequence
  Synthesized
FLRF

SEQ ID NO: 30 - protein, 4 aa, Artificial Sequence
  Synthesized
WLRF

SEQ ID NO: 31 - protein, 5 aa, Artificial Sequence
  Synthesized
FHLRF

SEQ ID NO: 32 - protein, 6 aa, Artificial Sequence
  Synthesized
FFKLRF

SEQ ID NO: 33 - protein, 6 aa, Artificial Sequence
  Synthesized
VFKLRF

SEQ ID NO: 34 - protein, 6 aa, Artificial Sequence
  Synthesized
HFKLRF

SEQ ID NO: 35 - protein, 6 aa, Artificial Sequence
  Synthesized
IFKLRF

SEQ ID NO: 36 - protein, 6 aa, Artificial Sequence
  Synthesized
KRKLRF

SEQ ID NO: 37 - protein, 6 aa, Artificial Sequence
  Synthesized
LFKLRF

SEQ ID NO: 38 - protein, 6 aa, Artificial Sequence
  Synthesized
YFKLRF

SEQ ID NO: 39 - protein, 7 aa, Artificial Sequence
  Synthesized
FHFKLRF

SEQ ID NO: 40 - protein, 7 aa, Artificial Sequence
  Synthesized
IHFKLRF

SEQ ID NO: 41 - protein, 6 aa, Artificial Sequence
  Synthesized
FRLKFH

SEQ ID NO: 42 - protein, 6 aa, Artificial Sequence
  Synthesized
RFKLRF

SEQ ID NO: 43 - protein, 6 aa, Artificial Sequence
  Synthesized
SFKLRF

SEQ ID NO: 44 - protein, 6 aa, Artificial Sequence
  Synthesized
MFKLRF

SEQ ID NO: 45 - protein, 6 aa, Artificial Sequence
  Synthesized
TFKLRF

SEQ ID NO: 46 - protein, 6 aa, Artificial Sequence
  Synthesized
QFKLRF

SEQ ID NO: 47 - protein, 6 aa, Artificial Sequence
  Synthesized
WFKLRF

SEQ ID NO: 48 - protein, 44 aa, Tachypleus tridentatus
  misc_feature: Tachystatin A Peptide
YSRCQLQGFNCVVRSYGLPTIPCCRGLTCRSYFPGSTYGRCQRY

SEQ ID NO: 49 - protein, 25 aa, Androctonus australis
RSVCRQIKICRRRGGCYYKCTNRPY

SEQ ID NO: 50 - protein, 13 aa, Unknown
  Synthetic Tritrpticin
VRRFPWWWPFLRR

SEQ ID NO: 51 - protein, 30 aa, Homo sapiens
  misc_feature: HNP 3 Defensin
DCYCRIPACIAGERRYGTCIYQGRLWAFCC

SEQ ID NO: 52 - protein, 38 aa, Phytolacca americana
AGCIKNGGRCNASAGPPYCCSSYCFQIAGQSYGVCKNR

SEQ ID NO: 53 - protein, 23 aa, Unknown
  Synthetic construct Magainin 2
GIGKYLHSAKKFGKAWVGEIMNS

SEQ ID NO: 54 - protein, 13 aa, Bos taurus
ILPWKWPWWPWRR

SEQ ID NO: 55 - protein, 44 aa, Heliothis virescens
DKLIGSCVWGAVNYTSDCNGECKRRGYKGGHCGSFANVNCWCET

SEQ ID NO: 56 - protein, 44 aa, Heliothis virescens; identical to SEQ ID NO: 55

SEQ ID NO: 57 - protein, 46 aa, Pisum sativum
  misc_feature: Seed of pea defensin 1 (psd1)
KTCEHLADTYRGVCFTNASCDDHCKNKAHLISGTCHNWKCFCTQNC

SEQ ID NO: 58 - protein, 18 aa, Unknown
  Synthetic Gomesin
QCRRLCYKQRCVTYCRGR

SEQ ID NO: 59 - protein, 25 aa, Bos taurus
  misc_feature: Lactoferricin B
FKCRRWQWRMKKLGAPSITCVRRAF

SEQ ID NO: 60 - protein, 12 aa, Unknown
  Synthetic PW2
HPLKQYWWRPSI

SEQ ID NO: 61 - protein, 20 aa, Homo sapiens
  misc_feature: Hepcidin 20
ICIFCCGCCHRSKCGMCCKT

SEQ ID NO: 62 - protein, 25 aa, Homo sapiens
  misc_feature: Hepcidin 25
DTHFPICIFCCGCCHRSKCGMCCKT

SEQ ID NO: 63 - protein, 30 aa, Amaranthus caudatus
VGECVRGRCPSGMCCSQFGYCGKGPKYCGR

SEQ ID NO: 64 - protein, 78 aa, Amaranthus caudatus
GYFCESCRKIIQKLEDMVGPQPNEDTVTQAASQVCDKLKILRGLCKKIMRSFLRRISWDILTGKKPQAICVDIKICKE

SEQ ID NO: 65 - protein, 23 aa, Xenopus laevis
  misc_feature: Magainin 2
GIGKFLHSAKKFGKAFVGEIMNS

SEQ ID NO: 66 - protein, 26 aa, Apis mellifera
  misc_feature: venom Melittin B
GIGAVLKVLTTGLPALISWIKRKRQQ

SEQ ID NO: 67 - protein, 21 aa, Podisus maculiventris
GSKKPVPIIYCNRRTGKCQRM

SEQ ID NO: 68 - protein, 38 aa, Mesembryanthemum crystallinum
  misc_feature: Antimicrobial peptide 1
AKCIKNGKGCREDQGPPFCCSGFCYRQVGWARGYCKNR

SEQ ID NO: 69 - protein, 13 aa, Bos taurus
  misc_feature: Melanotropin alpha (Alpha MSH)
SYSMEHFRWGKPV

SEQ ID NO: 70 - protein, 33 aa, Oryctolagus cuniculus
  misc_feature: Corticostatin III (MCP 1)
VVCACRRALCLPRERRAGFCRIRGRIHPLCCRR

SEQ ID NO: 71 - protein, 33 aa, Oryctolagus cuniculus
  misc_feature: Corticostatin IV (MCP 2)
VVCACRRALCLPLERRAGFCRIRGRIHPLCCRR

SEQ ID NO: 72 - protein, 35 aa, Antheraea pernyi
  misc_feature: Cecropin B
KWKIFKKIEKVGRNIRNGIIKAGPAVAVLGEAKAL

SEQ ID NO: 73 - protein, 48 aa, Bos taurus
  misc_feature: Seminalplasmin
SDEKASPDKHHRFSLSRYAKLANRLANPKLLETFLSKWIGDRGNRSVK

SEQ ID NO: 74 - protein, 34 aa, Oryctolagus cuniculus
  misc_feature: NP 3A defensin
GICACRRRFCPNSERFSGYCRVNGARYVRCCSRR

SEQ ID NO: 75 - protein, 30 aa, Homo sapiens
  misc_feature: HNP 1 Defensin
ACYCRIPACIAGERRYGTCIYQGRLWAFCC

SEQ ID NO: 76 - protein, 29 aa, Homo sapiens
  misc_feature: HNP 2 Defensin
CYCRIPACIAGERRYGTCIYQGRLWAFCC

SEQ ID NO: 77 - protein, 33 aa, Homo sapiens
  misc_feature: HNP 4 Defensin
VCSCRLVFCRRTELRVGNCLIGGVSFTYCCTRV

SEQ ID NO: 78 - protein, 24 aa, Homo sapiens
  misc_feature: Histatin 5
DSHAKRHHGYKRKFHEKHHSHRGY

SEQ ID NO: 79 - protein, 32 aa, Homo sapiens
  misc_feature: Histatin 3
DSHAKRHHGYKRKFHEKHHSHRGYRSNYLYDN

SEQ ID NO: 80 - protein, 12 aa, Unknown
  Histatin 8
KFHEKHHSHRGY

SEQ ID NO: 81 - protein, 38 aa, Bos taurus
  misc_feature: Tracheal antimicrobial peptide
NPVSCVRNKGICVPIRCPGSMKQIGTCVGRAVKCCRKK

SEQ ID NO: 82 - protein, 37 aa, Mirabilis jalapa
  misc_feature: antimicrobial peptidic agent1 (MJ antimicrobial peptidic agent1)
QCIGNGGRCNENVGPPYCCSGFCLRQPGQGYGYCKNR

SEQ ID NO: 83 - protein, 36 aa, Mirabilis jalapa
  misc_feature: antimicrobial peptidic agent2 (MJ antimicrobial peptidic agent2)
CIGNGGRCNENVGPPYCCSGFCLRQPNQGYGVCRNR

SEQ ID NO: 84 - protein, 33 aa, Zea mays
  misc_feature: MBP 1
RSGRGECRRQCLRRHEGQPWETQECMRRCRRRG

SEQ ID NO: 85 - protein, 23 aa, Brassica napus
  misc_feature: AFP2
QKLCERPSGTWSGVCGNNNACKN

SEQ ID NO: 86 - protein, 27 aa, Brassica rapa
  misc_feature: AFP1
QKLCERPSGTWSGVCGNNNACKNQCIN

SEQ ID NO: 87 - protein, 27 aa, Brassica rapa
  misc_feature: AFP2
QKLCERPSGTWSGVCGNNNACKNQCIR

SEQ ID NO: 88 - protein, 33 aa, Phyllomedusa bicolor
  misc_feature: Adenoregulin
GLWSKIKEVGKEAAKAAAKAAGKAALGAVSEAV

SEQ ID NO: 89 - protein, 16 aa, Sus scrofa
  misc_feature: Protegrin 2
RGGRLCYCRRRFCICV

SEQ ID NO: 90 - protein, 18 aa, Sus scrofa
  misc_feature: Protegrin 3
RGGGLCYCRRRFCVCVGR

SEQ ID NO: 91 - protein, 38 aa, Macaca fascicularis
  misc_feature: Histatin 1
DSHEERHHGRHGHHKYGRKFHEKHHSHRGYRSNYLYDN

SEQ ID NO: 92 - protein, 24 aa, Xenopus laevis
  misc_feature: Peptide PGQ
GVLSNVIGYLKKLGTGALNAVLKQ

SEQ ID NO: 93 - protein, 20 aa, Rana catesbeiana
  misc_feature: Ranalexin
FLGGLIKIVPAMICAVTKKC

SEQ ID NO: 94 - protein, 30 aa, Cavia cutleri
  misc_feature: GNCP 2
RCICTTRTCRFPYRRLGTCLFQNRVYTFCC

SEQ ID NO: 95 - protein, 18 aa, Sus scrofa
  misc_feature: Protegrin 4
RGGRLCYCRGWICFCVGR

SEQ ID NO: 96 - protein, 18 aa, Sus scrofa
  misc_feature: Protegrin 5
RGGRLCYCRPRFCVCVGR

SEQ ID NO: 97 - protein, 27 aa, Bos taurus
  misc_feature: BMAP 27
GRFKRFRKKFKKLFKKLSPVIPLLHLG

SEQ ID NO: 98 - protein, 28 aa, Bos taurus
  misc_feature: BMAP 28
GGLRSLGRKILRAWKKYGPIIVPIIRIG

SEQ ID NO: 99 - protein, 39 aa, Bufo bufo gargarizans
  misc_feature: Buforin 1
AGRGKQGGKVRAKAKTRSSRAGLQFPVGRVHRLLRKGNY

SEQ ID NO: 100 - protein, 21 aa, Bufo bufo gargarizans
  misc_feature: Buforin II
TRSSRAGLQFPVGRVHRLLRK

SEQ ID NO: 101 - protein, 34 aa, Bos taurus
  misc_feature: BMAP 34
GLFRRLRDSIRRGQQKILEKARRIGERIKDIFRG

SEQ ID NO: 102 - protein, 19 aa, Trichoderma longibrachiatum
  misc_feature: Tricholongin
AGFAAQAAASLAPVAAQQL

SEQ ID NO: 103 - protein, 34 aa, Phyllomedusa sauvagei
  misc_feature: Dermaseptin 1
ALWKTMLKKLGTMALHAGKAALGAAADTISQGTQ

SEQ ID NO: 104 - protein, 45 aa, Hevea brasiliensis
  misc_feature: Pseudo hevein (Minor hevein)
EQCGRQAGGKLCPNNLCCSQYGWCGSSDDYCSPSKNCQSNCKGGG

SEQ ID NO: 105 - protein, 33 aa, Rana rugosa
  misc_feature: Gaegurin 1
SLFSLIKAGAKFLGKNLLKQGACYAACKASKQC

SEQ ID NO: 106 - protein, 36 aa, Phyllomedusa bicolor
  misc_feature: Skin peptide tyrosine tyrosine
YPPKPESPGEDASPEEMNKYLTALRHYINLVTRQRY

SEQ ID NO: 107 - protein, 50 aa, Penaeus vannamei
  misc_feature: Penaeidin 1
YRGGYTGPIPRPPPIGRPPLRLVVCACYRLSVSDARNCCIKFGSCCHLVK

SEQ ID NO: 108 - protein, 33 aa, Mesocricetus auratus
  misc_feature: Neutrophil defensin 1 (HANP 1)
VTCFCRRRGCASRERHIGYCRFGNTIYRLCCRR

SEQ ID NO: 109 - protein, 33 aa, Mesocricetus auratus
  misc_feature: Neutrophil defensin 3 (HANP 3)
VTCFCRRRGCASRERLIGYCRFGNTIYGLCCRR

SEQ ID NO: 110 - protein, 21 aa, Misgurnus anguillicaudatus
  misc_feature: Misgurin
RQRVEELSKFSKKGAAARRRK

SEQ ID NO: 111 - protein, 41 aa, Pharbitis nil
  misc_feature: PN antimicrobial peptidic agent
QQCGRQASGRLCGNRLCCSQWGYCGSTASYCGAGCQSQCRS

SEQ ID NO: 112 - protein, 19 aa, Oncorhynchus mykiss
  misc_feature: Histone H2B 1(HLP 1)(Fragment)
PDPAKTAPKKGSKKAVTKA

SEQ ID NO: 113 - protein, 17 aa, Oncorhynchus mykiss
  misc_feature: Histone H2B 3(HLP 3)(Fragment)
PDPAKTAPKKKSKKAVT

SEQ ID NO: 114 - protein, 30 aa, Macaca mulatta
  misc_feature: neutrophil defensin 2 (RMAD 2)
ACYCRIPACLAGERRYGTCFYMGRVWAFCC

SEQ ID NO: 115 - protein, 36 aa, Pseudacanthotermes spiniger
  misc_feature: Termicin
ACNFQSCWATCQAQHSIYFRRAFCDRSQCKCVFVRG

SEQ ID NO: 116 - protein, 25 aa, Pseudacanthotermas spiniger
  misc_feature: Spingerin
HVDKKVADKVLLLKQLRIMRLLTRL

SEQ ID NO: 117 - protein, 13 aa, Litoria raniformis
  misc_feature: Aurein 1.1
GLFDIIKKIAESI

SEQ ID NO: 118 - protein, 30 aa, Pachycondyla goeldii
  misc_feature: Ponericin G1
GWKDWAKKAGGWLKKKGPGMAKAALKAAMQ

SEQ ID NO: 119 - protein, 24 aa, Rana berlandieri
  misc_feature: Brevinin 1BB
FLPAIAGMAAKFLPKIFCAISKKC

SEQ ID NO: 120 - protein, 20 aa, Rana clamitans
  misc_feature: Ranalexin 1CB
FLGGLMKAFPAIICAVTKKC

SEQ ID NO: 121 - protein, 31 aa, Rana clamitans
  misc_feature: Ranatuerin 2CA
GLFLDTLKGAAKDVAGKLLEGLKCKIAGCKP

SEQ ID NO: 122 - protein, 27 aa, Rana clamitans
  misc_feature: Ranatuerin 2CB
GLFLDTLKGLAGKLLQGLKCIKAGCKP

SEQ ID NO: 123 - protein, 40 aa, Ginkgo biloba
  misc_feature: Ginkbilobin
ANTAFVSSAHNTQKIPAGAPFNRNLRAMLADLRQNAAFAG

SEQ ID NO: 124 - protein, 20 aa, Basella alba
  misc_feature: Alpha basrubrin (Fragment)
GADFQECMKEHSQKQHQHQG

SEQ ID NO: 125 - protein, 24 aa, Pseudis paradoxa
  misc_feature: Pseudin 1
GLNTLKKVFQGLHEAIKLINNHVQ

SEQ ID NO: 126 - protein, 45 aa, Parabuthus schlechteri
  misc_feature: Parabutoporin
FKLGSFLKKAWKSKLAKKLRAKGKEMLKDYAKGLLEGGSEEVPGQ

SEQ ID NO: 127 - protein, 44 aa, Opistophthalmus carinatus
  misc_feature: Opistoporin 1
GKVWDWIKSTAKKLWNSEPVKELKNTALNAAKNLVAEKIGATPS

SEQ ID NO: 128 - protein, 44 aa, Opistophthalmus carinatus
  misc_feature: Opistoporin 2
GKVWDWIKSTAKKLWNSEPVKELKNTALNAAKNFVAEKIGATPS

SEQ ID NO: 129 - protein, 12 aa, Oncorhynchus mykiss
  misc_feature: Histone H2A (Fragment)
AERVGAGAPVYL

SEQ ID NO: 130 - protein, 33 aa, Dolabella auricularia
  misc_feature: Dolabellanin B2
SHQDCYEALHKCMASHSKPFSCSMKFHMCLQQQ

SEQ ID NO: 131 - protein, 35 aa, Heliothis virescens
  misc_feature: Cecropin A
RWKVFKKIEKVGRNIRDGVIKAAPAIEVLGQAKAL

SEQ ID NO: 132 - protein, 35 aa, Homo sapiens
  misc_feature: HNP 5 Defensin
QARATCYCRTGRCATRESLSGVCEISGRLYRLCCR

SEQ ID NO: 133 - protein, 35 aa, Homo sapiens
  misc_feature: HNP 6 Defensin
STRAFTCHCRRSCYSTEYSYGTCTVMGINHRFCCL

SEQ ID NO: 134 - protein, 84 aa, Holotrichia diomphalia
  misc_feature: Holotricin 3
YGPGDGHGGGHGGGHGGGHGNGQGGGHGHGPGGGFGGGHGGGHGGGGRGGGGSGGGGSPGHGAGGGYPGGHGGGHHGGYQTHGY

SEQ ID NO: 135 - protein, 45 aa, Bos taurus
  misc_feature: Lingual antimicrobial peptide
GFTQGVRNSQSCRRNKGICVPIRCPGSMRQIGTCLGAQVKCCRRK

SEQ ID NO: 136 - protein, 29 aa, Rattus norvegicus
  misc_feature: RatNP 3
CSCRTSSCRFGERLSGACRLNGRIYRLCC

SEQ ID NO: 137 - protein, 31 aa, Cavia cutleri
  misc_feature: GNCP 1
RRCICTTRTCRFPYRRLGTCIFQNRVYTFCC

SEQ ID NO: 138 - protein, 47 aa, Penaeus vannamei
  misc_feature: Penaeidin 4a
HSSGYTRPLPKPSRPIFIRPIGCDVCYGIPSSTARLCCFRYGDCCHR

SEQ ID NO: 139 - protein, 6 aa, Bos taurus
  misc_feature: hexapeptide
RRWQWR

SEQ ID NO: 140 - protein, 18 aa, Penaeus vannamei
KWKLFKKIPKFLHLAKKF

SEQ ID NO: 141 - protein, 20 aa, Homo sapiens
  misc_feature: MUC7 20 Mer
LAHQKPFIRKSYKCLHKRCR

SEQ ID NO: 142 - protein, 21 aa, Rana nigromaculata
  misc_feature: Nigrocin 2
GLLSKVLGVGKKVLCGVSGLC

SEQ ID NO: 143 - protein, 33 aa, Rana nigromaculata
  misc_feature: Nigrocin 1
GLLDSIKGMAISAGKGALQNLLKVASCKLDKTC

SEQ ID NO: 144 - protein, 10 aa, Unknown
  lactoferrin (Lf) peptide 2
FKCRRWQWRM

SEQ ID NO: 145 - protein, 18 aa, Impatiens balsamina
  misc_feature: Ib antimicrobial peptidic agent3
RHRCCAWGPGRKYCKRWC

SEQ ID NO: 146 - protein, 18 aa, Impatiens balsamina
  misc_feature: Ib antimicrobial peptidic agent4
GRRCCGWGPGRRYCRRWC

SEQ ID NO: 147 - protein, 14 aa, Unknown
  Synthesis dhvar4
KRLFKKLLFSLRKY

SEQ ID NO: 148 - protein, 14 aa, Unknown
  Synthesis dhvar5
LLLFLLKKRKKRKY

SEQ ID NO: 149 - protein, 6 aa, Artificial Sequence
  Synthesized
  Misc_feature (1)..(6): X = any amino acid
XXXXXC

SEQ ID NO: 150 - protein, 6 aa, Artificial Sequence
  Synthesized
  Misc_feature (1)..(6): X = any amino acid
XXXXFC

SEQ ID NO: 151 - protein, 6 aa, Artificial Sequence
  Synthesized
  Misc_feature (1)..(6): X = any amino acid
XXXXNC

SEQ ID NO: 152 - protein, 6 aa, Artificial Sequence
  Synthesized
  Misc_feature (1)..(6): X = any amino acid
XXXXWC

SEQ ID NO: 153 - protein, 6 aa, Artificial Sequence
  Synthesized
  Misc_feature (1)..(6): X = any amino acid
XXXXIC

SEQ ID NO: 154 - protein, 6 aa, Artificial Sequence
  Synthesized
  Misc_feature (1)..(6): X = any amino acid
XXXXTC

SEQ ID NO: 155 - protein, 6 aa, Artificial Sequence
  Synthesized
  Misc_feature (1)..(6): X = any amino acid
XXXXYC

SEQ ID NO: 156 - protein, 6 aa, Artificial Sequence
  Synthesized
  Misc_feature (1)..(6): X = any amino acid
XXXXVC

SEQ ID NO: 157 - protein, 6 aa, Artificial Sequence
  Synthesized
  Misc_feature (1)..(6): X = any amino acid
XXXXMC

SEQ ID NO: 158 - protein, 6 aa, Artificial Sequence
  Synthesized
  Misc_feature (1)..(6): X = any amino acid
XXXXGC

SEQ ID NO: 159 - protein, 6 aa, Artificial Sequence
  Synthesized
  Misc_feature (1)..(6): X = any amino acid
XXXXEC

SEQ ID NO: 160 - protein, 6 aa, Artificial Sequence
  Synthesized
  Misc_feature (1)..(6): X = any amino acid
XXXXSC

SEQ ID NO: 161 - protein, 6 aa, Artificial Sequence
  Synthesized
  Misc_feature (1)..(6): X = any amino acid
XXXXLC

SEQ ID NO: 162 - protein, 6 aa, Artificial Sequence
  Synthesized
  Misc_feature (1)..(6): X = any amino acid
XXXXPC

SEQ ID NO: 163 - protein, 6 aa, Artificial Sequence
  Synthesized
  Misc_feature (1)..(6): X = any amino acid
XXXXHC

SEQ ID NO: 164 - protein, 6 aa, Artificial Sequence
  Synthesized
  Misc_feature (1)..(6): X = any amino acid
XXXXCC

SEQ ID NO: 165 - protein, 6 aa, Artificial Sequence
  Synthesized
  Misc_feature (1)..(6): X = any amino acid
XXXXAC

SEQ ID NO: 166 - protein, 6 aa, Artificial Sequence
  Synthesized
  Misc_feature (1)..(6): X = any amino acid
XXXXKC

SEQ ID NO: 167 - protein, 6 aa, Artificial Sequence
  Synthesized
  Misc_feature (1)..(6): X = any amino acid
XXXXQC

SEQ ID NO: 168 - protein, 6 aa, Artificial Sequence
  Synthesized
  Misc_feature (1)..(6): X = any amino acid
XXXXRC

SEQ ID NO: 169 - protein, 6 aa, Artificial Sequence
  Synthesized
  Misc_feature (1)..(6): X = any amino acid
XXXXDC

SEQ ID NO: 170 - protein, 6 aa, Artificial Sequence
  Synthesized
  Misc_feature (1)..(6): X = any amino acid
XXXXCY

SEQ ID NO: 171 - protein, 6 aa, Artificial Sequence
  Synthesized
  Misc_feature (1)..(6): X = any amino acid
XXXXCQ

SEQ ID NO: 172 - protein, 6 aa, Artificial Sequence
  Synthesized
  Misc_feature (1)..(6): X = any amino acid
XXXXYS

SEQ ID NO: 173 - protein, 6 aa, Artificial Sequence
  Synthesized
  Misc_feature (1)..(6): X = any amino acid
XXXXYD

SEQ ID NO: 174 - protein, 6 aa, Artificial Sequence
  Synthesized
  Misc_feature (1)..(6): X = any amino acid
XXXXTQ

SEQ ID NO: 175 - protein, 6 aa, Artificial Sequence
  Synthesized
WTFRYC

SEQ ID NO: 176 - protein, 6 aa, Artificial Sequence
  Synthesized
CYRFTW

SEQ ID NO: 177 - protein, 11 aa, Glomerella cingulata
GYFSYPHGNLF

SEQ ID NO: 178 - protein, 13 aa, Saccharomyces cerevisiae
WHWLQLKPGQPMY

SEQ ID NO: 179 - protein, 13 aa, Saccharomyces kluyveri
WHWLSFSKGQPMY

SEQ ID NO: 180 - protein, 19 aa, Artificial Sequence
  Synthesized
  MOD_RES (3)..(3): Nle
YNLEDHPQGDHPKLQLWHW

SEQ ID NO: 181 - protein, 15 aa, Artificial Sequence
  Synthesized
  MOD_RES (3)..(3): Nle
YNLEPQGPKLQLWHW

SEQ ID NO: 182 - protein, 13 aa, Artificial Sequence
  Synthesized
YMPQGPKLQLFHW

SEQ ID NO: 183 - protein, 12 aa, Artificial Sequence
  Synthesized
YMPQGPKLQLWH

SEQ ID NO: 184 - protein, 13 aa, Artificial Sequence
  Synthesized
YMPQGPRLNLWHW

SEQ ID NO: 185 - protein, 26 aa, Magnaporthe grisea
MSPSTKNIPAPVAGARAGPIHYCVIM

SEQ ID NO: 186 - protein, 24 aa, Neurospora crassa
MPSTAASTRVPQTTMNFNGYCVVM

SEQ ID NO: 187 - protein, 23 aa, Cryphonectria parasitica
MPSNTQTSNSSMGVNGYSYCVVM

SEQ ID NO: 188 - protein, 11 aa, Magnaporthe grisea
QWCPRRGQPCW

SEQ ID NO: 189 - protein, 11 aa, Neurospora crassa
QWCRIHGQSCW

SEQ ID NO: 190 - protein, 13 aa, Saccharomyces cerevisiae
WHWLQLKPGQPMY

SEQ ID NO: 191 - protein, 10 aa, Cryphonectria parasitica
WCLFHGEGCW

SEQ ID NO: 192 - protein, 4 aa, Artificial Sequence
  Synthesized
  Misc_feature (1)..(4): X = any amino acid
XAAC

SEQ ID NO: 193 - protein, 10 aa, Artificial Sequence
  Synthesized
  Misc_feature (1)..(10): X = any amino acid
WCXXGXXXCW

SEQ ID NO: 194 - protein, 6 aa, Artificial Sequence
  Synthesized
  Misc_feature (1)..(6): X = any amino acid
XXXXCI

SEQ ID NO: 195 - protein, 10 aa, Fusarium graminearum
WCQQKGQPCW

SEQ ID NO: 196 - protein, 10 aa, Fusarium graminearum
WCTWKGQPCW

SEQ ID NO: 197 - protein, 7 aa, Artificial Sequence
  Synthesized
FRLKFHF

SEQ ID NO: 198 - protein, 6 aa, Artificial Sequence
  Synthesized
FRLKHI

SEQ ID NO: 199 - protein, 5 aa, Artificial Sequence
  Synthesized
FRLHF

SEQ ID NO: 200 - protein, 6 aa, Artificial Sequence
  Synthesized
  MISC_FEATURE (1)..(6): X = any amino acid
FRXXXX

SEQ ID NO: 201 - protein, 6 aa, Artificial Sequence
  Synthesized
  MISC_FEATURE (1)..(6): X = any amino acid
FRLXXX

SEQ ID NO: 202 - protein, 6 aa, Artificial Sequence
  Synthesized
HFKLRF

SEQ ID NO: 203 - protein, 5 aa, Artificial Sequence
  Synthesized
FRLHF

SEQ ID NO: 204 - protein, 4 aa, Artificial Sequence
  Synthesized
  Misc_feature (1)..(4): X = any amino acid
XXCC

SEQ ID NO: 205 - protein, 5 aa, Artificial Sequence
  Synthesized
  Misc_feature (1)..(5): X = any amino acid
CCXCC

SEQ ID NO: 206 - protein, 15 aa, Artificial Sequence
  Synthesized
CGCCGCGCCGCGCCG

SEQ ID NO: 207 - protein, 5 aa, Artificial Sequence
  Synthesized
CGCCG

SEQ ID NO: 208 - protein, 4 aa, Artificial Sequence
  Synthesized
  Misc_feature (1)..(4): X = any amino acid
DAHX

SEQ ID NO: 209 - protein, 4 aa, Artificial Sequence
  Synthesized
  Misc_feature (1)..(4): X = any amino acid
EXXG

SEQ ID NO: 210 - protein, 9 aa, Artificial Sequence
  Synthesized
  Misc_feature (1)..(8): X = any amino acid
  Misc_feature (1)..(8): One or all of amino acids 1-8 can be either present or absent
XXXXXXXXH

SEQ ID NO: 211 - protein, 7 aa, Artificial Sequence
  Synthesized
  Misc_feature (1)..(7): X = any amino acid
CXXCXXC

SEQ ID NO: 212 - protein, 12 aa, Artificial Sequence
  Synthesized
  Misc_feature (1)..(5): X = any amino acid
  Misc_feature (1)..(5): One or all of amino acids 7-9 can be either present or absent
  misc_feature (6)..(9): Xaa can be any naturally occurring amino acid
  misc_feature (11)..(11): Xaa can be any naturally occurring amino acid
CXCXXXXXXCXC

SEQ ID NO: 213 - protein, 6 aa, Artificial Sequence
  Synthesized
  Misc_feature (1)..(5): X = any amino acid
  Misc_feature (1)..(5): One or all of amino acids 2-5 can be either present or absent
CXXXXC

SEQ ID NO: 214 - protein, 4 aa, Artificial Sequence
  Synthesized
  Misc_feature (1)..(4): X = any amino acid
EXXH

SEQ ID NO: 215 - protein, 19 aa, Artificial Sequence
  Synthesized
  Misc_feature (1)..(19): X = any amino acid
CXXXXCXXXXXXXXHXXXC

SEQ ID NO: 216 - protein, 6 aa, Artificial Sequence
  Synthesized
  Misc_feature (1)..(6): X = any amino acid
QCXKKG

SEQ ID NO: 217 - protein, 6 aa, Artificial Sequence
  Synthesized
  Misc_feature (1)..(5): X = any amino acid
  Misc_feature (1)..(5): One or all of amino acids 4-5 can be either present or absent
HXXXXC

SEQ ID NO: 218 - protein, 6 aa, Artificial Sequence
  Synthesized
AHGHRP

SEQ ID NO: 219 - protein, 6 aa, Artificial Sequence
  Synthesized
GHGHGH

SEQ ID NO: 220 - protein, 12 aa, Homo sapiens
  misc_feature: Histidine-rich glycoprotein
GHHPHGGHHPHG

SEQ ID NO: 221 - protein, 6 aa, Homo sapiens
  misc_feature: Histidine-rich glycoprotein
GHHPHG

SEQ ID NO: 222 - protein, 11 aa, Artificial Sequence
  Synthesized
  Misc_feature (1)..(11): X = any amino acid
HEXXHXXGXXD

SEQ ID NO: 223 - protein, 11 aa, Artificial Sequence
  Synthesized
  Misc_feature (1)..(11): X = any amino acid
HEXXHXXGXXH

SEQ ID NO: 224 - protein, 7 aa, Artificial Sequence
  Synthesized
HWHWHWH

SEQ ID NO: 225 - protein, 6 aa, Artificial Sequence
  Synthesized
  Misc_feature (1)..(5): X = any amino acid
  Misc_feature (1)..(5): One or all of amino acids 2-5 can be either present or absent
HXXXXH

SEQ ID NO: 226 - protein, 5 aa, Artificial Sequence
  Synthesized
  Misc_feature (1)..(5): X = any amino acid
HXXEH

SEQ ID NO: 227 - protein, 5 aa, Artificial Sequence
  Synthesized
  Misc_feature (1)..(5): X = any amino acid
HEXXH

SEQ ID NO: 228 - protein, 4 aa, Artificial Sequence
  Synthesized
HHHH

SEQ ID NO: 229 - protein, 4 aa, Artificial Sequence
  Synthesized
  Misc_feature (1)..(4): X = any amino acid
HXXE

SEQ ID NO: 230 - protein, 8 aa, Homo sapiens
  misc_feature: prion
PHGGGWGQ

SEQ ID NO: 231 - protein, 7 aa, Artificial Sequence
  Synthesized
PHHHHHP

SEQ ID NO: 232 - protein, 6 aa, Artificial Sequence
  Synthesized
SYHHHH

SEQ ID NO: 233 - protein, 6 aa, Artificial Sequence
  Synthesized
HWHHHP

SEQ ID NO: 234 - protein, 6 aa, Artificial Sequence
  Synthesized
HSQKVK

SEQ ID NO: 235 - protein, 6 aa, Artificial Sequence
  Synthesized
HSQKVF

SEQ ID NO: 236 - protein, 5 aa, Artificial Sequence
  Synthesized
HHHHH

SEQ ID NO: 237 - protein, 6 aa, Artificial Sequence
  Synthesized
HHHHHH

SEQ ID NO: 238 - protein, 7 aa, Artificial Sequence
  Synthesized
HHHHHHH

SEQ ID NO: 239 - protein, 8 aa, Artificial Sequence
  Synthesized
HHHHHHHH

SEQ ID NO: 240 - protein, 9 aa, Artificial Sequence
  Synthesized
HHHHHHHHH

SEQ ID NO: 241 - protein, 10 aa, Artificial Sequence
  Synthesized
HHHHHHHHHH

SEQ ID NO: 242 - protein, 19 aa, Gallus gallus
  misc_feature: L-lactate dehydrogenase
KDHLIHNVHKEFHAHAHNK

SEQ ID NO: 243 - protein, 5 aa, Artificial Sequence
  Synthesized
  Misc_feature (1)..(5): X = any amino acid
NEXXS

SEQ ID NO: 244 - protein, 6 aa, Artificial Sequence
  Synthesized
  misc_feature: Thrombin cleavage site
LVPRGS

SEQ ID NO: 245 - protein, 5 aa, Homo sapiens
  misc_feature: Prothrombin; Factor Xa cleavage site
  Misc_feature (1)..(5): X = any amino acid
IEGRX

SEQ ID NO: 246 - protein, 5 aa, Artificial Sequence
  Synthesized
  misc_feature: Subtilisin cleavage site
  Misc_feature (1)..(5): X = any amino acid
FAHYX

SEQ ID NO: 247 - protein, 6 aa, Artificial Sequence
  Synthesized
  misc_feature: Enterokinase cleavage site
  Misc_feature (1)..(6): X = any amino acid
DDDDKX

SEQ ID NO: 248 - protein, 7 aa, Artificial Sequence
  Synthesized
  misc_feature: protease 3C cleavage site
ETLFQGP

SEQ ID NO: 249 - protein, 7 aa, Artificial Sequence
  Synthesized
  misc_feature: protease 3C cleavage site
EALFQGP

SEQ ID NO: 250 - protein, 6 aa, Artificial Sequence
  Synthesized
YLPKNG

SEQ ID NO: 251 - protein, 6 aa, Artificial Sequence
  Synthesized
TTSWVV

SEQ ID NO: 252 - protein, 6 aa, Artificial Sequence
  Synthesized
SANVIH

SEQ ID NO: 253 - protein, 6 aa, Artificial Sequence
  Synthesized
PFKDWY

SEQ ID NO: 254 - protein, 6 aa, Artificial Sequence
  Synthesized
PMGVAS

SEQ ID NO: 255 - protein, 6 aa, Artificial Sequence
  Synthesized
PIFSGG

SEQ ID NO: 256 - protein, 6 aa, Artificial Sequence
  Synthesized
FTKFPT

SEQ ID NO: 257 - protein, 6 aa, Artificial Sequence
  Synthesized
KSVVLS

SEQ ID NO: 258 - protein, 6 aa, Artificial Sequence
  Synthesized
KAASNS

SEQ ID NO: 259 - protein, 6 aa, Artificial Sequence
  Synthesized
LLLGVD

SEQ ID NO: 260 - protein, 6 aa, Artificial Sequence
  Synthesized
LLLGLD

SEQ ID NO: 261 - protein, 6 aa, Artificial Sequence
  Synthesized
LLHGLN

SEQ ID NO: 262 - protein, 6 aa, Artificial Sequence
  Synthesized
ITTNEN

SEQ ID NO: 263 - protein, 4 aa, Artificial Sequence
  Synthesized
AAHH

SEQ ID NO: 264 - protein, 6 aa, Artificial Sequence
  Synthesized
HDGRSS

SEQ ID NO: 265 - protein, 6 aa, Artificial Sequence
  Synthesized
GVSAPP

SEQ ID NO: 266 - protein, 6 aa, Artificial Sequence
  Synthesized
ELRPHT

SEQ ID NO: 267 - protein, 6 aa, Artificial Sequence
  Synthesized
RYTIAG

SEQ ID NO: 268 - protein, 6 aa, Artificial Sequence
  Synthesized
RLVHIK

SEQ ID NO: 269 - protein, 6 aa, Artificial Sequence
  Synthesized
RGNSLR

SEQ ID NO: 270 - protein, 6 aa, Artificial Sequence
  Synthesized
RELRPE

SEQ ID NO: 271 - protein, 6 aa, Artificial Sequence
  Synthesized
REELTL

SEQ ID NO: 272 - protein, 6 aa, Artificial Sequence
  Synthesized
HKHHHN

SEQ ID NO: 273 - protein, 6 aa, Artificial Sequence
  Synthesized
HHHWLH

SEQ ID NO: 274 - protein, 6 aa, Artificial Sequence
  Synthesized
HHSHPH

SEQ ID NO: 275 - protein, 6 aa, Artificial Sequence
  Synthesized
HHHMVH

SEQ ID NO: 276 - protein, 6 aa, Artificial Sequence
  Synthesized
YHHHHT

SEQ ID NO: 277 - protein, 6 aa, Artificial Sequence
  Synthesized
HHHGAH

SEQ ID NO: 278 - protein, 6 aa, Artificial Sequence
  Synthesized
HHHHGR

SEQ ID NO: 279 - protein, 5 aa, Artificial Sequence
  Synthesized
AAAHH

SEQ ID NO: 280 - protein, 6 aa, Artificial Sequence
  Synthesized
AAAAHH

SEQ ID NO: 281 - protein, 6 aa, Artificial Sequence
  Synthesized
AAAHAH

SEQ ID NO: 282 - protein, 6 aa, Artificial Sequence
  Synthesized
AAHAAH

SEQ ID NO: 283 - protein, 6 aa, Artificial Sequence
  Synthesized
AHAAAH

SEQ ID NO: 284 - protein, 6 aa, Artificial Sequence
  Synthesized
HAAAAH

SEQ ID NO: 285 - protein, 6 aa, Artificial Sequence
  Synthesized
HAHAAH

SEQ ID NO: 286 - protein, 6 aa, Artificial Sequence
  Synthesized
HAAHAH

SEQ ID NO: 287 - protein, 7 aa, Artificial Sequence
  Synthesized
  Misc_feature (1)..(7): X = any amino acid
HEXXHXH

SEQ ID NO: 288 - protein, 22 aa, Artificial Sequence
  Synthesized
  Misc_feature (1)..(22): X = any amino acid
CXXXXXXXXXXXXXXXXXXXXH

SEQ ID NO: 289 - protein, 11 aa, Artificial Sequence
  Synthesized
  Misc_feature (1)..(11): X = any amino acid
HXXXHXXXXXH

SEQ ID NO: 290 - protein, 5 aa, Artificial Sequence
  Synthesized
CLEEH

SEQ ID NO: 291 - protein, 5 aa, Artificial Sequence
  Synthesized
CLKEH

SEQ ID NO: 292 - protein, 4 aa, Artificial Sequence
  Synthesized
  Misc_feature (1)..(4): X = any amino acid
EXXS

SEQ ID NO: 293 - protein, 4 aa, Artificial Sequence
  Synthesized
  Misc_feature (1)..(4): X = any amino acid
XXDX

SEQ ID NO: 294 - protein, 4 aa, Artificial Sequence
  Synthesized
  Misc_feature (1)..(4): X = any amino acid
XXCX

SEQ ID NO: 295 - protein, 4 aa, Artificial Sequence
  Synthesized
  Misc_feature (1)..(4): X = any amino acid
XXHX

SEQ ID NO: 296 - protein, 4 aa, Artificial Sequence
  Synthesized
  Misc_feature (1)..(4): X = any amino acid
XXMX

SEQ ID NO: 297 - protein, 6 aa, Artificial Sequence
  Synthesized
HNHNHN

SEQ ID NO: 298 - protein, 6 aa, Artificial Sequence
  Synthesized
GGGGGH

SEQ ID NO: 299 - protein, 5 aa, Artificial Sequence
  Synthesized
GGGGH

SEQ ID NO: 300 - protein, 7 aa, Artificial Sequence
  Synthesized
GMTCAAC

SEQ ID NO: 301 - protein, 11 aa, Artificial Sequence
  Synthesized
MSCCGGNCGCS

SEQ ID NO: 302 - protein, 8 aa, Artificial Sequence
  Synthesized
LEVLFQGP

SEQ ID NO: 303 - protein, 5 aa, Artificial Sequence
  Synthesized
  Misc_feature (1)..(5): X = any amino acid
IDGRX

SEQ ID NO: 304 - protein, 7 aa, Artificial Sequence
  Synthesized
ENLYFQG

(19) United States
(12) Patent Application Publication
(10) Pub. No.: US 2021/0017402 A1
(43) Pub. Date: Jan. 21, 2021

(54) ANTI-FOULING PAINTS AND COATINGS

(71) Applicant: REACTIVE SURFACES, LTD., LLP, Austin, TX (US)

(72) Inventors: Claude Steven McDaniel, Austin, TX (US); Melinda Wales, Bryan, TX (US)

(73) Assignee: REACTIVE SURFACES, LTD., LLP, Austin, TX (US)

(21) Appl. No.: 17/000,906

(22) Filed: Aug. 24, 2020

Related U.S. Application Data
(63) Continuation of application No. 14/093,347, filed on Nov. 29, 2013, now abandoned.
Application No. 14/093,347 is a continuation of application No. 12/882,563, filed on Sep. 15, 2010, now abandoned.
(60) Provisional application No. 61/242,485, filed on Sep. 15, 2009.

(51) Int. Cl. C09D 5/16; C07K 7/06; A01N 63/00; C09D 5/14; B08B 17/02; C07K 7/08

(52) U.S. Cl. CPC C09D 5/1637; C07K 7/06; A01N 63/00; C07K 7/08; C09D 5/1693; B08B 17/02; C09D 5/14

(57) ABSTRACT

Disclosed herein are a materials such as a coating, an elastomer, an adhesive, a sealant, a textile finish, a wax, and a filler for such a material, wherein the material includes an proteinaceous molecule such as a peptide and/an enzyme that confer a metal binding, an anti-fouling and/or an antibiotic property to the material. In particular, disclosed herein are marine coatings such as a marine paint that comprise an anti-fouling peptide sequence that reversibly binds a metal cation that is toxic to a fouling organism. Also disclosed herein are methods of reducing fouling on a surface by treating the surface with a metal binding peptide.

### A. PRIORITY CLAIM

This U.S. patent application claim priority as a continuation to U.S. patent application Ser. No. 14/093,347 filed on Nov. 29, 2013, where U.S. patent application Ser. No. 14/093,347 claims priority as a continuation to U.S. patent application Ser. No. 12/882,563 filed on Sep. 15, 2010, where U.S. patent application Ser. No. 12/882,563 claims priority to U.S. Provisional Application No. 61/242,485, filed Sep. 15, 2009.

### B. BACKGROUND OF THE INVENTION

### 1. Field of the Invention

The present invention generally relates to anti-fouling compositions (e.g., coatings, paints, elastomers, adhesives) comprising a metal binding, an anti-fouling, and/or an anti-biological proteinaceous molecule (e.g., a peptide, a polypeptide, a protein, an enzyme) and methods employing such compositions to deter or prevent fouling or another biological infestation on a susceptible surface.

### 2. Description of the Related Art

The surface of a material may be subject to addition of a surface treatment such as a coating, an adhesive, a sealant, a textile finish, and/or a wax, with a surface treatment typically used, for example, to protect, decorate, attach, and/or seal a surface and/or the underlying material. An example of a surface that may be treated with a surface treatment includes a marine surface, such as surface frequently or continuously in contact with water. An example of such a marine surface may include the bottom of a ship.

A biomolecule comprises a molecule often produced and isolated from an organism, such as proteinaceous molecule. Examples of a proteinaceous molecule include a peptide, a polypeptide, a protein, or an enzyme. A proteinaceous molecule such as a peptide may possess a binding function to another atom or molecule, including a metal, a biomolecule such as a lipid, or another proteinaceous sequence. An example of an enzyme comprises a lipolytic enzyme (e.g., a lipase) that catalyzes a reaction on a lipid substrate, such as a vegetable oil, a phospholipid, a sterol, and other hydrophobic molecule; or an enzyme that detoxifies an organophosphorus compound (“organophosphate compound,” “OP compound”) include an organophosphorus hydrolase (“OPH”), an organophosphorus acid anhydrolase (“OPAA”), and a DFPase.

### C. BRIEF SUMMARY OF THE EMBODIMENTS

The embodiments of the invention provides a marine coating composition comprising, a marine coating and a proteinaceous molecule, wherein the proteinaceous molecule comprises a metal binding sequence binding a metal ligand in a sufficient amount to inhibit the accumulation of biofouling on an inanimate surface coated with the marine coating composition; wherein the metal binding sequence comprises at least one member of the group consisting of SEQ ID Nos. 204-243 and 250-302 and functionally equivalent substituted metal binding amino acid sequences thereof. In some embodiments, the proteinaceous molecule is a peptide between 3 and 30 amino acids in length. In other embodiments, about 5% to about 100% of the amino acids of the metal binding sequence are electron donor amino acids. In further embodiments, at least 25% of the electron donor amino acids are one or more amino acids selected from the group consisting of a histidine, a lysine, and an arginine. In other aspects, the proteinaceous molecule is cysteine-free. In some aspects, each amino acid of the proteinaceous molecule is independently selected from the group consisting of the D-stereoisomer and L-stereoisomer forms of the amino acids. In further aspects, the marine coating possesses an enhanced metal binding activity. In additional aspects, the metal binding activity is reversible.

In some embodiments, the marine coating comprises an immobilization agent, and wherein the proteinaceous molecule is immobilized to the immobilation agent. In other embodiments, the immobilization of the proteinaceous molecule by the immobilization agent is reversible so that the proteinaceous molecule may be released from the immobilization agent, and wherein upon release of the proteinaceous molecule the immobilization agent may immobilize another like or different proteinaceous molecule. In additional embodiments, the marine coating comprises at least one anti-biological agent selected from the group consisting of a peptidic agent of SEQ ID Nos 1-203, a peptidic agent having functionally equivalent amino acid substituted sequences having no more than a +/−2 difference in hydropathic value of the Kyte-Doolittle scale relative thereto SEQ ID Nos. 1-203, a preservative, an anti-fouling agent, an anti-microbial agent, a phosphoric triester hydrolase, a phytochelatin, a lysozyme, a lysostaphin, a libiase, a lysyl endopeptidase, a mutanolysin, a cellulase, a chitinase, an α-agarase, an β-agarase, a N-acetylmuramoyl-L-alanine amidase, a lytic transglycosylase, a glucan endo-1,3-β-D-glucosidase, an endo-1,3(4)-β-glucanase, a β-lytic metalloendopeptidase, a 3-deoxy-2-octulosonidase, a peptide-N4-(N-acetyl-β-glucosaminyl)asparagine amidase, a mannosyl-glycoprotein endo-β-N-acetylglucosaminidase, a t-carrageenase, a κ-carrageenase, a λ-carrageenase, an α-neoagaro-oligosaccharide hydrolase, an endolysin, an autolysin, a mannoprotein protease, a glucanase, a mannose, a zymolase, a lyticase, a lipolytic enzyme, and a cell-based particulate material. In further embodiments, the accumulation of biofouling on the inanimate surface is inhibited by a reduced adherence to the marine coating, an enhanced ease of washing to remove the biofouling, or a combination thereof. In certain aspects the biofouling accumulation that is inhibited comprises the accumulation of at least one fouling organism select from the group consisting of a soft fouling microorganism, a hard fouling organism, a small brush/grass type organism, and a spineless organism. In other facets, the coating is a paint or a clear coating. In specific facets, the marine coating comprises a multicoat system. In particular facets, the marine coating is a multipack coating.

Also provided is a composition comprising, at least one material selected from the group consisting of: a coating, an elastomer, an adhesive, a sealant, a textile finish, a wax, a thermoplastic, and a thermoset; wherein the coating is at least one coating selected from the group consisting of an architectural coating, a pipeline coating, an automotive coating, a can coating, a chemical agent resistant coating, a camouflage coating, a traffic marker coating, an aircraft coating, and a nuclear power plant coating; wherein the material comprises a proteinaceous molecule, wherein the proteinaceous molecule comprises a metal binding sequence binding a metal ligand in a sufficient amount to inhibit the biological infestation of the material, wherein the metal binding sequence comprises at least one member of the group consisting of SEQ ID Nos. 204-243 and 250-302 and functionally equivalent substituted metal binding amino acid sequences thereof. In some embodiments, the material comprises at least one anti-biological agent selected from the group consisting of a peptidic agent of SEQ ID Nos 1-203, a peptidic agent having functionally equivalent amino acid substituted sequences having no more than a +/−2 difference in hydropathic value of the Kyte-Doolittle scale relative thereto SEQ ID Nos. 1-203, a preservative, an anti-fouling agent, an anti-microbial agent, a phosphoric triester hydrolase, a phytochelatin, a lysozyme, a lysostaphin, a libiase, a lysyl endopeptidase, a mutanolysin, a cellulase, a chitinase, an α-agarase, an β-agarase, a N-acetylmuramoyl-L-alanine amidase, a lytic transglycosylase, a glucan endo-1,3-β-D-glucosidase, an endo-1,3(4)-β-glucanase, a β-lytic metalloendopeptidase, a 3-deoxy-2-octulosonidase, a peptide-N4-(N-acetyl-β-glucosaminyl)asparagine amidase, a mannosyl-glycoprotein endo-β-N-acetylglucosaminidase, a ι-carrageenase, a κ-carrageenase, a λ-carrageenase, an α-neoagaro-oligosaccharide hydrolase, an endolysin, an autolysin, a mannoprotein protease, a glucanase, a mannose, a zymolase, a lyticase, a lipolytic enzyme, and a cell-based particulate material.

Also provided is a method of inhibiting the accumulation of a fouling biofilm on a surface of an inanimate object, comprising obtaining a marine coating comprising a proteinaceous molecule, wherein the proteinaceous molecule comprises a metal binding sequence binding a metal ligand in a sufficient amount to inhibit the accumulation of a fouling biofilm on an inanimate surface coated with the marine coating composition; wherein the metal binding sequence comprises at least one member of the group consisting of SEQ ID Nos. 204-243 and 250-302 and functionally equivalent substituted metal binding amino acid sequences thereof; and applying the marine coating to the surface of an inanimate object. In some embodiments, the method further comprises application of an additional anti-fouling composition, fouling removal technique, or a combination thereof, to the surface upon accumulation of a fouling biofilm.

Product a composition. Product a material formulation. Product a composition comprising a material formulation. Product a composition, comprising a marine coating composition comprising, a marine coating and a proteinaceous molecule, wherein the proteinaceous molecule comprises a metal binding sequence binding a metal ligand in a sufficient amount to inhibit the accumulation of biofouling on an inanimate surface coated with the marine coating composition; wherein the metal binding sequence comprises at least one member of the group consisting of SEQ ID Nos. 204-243 and 250-302 and functionally equivalent substituted metal binding amino acid sequences thereof. Product a composition, obtainable by process of incorporation of a proteinaceous molecule, wherein the proteinaceous molecule comprises a metal binding sequence binding a metal ligand in a sufficient amount to inhibit the accumulation of biofouling on an inanimate surface coated with a marine coating composition; wherein the metal binding sequence comprises at least one member of the group consisting of SEQ ID Nos. 204-243 and 250-302 and functionally equivalent substituted metal binding amino acid sequences thereof into a marine coating. A method for manufacturing product a marine coating composition comprising, a marine coating and a proteinaceous molecule, wherein the proteinaceous molecule comprises a metal binding sequence binding a metal ligand in a sufficient amount to inhibit the accumulation of biofouling on an inanimate surface coated with the marine coating composition; wherein the metal binding sequence comprises at least one member of the group consisting of SEQ ID Nos. 204-243 and 250-302 and functionally equivalent substituted metal binding amino acid sequences thereof; comprising the steps of incorporating the proteinaceous molecule into the marine coating. Provided is a marine coating; characterized in that a proteinaceous molecule, wherein the proteinaceous molecule comprises a metal binding sequence binding a metal ligand in a sufficient amount to inhibit the accumulation of biofouling on an inanimate surface coated with the marine coating composition; wherein the metal binding sequence comprises at least one member of the group consisting of SEQ ID Nos. 204-243 and 250-302 and functionally equivalent substituted metal binding amino acid sequences thereof; is included as a component of the marine coating. Use of a marine coating; for the purpose of reducing the concentration of a fouling agent on a surface. Product a composition, comprising at least one material selected from the group consisting of: a coating, an elastomer, an adhesive, a sealant, a textile finish, a wax, a thermoplastic, and a thermoset; wherein the coating is at least one coating selected from the group consisting of an architectural coating, a pipeline coating, an automotive coating, a can coating, a chemical agent resistant coating, a camouflage coating, a traffic marker coating, an aircraft coating, and a nuclear power plant coating; comprising, the at least one material and a proteinaceous molecule, wherein the proteinaceous molecule comprises a metal binding sequence binding a metal ligand in a sufficient amount to inhibit the accumulation of a biological entity on an inanimate surface comprising the material composition; wherein the metal binding sequence comprises at least one member of the group consisting of SEQ ID Nos. 204-243 and 250-302 and functionally equivalent substituted metal binding amino acid sequences thereof. Product a composition, obtainable by process of incorporation of a proteinaceous molecule, wherein the proteinaceous molecule comprises a metal binding sequence binding a metal ligand in a sufficient amount to inhibit the accumulation of a biological entity on an inanimate surface comprising the composition; wherein the metal binding sequence comprises at least one member of the group consisting of SEQ ID Nos. 204-243 and 250-302 and functionally equivalent substituted metal binding amino acid sequences thereof; into at least one material selected from the group consisting of: a coating, an elastomer, an adhesive, a sealant, a textile finish, a wax, a thermoplastic, and a thermoset; wherein the coating is at least one coating selected from the group consisting of an architectural coating, a pipeline coating, an automotive coating, a can coating, a chemical agent resistant coating, a camouflage coating, a traffic marker coating, an aircraft coating, and a nuclear power plant coating. A method for manufacturing product at least one material selected from the group consisting of: a coating, an elastomer, an adhesive, a sealant, a textile finish, a wax, a thermoplastic, and a thermoset; wherein the coating is at least one coating selected from the group consisting of an architectural coating, a pipeline coating, an automotive coating, a can coating, a chemical agent resistant coating, a camouflage coating, a traffic marker coating, an aircraft coating, and a nuclear power plant coating, comprising the at least one material and a proteinaceous molecule, wherein the proteinaceous molecule comprises a metal binding sequence binding a metal ligand in a sufficient amount to inhibit the accumulation of a biological entity on a an inanimate surface coated with the marine coating composition; wherein the metal binding sequence comprises at least one member of the group consisting of SEQ ID Nos. 204-243 and 250-302 and functionally equivalent substituted metal binding amino acid sequences thereof; comprising the steps of incorporating the proteinaceous molecule into the at least one material. Provided is at least one material selected from the group consisting of: a coating, an elastomer, an adhesive, a sealant, a textile finish, a wax, a thermoplastic, and a thermoset; wherein the coating is at least one coating selected from the group consisting of an architectural coating, a pipeline coating, an automotive coating, a can coating, a chemical agent resistant coating, a camouflage coating, a traffic marker coating, an aircraft coating, and a nuclear power plant coating; characterized in that a proteinaceous molecule, wherein the proteinaceous molecule comprises a metal binding sequence binding a metal ligand in a sufficient amount to inhibit the accumulation of a biological entity on an inanimate surface comprising the composition; wherein the metal binding sequence comprises at least one member of the group consisting of SEQ ID Nos. 204-243 and 250-302 and functionally equivalent substituted metal binding amino acid sequences thereof; is included as a component of the composition. Use of at least one material selected from the group consisting of: a coating, an elastomer, an adhesive, a sealant, a textile finish, a wax, a thermoplastic, and a thermoset; wherein the coating is at least one coating selected from the group consisting of an architectural coating, a pipeline coating, an automotive coating, a can coating, a chemical agent resistant coating, a camouflage coating, a traffic marker coating, an aircraft coating, and a nuclear power plant coating; for the purpose of reducing the concentration of a biological entity on a surface.

### D. DETAILED DESCRIPTION OF THE EMBODIMENTS

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent therein. It is to be understood, however, that the present invention may be embodied in various forms. Therefore, specific details disclosed herein are not to be interpreted as limiting, but rather as a basis for the claims and as a representative basis for teaching to employ the present invention in virtually any appropriately detailed system, structure or manner. It should be understood that the biomolecular composition(s), proteinaceous molecule(s), material formulation(s), surface treatment(s), filler(s), material(s), compound(s), method(s), procedure(s), and technique(s) described herein are presently representative of various embodiment(s). Other feature(s) will be readily apparent from the following detailed description; specific example(s) and claim(s); and various adaptation(s), change(s), equivalent(s), modification(s), substitution(s), deletion(s), and/or addition(s) of material(s), procedure(s) and/or protocol(s) other uses and modification(s) that may be made to the embodiment(s) disclosed herein without departing from the scope and spirit of the invention or as defined by the scope of the appended claim(s).

As used herein other than the claims, the terms “a,” “an,” “the,” and/or “said” means one or more. As used herein in the claim(s), when used in conjunction with the word(s) “comprise,” “comprises” and/or “comprising,” the word(s) “a,” “an,” “the,” and/or “said” may mean one or more than one. As used herein and in the claim(s), the terms “having,” “has,” “have,” “including,” “includes,” and/or “include” has the same meaning as “comprising,” “comprises,” and “comprise.” As used herein and in the claims “another” may mean at least a second or more. Various genera and sub-genera described herein are contemplated both as individual components, as well as and mixtures and combinations, and may be described in the claims as “at least one selected from,” “a mixture thereof” and/or “a combination thereof,” and such like. The phrase “a combination thereof” “a mixture thereof” and such like following a listing; the use of “and/or” in a listing; the phrase “such as,” followed by a listing; and/or a listing within brackets with “e.g.,” and/or “i.e.,” refers to any combination (e.g., any sub-set) of a set of listed component(s). For example, the phrase “such as ‘A,’ ‘B,’ or ‘C’” refers to various combinations that include, for example, the combination “A” and “B” as well as a combination “A” and “C” and/or combination “B” and “C.” Combinations of related species described herein though not directly placed in such a listing are also contemplated. For example, composition(s) described as a coating suitable for use on a marine surface described in different sections of the specification may be claimed individually and/or as a combination, as they are part of the same genera of marine coating(s). In another example, various monomer(s) of a chemical type such as “amino acid” may be described in various part(s) of the specification, and such amino acid monomer(s) may be claimed individually and/or in various combination(s).

In various embodiments described herein, exemplary values are specified as a range. It will be understood that herein that a given range includes all integers and sub-ranges comprised within a cited range. For example, citation of a range “0.03% to 0.07%” provides specific values within the cited range, such as, for example, 0.03%, 0.04%, 0.05%, 0.06%, and 0.07%, as well as various combinations of such specific values, such as, for example, 0.03%, 0.06% and 0.07%, 0.04% and 0.06%, or 0.05% and 0.07%, as well as sub-ranges such as 0.03% to 0.05%, 0.04% to 0.07%, or 0.04% to 0.06%, etc. Example 26 provides additional descriptions of specific numeric values within any cited range. As used herein and in the claim(s), “about” refers to any inherent measurement error or a rounding of digit(s) for a value (e.g., a measured value, calculated value such as a ratio), and thus the term “about” may be used with any value and/or range.

As used herein an “article” “article of manufacture” or “manufactured article” refers to a product (e.g., a textile, a spoon) that is made and/or altered by the hand of man, other than a composition of matter (e.g., a chemical composition). Unlike a machine, an article of manufacture lacks moving part(s). All patent(s) and publication(s) mentioned in this specification are herein incorporated by reference to the same extent as if each individual publication was specifically and individually indicated to be incorporated by reference.

1. Biomolecular Compositions and Biomolecules

As used herein, a “biomolecular composition” (“biomolecule composition”) refers to a composition comprising a biomolecule (e.g., a metal-binding proteinaceous molecule, an anti-fouling proteinaceous molecule, an anti-biological proteinaceous molecule, an enzyme). As used herein, a “biomolecule” refers to a molecule (e.g., a compound) comprising of one or more chemical moiety(s) [“specie(s),” “group(s),” “functionality(s),” “functional group(s)”] typically synthesized in living organisms, including but not limited to, an amino acid, a nucleotide, a polysaccharide, a simple sugar, a lipid, or a combination thereof. An example of a biomolecule includes a proteinaceous molecule (“proteinaceous composition,” “peptidic agent”), which comprises a polymer formed from one or more amino acid(s), such as a peptide (i.e., about 3 to about 100 amino acids), a polypeptide (i.e., about 101 or more amino acids, such as about 50,000 or more amino acids), and/or a protein. As used herein a “protein” comprises a proteinaceous molecule comprising a contiguous molecular proteinaceous sequence three amino acids or greater in length, matching the length of a biologically produced proteinaceous molecule encoded by the genome of an organism. Examples of a proteinaceous molecule include an enzyme, an antibody, a receptor, a transport protein, a structural protein, a prion, or a combination thereof. Examples of a peptide (e.g., an inhibitory peptide, an antifungal peptide) of about 3 to about 100 amino acids include those of about 3 to about 15 amino acids; about 3 to about 40 amino acids, etc. For ease of reference, a proteinaceous molecule (e.g., a peptide) referred to herein is written in the C-terminal to N-terminal direction to denote the sequence of synthesis (e.g., biological production, chemical synthesis). However, the conventional N-terminal to C-terminal manner of reporting amino acid sequences is utilized in the Sequence Listings. In some embodiments, a sequence may be produced and used in the forward and/or reverse pattern (e.g., synthesized C-terminal to N-terminal manner, or the reverse N-terminal to C-terminal). A proteinaceous molecule may comprise a mixture of proteinaceous molecules, such as a mixture of peptide(s) (e.g., an aliquot of a peptide library), polypeptide(s) and/or protein(s), and may also include a material (e.g., a chemical) in the art such as an associated immobilization agent(s), stabilizer(s), carrier(s), and/or inactive peptide(s), polypeptide(s), and/or protein(s). A biomolecular composition may comprise, for example, a chemically synthesized (“synthetic”) biomolecule molecule; a biologically manufactured biomolecule such as an endogenously produced and isolated and/or purified biomolecule, a recombinant biomolecule (e.g., a recombinant protein, polypeptide and/or peptide that may be produced using an expression vector in a host cell); a chemically modified biomolecule; or a combination thereof. For example, a peptide composition comprises a peptide derived from amino acids of a length readily accomplished using standard peptide synthesis procedures, such as, for example, between about 3 to about 100 amino acids in length (e.g., about 3 to about 25 residues in length, about 6 residues in length, etc.). In some aspects, one or more peptides may be prepared as a peptide library, which typically comprises a plurality (e.g., about 2 to about 10¹⁰ peptides). A peptide library may comprise a synthetically produced peptide and/or a biologically produced peptide (e.g., a recombinantly produced peptide, see for example U.S. Pat. No. 4,935,351). For example, a synthetic peptide combinational library typically comprises a mixture (e.g., an equimolar mixture) of free peptide(s).

In addition to the sources described herein for a biomolecule, a reagent, a living cell, etc., such a material and/or a chemical formula thereof may be obtained from convenient source such as a public database, a biological depository, and/or a commercial vendor. For example, various nucleotide sequences, including those that encode amino acid sequences, may be obtained at a public database, such as the Entrez Nucleotides database, which includes sequences from other databases including GenBank (e.g., CoreNucleotide), RefSeq, and PDB. Another example of a public databank for nucleotide and amino acid sequences includes the Kyoto Encyclopedia of Genes and Genomes (“KEEG”) (Kanehisa, M. et al., 2008; Kanehisa, M. et al., 2006; Kanehisa, M. and Goto, S., 2000). In another example, various amino acid sequences may be obtained at a public database, such as the Entrez databank, which includes sequences from other databases including SwissProt, PIR, PRF, PDB, Gene, GenBank, and RefSeq. Numerous nucleic acid sequences and/or encoded amino acid sequences can be obtained from such sources. In a further example, a biological material comprising, or are capable of comprising such a biomolecule (e.g., a living cell, a virus), may be obtained from a depository such as the American Type Culture Collection (“ATCC”), P.O. Box 1549 Manassas, Va. 20108, USA. In an additional example, a biomolecule, a chemical reagent, a biological material, and/or an equipment may be obtained from a commercial vendor such as Amersham Biosciences®, 800 Centennial Avenue, P.O. Box 1327, Piscataway, N.J. 08855-1327 USA”; BD Biosciences®, including Clontech Discovery Labware®, Immunocytometry Systems® and Pharmingen®, 1020 East Meadow Circle, Palo Alto, Calif. 94303-4230 USA”; Invitrogen™, 1600 Faraday Avenue, PO Box 6482, Carlsbad, Calif. 92008 USA”; New England Biolabs®, 32 Tozer Road, Beverly, Mass. 01915-5599 USA”; Merck®, One Merck Drive, P.O. Box 100, Whitehouse Station, N.J. 08889-0100 USA”; Novagene®, 441 Charmany Dr., Madison, Wis. 53719-1234 USA”; Promega, 2800 Woods Hollow Road, Madison Wis. 53711 USA”; Pfizer®, including Pharmacia®, 235 East 42nd Street, New York, N.Y. 10017 USA”; Quiagen®, 28159 Avenue Stanford, Valencia, Calif. 91355 USA”; Sigma-Aldrich®, including Sigma, Aldrich, Fluka, Supelco and Sigma-Aldrich Fine Chemicals, PO Box 14508, Saint Louis, Mo. 63178 USA”; Wako Pure Chemical Industries, Ltd, 1-2 Doshomachi 3-Chome, Chuo-ku, Osaka 540-8605, Japan; TCI America, 9211 N. Harborgate Street, Portland, Oreg. 97203, U.S.A.; Reactive Surfaces, Ltd, 300 West Avenue Ste #1316, Austin, Tex. 78701; Stratagene®, 11011 N. Torrey Pines Road, La Jolla, Calif. 92037 USA, etc. In a further example, a biomolecule, a chemical reagent, a biological material, and/or an equipment may be obtained from commercial vendors such as Amersham Biosciences®, 800 Centennial Avenue, P.O. Box 1327, Piscataway, N.J. 08855-1327 USA”; Allen Bradley, 1201 South Second Street, Milwaukee, Wis. 53204-2496, USA”; BD Biosciences®, including Clontech®, Discovery Labware®, Immunocytometry Systems® and Pharmingen®, 1020 East Meadow Circle, Palo Alto, Calif. 94303-4230 USA”; Baker, Mallinckrodt Baker, Inc., 222 Red School Lane, Phillipsburg N.J. 08865, U.S.A.“; Bioexpression and Fermentation Facility, Life Sciences Building, 1057 Green Street, University of Georgia, Athens, Ga. 30602, USA”; Bioxpress Scientific, PO Box 4140, Mulgrave Victoria 3170”; Boehringer Ingelheim GmbH, Corporate Headquarters, Binger Str. 173, 55216 Ingelheim, Germany Chem Service, Inc, PO Box 599, West Chester, Pa. 19381-0599, USA”; Difco, Voigt Global Distribution Inc., P.O. Box 1130, Lawrence, Kans. 66044-8130, USA”; Fisher Scientific, 2000 Park Lane Drive, Pittsburgh, Pa. 15275, USA”; Invitrogen™, 1600 Faraday Avenue, PO Box 6482, Carlsbad, Calif. 92008 USA”; Ferro Pfanstiehl Laboratories, Inc., 1219 Glen Rock Avenue, Waukegan, Ill. 60085-0439, USA”; New England Biolabs®, 32 Tozer Road, Beverly, Mass. 01915-5599 USA”; Merck®, One Merck Drive, P.O. Box 100, Whitehouse Station, N.J. 08889-0100 USA”; Novozymes North America Inc., PO BOX 576, 77 Perry Chapel Church Road, Franklinton N.C. 27525 United States; Millipore Corporate Headquarters,290 Concord Rd., Billerica, Mass. 01821, USA”; Nalgene®Labware, Nalge Nunc International, International Department, 75 Panorama Creek Drive, Rochester, N.Y. 14625. U.S.A.“; New Brunswick Scientific Co., Inc., 44 Talmadge Road, Edison, N.J. 08817 USA”; Novagene®, 441 Charmany Dr., Madison, Wis. 53719-1234 USA”; NCSRT, Inc., 1000 Goodworth Drive, Apex, N.C. 27539, USA”; Promega®, 2800 Woods Hollow Road, Madison Wis. 53711 USA”; Pfizer®, including Pharmacia®, 235 East 42nd Street, New York, N.Y. 10017 USA”; Quiagen®, 28159 Avenue Stanford, Valencia, Calif. 91355 USA”; SciLog, Inc., 8845 South Greenview Drive, Suite 4, Middleton, Wis. 53562, USA”; Sigma-Aldrich®, including Sigma, Aldrich, Fluka, Supelco, and Sigma-Aldrich Fine Chemicals, PO Box 14508, Saint Louis“; USB Corporation, 26111 Miles Road, Cleveland, Ohio 44128, USA”; Sherwin Williams Company, 101 Prospect Ave., Cleveland, Ohio, USA”; Lightnin, 135 Mt. Read Blvd., Rochester, N.Y. 14611 U.S.A.“; Amano Enzyme, USA Co., Ltd. 2150 Point Boulevard Suite 100 Elgin, Ill. 60123 U.S.A.”; Novozymes North America Inc., 77 Perry Chapel Church Road, Franklinton, N.C. 27525, U.S.A.”; and WB Moore, Inc., 1049 Bushkill Drive, Easton, Pa. 18042.

In addition to those techniques specifically described herein, a cell, nucleic acid sequence, amino acid sequence, and the like, may be manipulated in light of the present disclosures, using standard techniques [see, for example, In “Molecular Cloning” (Sambrook, J., and Russell, D. W., Eds.) 3rd Edition, Cold Spring Harbor, N.Y.: Cold Spring Harbor Laboratory Press, 2001”; In “Current Protocols in Molecular Biology” (Chanda, V. B. Ed.) John Wiley & Sons, 2002”; In “Current Protocols in Nucleic Acid Chemistry” (Harkins, E. W. Ed.) John Wiley & Sons, 2002”; In “Current Protocols in Protein Science” (Taylor, G. Ed.) John Wiley & Sons, 2002”; In “Current Protocols in Cell Biology” (Morgan, K. Ed.) John Wiley & Sons, 2002”; In “Current Protocols in Pharmacology” (Taylor, G. Ed.) John Wiley & Sons, 2002”; In “Current Protocols in Cytometry” (Robinson, J. P. Ed.) John Wiley & Sons, 2002”; In “Current Protocols in Immunology” (Coico, R. Ed.) John Wiley & Sons, 2002].

2. Material Formulations Comprising a Biomolecular Composition

The compositions and methods herein may produce a material (“material formulation”) comprising a biomolecular composition comprising a biomolecule. The material formulation may possess an activity (e.g., a proteinaceous molecule's metal-binding activity, a proteinaceous molecule's anti-biofouling activity, a peptide's anti-biological activity, an enzyme's catalytic reaction) conferred by the inclusion of the biomolecule as a component of the material formulation. A material formulation may comprise a biomolecular composition by being formulated, prepared, processed, post-cured processed, manufactured, and/or applied (e.g., applied to a surface), in a fashion to be suitable to possess an activity and/or function of a biomolecule. The activity may modify (e.g., maintain a property, alter a property, confer a property) of the material formulation. Examples of a property that may be modified in a material formulation include reversibly binding a metal ion upon incorporation of a metal binding proteinaceous sequence as part of the material formulation, resistance (e.g., biofouling resistance) to a living cell (e.g., a microorganism) upon incorporation of an anti-biological proteinaceous molecule (e.g., an anti-fouling proteinaceous molecule, an anti-biological proteinaceous molecule) as part of the material formulation, and/or catalytic activity upon incorporation of an enzyme as part of the material formulation. In an additional example, material formulation (e.g., a marine coating) comprising an anti-biological proteinaceous molecule (e.g., an OP degrading enzyme) may be capable of reducing inhibiting or preventing adherence (e.g., enhancing ease of wash removal) and/or growth of the biological entity and/or fouling molecule, and may do so for extended periods of time (e.g., greater than a week, a month, a year, etc.) relative to like formulation with a reduced (e.g., absent) content of the proteinaceous molecule. An anti-fouling, metal-binding and/or anti-biological material (e.g., an surface treatment, a surface covered with a surface treatment, a polymer-based material such as a plastic) may, for example, undergo a reduced amount of additional treatment(s) with another anti-biological, metal-binding and/or antifouling composition, a reduced amount of clean-up treatment to effect decontamination and/or cosmetic repair (e.g., painting), thereby simplifying upkeep of the physical condition and appearance of biological entity infestation prone material (e.g., a marine surface, a building's exterior). For example, a surface treatment may be applied to susceptible surfaces in advance of and/or during exposure to an organism (e.g., a fouling organism), such as for a method of treating or preventing growth of a biological entity and/or accumulation of fouling molecule(s) on a susceptible surface. In an embodiment, a polymer-based compound that prophylactically and continuously deters biological infestation, inhibits and/or kills cells and/or viruses is provided.

In the context of a biomolecule, “active” or “bioactive” refers to the effect of biomolecule, such as conferring and/or altering a property of a material formulation. For example, a material formulation comprising an “active” or “bioactive” anti-biological proteinaceous molecule refers to the material formulation possessing altered and/or conferred anti-biological effect (e.g., a biocidal effect, a biostatic effect) on a living cell and/or a virus relative to a like material formulation lacking a similar content of the anti-biological proteinaceous molecule. In another example, as used herein, the term “bioactive” or “active” refers to the ability of an enzyme to accelerate a chemical reaction differentiating such activity from a like ability of a composition, an article, a method, etc. that does not comprise an enzyme to accelerate a chemical reaction. An “effective amount” refers to a concentration of component of a material formulation (e.g., a metal binding peptide, an anti-biological peptide, etc) and/or the material formulation itself capable of exerting a desired effect (e.g., an anti-fouling effect, an anti-biological effect). In an example, a proteinaceous molecule may be incorporated into a material formulation, and may confer one or more properties (e.g., one or more enzymatic activities, one or more binding activities, one or more anti-biological activities, one or more metal binding activities, etc) to the material formulation, that is detectable by an assay relative to a like material having a reduced concentration (e.g., lacking) such a proteinaceous molecule. In another example, an effective amount of proteinaceous molecule comprising a metal binding sequence incorporated into a marine coating may confer a reversible metal binding activity and an anti-fouling activity due to an increased local concentration of a metal (e.g., a metal cation) at or near a marine surface coated with the marine coating. In a further example, one or more layers of material formulation such as a multicoat system may comprise one or more different biomolecular compositions to confer differing properties between one layer and at least a second layer of the multicoat system. An example of a multicoat system is a plurality of coating layers. The coating selected for use in a specific layer may differ from an additional layer of the multicoat system. Examples of a coating that may be selected for use, either alone or in a multicoat system, include a sealer, a water repellent, a primer, an undercoat, a topcoat, or a combination thereof. In specific aspects, a multicoat system may comprise a plurality of layers of the same and/or differing type, such as, for example, 1 to 10 layers. In an additional example, one may select a combination of biomolecules to confer a plurality and/or expanded range of properties to a composition. In various embodiments, a composition (e.g., a material formulation), an article (e.g., an article of manufacture comprising a material formulation), a device (e.g., a device comprising a material formulation), an apparatus (e.g., an apparatus comprising a material formulation), a method, etc. may comprise one or more selected biomolecule(s) (e.g., about 1 to about 1000 or more) in various combinations thereof, wherein one or more biomolecule(s) may confer various property(s). In some aspects, it is contemplated that an additive and/or synergistic anti-biological property may occur when a metal-binding proteinaceous molecule, an anti-fouling proteinaceous molecule, an anti-biological proteinaceous molecule, an anti-biological enzyme, another enzyme, and/or an additional anti-biological agent (e.g., an anti-fouling anti-biological agent, a preservative, an antimicrobial agent) are combined.

In some embodiments, the concentration of any individual selected biomolecule (e.g., an enzyme, a peptide, a polypeptide) of a material formulation (e.g., the wet weight of a biomolecular composition, the dry weight of a biomolecular composition, the average content in the primary particles of a biomolecular composition, such as the primary particles of a cell-based particulate material) comprises about 0.000000001% to about 100%, of the biomolecular composition and/or a material formulation. For example, a cell-based particulate material may function as a filler, and may comprise up to about 80% of the volume of material formulation (e.g., a coating, a surface treatment), in some embodiments. In another example, a proteinaceous molecule (e.g., a peptide) may comprise, for example, about 0.000000001% to about 40%, 20%, 10%, or 5% of a material formulation. In another example, a material formulation (e.g., a marine coating) may comprise a biomolecular composition comprising, for example, an metal-binding proteinaceous molecule, an anti-fouling proteinaceous molecule, an anti-biological proteinaceous molecule, an anti-biological enzyme, another enzyme, and/or comprise an additional anti-biological agent (e.g., an anti-fouling anti-biological agent, a preservative, an antimicrobial agent), wherein the total content of the biomolecular composition and/or the additional anti-biological agent is between about 0.000001% to about 80% or more by weight and/or volume (e.g., about 0.5% to about 80%; about 25% to about 45%, about 70% to about 80%, etc).

An example of a material formulation comprises a “surface treatment,” which refers to a composition applied to a surface, and examples of such compositions include a coating (e.g., a paint, a clear coat), a textile finish, a wax, an elastomer, an adhesive, a filler, and/or a sealant. In some embodiments, such a surface treatment may be prepared as an amorphous material (e.g., a liquid, a semisolid) and/or a simple geometric shape (e.g., a planar material) to allow ease of application to a surface. Such a surface treatment may provide function(s) upon application to a surface such as, for example, confer an metal binding, an anti-fouling, an anti-biological, and/or an enzymatic property to the surface (e.g., a marine surface). For example, a surface treatment comprising a biomolecular composition conferring a reversible metal binding property may possess an anti-fouling activity due to a localized concentration of a metal cation at or near the surface treatment that reduces adhesion and/or growth a fouling organism in and/or upon the surface treatment and/or the underlying surface. In another example, a terrestrial surface treatment (i.e., a surface treatment formulated for a non-marine environment) comprises a biomolecular composition that confers such activity(s).

An adhesive refers to a composition capable of attachment to one or more surface(s) [“substrate(s)”] of one or more object(s) [“adherent(s)”], wherein the composition comprises a solid or is capable of converting into the solid, wherein the solid is capable of holding a plurality of adherents together by attachment to the surface of the adherents while withstanding a normal operating stress load placed upon the adherents and the solid adhesive. For example, an adhesive (e.g., a glue, a cement, an adhesive paste) may be capable of uniting, bonding and/or holding at least two surfaces together, usually in a strong and permanent manner. A sealant comprises a composition capable of attachment to a plurality of surfaces (e.g., at least two surfaces) to fill a space and/or a gap between the plurality of surfaces and form a barrier to a gas, a liquid, a solid particle, an insect, or a combination thereof. An adhesive generally functions to prevent movement of the adherents, while a sealant typically functions to seal adherents that move. A sealant comprises a subtype of an adhesive based on purpose/function (i.e., a flexible adhesive), and a sealant typically possesses lower strength, greater flexibility, or a combination thereof, than many other types of adhesive(s) (e.g., a structural adhesive). In contrast to adhesive and/or a sealant, an abhesive comprises a material (e.g., a clear coating, a paint; a mold release agent such as a plastic release film) applied to a surface to inhibit adhesion/sticking of an additional material to the abhesive and/or a surface the abhesive covers.

An elastomer (“elastomeric material”) comprises a “macromolecular material that returns rapidly to approximately the initial dimensions and shape after substantial deformation by a weak stress and release of the stress” while a rubber comprises a material “capable of recovering from a large deformation quickly and forcibly, and can be, and/or are already is, modified to a state in which it is essentially insoluble (but can swell) in a solvent.” Examples of a solvent commonly used to swell a rubber include benzene, methyl ethyl ketone, and/or ethanol toluene azeotrope (see, for example, definitions in ASTM D 1566). A rubber retracts within about one minute to less than about 1.5 times its original length after being held for about one minute at about twice its length at room temperature, while an elastomer retracts within about five minutes to within about 10% original length after being held for about five minutes at about twice its length at room temperature. Often cross-linking/vulcanization may be used to confer an elastomeric property, as the cross-links promote maintenance of a material's dimensions. A plastic comprises a polymeric material solid at room temperature (i.e., about 23° C.) in a finished state, and at some stage of the plastic's manufacture and/or processing was capable of being shaped by flow and/or molding into a finished article. A material such as an elastomer, a textile, an adhesive, or a paint, which may in some cases meet this definition, are not considered to be a plastic. All plastics comprise a polymer, but not all polymers are a plastic, such as, for example, a cellulose that lacks a chemical modification to allow it to be processed as a plastic during manufacture, or a polymer that possesses an elastomeric property. All polymeric materials comprise a polymer, but not all polymers possess the physical/chemical properties to be classified as a specific material type (e.g., a plastic, an elastomer, etc), particularly when such a material type comprises another component in addition to the polymer to produce a compositional/structural difference that produces a material of particular property(s) and purpose of use.

As some terms often have different meanings for different material types and/or uses being described, and the meaning applicable to the material should be applied as appropriate in the context, as understood in the applicable art. For example, a “cell” in a biotechnology art described for production of a biomolecule refers to the smallest unit of living matter (viruses not withstanding), while a “cell” in a material art (e.g., an elastomer art) refers to a void in a material to produce a solid foam material (e.g., elastomer foam material). In another example, the word “mold” may be used in the context of a fungal cell, while in other context “mold” refers to a solid structure used to shape a material, such as a mold used to shape an elastomeric material into a geometric shape. In such instances, the appropriate definition and/or meaning for the term (e.g., a biomolecular composition produced from a cell vs. a void, a solid foamed material vs. a liquid or gas foam; a biological cell/organism vs. a device for material manufacture) should be applied in accordance with the context of the term's use in light of the present disclosures.

A coating (“coat,” “surface coat,” “surface coating”) refers to “a liquid, liquefiable or mastic composition that is converted to a solid protective, decorative, or functional adherent film after application as a thin layer” (“Paint and Coating Testing Manual, Fourteenth Edition of the Gardner-Sward Handbook” (Koleske, J. V. Ed.), p. 696, 1995; and in “ASTM Book of Standards, Volume 06.01, Paint—Tests for Chemical, Physical, and Optical Properties; Appearance,” D16-00, 2002). Additionally, a thin layer comprises about 5 um to about 1500 um thick. In many embodiments, a coating forms a thin layer about 15 um to about 150 um thick. Examples of a coating include a clear coating or a paint. In some embodiments, a coating may comprise a water-based coating, a solvent-based coating, and/or a powder coating. Material formulations such as a coating, an elastomer, an sealant, an adhesive, a wax, a filler, a surface treatment, a plastic (e.g., a thermoplastic, a thermoset), a reinforced polymeric material, a composite, a laminate, a polymeric material, other biomolecule(s), peptide composition(s), enzyme(s), and their preparation, which may be used in light of the present disclosure(s) have been described in U.S. patent application Ser. Nos. 10/655,345, 10/792,516, 10/884,355, 11/865,514, 12/243,755, 12/474,921 and 12/696,651).

However, a material may comprise a thinner layer upon the surface of another material, such as from about a molecular layer (e.g., about 32 pm to about 10,000 pm) to about 5 μm thick. Such thinner material layer(s) may be referred to as a “coat,” “coating,” and/or a “film” but are not considered herein to be a coat, coating and/or a film such as in the art of a paint or a clear coating, due to differences such as formulation, preparation, processing, application, function, or a combination thereof. For example, a layer of hydrophobic molecules loosely adhering to a hydrophobic biomolecule may be referred to as a “coat,” “coating,” and/or a “film,” but does not fall into the art of a coating such as a paint applied to a wall. Examples of such thinner material layers often referred to as a “coat,” “coating,” and/or a “film” includes a molecular scale layer, a microencapsulating material, a seed “coating,” a textile finish, a pharmaceutical encapsulating material, and the like. As used herein and in the claim(s), a coating, a coat, a surface coat, a surface coating, a film, and/or a surface film refers to a coating and/or a coating produced film, as would be understood in the arts of a clear coating and/or a paint, unless otherwise specified in the claims(s) or by the context herein, as would be understood in the respective art(s).

Where the context so indicates, the term “coating” refers to the coating that is applied. For example, a coating may be capable of undergoing a change from a fluent to a nonfluent condition by removal of solvents, vehicles and/or carriers, by setting, by a chemical reaction and/or conversion, and/or by solidification from a molten state. The coating and/or the film that is formed may be hard or soft, elastic or inelastic, permanent or transitory, or a combination thereof. Where the context so indicates, the term “coating” includes the process of applying (e.g., brushing, dipping, spreading, spraying) or otherwise producing a coated surface, which may also be referred to as a coating, coat, covering, film or layer on a surface. Where the context allows, the act of coating also includes impregnating a surface and/or an object by causing a material to extend or penetrate into the object, or into the interstices of a porous, a cellular and/or a foraminous material. For example, a surface treatment such as a coating may impregnate a porous and/or semi-porous material such as the surface of an object (e.g., high surface area porous stone structure) that may be capable of supporting biological entity growth. Circumstances requiring treatment of a porous surface may benefit from using a relatively thin coating material rather than a thick, pigmented paint, to facilitate penetration of the pores. In another example, a surface treatment such as a coating may be applied prophylactically over a “clean” surface not contaminated by a biological entity and/or applied to a surface already suspected of being contaminated by a biological entity.

A surface comprises the outer layer of any solid object. The term “substrate” or “base” in the context of a coating, may be synonymous with the term “surface.” However, as “substrate” has a different meaning in the arts of enzymology and coatings, the term “surface” may be preferentially used herein for clarity. A surface wherein a coating has been applied, whether or not film formation has occurred, may be known herein as a “coated surface.”

A paint generally refers to a “pigmented liquid, liquefiable or mastic composition designed for application to a substrate in a thin layer which is converted to an opaque solid film after application. Used for protection, decoration or identification, or to serve some functional purpose such as the filling or concealing of surface irregularities, the modification of light and heat radiation characteristics, etc.” [“Paint and Coating Testing Manual, Fourteenth Edition of the Gardner-Sward Handbook” (Koleske, J. V. Ed.), p. 696, 1995]. However, as certain coatings disclosed herein are non-film forming coatings, this definition is modified herein to encompass a coating with the same properties of a film forming paint, with the exception that it does not produce a solid film. In particular embodiments, a non-film forming paint possesses a hiding power sufficient to concealing surface feature comparable to an opaque film.

A clear-coating refers to a coating that is not opaque and/or does not produce an opaque solid film after application. A clear-coating and/or film may be transparent or semi-transparent (e.g., translucent). A clear-coating may be colored or non-colored. In certain embodiments, reducing the content of a pigment in a paint composition may produce a clear-coating. Additionally, a clear-coating may comprise a lacquer, a varnish, a shellac, a stain, a water repellent coating, or a combination thereof. Though some opaque coatings are referred to in the art as a lacquer, a varnish, a shellac, or a water repellent coating, all such opaque coatings are considered as paints herein (e.g., a lacquer-paint, a varnish-paint, a shellac-paint, a water repellent paint).

In certain embodiments, a material formulation such as a surface treatment (e.g., a coating, a film) may be removed from use. For example, a non-film forming coating, a temporary film, a self-cleaning film, a coating and/or a film that has been damaged, may be otherwise no longer desired and/or no longer suitable for use, may be removed from a surface using the techniques of the art.

3. Material Formulations Comprising an Anti-Biological Compositions

The biomolecular composition(s) such as a proteinaceous molecule may be used (e.g., incorporated) with any of the various types of water contacting (e.g., marine, pipeline) material formulation(s) described herein or as would be known in the art, alone or in various combinations. Further, such biomolecular composition(s) are contemplated to be combinable with a terrestrial material formulation(s) (e.g., an architectural coating, an architectural elastomer, etc) described herein or as would be known in the art, alone or in various combinations. In specific aspects, a material formulation comprising an anti-biological agent (e.g., an anti-biological proteinaceous molecule) may act (e.g., inhibit growth) against a biological unit (“biological entity”) such as a cell (e.g., a prokaryotic cell, a eukaryotic cell) and/or a virus that contacts (e.g., a surface contact, an internal incorporation, an infiltration, an infestation) the material formulation. For example, a material formulation may comprise an anti-biological agent such as an anti-biological proteinaceous molecule such as a peptide, a polypeptide, a protein, an enzyme, or a combination thereof, having an anti-biological property. An anti-biological agent generally binds a biomolecule ligand to act on the biological entity, such as, for example an enzyme cleaving a cellular biomolecule and/or a peptide associating with and disrupting a cellular membrane. In other embodiments, it is contemplated that an anti-biological agent may act on a molecule secreted by a cell to prevent the molecule from contacting and affecting an organism and/or alter the effect of the molecule upon contact with an organism, such as promoting negative chemotaxis of a fouling organism away from a surface. A specific anti-biological property would be anti-fouling activity against a biofouling organism and/or a biomolecule produced by a biofouling organism. Another example of an anti-biological activity would be activity against a terrestrial organism, such as a terrestrial microorganism (e.g., a fungus, a bacterium), such as a microorganism that infests a terrestrial coating.

In an example, an effective amount of an anti-biological agent may demonstrate an anti-biological activity such as by treating an infestation, preventing infestation, deterring infestation, inhibiting infestation (e.g., preventing cell attachment), treating growth, inhibiting growth, preventing growth, deterring growth, lysing, and/or killing; a biological entity (e.g., one or more genera and/or species of a cell and/or a virus). Thus, some embodiments comprise a process for treating an infestation, preventing infestation, inhibiting infestation (e.g., preventing cell attachment), inhibiting growth, preventing growth, lysing, and/or killing a biological entity comprising contacting the biological entity with a material formulation (e.g., a paint, a coating) comprising an effective amount of at least one anti-biological agent. In some aspects, such an anti-biological agent may possess a biocidal and/or a biostatic activity. For example, an anti-biological and/or an anti-fouling enzyme may act as a biocide and/or a biostatic. In another example, a biostatic proteinaceous molecule may inhibit growth of a biological entity, which refers to cessation and/or reduction of cell and/or viral proliferation, and can also include inhibition of expression of cellullarly produced proteins in a static cell colony. For example, a coating comprising an anti-biological agent may act against a biological entity adapted for growth in a non-marine environment and/or does not produces fouling; while a coating comprising an anti-fouling agent may act against a marine cell that produces fouling. In another example, a virus may be a target of such an anti-biological agent, as the virus (e.g., a membrane enveloped virus) may comprise a biomolecule target of an anti-biological agent (e.g., an enzyme, an anti-biological proteinaceous molecule such as a peptide). In a further example, a method of using the anti-biological and/or anti-fouling proteinaceous additive and compositions may be used for treating existing biological (e.g., fungal, bacterial, biofouling) colonies and/or for deterring or preventing biological entity (e.g., fungal, bacterial, biofouling) infestation and inhibiting cell growth or proliferation on a variety of inanimate object(s) such as interior and exterior architectural surfaces (e.g., a hospital coating) and building materials. In another example, the associated discoloration, disfiguration and/or degradation of the supporting substrate and/or surface may be avoided and/or reduced by use of an anti-biological and/or anti-fouling proteinaceous agent. In a further example, the compositions disclosed herein may be useful for a material (e.g., on a surface) where conditions are conducive to deposition and development of biological entity (e.g., a cell of a pest organism, a virus), and where control of cellular and/or viral growth may be accomplished with a proteinaceous composition that may be relatively non-toxic (e.g., non toxic) to humans, pets and other animals or harmful to the environment.

4. Fouling of Surfaces

Fouling refers to the adhesion to a surface of fouling molecule(s) such as protein(s), glycoprotein(s) (e.g., a proteoglycan), polysaccharide(s), and some inorganic molecule(s), and organism(s) upon contact by the surface with the water (e.g., sea water, fresh water) that has such a fouling molecule. A fouled surface is typically rougher and/or has a higher fictional resistance property. For example, a fouled surface may reduce the speed of a vessel (e.g., a ship) in water, reduce a vessel's maneuverability, increase a vessel's weight, increase a vessel's fuel consumption (e.g., up to 40%), increase a vessel's maintenance time and/or repair cost in dry dock, reduce the use time of a vessel, enhance corrosion, alter a surface's electrical conductivity, discolor a coating and/or surface, or a combination thereof. Fouling produced by biomolecule(s) and/or organism(s), as opposed to inorganic material(s) (e.g., a layer of mineral precipitation upon an aqueous surface), may be referred to herein as “biofouling.”

Fouling typically occurring within minutes upon contact with water comprising such fouling molecules to produce an initial fouling biofilm (“conditioning film”). Fouling microorganism(s) generally incorporate into the fouling biofilm within 24 hrs. Examples of a common fouling microorganism include a bacterium, a diatom, an alga, a marine fungus, a protozoan (e.g., Vaginicola sp., Vorticella sp., Zoolhamnium sp.), a barnacle cyprid and/or a rotifer, while examples of a fouling macro-organism include an animal such as a barnacle, a tunicate, a mollusk and/or a bryozoan. An incorporated alga (e.g., Ulothrix zonata, Enteromorpha intestinalis) and/or protozoan(s) may produce spores that become part of the fouling biofilm. Larvae of a marine macro-organism (i.e., a visible multicellular organism), including Balanus amphitrite, Laomedia flexuosa, Electra crustulenta, Spirorbis borealis, Mytilus edulis, and Styela coriacea, may become attached within a few weeks. A marine invertebrate and/or a macroalga such as seaweed may also adhere to the fouling biofilm. Specific examples of fouling organisms include Achnantes brevipes, Amphiprora paludosa, Amphora coffeaeformis, Enteromorpha intestinalis, Licmophora abbreviate, Nifzschia pusilla, Pseudomonas putrefaciens, Vaginicola sp., Vibrio alginofyticos, Vorticella sp., Zoolhamnium sp., Ulothrix zonata, or a combination thereof.

In some instances, biofouling may be differentiated by the type of organism contributing the fouling layer, such as a “soft” fouling microorganism as a green alga (e.g., Cladophora spp., Enteromorpha spp., Ulva spp.), a “hard fouling” organism having a hard shell (e.g., a balanus, a barnacle, a bryozoan, a mollusk), a small brush and grass type organism (e.g., hydroids, bryozoans), or a spineless organism (e.g., an ascidian, a sea anemone, a sponge). A surface in contact with water that is stationary or generally moves at slower speeds (e.g., less than about 4 knots) tend to foul faster, particularly with a macro-organism, than a faster moving surface. Fouling tends to increase with a higher water temperature, a neutral or an acidic condition, and/or salinity suitable for many fouling organism(s).

5. Material Formulations Comprising a Metal Binding and/or Anti-Fouling Proteinaceous Molecule

In some embodiments, a material formulation (e.g., a marine coating) comprising a metal binding proteinaceous molecule may accumulate an increased concentration of a metal atom(s) upon contact with a metal. For example, a material formulation may bind a metal atom upon contacting an aqueous solution (e.g., fresh water, salt water, a solution applied to the material formulation) comprising dissolved metal atom(s), and though some metal atom(s) and/or proteinaceous molecule(s) may disassociate from the material formulation, additional and/or continuous contact with the aqueous solution renews the metal content in material formulation. In some embodiments, a metal (e.g., a metallic pigment) may be a component of a material formulation to provide metal for binding to the metal binding proteinaceous molecule. The metal binding proteinaceous molecule may also possess an anti-biological activity (e.g., an anti-fouling activity) that is conferred to the material formulation due to the binding of a metal toxic to an organism (e.g., a biofouling organism).

About 40% of proteins (e.g., a metalloprotein) bind a metal cation ligand such as a Ca²⁺, a Co²⁺/³⁺, a Cu²⁺/²⁺, a Fe²⁺/³⁺, a K⁺, a Mg²⁺, a Mn²⁺, a Na⁺, a Ni³⁺, and/or a Zn²⁺. An example of a metalloprotein and ligand includes a cyanobacteria's and/or a plant's plastocyanin that binds copper; a plant's and/or a bacteria's ascorbate oxidase that binds copper; an animal's hemocuprein that binds copper; an animal's albumin that binds iron, lead, and/or manganese; an animal's carboxypeptidase A that binds zinc; an animal's casein that binds iron; an animal's ceruloplasmin that binds copper; an animal's cytochrome that binds iron; an animal's cytochrome oxidase that binds copper; an animal's ferritin that binds iron; an animal's glutamine synthetase that binds manganese; an animal's glutathione peroxidase that binds selenium; an animal's haemoglobin that binds iron and/or lead; an animal's hemosiderin that binds iron; an animal's insulin that binds zinc; an animal's myoglobin that binds iron; an animal's ovotrasferrin that binds iron; an animal's oxalacetate decarboxylase that binds manganese; an animal's pyruvate carboxylase that binds manganese; an animal's superoxide dismutase that binds copper; an animal's transferrin that binds chromium and/or iron; an animal's tyrosinase that binds copper; an animal's β-globulin that binds manganese; or a combination thereof (Garcia, J. S. et al., 2006).

An example of a metal (e.g., a metal ion) that may be bound by a proteinaceous metal binding sequence includes an alkali metal (e.g., caesium, francium, lithium, potassium, rubidium, sodium); an alkaline earth metal (e.g., barium, beryllium, calcium, magnesium, radium, strontium); a transition metal (e.g., bohrium, cadmium, chromium, cobalt, copper, darmstadtium, dubnium, gold, hafnium, hassium, iridium, iron, manganese, meitnerium, mercury, molybdenum, nickel, niobium, osmium, palladium, platinum, rhenium, rhodium, roentgenium, ruthenium, rutherfordium, scandium, seaborgium, silver, tantalum, technetium, titanium, tungsten, ununbium, vanadium, yttrium, zinc, zirconium); a post-transition metal (e.g., aluminum, bismuth, gallium, indium, lead, thallium, tin, ununhexium, ununpentium, ununquadium, ununtrium); an anthanoid (e.g., cerium, dysprosium, erbium, europium, gadolinium, holmium, lanthanum, lutetium, neodymium, praseodymium, promethium, samarium, terbium, thulium, ytterbium); an actinoid (e.g., actinium, americium, berkelium, californium, curium, einsteinium, fermium, lawrencium, mendelevium, neptunium, nobelium, plutonium, protactinium, thorium, uranium); or a combination thereof. An example of a metal in the form of a cation that may be bound by a metal binding sequence includes an Ag⁺, an Al³⁺, an As³⁺, an Au^{+/3+}, a Ba²⁺, a Be²⁺, a Bi³⁺, a Ca²⁺, a Cd²⁺, a Co²⁺/³⁺, a Cs⁺, a Cu⁺/²⁺, a Fe³⁺/³⁺, a Hg²⁺, a K⁺, a Li⁺, a Ln³⁺, a Mg²⁺, a Mn²⁺, a Na⁺, a Ni²⁺, a Pb²⁺, a Pt^{2+/4+}, a Rb⁺, a Sr²⁺, a Zn²⁺, or a combination thereof. In some instances, a metal binding site in a proteinaceous molecule (e.g., a protein) may bind a heavy and/or a more generically toxic metal such as an Al³⁺, a Cd²⁺, a Hg²⁺, a Ln³⁺, and/or a Pb²⁺. For example, a Zn²⁺ binding protein may bind a Hg²⁺, while a protein that binds a Mg²⁺ may bind an Al³⁺.

As a cation (e.g., a metal cation) is positively charged, a proteinaceous molecule's binding affinity for a cation may be improved by the proteinaceous molecule comprising a negatively charged moiety such as an HCOO⁻ (e.g., a glutamate's carboxyl, an aspartate's carboxyl, a C-terminal carboxyl) and/or a CH₃S⁻ (e.g., a cysteine); an increased dipole moment and/or polarity of a moiety (e.g., a hydroxyl moiety, particularly an aromatic associated hydroxyl); the ability of a moiety to donate electrical charge to an ion (e.g., a cation); and/or the availability of a greater number of available moiety(s) that have affinity to bind an ion such as a metal cation (i.e., denticity) to form a binding coordination complex (Dudev, T. and Lim, C. 2008). An imidazole nitrogen of a histidine and/or a thiol sulfur of a cysteine often function as a donor atom in binding a cation, and a side chain of a serine, a threonine, a tyrosine, a tryptophan, a lysine, an arginine, an aspartic acid, a glutamic acid, an asparagine, a glutamine, and/or a methionine may also function as donor moiety(ies) (Sovago, I. and Osz, K., 2006; Matthews, D. J., 1995). A proteinaceous molecule comprising a metal binding proteinaceous sequence may comprise about 5% to about 100% of such electron donor amino acid(s) in the metal binding sequence. A metal binding proteinaceous sequence may comprise electron donor amino acid(s) consecutively (e.g., His-Cys-His) or non-consecutively (e.g., His-Val-Cys) arrangement(s). In many embodiments, a metal-binding proteinaceous sequence (e.g., a peptide) may comprise about 1 to about 100 (e.g., about 1 to about 40, about 1 to about 20) electron donating amino acid(s) in a consecutive sequence followed by a non-electron donor amino acid or termination of a sequence. In many embodiments, a metal-binding proteinaceous sequence may comprise about 1, about 2, about 3, about 4, about 5, about 6, about 7 about, 8, about 9, to about 10 non-electron donating amino acid(s) interspersed between one or more electron donor amino acid(s). One or more amino acid substitution(s) may occur among amino acid(s) that may donate an electrical charge to an ion (e.g., a metal cation). Examples of amino acid(s) that may donate an electrical charge to an ion, and are contemplated as “functionally equivalent” or a “conservative modified variant” in the context of such substitutions in a metal binding proteinaceous sequence include a histidine, a cysteine, a serine, a threonine, a tyrosine, a lysine, an arginine, an aspartic acid, a glutamic acid, an asparagine, a glutamine, and/or a methionine. For example, an electrical charge donating histidine may be substituted for with an electrical charge donating cysteine, a serine, a threonine, a tyrosine, a lysine, an arginine, an aspartic acid, a glutamic acid, an asparagine, a glutamine, and/or a methionine. In another example, it is contemplated that to reduce or eliminate cysteine from a metal binding sequence and produce a functional equivalent metal binding sequence, another electrical charge donor/metal binding amino acid (e.g., His, Asp, Lys) may substitute for a cysteine.

A positively charged side chain of a lysine and/or an arginine; a terminal amino moiety; and/or a partially charged peptide backbone amide moiety may promote an increased number of binding interactions (e.g., about 1 or 2 additional interactions) of a cation to a negatively charged and/or partially negatively charged moiety(s) (e.g., a carboxylate moiety). The action of such a positively charged moiety in promoting such binding interactions between a proteinaceous molecule and a cation may be due to partial charge neutralization of the cation and/or associated charged and/or partly charged ion(s) and/or water molecule(s) (Dudev, T. and Lim, C. 2008). Accordingly, amino acid substitutions among amino acids that may contribute a positive charge (e.g., a side chain positive charge) are contemplated as “functionally equivalent” in the context of a metal binding proteinaceous sequence. An example of such a positive charge contributing amino acid includes a lysine, an arginine, and/or a histidine, and each may substitute for the other(s). For example, a positive charge contributing lysine may be substituted for with a positive charge contributing arginine and/or a histidine.

An aromatic side chain may offer thermodynamic stability for a peptide binding structure, and a tryptophan may also have metal binding affinity [Sovago, I. and Osz, K., 2006; Proteins Labfax (Price, N.C., Ed.), pp. 52-53, 1996]. Accordingly, amino acid substitutions among aromatic amino acids are contemplated as “functionally equivalent” in the context of a metal binding proteinaceous sequence. An example of such an aromatic amino acid includes a phenylalanine, a tryptophan, and/or a tyrosine. For example, a tryptophan may be substituted for with a phenylalanine and/or a tyrosine.

Though a protein typically possesses secondary and/or tertiary structural characteristics that promote positioning of such moiety(s) into a configuration suitable for a metal binding interaction, a less structured peptide sequence often possesses affinity for metal. For example a dipeptide comprising a tyrosine and/or a lysine may have a metal binding property (Sovago, I. and Osz, K., 2006). In another example, peptide sequences comprising a histidine, a tryptophan and/or a cysteine also possess affinity for metal ion [Proteins Labfax (Price, N.C., Ed.), pp. 52-53, 1996]. A metal binding peptide in some case may be associated with possessing secondary structure, such as a His-Xaa-Xaa-Xaa-Xaa-His (SEQ ID No. 225) sequence that may be part of an alpha helix, a His-Xaa-His sequence that may be part of a beta strand, and/or a His-Xaa-Xaa-His (SEQ ID No. 225) sequence that may be part of a beta turn (Matthews, D. J., 1995).

Often a metal binding peptide sequence is used as part of a fusion protein at the C-terminus, the N-terminus, and/or as an internal sequence. For example, a histidine's de-protonated (pKa of about 6) imidazole side chain residue generally possesses the metal binding property, and a peptide polymer comprising one or more histidine residue(s) may be used, for example, alone and/or as part of a fusion protein. In a specific example, a histidine rich polymer peptide sequence (e.g., a poly-histidine, a histidine copolymer), often about 2 to about 20 amino acids long, may be part of a fusion protein and used as an affinity tag to purify the fusion protein due to its ability to bind a transition metal ion (e.g., Co²⁺, Cu²⁺, Ni²⁺, Zn²⁺, Fe₃₊). An example of a histidine rich peptide comprises a peptide alternating in histidines, such as a 6×HN tag, which comprises six repeated His-Asn units (i.e., His-Asn-His-Asn-His-Asn; SEQ ID No. 297). Other examples of a histidine rich polymer peptide sequence that has been used as an affinity tag sequence include a His-Trp dipeptide tag; a plurality of copies of an Ala-His-Gly-His-Arg-Pro (SEQ ID No. 218) sequence; a Pro-His-His-His-His-His-Pro (SEQ ID No. 231) peptide; and/or a His-Trp-His-Trp-His-Trp-His (SEQ ID No. 224) tag (Ljungquist, C. et al. Eur., 1989). Another example of a histidine rich affinity tag comprises a HAT tag sequence Lys-Asp-His-Leu-Ile-His-Asn-Val-His-Lys-Glu-Phe-His-Ala-His-Ala-His-Asn-Lys (SEQ ID No. 242), which is derived from the N terminus of the chicken lactate dehydrogenase. A HAT tag may bind a metal ion, including a conjugated metal ion such as Co²⁺-carboxymethylaspartate [“High Throughput Protein Expression and Purification Methods and Protocols,” (Doyle, S. A., Ed.), pp. 129-132, 2009]. In another example, a His-Trp-His-His-His-Pro tag (SEQ ID No. 233) has been used to bind a fusion protein to a mica surface treated with nickel (Matthews, D. J., 1995).

Various metal-binding peptide sequences from metalloproteins such as a synthetic consensus sequence, as well as synthetic sequences from a peptide expression library, may be used. For example, a synthetic consensus metalopeptidase metal binding peptide sequence generally binds zinc, nickel, cobalt, or manganese, and examples includes a His-Xaa-Xaa-Glu-His (SEQ ID No. 226) consensus sequence derived from various inverzincins' sequences; a His-Glu-Xaa-Xaa-His (SEQ ID No. 227) consensus sequence derived from various zincins', gluzincins', thermolysins', and aspzincins' consensus sequences, with an optional Glu and/or Asp placed distal to the peptide sequence; a His-Xaa-Xaa-Glu (SEQ ID No. 229) consensus sequence derived from various funnelins' sequences; a His-Glu-Xaa-Xaa-His-Xaa-Xaa-Gly-Xaa-Xaa-His (SEQ ID No. 223) and/or a His-Glu-Xaa-Xaa-His-Xaa-Xaa-Gly-Xaa-Xaa-Asp (SEQ ID No. 222) consensus sequences derived from various metzincins' sequences; an Asn-Glu-Xaa-Xaa-Ser (SEQ ID No. 243) consensus sequence derived from various thermolysins' sequences; a Glu-Xaa-Xaa-Ser (SEQ ID No. 93) and/or a Glu-Xaa-Xaa-Gly (SEQ ID No. 209) consensus sequence derived from various cowrins' sequences; or a combination thereof (Gomis-Ruth, F. X., 2008).

In another example, a metal binding sequence may comprise a Xaa-Xaa-His sequence; an Xaa-Xaa-Cys sequence; a Xaa-Xaa-Xaa₍₁₋₅₎-His (SEQ ID No. 210) sequence; a Gly-Gly-Gly-Gly-Gly-His (SEQ ID No. 298) sequence; a Gly-Gly-Gly-Gly-His (SEQ ID No. 299) sequence; a His-Xaa sequence; a Xaa-His sequence (e.g., an N-terminal sequence Xaa-His sequence that may be part of a longer sequence); and/or a Pro-His-Xaa sequence, which often is part of an octapeptide (e.g., a Pro-His-Gly-Gly-Gly-Trp-Gly-Gln; SEQ ID No. 230). A metal binding sequence may be incorporated as part of a larger sequence. For example, a Xaa-Xaa-His motif (e.g., an Ala-Ala-His) may be part of a larger sequence (e.g., an Asp-Ala-His-Xaa; SEQ ID No. 208) (Sovago, I. and Osz, K., 2006).

In a further example, a synthetic consensus metal binding sequence derived from various ferredoxins, rubredoxins and/or Ni—Fe hydrogenases generally comprises a Cys-Xaa-Xaa-Cys-Xaa-Xaa-Cys (SEQ ID No. 211) sequence. An additional metal binding motif comprises a Gly-Met-Thr-Cys-Ala-Ala-Cys (SEQ ID No. 300) from the active site of mercuric ion binding protein (“MerP”). A consensus sequence Glu-Xaa-Xaa-His (SEQ ID No. 214) motif may also bind metal, and may be part of a larger peptide and/or a repeated sequence (Xing, G. and DeRose, V. J., 2001). In an additional example, a prion protein generally comprises a terminal copper binding motif Pro-His-Gly-Gly-Gly-Trp-Gly-Gln (SEQ ID No. 230) (Liu, C. and Xu, H., 2002).

Additional examples of metal binding sequences include a Cys-Xaa₍₀₋₄₎-Cys (SEQ ID No. 213) consensus sequence derived from an aspartate transcarbamoylase, and/or a rubredoxin; a His-Xaa₍₃₋₄₎-Cys (SEQ ID No. 217) motif; and/or a Cys-Xaa-Xaa-Xaa-Xaa-Cys-Xaa-Xaa-Xaa-Xaa-Xaa-Xaa-Xaa-Xaa-His-Xaa-Xaa-Xaa-Cys (SEQ ID No. 215) consensus sequence derived from various CREB-binding proteins (Michel, S. L. J., and Berg, J. M., 2002). Another example includes a Cys-Xaa-Cys-Xaa₍₄₋₆₎-Cys-Xaa-Cys (SEQ ID No. 212) consensus sequence has been identified in about 40 related organisms that may be that binds Zn²⁺ and/or Cu¹⁺ (Pazehoski, P. O., 2008).

In some embodiments, a Met, a Cys, and/or an Asp may be used to substitute a His in a 2^{nd} or 3^{rd} position of an end terminal (e.g., a C-terminal, an N-terminal) amino acid sequence, as such a substitution may produce a sequence with metal binding affinity (Sovago, I. and Osz, K., 2006).

A metal binding motif may function in a plurality of proteinaceous molecules, such as separate proteinaceous molecules that may simultaneously bind a metal ion (e.g., a protein dimer). A proteinaceous molecule may comprise a plurality of metal binding motifs. A proteinaceous molecule comprising a metal binding sequence may be produced by chemical synthesis and/or biological production. For example, a Brassica fuceal (“India mustard”) expressing an Escherichia coli glutamate-cysteine ligase and glutathione synthase that catalyzes production of glutathione, which then enhances production of phytochelatin via a reaction catalyzed by an endogenously expressed glutathione γ-glutamylcysteinyltransferase (“phytochelatin synthatase”). A synthetic metal binding peptide His-Ser-Gln-Lys-Val-Phe (SEQ ID No. 235) identified from a phage-display peptide library has also been expressed in tobacco plants (Mejáre, M. and Billow, L., 2001). In additional examples, a peptide comprising histidine and/or a cysteine such as a Gly-His-His-Pro-His-Gly-Gly-His-His-Pro-His-Gly (SEQ ID No. 220), a Ser-Tyr-His-His-His-His (SEQ ID No. 232), and/or a Cys-Gly-Cys-Cys-Gly-Cys-Gly-Cys-Cys-Gly-Cys-Gly-Cys-Cys-Gly (SEQ ID No. 206) have been recombinantly expressed as fusion proteins (Sousa, C. et al., 1998; Kotrba, P. et al., 1999; Jacobs, F. A. et al., 1989; Mauro, J. M. and Pazirandeh, M., 2000; Kotrba, P. et al., 1999; Pazirandeh, M. et al., 1998; Sousa, C. et al., 1996; Mejáre, M. et al., 1998).

A phytochelatin comprises a peptide typically produced by enzymatic synthesis rather than translation of an mRNA. A phytochelatin generally may bind a heavy metal such as a Ni²⁺, a Cu⁺/²⁺, a Cd²⁺, an Ag⁺, an As³⁺, a Zn²⁺, and/or a Hg²⁺. A phytochelatin may be endogenously produced by cells of algae, an autotrophic plant, some worms, some prokaryotic organisms, and fungi. The sequence of a phytochelatin generally comprises (γ-Glu-Cys)ₙ-Xaa, wherein “n” is about to 1 to about 12, and wherein Xaa comprises a Gly, a gamma-Ala, a Glu, and/or a Ser (Hirata, K. et al., 2005; Mejáre, M. and Billow, L., 2001). The production of a phytochelatin may be induced by contact with a metal cation such as a Bi³⁺, a Cd²⁺, a Hg²⁺, an Ag⁺, a Cu⁺/²⁺, a Ni²⁺, an Au^{+/3+}, a Pb²⁺, and/or a Zn²⁺ cation, which activates γ-glutamylcysteinyl dipeptidyl transpeptidase (“PC synthase”). One or more enzyme(s) (e.g., a glutamate-cysteine ligase, a glutathione synthase, a glutathione γ-glutamylcysteinyltransferase) in the metabolic pathway for the production of a phytochelatin may be recombinantly expressed in various cell types (e.g., a prokaryotic cell, a plant cell) for production of a phytochelatin (Mejáre, M. and Billow, L., 2001; Rauser, W. E., 1995; Vatamaniuk, O. K, et al., 1999; Ha, S.-B., et al., 1999; Clemens, S. et al., 1999; Cobbett, C and Goldsbrough, P., 2002). As with other proteinaceous molecules described herein, a phytochelatin, such as part of a biomolecular composition (e.g., a cell-based particulate material, a purified phytochelatin), may be used as a component of a material formulation. Examples of enzymes involved in phytochelatin production that may be endogenously and/or recombinantly used to produce phytochelatin are described below.

Glutamate-cysteine ligase (EC 6.3.2.2; CAS reg. no. 9023-64-7) catalyzes the reaction: L-glutamate+L-cysteine+ATP=γ-L-glutamyl-L-cysteine+phosphate+ADP. An L-aminohexanoate may be used as a substrate instead of and/or in combination with a glutamate. Glutamate-cysteine ligase producing cells and methods for isolating a glutamate-cysteine ligase from a cellular material and/or a biological source have been described (see, for example, Mackinnon, C. M. et al., 1987; Snoke, J. E. et al., 1953; Mandeles, S. and Bloch, K., 1955).

Glutathione synthase (EC 6.3.2.3; CAS reg. no. 9023-62-5) catalyzes the reaction: γ-L-glutamyl-L-cysteine+ATP+glycine=glutathione+ADP+phosphate. Glutathione synthase producing cells and methods for isolating a glutathione synthase from a cellular material and/or a biological source have been described [see, for example, Law, M. Y. and Halliwell, B., 1986; Macnicol, P. K., 1987).

Glutathione γ-glutamylcysteinyltransferase (EC 2.3.2.15; CAS reg. no. 125390-02-5) catalyzes the reaction: (Glu-Cys)ₙ-Gly+glutathione=+Gly. A glutathione γ-glutamylcysteinyltransferase producing cells and methods for isolating a glutathione γ-glutamylcysteinyltransferase from a cellular material and/or a biological source have been described [see, for example, Grill, E. et al., 1989).

The Tables below lists various exemplary metal binding sequences.

TABLE 1
Exemplary Metal Binding Sequences

| | SEQ | | | |
|---|---|---|---|---|
| | ID | | | |
| Sequence | No. | Protein | Species | Reference |
| Xaa-Xaa-Cys-Cys | 204 | | Synthetic | Theologis, A. et al., 2000. |
| Cys-Cys-Xaa-Cys-Cys | 205 | | Synthetic | Kroczynska, B. et al., 2004. |
| Cys-Gly-Cys-Cys-Gly- | 206 | | Synthetic | Pazirandeh, M. et al., 1998. |
| Cys-Gly-Cys-Cys-Gly- | | | | |
| Cys-Gly-Cys-Cys-Gly | | | | |
| Cys-Gly-Cys-Cys-Gly | 207 | | Synthetic | Pazirandeh, M. et al., 1998. |
| Asp-Ala-His-Xaa | 208 | | Synthetic | Sovago, I. and Osz, K., 2006 |
| Glu-Xaa-Xaa-Gly | 209 | | Synthetic | Gomis-Ruth, F.X, 2008 |
| Xaa(1-8)-His | 210 | | Synthetic | Sovago, I. and Osz, K., 2006. |
| Cys-Xaa-Xaa-Cys- | 211 | | Synthetic | Xing, G. and DeRose, V.J., |
| Xaa-Xaa-Cys | | | | 2001 |
| Cys-Xaa-Cys-Xaa₍₄₋₆₎- | 212 | | Synthetic | Pazehoski, P.O., 2008 |
| Cys-Xaa-Cys | | | | |
| Cys-Xaa(0-4)-Cys | 213 | | Synthetic | Michel, S.L.J., and Berg, J.M., |
| | | | | 2002; Mejáre, M. and Bülow, |
| | | | | L., 2001; Hirata, K. et al., |
| | | | | 2005. |
| Glu-Xaa-Xaa-His | 214 | | Synthetic | Xing, G. and DeRose, V.J., |
| | | | | 2001. |
| Cys-Xaa-Xaa-Xaa- | 215 | | Synthetic | Michel, S.L.J., and Berg, J.M., |
| Xaa-Cys-Xaa-Xaa- | | | | 2002 |
| Xaa-Xaa-Xaa-Xaa- | | | | |
| Xaa-Xaa-His-Xaa- | | | | |
| Xaa-Xaa-Cys | | | | |
| Gln-Cys-Xaa-Lys-Lys- | 216 | | Synthetic | Cobbett, C and Goldsbrough, |
| Gly | | | | 2002 |
| His-Xaa(3-4)-Cys | 217 | | Synthetic | Michel, S.L.J., and Berg, J.M., |
| | | | | 2002 |
| Ala-His-Gly-His-Arg- | 218 | | Synthetic | Ljungquist, C. et al., 1989. |
| Pro | | | | |
| Gly-His-Gly-His-Gly- | 219 | | Synthetic | Skerra, A. et al., 1991. |
| His | | | | |
| Gly-His-His-Pro-His- | 220 | Histidine- | Homo sapiens | Kotrba, P. et al., 1999. |
| Gly-Gly-His-His-Pro- | | rich | | |
| His-Gly | | glycoprotein | | |
| Gly-His-His-Pro-His- | 221 | Histidine- | Homo sapiens | Kotrba, P. et al., 1999. |
| Gly | | rich | | |
| | | glycoprotein | | |
| His-Glu-Xaa-Xaa-His- | 222 | | Synthetic | Gomis-Ruth, F.X, 2008. |
| Xaa-Xaa-Gly-Xaa- | | | | |
| Xaa-Asp | | | | |
| His-Glu-Xaa-Xaa-His- | 223 | | Synthetic | Gomis-Ruth, F.X, 2008. |
| Xaa-Xaa-Gly-Xaa- | | | | |
| Xaa-His | | | | |
| His-Trp-His-Trp-His- | 224 | | Synthetic | Skerra, A. et al., 1991. |
| Trp-His | | | | |
| His-Xaa(0-4)-His | 225 | | Synthetic | Michel, S.L.J., and Berg, J.M., |
| | | | | 2002 |
| His-Xaa-Xaa-Glu-His | 226 | | Synthetic | Gomis-Ruth, F.X, 2008. |
| His-Glu-Xaa-Xaa-His | 227 | | Synthetic | Gomis-Ruth, F.X, 2008. |
| His-His-His-His | 228 | | Synthetic | Hochuli, E. et al., 1988. |
| His-Xaa-Xaa-Glu | 229 | | Synthetic | Gomis-Ruth, F.X, 2008. |
| Pro-His-Gly-Gly-Gly- | 230 | Prion | Homo sapiens | Sovago, I. and Osz, K., 2006 |
| Trp-Gly-Gln | | | | |
| Pro-His-His-His-His- | 231 | | Synthetic | Skerra, A. et al., 1991. |
| His-Pro | | | | |
| Ser-Tyr-His-His-His- | 232 | | Synthetic | Mejáre, M. et al., 1998. |
| His | | | | |
| His-Trp-His-His-His- | 233 | | Synthetic | III, C.R. et al., 1993 |
| Pro | | | | |
| His-Ser-Gln-Lys-Val- | 234 | | Synthetic | Mejáre, M. and Bülow, 2001. |
| Lys | | | | |
| His-Ser-Gln-Lys-Val- | 235 | | Synthetic | Mejáre, M. et al., 1998. |
| Phe | | | | |
| His-His-His-His-His | 236 | | Synthetic | Hochuli, E. et al., 1988. |
| His-His-His-His-His- | 237 | | Synthetic | Hochuli, E. et al., 1988. |
| His | | | | |
| His-His-His-His-His- | 238 | | Synthetic | Knech, S. et al., 2009. |
| His-His | | | | |
| His-His-His-His-His- | 239 | | Synthetic | Ljungquist, C. et al., 1989. |
| His-His-His | | | | |
| His-His-His-His-His- | 240 | | Synthetic | Skerra, A. et al., 1991. |
| His-His-His-His | | | | |
| His-His-His-His-His- | 241 | | Synthetic | Knech, S. et al., 2009. |
| His-His-His-His-His | | | | |
| Lys-Asp-His-Leu-Ile- | 242 | L-lactate | Gallus gallus | Hirota, Y. et al., 1990. |
| His-Asn-Val-His-Lys- | | dehydrogenase | | |
| Glu-Phe-His-Ala-His- | | A | | |
| Ala-His-Asn-Lys | | | | |
| Asn-Glu-Xaa-Xaa-Ser | 243 | | Synthetic | Gomis-Ruth, F.X, 2008. |
| Tyr-Leu-Pro-Lys-Asn- | 250 | | Synthetic | Mejare, M. et al., 1998. |
| Gly | | | | |
| Thr-Thr-Ser-Trp-Val- | 251 | | Synthetic | Mejare, M. et al., 1998. |
| Val | | | | |
| Ser-Ala-Asn-Val-Ile- | 252 | | Synthetic | Mejare, M. et al., 1998. |
| His | | | | |
| Pro-Phe-Lys-Asp-Trp- | 253 | | Synthetic | Mejare, M. et al., 1998. |
| Tyr | | | | |
| Pro-Met-Gly-Val-Ala- | 254 | | Synthetic | Mejare, M. et al., 1998. |
| Ser | | | | |
| Pro-Ile-Phe-Ser-Gly- | 255 | | Synthetic | Mejare, M. et al., 1998. |
| Gly | | | | |
| Phe-Thr-Lys-Phe-Pro- | 256 | | Synthetic | Mejare, M. et al., 1998. |
| Thr | | | | |
| Lys-Ser-Val-Val-Leu- | 257 | | Synthetic | Mejare, M. et al., 1998. |
| Ser | | | | |
| Lys-Ala-Ala-Ser-Asn- | 258 | | Synthetic | Mejare, M. et al., 1998. |
| Ser | | | | |
| Leu-Leu-Leu-Gly-Val- | 259 | | Synthetic | Mejare, M. et al., 1998. |
| Asp | | | | |
| Leu-Leu-Leu-Gly-Leu- | 260 | | Synthetic | Mejare, M. et al., 1998. |
| Asp | | | | |
| Leu-Leu-His-Gly-Leu- | 261 | | Synthetic | Mejare, M. et al., 1998. |
| Asn | | | | |
| Ile-Thr-Thr-Asn-Glu- | 262 | | Synthetic | Mejare, M. et al., 1998. |
| Asn | | | | |
| Ala-Ala-His-His | 263 | | Synthetic | Knech, S. et al. J, 2009. |
| His-Asp-Gly-Arg-Ser- | 264 | | Synthetic | Mejare, M. et al., 1998. |
| Ser | | | | |
| Gly-Val-Ser-Ala-Pro- | 265 | | Synthetic | Mejare, M. et al., 1998. |
| Pro | | | | |
| Glu-Leu-Arg-Pro-His- | 266 | | Synthetic | Mejare, M. et al., 1998. |
| Thr | | | | |
| Arg-Tyr-Thr-Ile-Ala- | 267 | | Synthetic | Mejare, M. et al., 1998. |
| Gly | | | | |
| Arg-Leu-Val-His-Ile- | 268 | | Synthetic | Mejare, M. et al., 1998. |
| Lys | | | | |
| Arg-Gly-Asn-Ser-Leu- | 269 | | Synthetic | Mejare, M. et al., 1998. |
| Arg | | | | |
| Arg-Glu-Leu-Arg-Pro- | 270 | | Synthetic | Mejare, M. et al., 1998. |
| Glu | | | | |
| Arg-Glu-Glu-Leu-Thr- | 271 | | Synthetic | Mejare, M. et al., 1998. |
| Leu | | | | |
| His-Lys-His-His-His- | 272 | | Synthetic | Mejare, M. et al., 1998. |
| Asn | | | | |
| His-His-His-Trp-Leu- | 273 | | Synthetic | Mejare, M. et al., 1998. |
| His | | | | |
| His-His-Ser-His-Pro- | 274 | | Synthetic | Mejare, M. et al., 1998. |
| His | | | | |
| His-His-His-Met-Val- | 275 | | Synthetic | Mejare, M. et al., 1998. |
| His | | | | |
| Tyr-His-His-His-His- | 276 | | Synthetic | Mejare, M. et al., 1998. |
| Thr | | | | |
| His-His-His-Gly-Ala- | 277 | | Synthetic | Mejare, M. et al., 1998. |
| His | | | | |
| His-His-His-His-Gly- | 278 | | Synthetic | Mejare, M. et al., 1998. |
| Arg | | | | |
| Ala-Ala-Ala-His-His | 279 | | Synthetic | Knech, S. et al., 2009. |
| Ala-Ala-Ala-Ala-His- | 280 | | Synthetic | Knech, S. et al., 2009. |
| His | | | | |
| Ala-Ala-Ala-His-Ala- | 281 | | Synthetic | Knech, S. et al., 2009. |
| His | | | | |
| Ala-Ala-His-Ala-Ala- | 282 | | Synthetic | Knech, S. et al., 2009. |
| His | | | | |
| Ala-His-Ala-Ala-Ala- | 283 | | Synthetic | Knech, S. et al., 2009. |
| His | | | | |
| His-Ala-Ala-Ala-Ala- | 284 | | Synthetic | Knech, S. et al., 2009. |
| His | | | | |
| His-Ala-His-Ala-Ala- | 285 | | Synthetic | Knech, S. et al., 2009. |
| His | | | | |
| His-Ala-Ala-His-Ala- | 286 | | Synthetic | Knech, S. et al., 2009. |
| His | | | | |
| His-Glu-Xaa-Xaa-His- | 287 | | Synthetic | Maret, W. J., 2004. |
| Xaa-His | | | | |
| Cys-Xaa-Xaa-Xaa- | 288 | | Synthetic | Maret, W. J., 2004. |
| Xaa-Xaa-Xaa-Xaa- | | | | |
| Xaa-Xaa-Xaa-Xaa- | | | | |
| Xaa-Xaa-Xaa-Xaa- | | | | |
| Xaa-Xaa-Xaa-Xaa- | | | | |
| Xaa-His | | | | |
| His-Xaa-Xaa-Xaa-His- | 289 | | Synthetic | Maret, W.J., 2004. |
| Xaa-Xaa-Xaa-Xaa- | | | | |
| Xaa-His | | | | |
| Cys-Leu-Glu-Glu-His | 290 | | Synthetic | Regan, L. and Clarke, N.D., |
| | | | | 1990 |
| Cys-Leu-Lys-Glu-His | 291 | | Synthetic | Regan, L. and Clarke, N.D., |
| | | | | 1990. |
| Glu-Xaa-Xaa-Ser | 292 | | Synthetic | Gomis-Ruth, F.X, 2008. |
| Xaa-Xaa-Asp-Xaa | 293 | | Synthetic | Sovago, I. and Osz, K., 2006. |
| Xaa-Xaa-Cys-Xaa | 294 | | Synthetic | Sovago, I. and Osz, K., 2006. |
| Xaa-Xaa-His-Xaa | 295 | | Synthetic | Sovago, I. and Osz, K., 2006. |
| Xaa-Xaa-Met-Xaa | 296 | | Synthetic | Sovago, I. and Osz, K., 2006. |

TABLE 2
Additional Exemplary Metal Binding Sequences

| | SEQ | | | |
|---|---|---|---|---|
| | ID | | | |
| Sequence | No. | Protein | Species | Reference |
| Gly-His | N/A | | Synthetic | Farkas, E. et al., 1984. |
| Gly-His-Gly | N/A | | Synthetic | Farkas, E. et al., 1984. |
| Gly-Gly-His | N/A | | Synthetic | Farkas, E. et al., 1984. |
| His-Xaa | N/A | | Synthetic | Sovago, I. and Osz, K., |
| | | | | 2006. |
| His-Trp | N/A | | Synthetic | Smith, M. et al., 1987. |
| His-Gly-His | N/A | | Synthetic | Smith, M. et al., 1987. |
| His-Tyr | N/A | | Synthetic | Smith, M. C. et al., 1988. |
| Pro-His-Xaa | N/A | | Synthetic | Sovago, I. and Osz, K., |
| | | | | 2006. |
| His-His | N/A | | Synthetic | Hochuli, E. et al., 1988. |
| His-His-His | N/A | | Synthetic | Hochuli, E. et al., 1988. |
| Ala-His-His | N/A | | Synthetic | Knech, S. et al., 2009. |

6. Anti-Biological Peptides and Polypeptides

Some proteinaceous molecules for use herein may comprise an anti-biological proteinaceous sequence that may be active against one or more biological entity(s) that may be same or different (e.g., a terrestrial biological cell) as described for a metal-binding and/or anti-biofouling proteinaceous molecule. Examples of an anti-biological proteinaceous molecule include the peptide sequences described in U.S. Pat. Nos. 6,020,312; 5,885,782; and 5,602,097, and patent application Ser. Nos. 10/884,355 and 11/368,086. These anti-biological peptides include those of SEQ ID No. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, or a combination thereof. For example, SEQ ID Nos. 1-47, which comprise sequences from a peptide library, may be used individually (e.g., SEQ ID No. 14, SEQ ID No. 41), or in a combination (e.g., a mixture of SEQ ID Nos. 25-47). These sequences establish a number of chemical compositions which possess anti-biological (e.g., anti-cellular, anti-viral) activity. Exemplary peptides that are expected to demonstrate anti-biological activity in a material formulation (e.g., a coating) are listed in the Table below, and one or more such peptide sequences may be used in any combination, including any combination of other proteinaceous sequence(s) (e.g., metal binding sequences), biomolecular composition(s), and/or anti-biological agent(s).

TABLE 3
Anti-Biological Peptides

| | | Seq. | Exemplary | |
|---|---|---|---|---|
| Name | Source | ID(s) | Activity | Reference |
| | Synthetic | 1-47 | Fungi | U.S. Pat. No. |
| | | | | 5,885,782 |
| Tachystatin A | Horseshoe | 48 | Gram+ & | Fujitani (2002) |
| | Crab | | Gram−, Fungi | |
| Androctonin | Androctonus | 49 | Gram+ & | Mandard (1999) |
| | Australis | | Gram−, Fungi | |
| Tritrpticin | Synthetic | 50 | Gram+ & | Schibli (1999) |
| | | | Gram−, Fungi | |
| HNP-3 | Human | 51 | Gram+ & | Hill (1991) |
| Defensin | | | Gram−, Virus, | |
| | | | Fungi | |
| Anti-fungal | Phytolacca | 52 | Fungi | Gao (2001) |
| protein 1 | Americana | | | |
| (pafp-s) | | | | |
| Magainin 2 | Synthetic | 53 | Gram+ & | Hara (2001) |
| | construct | | Gram−, Fungi | |
| Indolicidin | Bos Taurus | 54 | Gram+ & | Rozek (2000) |
| | | | Gram−, Virus, | |
| | | | Fungi | |
| Defensin | Heliothis | 55 | Fungi | Lamberty (2001) |
| heliomicin | virescens | | | |
| Defensin | Heliothis | 56 | Gram+ & | Lamberty (2001) |
| heliomicin | virescens | | Gram−, Fungi | |
| Sativum | Seed of Pea | 57 | Fungi | Almeida (2002) |
| defensin 1 | | | | |
| (psd1) | | | | |
| Gomesin | Synthetic | 58 | Gram+ & | Mandard (2002) |
| | | | Gram−, Fungi, | |
| | | | Mammalian | |
| | | | cells | |
| Lactoferricin | Bovine | 59 | Gram+ & | Hwang (1998) |
| B | | | Gram−, Virus, | |
| | | | Fungi, Cancer | |
| | | | cells | |
| PW2 | Synthetic | 60 | Fungi | Tinoco (2002) |
| Hepcidin 20 | Human | 61 | Fungi | Hunter (2002) |
| Hepcidin 25 | Human | 62 | Fungi | Hunter (2002) |
| AC-AMP2 | Amaranthus | 63 | Gram+, Fungi | Martins (1996) |
| | caudatus | | | |
| NK-Lysin | Sus scrofa | 64 | Gram+ & | Liepinsh (1997) |
| | | | Gram−, Fungi | |
| Magainin 2 | African | 65 | Gram+ & | Gesell (1997) |
| | clawed | | Gram−, Fungi, | |
| | frog | | cancer cells | |
| Melittin B | Honey bee | 66 | Gram+ & | Eisenberg |
| | venom | | Gram−, Fungi, | |
| | | | Mammalian | |
| | | | cells | |
| Thanatin | Podisus | 67 | Gram+ & | Mandard (1998) |
| | maculiventris | | Gram−, Fungi | |
| Antimicrobial | Common | 68 | Gram+ & | Michalowski |
| peptide 1 | ice plant | | Gram−, Fungi | (1998) |
| Melanotropin | Bovine | 69 | Gram+, Fungi | Cutuli (2000) |
| alpha (Alpha- | | | | |
| MSH) | | | | |
| CORTICOSTATIN | Rabbit | 70 | Gram+ & | Selsted (1988) |
| III (MCP-1) | | | Gram−, Virus, | |
| | | | Fungi | |
| CORTICOSTATIN | Rabbit | 71 | Gram+ & | Selsted (1988) |
| III (MCP-1) | | | Gram−, Virus, | |
| | | | Fungi | |
| Cecropin B | Chinese oak | 72 | Gram+ & | Qu (1982) |
| | silk moth | | Gram−, Fungi | |
| Seminalplasmin | Bovine | 73 | Gram+ & | Theil (1983) |
| | | | Gram−, Fungi, | |
| | | | Mammalian | |
| | | | cells | |
| NP-3A defensin | Rabbit | 74 | Gram+ & | Zhu (1992) |
| | | | Gram−, Virus, | |
| | | | Fungi | |
| HNP-1 Defensin | Human | 75 | Gram+ & | Zhang (1992) |
| | | | Gram−, Virus, | |
| | | | Fungi | |
| HNP-2 Defensin | Human | 76 | Gram+ & | Selsted (1989) |
| | | | Gram−, Virus, | |
| | | | Fungi | |
| HNP-4 Defensin | Human | 77 | Gram+ & | Wilde (1989) |
| | | | Gram−, Fungi | |
| Histatin 5 | Human | 78 | Gram+ & | Raj (1998) |
| | | | Gram−, Fungi | |
| Histatin 3 | Human | 79 | Gram+ & | Oppenheim |
| | | | Gram−, Fungi | (1988) |
| Histatin 8 | | 80 | Gram+ & | Yin (2003) |
| | | | Gram−, Fungi | |
| Tracheal | Bovine | 81 | Gram+ & | Zimmermann |
| antimicrobial | | | Gram−, Fungi | (1995) |
| peptide | | | | |
| AMP1 (MJ-AMP1) | Garden four- | 82 | Gram+, Fungi | Cammue (1992) |
| | o'clock | | | |
| AMP2 (MJ-AMP2) | Garden four- | 83 | Gram+, Fungi | Cammue (1992) |
| | o'clock | | | |
| MBP-1 | Maize | 84 | Gram+ & | Duvick (1992) |
| | | | Gram−, Fungi | |
| AFP2 | Rape | 85 | Fungi | Terras (1993) |
| AFP1 | Turnip | 86 | Fungi | Terras (1993) |
| AFP2 | Turnip | 87 | Fungi | Terras (1993) |
| ADENOREGULIN | Two coloured | 88 | Gram+ & | Mor (1994) |
| | leaf frong | | Gram−, Fungi | |
| Protegrin 2 | Pig | 89 | Gram+ & | Kokryakov (1993) |
| | | | Gram−, Virus, | |
| | | | Fungi | |
| Protegrin 3 | Pig | 90 | Gram+ & | Kokryakov (1993) |
| | | | Gram−, Virus, | |
| | | | Fungi | |
| Histatin 1 | Crab eating | 91 | Gram+ & | Xu (1990) |
| | macaque | | Gram−, Fungi | |
| Peptide PGQ | African | 92 | Gram+ & | Moore (1991) |
| | clawed frog | | Gram−, Fungi | |
| Ranalexin | Bull frog | 93 | Gram+ & | Halverson (2000) |
| | | | Gram−, Fungi | |
| GNCP-2 | Guinea pig | 94 | Gram+ & | Nagaoka (1991) |
| | | | Gram−, Virus, | |
| | | | Fungi | |
| Protegrin 4 | Pig | 95 | Gram+ & | Zhao (1994) |
| | | | Gram−, Virus, | |
| | | | Fungi | |
| Protegrin 5 | Pig | 96 | Gram+ & | Zhao (1995) |
| | | | Gram−, Virus, | |
| | | | Fungi | |
| BMAP-27 | Bovine | 97 | Gram+ & | Skerlavaj (1996) |
| | | | Gram−, Fungi | |
| BMAP-28 | Bovine | 98 | Gram+ & | Skerlavaj (1996) |
| | | | Gram−, Fungi | |
| Buforin I | Asian toad | 99 | Gram+ & | Park (1996) |
| | | | Gram−, Fungi | |
| Buforin II | Asian toad | 100 | Gram+ & | Yi (1996) |
| | | | Gram−, Fungi | |
| BMAP-34 | Bovine | 101 | Gram+ & | Scocchi (1997) |
| | | | Gram−, Fungi | |
| Tricholongin | Trichoderma | 102 | Gram+ & | Rebuffat (1991) |
| | longibrachiatum | | Gram−, Fungi | |
| Dermaseptin 1 | Sauvage's | 103 | Gram+ & | Mor (1994) |
| | leaf frog | | Gram−, Fungi | |
| Pseudo-hevein | Para rubber | 104 | Fungi | Soedjanaatmadja |
| (Minor hevin) | tree | | | (1994) |
| Gaegurin-1 | Wrinkled frog | 105 | Gram+ & | Park (1994) |
| | | | Gram−, Fungi | |
| Skin peptide | Two-colored | 106 | Gram+ & | Mor (1994) |
| tyrosine-tyrosine | leaf frog | | Gram−, Fungi | |
| Penaeidin-1 | Penoeid shrimp | 107 | Gram+ & | Destoumieux |
| | | | Gram−, Fungi | (2000) |
| Neutrophil | Golden hamster | 108 | Gram+, Fungi | Mak (1996) |
| defensin 1 | | | | |
| (HANP-1) | | | | |
| Neutrophil | Golden hamster | 109 | Gram+, Fungi | Mak (1996) |
| defensin 3 | | | | |
| (HANP-3) | | | | |
| Misgurin | Oriental | 110 | Gram+ & | Park (1997) |
| | weatherfish | | Gram−, Fungi | |
| PN-AMP | Japenese | 111 | Gram+, Fungi | Koo (1998) |
| | morning glory | | | |
| Histone H2B-1 | Rainbow trout | 112 | Gram+ & | Robinette (1998) |
| (HLP-1) | | | Gram−, Fungi | |
| (Fragment) | | | | |
| Histone H2b-3 | Rainbow trout | 113 | Fungi | Robinette (1998) |
| (HLP-3) | | | | |
| (Fragment) | | | | |
| Neutrophil | Rhesus | 114 | Gram+ & | Tang (1999) |
| defensin 2 | macaque | | Gram−, Fungi | |
| (RMAD-2) | | | | |
| Termicin | Pseudacanthotermes | 115 | Gram+, Fungi | Lamberty (2001) |
| | spiniger | | | |
| Spingerin | Pseudacanthotermes | 116 | Gram+ & | Lamberty (2001) |
| | spiniger | | Gram−, Fungi | |
| Aurein 1.1 | Southern bell frog | 117 | Gram+ & | Rozek (2000) |
| | | | Gram−, Fungi | |
| Ponericin G! | Ponerine ant | 118 | Gram+ & | Orivel (2001) |
| | | | Gram−, Fungi | |
| Brevinin-1BB | Rio Grande | 119 | Gram+ & | Goraya (2000) |
| | leopard frog | | Gram−, Fungi | |
| Ranalexin-1CB | Gree frog | 120 | Gram+ & | Halverson (2000) |
| | | | Gram−, Fungi | |
| Ranatuerin-2CA | Green frog | 121 | Gram+ & | Halverson (2000) |
| | | | Gram−, Fungi | |
| Ranatuerin-2CB | Green frog | 122 | Gram+ & | Halverson (2000) |
| | | | Gram−, Fungi | |
| Ginkbilobin | Ginkgo | 123 | Gram+ & | Wang (2000) |
| | | | Gram−, Virus, | |
| | | | Fungi | |
| Alpha-basrubrin | Malabar spinach | 124 | Virus, Fungi | Wang (2001) |
| (Fragment) | | | | |
| Pseudin 1 | Paradoxical frog | 125 | Gram+ & | Olson (2001) |
| | | | Gram−, Fungi | |
| Parabutoporin | Scorpion | 126 | Gram+ & | Moerman (2002) |
| | | | Gram−, Fungi, | |
| | | | Mammalian | |
| | | | cells | |
| Opistoporin 1 | African yellow leg | 127 | Gram+ & | Moerman (2002) |
| | scorpion | | Gram−, Fungi, | |
| | | | Mammalian | |
| | | | cells | |
| Opistoporin 2 | African yellow leg | 128 | Gram+ & | Moerman (2002) |
| | scorpion | | Gram−, Fungi, | |
| | | | Mammalian | |
| | | | cells | |
| Histone H2A | Rainbow trout | 129 | Gram+, Fungi | Fernandes (2002) |
| (fragment) | | | | |
| Dolabellanin B2 | Sea hare | 130 | Gram+ & | Iijima (2002) |
| | | | Gram−, Fungi | |
| Cecropin A | Nocutuid moth | 131 | Gram+ & | Bulet (2002) |
| | | | Gram−, Fungi | |
| HNP-5 Defensin | Human | 132 | Gram+ & | Jones (1992) |
| | | | Gram−, Fungi | |
| HNP-6 Defensin | Human | 133 | Gram+ & | Jones (1993) |
| | | | Gram−, Fungi | |
| Holotricin 3 | Holotrichia | 134 | Fungi | Lee (1995) |
| | diomphalia | | | |
| Lingual | Bovine | 135 | Gram+ & | Schonwetter |
| antimicrobial | | | Gram−, Fungi | (1995) |
| peptide | | | | |
| RatNP-3 | Rat | 136 | Gram+ & | Yount (1995) |
| | | | Gram−, Virus, | |
| | | | Fungi | |
| GNCP-1 | Guinea pig | 137 | Gram+ & | Nagaoka (1993) |
| | | | Gram−, Virus, | |
| | | | Fungi | |
| Penaeidin-4a | Penoeid shrimp | 138 | Gram+ & | Destoumieux |
| | | | Gram−, Fungi | (2000) |
| Hexapeptide | Bovine | 139 | Gram+ & | Vogle (2002) |
| | | | Gram−, Virus, | |
| | | | Fungi, Cancer | |
| | | | cells | |
| P-18 | | 140 | Gram+ & | Lee (2002) |
| | | | Gram−, Fungi, | |
| | | | Cancer cells | |
| MUC7 20- Mer | Human | 141 | Gram+ & | Bobek (2003) |
| | | | Gram−, Fungi | |
| Nigrocin 2 | Rana | 142 | Gram+ & | Park (2001) |
| | nigromaculata | | Gram−, Fungi | |
| Nigrocin 1 | Rana | 143 | Gram+ & | Park (2001) |
| | nigromaculata | | Gram−, Fungi | |
| Lactoferrin (Lf) | | 144 | Fungi | Ueta (2001) |
| peptide 2 | | | | |
| Ib-AMP3 | Impatiens | 145 | Gram+, Fungi | Ravi (1997) |
| | balsamina | | | |
| Ib-AMP4 | Impatiens | 146 | Gram+ Fungi | Ravi (1997) |
| | balsamina | | | |
| Dhvar4 | Synthesis | 147 | Gram+ & | Ruissen (2002) |
| | | | Gram−, Fungi | |
| Dhvar5 | Synthesis | 148 | Gram+ & | Ruissen (2002) |
| | | | Gram−, Fungi | |
| | Synthetic | 149-196 | Fungi | U.S. Application |
| | | | | Ser. No. 10/601,207 |
| | Synthetic | 197-199 | Gram+ & | U.S. Application |
| | | | Gram−, Fungi | Ser. No. 10/601,207 |

In some embodiments, a relatively variable composition (e.g., “XXXXRF”; SEQ ID No. 1) may be described as, for example, an anti-biological proteinaceous composition, even though it may be possible that not every proteinaceous sequence encompassed by that general sequence possesses the same or any anti-biological activity. A proteinaceous composition (e.g., a peptide composition) may exhibit variable anti-biological abilities to, for example, prevent and/or inhibit growth as adjudged by the minimal inhibitory concentrations (MIC mg/ml) and/or the concentrations necessary to inhibit growth of fifty percent of a population of cells (e.g., a fungal spore, a cell, a mycelia) (IC₅₀ mg/ml). For example, a peptide of about 8 to about 10 amino acid residues long may also have the property of inhibiting the growth of bacteria, including disease-causing bacteria (e.g., hospital environment bacterial, antibiotic resistant bacteria) such as a Staphylococcus and a Streptococcus. In a further example, a peptide sequence such as SEQ ID Nos. 6, 7, 8, 9, and/or 10, may act on a cell such as a bacterium and a fungus. In a specific example, a peptide sequence such as SEQ ID Nos. 41, 197, 198, and 199, can inhibit growth of an Erwinia amylovora, an Erwinia carotovora, an Escherichia coli, an Ralstonia solanocerum, an Staphylococcus aureus, and/or an Streptococcus faecalis in standard media at IC₅₀'s of between about 10 to about 1100 mg/ml and MIC's of between about 20 to about 1700 mg/ml. However, in general embodiments, it is contemplated that the anti-biological proteinaceous compositions may have biocidal and/or biostatic activity for various organisms.

For the purposes of preparing and using a proteinaceous molecule as an active anti-biological agent, such as an anti-biological agent used in a material formulation (e.g., a paint, a coating composition), it may not be necessary to understand the mechanism by which the desired anti-biological effect is exerted on a biological entity. For example, a biomolecular composition may comprise one or more peptide(s), polypeptide(s), protein(s) and/or enzyme(s), wherein one or more proteinaceous molecules may be selected for a mixture due to related anti-biological activity(s), regardless of the mechanism of anti-biological activity. However, for example, possible modes of action of a peptide, a polypeptide, and/or a protein, by which they exert their anti-biological effect(s) may include, for example, destabilizing a cellular membrane (e.g., perturb membrane functions responsible for osmotic balance); a disruption of macromolecular synthesis (e.g., cell wall biosynthesis) and/or metabolism; disruption of appressorium formation; or a combination thereof. (see, for example, Fiedler, H. P., et al. 1982; Isono, K. and S. Suzuki. 1979; Zasloff, M. 1987; U.S. patent application Ser. No. 10/601,207).

In certain instances, proteinaceous molecule (e.g., a peptide) may have a completely defined sequence. For example, an anti-biological (e.g., anti-fungal) peptidic agent may comprise a single peptide of a defined sequence (e.g., the hexapeptide of SEQ ID No. 198, SEQ ID No. 41, SEQ ID No. 197, SEQ ID No. 198, SEQ ID No. 199, etc.). However, it is not necessary for a proteinaceous composition (e.g., a peptide), that may possess a demonstrable activity (e.g., antibiotic activity, antifungal activity), to be completely defined as to each residue. For example, an alternative to using one or more isolated anti-microbial peptides as a peptide composition, the peptide composition may instead comprise a mixture of peptides (e.g., an aliquot of a peptide library, a mixture of isolated peptides). In such an example, the peptide composition comprising a mixture of peptides may comprise at least one active peptide (e.g., a peptide having anti-biological activity). In another example, a peptide composition may comprise an active (e.g., an anti-biological) peptide, wherein the peptide composition may be impure to the extent that the peptide composition may comprise one or more peptides of undefined and/or partly defined sequence which may or may not have activity. In a further example, a mixed proteinaceous composition (e.g., a mixed polypeptide composition) may be used treat (e.g., reduce infestation, prevent infestation) a biological entity target with lower concentrations of numerous active additives (e.g., a plurality of active peptides) rather than a higher concentration of a single chemical composition (e.g., a single polypeptide sequence); a mixed proteinaceous composition may be used to treat an array of targets (e.g., a plurality of target organisms) each with a different causative agent; or combination thereof. In certain embodiments, a proteinaceous composition (e.g., a peptide mixture, a synthetic peptide combinatorial library) comprises an equimolar mixture of proteinaceous molecules (e.g., an equimolar mixture of peptides).

In some embodiments, at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more such as to about 10,000 amino acids) of the amino acid residue(s) (e.g., an N-terminal amino acid residue, a C-terminal amino acid residue) is defined for a proteinaceous molecule in a proteinaceous molecule mixture (e.g., a peptide mixture such as a peptide library). For example, the peptidic agent may comprise a peptide library aliquot comprising a mixture of peptides in which at least two, three and/or four or more of the N-terminal amino acid residues are defined. In some aspects wherein one or more amino acid residues(s) are defined for a proteinaceous molecule (e.g., a peptide) in a mixture, the amino acid residue(s) may be in common for a plurality of proteinaceous molecules (e.g., for each peptide) in the mixture. In some aspects, a proteinaceous composition comprises one or more variable amino acid residue(s) (“mixed peptide composition”) and such a mixed proteinaceous composition (e.g., a peptide mixture, a peptide library) may be selected for use due to the increased cost of testing and/or the cost of producing a completely defined proteinaceous molecule (e.g., an defined antibiotic peptide).

For example, the sequence of a peptide may be defined for only certain of the terminal (e.g., C-terminal amino acid residues, N-terminal amino acid residues) leaving the remaining amino acid residues defined as equimolar ratios. In a further example, certain of the peptides of SEQ ID Nos. 1 to 203 have somewhat variable amino acid compositions. Thus, in certain aspects, in each aliquot of the SPCL comprising a given SEQ ID Nos. having a variable residue, the variable residue(s) may each be uniformly represented in equimolar amounts by one of nineteen different naturally-occurring amino acids in one or the other stereoisomeric form. However, the variable residue(s) may be rapidly defined using the method in the art and/or described in one or more of U.S. Pat. Nos. 6,020,312; 5,885,782; and 5,602,097, and patent application Ser. Nos. 10/884,355 and 11/368,086 to identify peptide(s) that possess activity (e.g., controlling fungal growth).

For example, in the cited patents it was demonstrated that peptides encompassed by the C-terminal sequence “XXXXRF” (SEQ ID No. 1) exhibited antifungal activity for a wide spectrum of fungi. In another example of peptide assaying and screening, for the identification of antifungal peptides encompassed by the general sequence “XXXXRF” (SEQ ID No. 1) parent composition of antifungal activity, “XXXLRF” (SEQ ID No. 9) peptides mixtures were found in the next round of identification and screening to exhibit antibiotic activity (also disclosed in U.S. Pat. Nos. 6,020,312; 5,885,782; and 5,602,097, and patent application Ser. Nos. 10/884,355 and 11/368,086). Similarly to the parent composition “XXXXRF” (SEQ ID No. 1), the “XXXLRF” (SEQ ID No. 9) peptides may have a mixed equimolar array of peptides representing the same nineteen amino acid residues, some of which may have anti-biological (e.g., antifungal activity) and some of which may not have such activity. Overall, however, the “XXXLRF” (SEQ ID No. 9) peptide composition comprises an anti-biological agent. This process may be carried out to the point where completely defined peptide(s) are produced and assayed for a desired anti-biological activity (i.e., anti-fungal activity). As a result, and as was accomplished for the representative peptide “FHLRF” (SEQ ID No. 31), all amino acid residues in a six residue peptide could be defined.

7. Anti-Biological Enzymes

In many aspects, an anti-biological agent that may be used herein comprises an enzyme (e.g., an anti-microbial enzyme, an anti-fungal enzyme, an anti-alga enzyme, an anti-bacterial enzyme, anti-mildew enzyme, an anti-fouling enzyme, etc.) that may catalyze a reaction. For example, an enzyme may promote cleavage of a chemical bond in a biological entity such as biological cell wall, a viral proteinaceous molecule, and/or a cellular membrane component (e.g., a viral envelope component). In other embodiments, an anti-biological proteinaceous molecule (e.g., a peptide) may compromise a cell wall and/or cellular membrane to allow for cell wall and/or viral proteinaceous molecule disruption by another anti-biological agent (e.g., an anti-biological enzyme, a chemical preservative) for an additive and/or synergistic effect. In an example, an anti-biological proteinaceous agent (e.g., ProteCoat™, a metal-binding peptide) may be efficacious against a Gram positive organism, and a combination with an anti-biological enzyme (e.g., an anti-fouling enzyme) and/or a non-biomolecular anti-biological agent (e.g., a chemical biocide) may demonstrates additive or synergistic anti-biological activity. In another example, a material formulation comprising a lipolytic enzyme such as a phospholipase and/or a cholesterol esterase that acts to compromise the integrity of a cell membrane, may allow ease of access for one or more enzyme(s) that degrade cell wall and/or viral proteinaceous coat component(s), and/or a non-biomolecular preservative to act in a biocidal and/or a biostatic function as well (e.g., acts against a cell component).

In many embodiments, an enzyme that possesses an anti-biological activity comprises a hydrolase (EC 3). In specific embodiments, the enzyme comprises a glycosylase (EC 3.2). In more specific embodiments, the enzyme comprises a glycosidase (EC 3.2.1), which comprises an enzyme that hydrolyses an O-glycosyl compound, a S-glycosyl compound, or a combination thereof. In particular aspects, the glycosidase acts on an O-glycosyl compound, and examples of such an enzyme include a lysozyme, an agarase, a cellulose, a chitinase, or a combination thereof. In other embodiments, an anti-biological enzyme acts on a cell wall, a viral proteinaceous molecule, and/or a cellular membrane component, and examples of such enzymes include a lysozyme, a lysostaphin, a libiase, a lysyl endopeptidase, a mutanolysin, a cellulase, a chitinase, an α-agarase, an β-agarase, a N-acetylmuramoyl-L-alanine amidase, a lytic transglycosylase, a glucan endo-1,3-β-D-glucosidase, an endo-1,3(4)-β-glucanase, a β-lytic metalloendopeptidase, a 3-deoxy-2-octulosonidase, a peptide-N4-(N-acetyl-β-glucosaminyl)asparagine amidase, a mannosyl-glycoprotein endo-β-N-acetylglucosaminidase, a ι-carrageenase, a κ-carrageenase, a λ-carrageenase, an α-neoagaro-oligosaccharide hydrolase, an endolysin, an autolysin, a mannoprotein protease, a glucanase, a mannose, a zymolase, a lyticase. a lipolytic enzyme, or a combination thereof. A commercially available enzyme may be used, such as, for example, a Viscozyme L carbohydrase produced from an Aspergillus spp. (Novozymes).

a. Lysozymes

Lysozyme (EC 3.2.1.17; CAS registry number: 9001-63-2) catalyzes the reaction: in a peptidoglycan, hydrolyzes a (1,4)-β-linkage between N-acetylmuramic acid and a N-acetyl-D-glucosamine; in a chitodextrin (a polymer of (1,4)-β-linked N-acetyl-D-glucosamine monomers), hydrolyzes the (1,4)-β-linkage. A lysozyme demonstrates endo-N-acetylmuramidase activity, and may cleave a glycan comprising linked peptides, but has little or no activity toward a glycan that lack linked peptide. Lysozyme producing cells and methods for isolating a lysozyme from a cellular material and/or a biological source have been described [see, for example, Blade, C. C. F. et al., 1967a; Blake, C. C. F. et al., 1967b; Jolles, P., 1969; Rupley, J. A., 1964; Holler, H., et al., 1975; Canfield, R. E., 1963; Davies, R. C., et al., 1969), and may be used in conjunction with the disclosures herein. Another example of a lysozyme comprises a commercially available lysozyme (e.g., Sigma Aldrich). Structural information for a wild-type lysozyme and/or a functional equivalent amino acid sequence for producing a lysozyme and/or a functional equivalent include Protein database bank entries: 1021, 1031, 1041, 1071, 1081, 1091, 1101, 1111, 1121, 1131, 1141, 1151, 1161, 1181, 1191, 1201, 1221, 1231, 1251, 1261, 1271, 1281, 1291, 1301, and 1711. Nucleotide and protein sequences for a lysozyme from various organisms are available via database such as, for example, KEGG. Examples of lysozyme and/or a functional equivalent KEEG sequences for production of wild-type and/or a functional equivalent nucleotide and protein sequence include: HSA—4069(LYZ); PTR—450190(LYZ); MCC—718361(LYZ); MMU—17105(Lyz2) 17110(Lyz1); RNO—25211(Lyz2); DPO—Dpse_GA11118 Dpse_GA20595; AGA—AgaP_AGAP005717 AgaP_AGAP007343 AgaP_AGAP007344 AgaP_AGAP007345 AgaP_AGAP007347 AgaP_AGAP007385; and/or PPH—Ppha_0875Protein.

b. Lysostaphins

Lysostaphin (EC 3.4.24.75; CAS registry number: 9011-93-2) catalyzes the reaction: in a staphylococcal (e.g., S. aureus) peptidoglycan, hydrolyzes a -GlyGly- bond in a pentaglycine inter-peptide link (e.g., cleaves the polyglycine cross-links in the peptidoglycan layer of the cell wall of a Staphylococcus sp.). A lysostaphin typically comprises a zinc-dependent, 25-kDa endopeptidase with an activity optimum of about pH 7.5. Lysostaphin producing cells (e.g., Staphylococcus simulans, ATCC 67080, 69764, 67079, 67076, and 67078) and methods for isolating a lysostaphin from a cellular material and/or a biological source have been described [see, for example, Recsei, P. A., et al., 1987; Thumm, G. and Gotz, F. 1997; Trayer, H. R., and Buckley, C. E., 1970; Browder, H. P., et al.., 19, 383, 1965; Baba, T. and Schneewind, 1996], and may be used in conjunction with the disclosures herein. An example of a lysostaphin comprises a commercially available lysostaphin (e.g., Sigma Aldrich).

Structural information for a wild-type lysostaphin and/or a functional equivalent amino acid sequence for producing a lysostaphin and/or a functional equivalent include Protein database bank entries: 1QWY, 2B0P, 2B13, and/or 2B44. Examples of a lysostaphin and/or a functional equivalent KEEG sequences for production of wild-type and/or a functional equivalent nucleotide and protein sequence include: HAR: HEAR2799; SAU: SA0265(lytM); SAV: SAV0276(lytM); SAW: SAHV 0274(lytM); SAM: MW0252(lytM); SAR: SAR0273(lytM); and/or AM1 B0175.

c. Libiases

Libiase comprises an enzyme obtained from Streptomyces fulvissimus (e.g., Streptomyces fulvissimus TU-6) that it typically used to promote the lysis of Gram-positive bacteria (e.g., a Lactobacillus, an Aerococcus, a Listeria, a Pneumococcus, a Streptococcus). A libiase possesses a lysozyme and a β-N-acetyl-D-glucosaminidase activity, with activity optimum of about pH 4, and a stability optimum of about pH 4 to about pH 8. Commercial preparations of a libiase are available (Sigma-Aldrich). Libiase producing cells and methods for isolating a libiase from a cellular material and/or a biological source have been described (see, for example, Niwa et al. 2005; Ohbuchi, K. et al., 2001), and may be used in conjunction with the disclosures herein.

d. Lysyl Endopeptidases

Lysyl endopeptidase (EC 3.4.21.50; CAS registry number: 123175-82-6) catalyzes the peptide cleavage reaction: at a Lys, including -LysPro-. In many embodiments, the lysyl endopeptidase comprises a (trypsin family) family 51 peptidase. Lysyl endopeptidase producing cells and methods for isolating a lysyl endopeptidase from a cellular material and/or a biological source (e.g., Achromobacter lyticus—ATCC 21457; Lysobacter enzymogenes ATCC 29488, 29487, 29486, Pseudomonas aeruginosa—ATCC 29511, 21472) have been described (see, for example, Ahmed et al, 2003; Chohnan et al. 2002; Elliott, B. W. and Cohen, C. 1986; Ezaki, T and Suzuki, S., 1982; Jekel, P. A., et al., 1983; Li et al. 1998; Masaki, T. et al. 1981a; Masaki, T. et al., 1981b; Ohara, T. et al., 1989; Tsunasawa, S. et al., 1989), and may be used in conjunction with the disclosures herein. An example of a lysyl endopeptidase comprises a 27 kDa “achromopeptidase” and a achromopeptidase is commercially available (e.g., Sigma Aldrich; Wako Pure Chemical Industries, Ltd.). Structural information for a wild-type lysyl endopeptidase and/or a functional equivalent amino acid sequence for producing a lysyl endopeptidase and/or a functional equivalent include Protein database bank entries: larb and/or larc. Examples of a lysyl endopeptidase and/or a functional equivalent KEEG sequences for production of wild-type and/or a functional equivalent nucleotide and protein sequence include: SRU: SRU 1622.

e. Mutanolysins

Mutanolysin (EC 3.4.99.-) comprises a 23 kD N-acetyl muramidase obtained from Streptomyces globisporus (e.g., ATCC 21553). A mutanolysin catalyzes the reaction: in a cell wall peptidoglycan-polysaccharide, cleavage of a N-acetylmuramyl-β(1-4)-N-acetylglucosamine bond. Examples of cells that mutanolysin acts on include Gram positive bacteria (e.g., a Listeria, a Lactobacillus, a Lactococcus). Mutanolysin producing cells and methods for isolating a mutanolysin from a cellular material and/or a biological source have been described (see, for example, Assaf, N. A., and Dick, W. A., 1993; Calandra, G. B., and Cole, R. M., 1980; Fliss, I., et al., Biotechniques, 1991; Yokogawa, K., et al., 1975), and may be used in conjunction with the disclosures herein. An example of a mutanolysin comprises a commercially available mutanolysin (e.g., Sigma Aldrich).

f. Cellulases

Cellulase (EC 3.2.1.4; CAS registry number: 9012-54-8) catalyzes the reaction: in a cellulose, endohydrolysis of a (1,4)-β-D-glucosidic linkage; in a lichenin, endohydrolysis of a (1,4)-β-D-glucosidic linkage; and/or in a cereal β-D-glucan, endohydrolysis of a (1,4)-β-D-glucosidic linkage. In additional aspects, a cellulase may possess the catalytic activity of: hydrolyse of a 1,4-linkage in a β-D-glucan also comprising a 1,3-linkage. Cellulase producing cells and methods for isolating a cellulase from a cellular material and/or a biological source have been described [see, for example, Datta, P. K., et al., 1963; Myers, F. L. and Northcote, D. H., 1959; Whitaker, D. R. et al., 1963; Hatfield, R. and Nevins, D. J., 1986; Inohue, M. et al., 1999], and may be used in conjunction with the disclosures herein. A commercially available cellulase preparation (e.g., Sigma-Aldrich), often comprises an additional enzyme retained and/or added during preparation, such as a hemicellulase, to aid digestion of cellulose comprising substrates. Structural information for a wild-type cellulase and/or a functional equivalent amino acid sequence for producing a cellulase and/or a functional equivalent include Protein database bank entries: 1A39; 1A3H; 1AIW; 1CEC; 1CEM; 1CEN; 1CEO; 1CLC; 1CX1; 1DAQ; 1DAV; 1DYM; 1DYS; 1E5J; 1ECE; 1EDG; 1EG1; 1EGZ; 1F9D; 1F90; and/or 8A3H. Examples of a cellulase and/or a functional equivalent KEEG sequences for production of wild-type and/or a functional equivalent nucleotide and protein sequence include: DFRU: 144551(NEWSINFRUG00000162829) 157531(NEWSINFRUG00000148215) 180346(NEWSINFRUG00000163275); DBMO: Bmb020157; CNE: CNH00790; CNB: CNBL0740; DPCH: 121193(e_gwh2.5.359.1) 129325(e_gwh2.2.646.1) 139079(e_gww2.2.208.1); LBC: LACBIDRAFT_294705 LACBIDRAFT_311963; DDI: DDB_0215351(celA) DDB_0230001; DPKN: and/or KCR: Kcr_0883 Kcr_1258.

g. Chitinases

Chitinase (EC 3.2.1.14; CAS registry number: 9001-06-3) catalyzes the reaction: random hydrolysis of a N-acetyl-β-D-glucosaminide (1→4)-β-linkage in a chitin; and random hydrolysis of a N-acetyl-β-D-glucosaminide (1→4)-β-linkage in a chitodextrin. In additional aspects, a chitinase may possess the catalytic activity of a lysozyme. Chitinase producing cells and methods for isolating a chitinase from a cellular material and/or a biological source have been described [see, for example, Fischer, E. H. and Stein, E. A. Cleavage of O- and S-glycosidic bonds (survey), in Boyer, P. D., Lardy, H. and Myrbäck, K. (Eds.), The Enzymes, 2nd end., vol. 4, pp. 301-312, 1960; Tracey, M. V., 1955], and may be used in conjunction with the disclosures herein. An example of a chitinase comprises a commercially available chitinase (e.g., Sigma Aldrich). Structural information for a wild-type chitinase and/or a functional equivalent amino acid sequence for producing a chitinase and/or a functional equivalent include Protein database bank entries: 1CNS; 1CTN; 1D2K; 1DXJ; 1E6Z; and/or 3CQL. Examples of a chitinase and/or a functional equivalent KEEG sequences for production of wild-type and/or a functional equivalent nucleotide and protein sequence include: HSA: 1118(CHIT1) 27159(CHIA); PTR: 457641(CHIT1); MCC: 703284(CHIA) 703286(CHIT1); and/or HAU: Haur_2750.

h. α-Agarases

α-agarase (EC 3.2.1.158; CAS no. 63952-00-1) catalyzes the reaction: in an agarose, endohydrolysis of a 1,3-α-L-galactosidic linkage, producing an agarotetraose. Porphyran, a sulfated agarose, may also be cleaved. In additional aspects, an α-agarase obtained from a Thalassomonas sp. may possess the catalytic activity on a substrate such as a neoagarohexaose (“3,6-anhydro-α-L-galactopyranosyl-(1,3)-D-galactose”) and/or an agarohexaose. α-agarase activity may be enhanced by Ca²⁺. α-agarase producing cells and methods for isolating an α-agarase from a cellular material and/or a biological source have been described (see, for example, Ohta, Y., et al., 2005a; Ohta, Y., et al., 2005b; Potin, P., et al., 1993), and may be used in conjunction with the disclosures herein.

i. β-agarases

β-agarase (EC 3.2.1.81; CAS registry number: 37288-57-6) catalyzes the reaction: in agarose, hydrolysis of a 1,4-β-D-galactosidic linkage, producing a tetramer. An AgaA derived from Zobellia galactanivorans produces a neoagarohexaose and a neoagarotetraose, while an AgaB produces a neoagarobiose and a neoagarotetraose. A β-agarase also cleaves a porphyran. β-agarase producing cells and methods for isolating a β-agarase from a cellular material and/or a biological source have been described (see, for example, Allouch, J., et al., 2003; Duckworth, M. and Turvey, J. R. 1969; Jam, M. et al., 2005; Ohta, Y. et al., 2004a; Ohta, Y. et al., 2004b; Sugano, Y. et al., 1993), and may be used in conjunction with the disclosures herein. Structural information for a wild-type β-agarase and/or a functional equivalent amino acid sequence for producing a β-agarase and/or a functional equivalent include Protein database bank entries: 104Y, 104Z, and/or 1URX. Examples of a β-agarase and/or a functional equivalent KEEG sequences for production of wild-type and/or a functional equivalent nucleotide and protein sequence include: PPF: Pput_1162; PAT: Patl_1904 Patl_1971 Patl_2341 Patl_2640 Patl_2642; SDE: Sde_1175 Sde_1176 Sde_2644 Sde_2650 Sde_2655; RPB: RPB_3029; RPD: RPD_2419; RPE: RPE_4620; SCO: SCO_3471(dagA); and/or RBA: RB3421(agrA).

j. N-Acetylmuramoyl-L-Alanine Amidases

N-acetylmuramoyl-L-alanine amidase (EC 3.5.1.28; CAS registry number: 9013-25-6) catalyzes the reaction: hydrolysis of a link between a L-amino acid residue and a N-acetylmuramoyl residue in some cell-wall glycopeptides. N-acetylmuramoyl-L-alanine amidase producing cells and methods for isolating a N-acetylmuramoyl-L-alanine amidase from a cellular material and/or a biological source have been described [see, for example, Ghuysen, J.-M. et al. 1969; Herbold, D. R. and Glaser, L. 1975; Ward, J. B. et al., 1982), and may be used in conjunction with the disclosures herein. Structural information for a wild-type N-acetylmuramoyl-L-alanine amidase and/or a functional equivalent amino acid sequence for producing a N-acetylmuramoyl-L-alanine amidase and/or a functional equivalent include Protein database bank entries: 1ARO, 1GVM, 1H8G, 1HCX, 1J3G, 1JWQ, 1LBA, 1X60, 1XOV, 2AR3, 2BGX, 2BH7, and/or 2BML. Examples of acetylmuramoyl-L-alanine amidase and/or a functional equivalent KEEG sequences for production of wild-type and/or a functional equivalent nucleotide and protein sequence include: HSA: 114770(PGLYRP2) 114771(PGLYRP3) 57115(PGLYRP4) 8993(PGLYRP1); PTR: 455797(PGLYRP2) 737434(PGLYRP3) 737562(PGLYRP4); MCC: 714583(LOC714583) 718287(PGLYRP2) 718480(LOC718480); MMU: 21946(Pglyrp1) 242100(Pglyrp3) 57757(Pglyrp2); and/or MMA: MM_2290

k. Lytic Transglycosylases

A lytic transglycosylase (“lytic murein transglycosylase,” EC 3.2.1.-) demonstrates exo-N-acetylmuramidase activity, and can cleave a glycan strand comprising linked a peptide and/or a glycan strand that lack linked peptides with similar efficiency. A lysozyme and a lytic transglycosylase cleaves the β1,4-glycosidic bond between a N-Acetyl-D-Glucosamine (“GlcNAc”) and a N-Acetylmuramic acid (“MurNAc”), but a lytic transglycosylase has a transglycosylation reaction producing a 1,6-anhydro ring at the MurNAc. Lytic transglycosylase producing cells and methods for isolating a lytic transglycosylase from a cellular material and/or a biological source have been described [see, for example, Holtje et al, 1975; Thunnissen et al. 1994; Scheurwater et al, 2007; Reid et al., 2004), and may be used in conjunction with the disclosures herein. Structural information for a wild-type lytic transglycosylase and/or a functional equivalent amino acid sequence for producing a lytic transglycosylase and/or a functional equivalent include Protein database bank entries: 1Q2R, 1Q2S, 2PJJ, 2PIC, 1QSA, 2PNW, 1QTE, 1QUS, 1QUT, 1QDR, 1SLY, 1DOK, IDOL, 1DOM, 3BKH, 3BKV, and/or 2AEO. Examples of lytic transglycosylase and/or a functional equivalent KEEG sequences for production of wild-type and/or a functional equivalent nucleotide and protein sequence include: ECO: b2701(m1tB); ECJ: JW2671(m1tB); ECE: Z4004(m1tB); ECS: ECs3558; ECC: c3255(m1tB); YPY: YPK_1464; YEN: YE1242(m1tB); and/or SYP: SYNPCC7002_A2370(m1tA).

1. Glucan Endo-1,3-β-D-Glucosidases

Glucan endo-1,3-β-D-glucosidase (EC 3.2.1.39; CAS registry number: 9025-37-0) catalyzes the reaction: hydrolysis of a (1,3)-β-D-glucosidic linkage in a (1,3)-β-D-glucan. In additional aspects, a glucan endo-1,3-β-D-glucosidase may possess the catalytic activity of hydrolyzing a laminarin, a pachyman, a paramylon, or a combination thereof, and also have a limited hydrolysis activity against a mixed-link (1,3-1,4-)-β-D-glucan. A glucan endo-1,313-D-glucosidase may be useful against fungal cell walls. Glucan endo-1,3-β-D-glucosidase producing cells and methods for isolating a glucan endo-1,3-β-D-glucosidase from a cellular material and/or a biological source have been described [see, for example, Chesters, C. G. C. and Bull, A. T., 1963; Reese, E. T. and Mandels, M., 1959; Tsuchiya, D., and Taga, M., 2001; Petit, J., et al., 10:4-5, 1994], and may be used in conjunction with the disclosures herein. An enzyme preparation comprising a glucan endo-1,3-β-D-glucosidase prepared from a Rhizoctonia solani (“Kitalase”), or a Trichoderma harzianum (Glucanex®) (Sigma-Aldrich). Structural information for a wild-type glucan endo-1,3-β-D-glucosidase and/or a functional equivalent amino acid sequence for producing a glucan endo-1,3-β-D-glucosidase and/or a functional equivalent include Protein database bank entries: 1GHS, 2CYG, 2HYK, and/or 3DGT. Examples of an endo-1,313-D-glucosidase and/or a functional equivalent KEEG sequences for production of wild-type and/or a functional equivalent nucleotide and protein sequence include: DBMO: Bmb007310; ATH: AT3G57260(BGL2); DPOP: 769807(fgenesh4_pg.C_LG_X001297); and/or FJO: Fjoh_2435.

m. Endo-1,3(4)-β-Glucanases

Endo-1,3(4)-β-glucanase (EC 3.2.1.6; CAS registry number: 62213-14-3) catalyzes the reaction: endohydrolysis of a (1,3)-linkage in a β-D-glucan and/or a (1,4)-linkage in a β-D-glucan, wherein the hydrolyzed link's glucose residue is substituted at a C-3 of the reducing moiety that is part of the substrate chemical linkage. Endo-1,3(4)-β-glucanase producing cells and methods for isolating an endo-1,3(4)-β-glucanase from a cellular material and/or a biological source have been described [see, for example, Barras, D. R. and Stone, B. A., 1969a; Barras, D. R. and Stone, B. A., 1969b; Cunningham, L. W. and Manners, D. J., 1961; Reese, E. T. and Mandels, M., 1959; Soya, V. V., Elyakova, L. A. and Vaskovsky, V. E., 1970], and may be used in conjunction with the disclosures herein. Structural information for a wild-type endo-1,3(4)-β-glucanase and/or a functional equivalent amino acid sequence for producing an endo-1,3(4)-β-glucanase and/or a functional equivalent include Protein database bank entries: 1UP4, 1UP6, 1UP7, and/or 2CL2. Examples of an endo-1,3(4)-β-glucanase and/or a functional equivalent KEEG sequences for production of wild-type and/or a functional equivalent nucleotide and protein sequence include: NCR: NCU04431 NCU07076; PAN: PODANSg699 PODANSg9033; FGR: FG04768.1 FG06119.1 FG08757.1; and/or NPH: NP4306A(celM).

n. β-Lytic Metalloendopeptidases

β-lytic metalloendopeptidase (EC 3.4.24.32; CAS no. 37288-92-9) catalyzes the reaction: a N-acetylmuramoyl Ala cleavage, as well as an insulin B chain cleavage. A β-lytic metalloendopeptidase may be used, for example, against a bacterial cell wall. β-lytic metalloendopeptidase producing cells and methods for isolating a β-lytic metalloendopeptidase from a cellular material and/or a biological source (e.g., an Achromobacter lyticus; a Lysobacter enzymogenes) have been described [see, for example, Whitaker, D. R. et al., 1965; Whitaker, D. R. and Roy, C., 1967; Li, S. L. et al., 1990; Altmann, F. et al., 1986; Plummer, T. H., Jr. and Tarentino, A. L., 1981; Takahashi, N., 1977; Takahashi, N. and Nishibe, H., 1978; Tarentino, A. L. et al., 1985.], and may be used in conjunction with the disclosures herein.

o. 3-Deoxy-2-Octulosonidases

3-deoxy-2-octulosonidase (EC 3.2.1.124; CAS no. 103171-48-8) catalyzes the reaction: endohydrolysis of the β-ketopyranosidic linkage of a 3-deoxy-D-manno-2-octulosonate in a capsular polysaccharide. A 3-deoxy-2-octulosonidase acts on a polysaccharide of a bacterial (e.g., an Escherichia coli) cell wall. 3-deoxy-2-octulosonidase producing cells and methods for isolating a 3-deoxy-2-octulosonidase from a cellular material and/or a biological source have been described [see, for example, Altmann, F. et al., 1986], and may be used in conjunction with the disclosures herein.

p. Peptide-N4-(N-acetyl-β-Glucosaminyl)asparagine Amidases

Peptide-N⁴-(N-acetyl-β-glucosaminyl)asparagine amidase (EC 3.5.1.52; CAS no. 83534-39-8) catalyzes the reaction: hydrolysis of a N⁴-(acetyl-β-D-glucosaminyl)asparagine residue. The reaction may promote the glycosylation of the glyglucosamine residue, and produce a peptide comprising an aspartate and a substituted N-acetyl-β-D-glucosaminylamine. Peptide-N⁴-(N-acetyl-β-glucosaminyl)asparagine amidase does not substantively act on (G1cNAc)Asn, as 3 or more amino acids in the substrate promotes the reaction. Peptide-N⁴-(N-acetyl-β-glucosaminyl)asparagine amidase producing cells and methods for isolating an eptide-N4-(N-acetyl-β-glucosaminyl)asparagine amidase from a cellular material and/or a biological source have been described [see, for example, Plummer, T. H., Jr. and Tarentino, A. L., 1981; Takahashi, N. and Nishibe, H., 1978; Takahashi, N., 1977; Tarentino, A. L. et al., 1985], and may be used in conjunction with the disclosures herein. Structural information for a wild-type peptide-N⁴-(N-acetyl-β-glucosaminyl) asparagine amidase and/or a functional equivalent amino acid sequence for producing a peptide-N4-(N-acetyl-β-glucosaminyl)asparagine amidase and/or a functional equivalent include Protein database bank entries: 1PGS, 1PNF, 1PNG, 1X3W, 1X3Z, 2D5U, 2F4M, 2F40, 2G9F, 2G9G, 2HPJ, 2HPL, and/or 2174. Examples of peptide-N⁴-(N-acetyl-β-glucosaminyl)asparagine amidase and/or a functional equivalent KEEG sequences for production of wild-type and/or a functional equivalent nucleotide and protein sequence include: HSA: 55768(NGLY1); PTR: 460233(NGLY1); MCC: 700842(LOC700842); DECB: 100059456(LOC100059456); OAA: 100075786(LOC100075786); GGA: 420655(NGLY1); DRE: 553627(zgc:110561); and/or DTPS: 35410(e gw1.7.250.1).

q. Mannosyl-Glycoprotein Endo-β-N-Acetylglucosaminidases

Mannosyl-glycoprotein endo-β-N-acetylglucosaminidase (EC 3.2.1.96; CAS no. 37278-88-9) catalyzes the reaction: a N,N-diacetylchitobiosyl unit endohydrolysis in a high-mannose glycoprotein and/or a glycopeptide comprising a -[Man(GlcNAc)₂]Asn- structure, wherein the intact oligosaccharide is released and a N-acetyl-D-glucosamine residue is still attached to the protein. Mannosyl-glycoprotein endo-β-N-acetylglucosaminidase producing cells and methods for isolating a mannosyl-glycoprotein endo-β-N-acetylglucosaminidase from a cellular material and/or a biological source have been described [see, for example, Chien, S., et al., 1977; Koide, N. and Muramatsu, T., 1974; Pierce, R. J. et al., 1979; Pierce, R. J. et al., 1980; Tai, T. et al., 1975; Tarentino, A. L., et al., 1974.], and may be used in conjunction with the disclosures herein. Structural information for a wild-type mannosyl-glycoprotein endo-β-N-acetylglucosaminidase and/or a functional equivalent amino acid sequence for producing a mannosyl-glycoprotein endo-3-N-acetylglucosaminidase and/or a functional equivalent include Protein database bank entries: 1C3F, 1C8X, 1C8Y, 1C90, 1C91, 1C92, 1C93, 1EDT, 1EOK, 1EOM, and/or 2EBN. Examples of mannosyl-glycoprotein endo-β-N-acetylglucosaminidase and/or a functional equivalent KEEG sequences for production of wild-type and/or a functional equivalent nucleotide and protein sequence include: HSA: 64772(FLJ21865); OAA: 100089364(LOC100089364); DCIN: 254322(gw1.55.22.1); and/or CHU: CHU_1472(flgJ).

r. ι-Carrageenases

ι-carrageenase (EC 3.2.1.157) catalyzes the reaction: in an ι-carrageenan, endohydrolysis of a 1,4-β-D-linkage between a 3,6-anhydro-D-galactose-2-sulfate and a D-galactose 4-sulfate. ι-carrageenase producing cells and methods for isolating an ι-carrageenase from a cellular material and/or a biological source have been described [see, for example, Barbeyron, T. et al., 2000; Michel, G. et al., 2001; Michel, G. et al., 2003], and may be used in conjunction with the disclosures herein. Structural information for a wild-type ι-carrageenase and/or a functional equivalent amino acid sequence for producing a ι-carrageenase and/or a functional equivalent include Protein database bank entries: 1H80 and/or 1KTW.

s. κ-Carrageenases

κ-carrageenase (EC 3.2.1.83; CAS no. 37288-59-8) catalyzes the reaction: in a κ-carrageenans, endohydrolysis of a 1,4-β-D-linkage between a 3,6-anhydro-D-galactose and a D-galactose 4-sulfate. κ-carrageenase often acts against an alga (e.g., red algae). κ-carrageenase producing cells and methods for isolating a κ-carrageenase from a cellular material and/or a biological source have been described [see, for example, Weigl, J. and Yashe, W., 1966; Potin, P. et al., 1991; Potin, P. et al., 1995; Michel, G. et al., 1999; Michel, G., et al., 2001), and may be used in conjunction with the disclosures herein. Structural information for a wild-type κ-carrageenase and/or a functional equivalent amino acid sequence for producing a κ-carrageenase and/or a functional equivalent include Protein database bank entries: 1DYP. Examples of κ-carrageenase and/or a functional equivalent KEEG sequences for production of wild-type and/or a functional equivalent nucleotide and protein sequence include: RBA: RB2702.

t. λ-Carrageenases

λ-carrageenase (EC 3.2.1.162) catalyzes the reaction: in a λ-carrageenan, endohydrolysis of a (1,4)-β-linkage, producing a α-D-Galp2,6S2-(1,3)-β-D-Galp2S-(1,4)-α-D-Galp2,6S2-(1,3)-D-Galp2S tetrasaccharide. λ-carrageenase producing cells and methods for isolating a λ-carrageenase from cellular materials (e.g., Pseudoalteromonas sp) and biological sources have been described [see, for example, Ohta, Y. and Hatada, 2006], and may be used in conjunction with the disclosures herein.

u. α-Neoagaro-Oligosaccharide Hydrolases

α-neoagaro-oligosaccharide hydrolase (EC 3.2.1.159) catalyzes the reaction: hydrolysis of a 1,3-α-L-galactosidic linkage in a neoagaro-oligosaccharide, wherein the substrate is a pentamer or smaller, producing a D-galactose and a 3,6-anhydro-L-galactose. α-neoagaro-oligosaccharide hydrolase producing cells and methods for isolating a α-neoagaro-oligosaccharide hydrolase from a cellular material and/or a biological source have been described [see, for example, Sugano, Y., et al. 1994], and may be used in conjunction with the disclosures herein.

v. Additional Anti-Biological Enzymes

An endolysin may be used for a Gram positive bacteria, such as one that may be resistant to a lysozyme. An endolysin comprises a phage encoded enzyme that fosters release of a new phage by destruction of a cell wall. An endolysin may comprise a N-acetylmuramidase, a N-acetylglucosaminidae, an emdopeptidase, and/or an amidase. An endolysin may be translocated by phage encoded holin protein in disrupting a cytosolic membrane (Wang et al., 2000). A LysK lysine from phage k and a Listeria monocytogenes bacteriophage-lysin have been recombinantly expressed in a Lactoccus lactus and/or an E. coli (Loessner et al. 1995; Gaeng et al. 2000; O'Flaherty et al. 2005). An autolysin such as, for example, from Staphylococcus aureus, Bacillus subtilis, or Streptococcus pneumonia, may also be used as an anti-biological and/or an anti-fouling enzyme (Smith et al, 2000; Lopez et al. 2000).

A protease may be used to cleave the mannoprotein outer cell wall layer, such as for a fungus such as a yeast. A glucanase such as, for example, a beta(1->6) glucanase, a glucan endo-1,3-β-D-glucosidase, and/or an endo-1,3(4)-β-glucanase can then more easily cleave glucan from the inner cell wall layer(s). Combinations of a protease and a glucanase may be used to produce an improved lytic activity. A reducing agent, such as a dithiothreitol of beta-mercaptoethanol, may aid in allowing enzyme contact with the inner cell wall by breaking a disulfide linkage, such as between a cell wall protein and a mannose. A mannose, a chitinase, a proteinase, a pectinase, an amylase, or a combination thereof may also be used, such as for aiding cell wall component cleavage. Examples of enzymes that degrade fungal cell walls include those produced by an Arthrobacter sp., a Celluloseimicrobium cellulans (“Oerskovia xanthineolytica LL G109”) (DSM 10297), a Cellulosimicrobium cellulans (“Arthobacter lueus 73/14”) (ATCC 21606), a Cellulosimicrobium cellulans TK-1, a Rarobacter faecitabidus, a Rhizoctonia sp., or a combination thereof. An Arthrobacter sp. produces a protease with a functional optimum of about pH 11 and about 55° C. (Adamitsch et al., 2003). A Celluloseimicrobium cellulans (ATCC 21606) produces a protease and a glucanase (“lyticase”) with a functional optimum of about pH 10 and about pH 8.0, respectively (Scott and Schekman, 1980; Shen et al., 1991). A Celluloseimicrobium cellulans (DSM 10297) produces a protease with functional optimums of about pH 9.5 to about pH 10, and a glucanase with a functional optimum of about pH 8.0 and about 40° C. (Ventom and Asenjo, 1990). A Rarobacter faecitabidus produces a protease effective against cell wall a component (Shimoi et al, 1992). A Rarobacter sp. produces a glucanase with a functional optimum of about pH 6 to about pH 7, and about 40° C. (Kobayashi et al.1981). In specific aspects, commercially available enzyme preparations such as a zymolase and/or a lyticase (Sigma-Aldrich), generally comprising a β-1,3-glucanase and another enzyme, may be used.

w. Phosphoric Triester Hydrolases

In certain embodiments, a material formulation comprises a hydrolase. A hydrolase may comprise an esterase. A type of an additional esterase comprises an esterase that catalyzes the hydrolysis of an organophosphorus compound. Examples of such an additional esterase include those identified by enzyme commission number EC 3.1.8, the phosphoric triester hydrolases. Examples of a the phosphoric triester hydrolase includes an aryldialkylphosphatase (EC 3.1.8.1) and/or an diisopropyl-fluorophosphatase (EC 3.1.8.2).

A phosphoric triester hydrolase catalyzes the hydrolytic cleavage of an ester from a phosphorus moiety, such as in an organophosphorus compound. An “organophosphorus compound” comprises a phosphoryl center, and further comprises two or three ester linkages. In some aspects, the type of phosphoester bond and/or additional covalent bond at the phosphoryl center classifies an organophosphorus compound. In embodiments wherein the phosphorus comprises a linkage to an oxygen by a double bond (P═O), the OP compound may be known as an “oxon OP compound” and/or “oxon organophosphorus compound.” In embodiments wherein the phosphorus comprises a linkage to a sulfur by a double bond (P═S), the OP compound may be known as a “thion OP compound” and/or “thion organophosphorus compound.” Additional examples of bond-type classified OP compounds include a phosphonocyanate, which comprises a β-CN bond; a phosphoroamidate, which comprises a β-N bond; a phosphotriester, which comprises a β-O bond; a phosphodiester, which comprises a β-O bond; a phosphonofluoridate, which comprises a β-F bond; and a phosphonothiolate, which comprises a β-S bond. A “dimethyl OP compound” comprises two methyl moieties covalently bonded to the phosphorus atom, such as, for example, a malathion. A “diethyl OP compound” comprises two ethoxy moieties covalently bonded to the phosphorus atom, such as, for example, a diazinon. Examples of a phosphoric triester hydrolase include an aryldialkylphosphatase (EC 3.1.8.1), a diisopropyl-fluorophosphatase (EC 3.1.8.2), or a combination thereof

An aryldialkylphosphatase (EC 3.1.8.1) catalyzes the following reaction: aryl dialkyl phosphate+H₂O=an aryl alcohol+dialkyl phosphate. Examples of an aryl dialkyl phosphate include an organophosphorus compound comprising a phosphonic acid ester, a phosphinic acid ester, or a combination thereof. Aryldialkylphosphatase producing cells and methods for isolating an aryldialkylphosphatase from a cellular material and/or a biological source have been described, [see, for example, Bosmann, H. B., 1972; and Mackness, M. I. et al., 1987.], and may be used in conjunction with the disclosures herein. Structural information for a wild-type aryldialkylphosphatase and/or a functional equivalent amino acid sequence for producing an aryldialkylphosphatase and/or a functional equivalent include Protein database bank entries: 1EYW, 1EZ2, 1HZY, 1I0B, 1I0D, 1JGM, 1P6B, 1P6C, 1P9E, 1QW7, 1V04, 2D2G, 2D2H, 2D2J, 2O4M, 2O4Q, 2OB3, 2OQL, 2R1K, 2R1L, 2R1M, 2R1N, 2R1P, 2VC5, 2VC7, 2ZC1, 3C86, 3CAK, and/or 3E3H. Examples of an aryldialkylphosphatase and/or a functional equivalent KEEG sequences for production of wild-type and/or a functional equivalent nucleotide and protein sequence include: HSA—5444(PON1), 5445(PON2), 5446(PON3); PTR—463547(PON1), 463548(PON3), 463549(PON2); and/or RXY—Rxyl_2340. Examples of an aryldialkylphosphatase include an organophosphorus hydrolase (E.C.3.1.8.1), which may be referred to herein as “organophosphorus hydrolase” and/or “OPH”; and a peraoxonase (E.C.3.1.8.1). The use of any opd gene (e.g., Genbank accession no. M20392; Genbank accession no. M22863) and/or the gene product in the described compositions, articles, methods, etc. is contemplated. Examples of an opd gene and a gene product that may be used include an Agrobacterium radiobacter P230 organophosphate hydrolase gene, opdA (Genbank accession no. AY043245; Entrez databank no. AAK85308); a Flavobacterium balustinum opd gene for parathion hydrolase (Genbank accession no. AJ426431; Entrez databank no. CAD19996); a Pseudomonas diminuta phosphodiesterase opd gene (Genbank accession no. M20392; Entrez databank no. AAA98299; Protein Data Bank entries 1JGM, 1DPM, 1EYW, 1EZ2, 1HZY, 1IOB, 1IOD, 1PSC and 1PTA); a Flavobacterium sp opd gene (Genbank accession no. M22863; Entrez databank no. AAA24931; ATCC 27551); a Flavobacterium sp. parathion hydrolase opd gene (Genbank accession no. M29593; Entrez databank no. AAA24930; ATCC 27551); or a combination thereof (Home, I. et al., 2002; Somara, S. et al., 2002; McDaniel, C. S. et al., 1988a; Harper, L. L. et al., 1988; Mulbry, W. W. and Karns, J. S., 1989). A peraoxonase such as a human paraoxonase (EC 3.1.8.1) comprises a calcium dependent protein, and may be also known as an “arylesterase” and/or “aryl-ester hydrolase” (Josse, D. et al., 1999; Vitarius, J. A. and Sultanos, L. G., 1995). Examples of the human paraoxonase (“HPON1”) gene and gene products may be accessed at (Genbank accession no. M63012; Entrez databank no. AAB59538) (Hassett, C. et al., 1991).

A diisopropyl-fluorophosphatase (EC 3.1.8.2) catalyzes the following reaction: diisopropyl fluorophosphate+H₂O=fluoride+diisopropyl phosphate. Examples of a diisopropyl fluorophosphate include an organophosphorus compound comprising a phosphorus-halide, a phosphorus-cyanide, or a combination thereof. Diisopropyl-fluorophosphatase producing cells and methods for isolating a diisopropyl-fluorophosphatase from a cellular material and/or a biological source have been described, [see, for example, Cohen, J. A. and Warring, M. G., 1957], and may be used in conjunction with the disclosures herein. Structural information for a wild-type diisopropyl-fluorophosphatase and/or a functional equivalent amino acid sequence for producing a diisopropyl-fluorophosphatase and/or a functional equivalent include Protein database bank entries: 1E1A, 1PJX, 2GVU, 2GVV, 2GVW, 2GVX, 21A0, 2IAP, 2IAQ, 2IAR, 2IAS, 2IAT, 2IAU, 2IAV, 2IAW, 2IAX, 2W43, and/or 3BYC.

Organophosphorus acid anhydrolases (E.C.3.1.8.2), known as “OPAAs,” have been isolated from microorganisms and identified as enzymes that detoxify OP compounds (Serdar, C. M. and Gibson, D. T., 1985; Mulbry, W. W. et al., 1986; DeFrank, J. J. and Cheng, T.-C., 1991). The better-characterized OPAAs have been isolated from an Altermonas species, such as an Alteromonas sp JD6.5, an Alteromonas haloplanktis, and an Altermonas undina (ATCC 29660) (Cheng, T.-C. et al., 1996; Cheng, T.-C. et al., 1997; Cheng, T. C. et al., 1999; Cheng, T.-C. et al., 1993). Examples of an OPAA gene and a gene product that may be used include an Alteromonas sp JD6.5 opaA gene, (GeneBank accession no. U29240; Entrez databank no. AAB05590); an Alteromonas haloplanktis prolidase gene (GeneBank accession no. U56398; Entrez databank AAA99824; ATCC 23821); or a combination thereof. The wild-type encoded OPAA from an Alteromonas sp JD6.5 comprises 517 amino acids, while the wild-type encoded OPAA from an Alteromonas haloplanktis comprises 440 amino acids (Cheng, T. C. et al., 1996; Cheng, T.-C. et al., 1997). The Alteromonas OPAAs accelerates the hydrolysis of a phosphotriester and/or a phosphofluoridate, including a cyclosarin, a sarin and/or a soman.

A “squid-type DFPase” (EC 3.1.8.2) refers to an enzyme that catalyzes the cleavage of both a DFP and a soman, and may be isolated from organisms of the Loligo genus. Generally, a squid-type DFPase cleaves a DFP at a faster rate than a soman. Squid-type DFPases include, for example, a DFPase obtained from a Loligo vulgaris, a Loligo pealei, a Loligo opalescens, or a combination thereof (Hoskin, F. C. G. et al., 1984; Hoskin, F. C. G. et al., 1993; Garden, J. M. et al., 1975). A gene encoding a squid-type DFP has been isolated, and may be accessed at GeneBank accession no. AX018860 (International patent publication: WO 9943791-A). Further, this enzyme's X-ray crystal structure has been determined (Protein Data Bank entry 1E1A) (Koepke, J. et al., 2002; Scharff, E. I. et al., 2001).

A “Mazur-type DFPase” (EC 3.1.8.2) refers to an enzyme that catalyzes the cleavage of both DFP and soman. Generally, a Mazur-type DFPase cleaves a soman at a faster rate than a DFP. Examples of a Mazur-type DFPase include the DFPase isolated from a mouse liver (Billecke, S. S. et al., 1999), which may be the same as the DFPase known as a SMP-30 (Fujita, T. et al., 1996; Billecke, S. S. et al., 1999; Genebank accession no. U28937; Entrez databank AAC52721); a DFPase isolated from a rat liver (Little, J. S. et al., 1989); a DFPase isolated from a hog kidney; a DFPase isolated from a Bacillus stearothermophilus strain OT; a DFPase isolated from an Escherichia coli (ATCC25922) (Hoskin, F. C. G. et al., 1993; Hoskin, F. C. G, 1985); or a combination thereof.

x. Lipolytic Enzymes

Additionally, a lipolytic enzyme may act on a biological entity's lipid (e.g., a lipid of a plasma membrane), and thus may possess anti-biological activity suitable for use in the composition(s) and method(s) herein. In general embodiments, a lipolytic enzyme comprises a hydrolase. A hydrolase generally comprises an esterase, a ceramidase (EC 3.5.1.23), or a combination thereof. Examples of an esterase comprise those identified by enzyme commission number (EC 3.1): a carboxylic ester hydrolase, (EC 3.1.3), a phosphoric monoester hydrolase (EC 3.1.3), a phosphoric diester hydrolase (EC 3.1.4), or a combination thereof. A carboxylic ester hydrolase catalyzes the hydrolytic cleavage of an ester to produce an alcohol and a carboxylic acid product. A phosphoric monoester hydrolase catalyzes the hydrolytic cleavage of an O—P ester bond. A “phosphoric diester hydrolase” catalyzes the hydrolytic cleavage of a phosphate group's phosphorus atom and two other moieties over two ester bonds. A “ceramidase” hydrolyzes the N-acyl bond of ceramide to release a fatty acid and sphingosine. Examples of a lipolytic esterase and a ceramidase include a carboxylesterase (EC 3.1.1.1), a lipase (EC 3.1.1.3), a lipoprotein lipase (EC 3.1.1.34), an acylglycerol lipase (EC 3.1.1.23), a hormone-sensitive lipase (EC 3.1.1.79), a phospholipase A₁ (EC 3.1.1.32), a phospholipase A₂ (EC 3.1.1.4), a phosphatidylinositol deacylase (EC 3.1.1.52), a phospholipase C (EC 3.1.4.3), a phospholipase D (EC 3.1.4.4), a phosphoinositide phospholipase C (EC 3.1.4.11), a phosphatidate phosphatase (EC 3.1.3.4), a lysophospholipase (EC 3.1.1.5), a sterol esterase (EC 3.1.1.13), a galactolipase (EC 3.1.1.26), a sphingomyelin phosphodiesterase (EC 3.1.4.12), a sphingomyelin phosphodiesterase D (EC 3.1.4.41), a ceramidase (EC 3.5.1.23), a wax-ester hydrolase (EC 3.1.1.50), a fatty-acyl-ethyl-ester synthase (EC 3.1.1.67), a retinyl-palmitate esterase (EC 3.1.1.21), a 11-cis-retinyl-palmitate hydrolase (EC 3.1.1.63), an all-trans-retinyl-palmitate hydrolase (EC 3.1.1.64), a cutinase (EC 3.1.1.74), an acyloxyacyl hydrolase (EC 3.1.1.77), a petroleum lipolytic enzyme, or a combination thereof.

8. Biomolecular Functional Equivalents

A “functional equivalent” (“conservative modified variant”) proteinaceous molecule comprising a structural analog and/or a sequence analog may possess an altered (e.g., an enhanced property, a reduced property), in comparison to the proteinaceous molecule upon which it is based. For example, a proteinaceous molecule comprising a chemical modification and/or a sequence modification that functions the same or similar (e.g., a modified enzyme of the same EC classification as the unmodified enzyme; a peptide with a similar binding activity for a ligand) comprises a “functional equivalent,” a “conservative modified variant” and/or “in accordance” with, an un-modified proteinaceous molecule. As used herein, a “structural analog” refers to one or more chemical modifications to the peptide backbone and/or non-side chain chemical moiety(s) of a proteinaceous molecule. In certain aspects, a subcomponent of a proteinaceous molecule such as an apo-enzyme, a prosthetic group, a co-factor, or a combination thereof, may be modified to produce a functional equivalent structural analog. In particular facets, such a proteinaceous molecule sub-component that does not comprise a proteinaceous molecule may be altered to produce a functional equivalent structural analog of a proteinaceous molecule when combined with the other sub-components. As used herein, a “sequence analog” refers to one or more chemical modifications to the side chain chemical moiety(s), also referred to herein as a “residue” of one or more amino acids that define a proteinaceous molecule's sequence. Often such a “sequence analog” comprises an amino acid substitution, which may be produced by recombinant expression of a nucleic acid comprising a genetic mutation to produce a mutation in the expressed amino acid sequence.

It is possible to alter a proteinaceous molecule, such as, for example one having a defined amino acid sequence and/or length, for one or more properties. In some aspects, using the methods of screening for activity, it is possible to produce and identify proteinaceous molecule(s) (e.g., a functional equivalent) for use in a material formulation (e.g., a paint, a coating) in a shorter time and/or with a higher-probability of success, than screening natural isolates for a proteinaceous molecule. As used herein “alter” or “alteration” may result in conferring, increasing and/or a decreasing the measured value for a particular property. Examples of a property, in the context of a proteinaceous molecule, includes, but is not limited to, a ligand binding property (e.g., association constant Kₐ; disassociation constant K_{d}), a catalytic property, a stability property, a property related to environmental safety, a charge property, or a combination thereof. Examples of a catalytic property that may be altered include a kinetic parameter, such as Kₘ, a catalytic rate (k_{cat}) for a substrate, an enzyme's specificity for a substrate (k_{cat}/Kₘ), or a combination thereof. Examples of a stability property that may be altered include thermal stability, half-life of activity, stability after exposure to a weathering condition, or a combination thereof. Examples of a property related to environmental safety include an alteration in toxicity, antigenicity, bio-degradability, or a combination thereof. For example, an alteration to increase an enzyme's catalytic rate for a substrate, an proteinaceous molecule's specificity and/or binding property(s) for a ligand, a proteinaceous molecule's thermal stability, a proteinaceous molecule's half-life of activity, and/or a proteinaceous molecule's stability after exposure to a weathering condition may be selected for some applications, while a decrease in toxicity and/or antigenicity for a proteinaceous molecule may be selected in additional applications. There may be a limit to the number of chemical modifications that may be made to a proteinaceous molecule before a property may be undesirably altered. However, in light of the disclosures herein of assays for determining whether a composition possesses one or more properties, including, for example, an anti-biological property, an anti-fouling property, a metal binding property, an enzymatic activity, a stability property, a binding property, etc., using, but not limited to the assays described herein, to determine whether a given chemical modification to a proteinaceous molecule (e.g., an enzyme, an antibody, a receptor, a peptide, a polypeptide) produces a molecule that still possesses a suitable set of properties for use in a particular application.

As used herein, an “amino acid” may comprise a common and/or an uncommon amino acid. An amino acid comprises a monomer (“precursor”) in a peptide and/or polypeptide polymer. The common amino acids include: alanine (Ala, A); arginine (Arg, R); aspartic acid (a.k.a. aspartate; Asp, D); asparagine (Asn, N); cysteine (Cys, C); glutamic acid (a.k.a. glutamate; Glu, E); glutamine (Gln, Q); glycine (Gly, G); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V). Common amino acids are often biologically produced in the biological synthesis of a peptide and/or a polypeptide. Various uncommon amino acids may be used, though general embodiments, an proteinaceous molecule may be biologically produced, and thus lack or possess relatively few uncommon amino acids prior to any subsequent non-mutation based chemical modifications. An uncommon amino acid refers to an analog of a common amino acid (e.g., a D isomer of an L-amino acid), as well as a synthetic amino acid whose side chain may be chemically unrelated to the side chains of the common amino acids (e.g., a norleucine). Thus, for example, a proteinaceous molecule may comprise an amino acid such as a common amino acid, an uncommon amino acid, an L-amino acid, a D-amino acid, a cyclic (non-racemic) amino, or a combination thereof.

A selected proteinaceous molecule (e.g., an active peptide), may be modified to comprise functionally equivalent amino acid substitutions and yet retain the same or similar characteristics (e.g., an anti-biological property). In embodiments wherein an amino acid of particular interest has been identified using such techniques, functional equivalents may be created using mutations that substitute a different amino acid for the identified amino acid of interest. Examples of substitutions of an amino acid side chain to produce a functional equivalent proteinaceous molecule are also known in the art, and may involve a conservative side chain substitution a non-conservative side chain substitution, or a combination thereof, to rationally alter a property of a proteinaceous molecule. Examples of conservative side chain substitutions include, when applicable, replacing an amino acid side chain with one similar in charge (e.g., an arginine, a histidine, a lysine); similar in hydropathic index; similar in hydrophilicity; similar in hydrophobicity; similar in shape (e.g., a phenylalanine, a tryptophan, a tyrosine); similar in size (e.g., an alanine, a glycine, a serine); similar in chemical type (e.g., acidic side chains, aromatic side chains, basic side chains); or a combination thereof. Conversely, when a change to produce a non-conservative substitution to alter a property of proteinaceous molecule, and still produce a “functional equivalent” proteinaceous molecule, these guidelines may be used to select an amino acid whose side-chains relatively non-similar in charge, hydropathic index, hydrophilicity, hydrophobicity, shape, size, chemical type, or a combination thereof.

Various amino acids have been given a numeric quantity based on the characteristics of charge and hydrophobicity, called the hydropathic index (Kyte, J. and Doolittle, R. F. 1982), which may be used as a criterion for a substitution (e.g., a substitution related to conferring or retaining a biological function). For example, the relative hydropathic character of the amino acid may determine the secondary structure of the resultant protein, which in turn defines the interaction of the protein with a ligand (e.g., a substrate) molecule. Similarly, in a proteinaceous molecule (e.g., a peptide, a polypeptide) whose secondary structure may not be a principal aspect of the interaction of the proteinaceous molecule (e.g., a peptide), position within the proteinaceous molecule (e.g., a peptide), and a characteristic of the amino acid residue may determine the interaction the proteinaceous molecule (e.g., a peptide) has in a biological system. An amino acid sequence may be varied in some embodiments. For example, certain amino acids may be substituted for other amino acids having a similar hydropathic index or score and still retain similar if not identical biological activity. The hydropathic index of the common amino acids are: Arg (−4.5); Lys (−3.9); Asn (−3.5); Asp (−3.5); Gln (−3.5); Glu (−3.5); His (−3.2); Pro (−1.6); Tyr (−1.3); Trp (−0.9); Ser (−0.8); Thr (−0.7); Gly (−0.4); Ala (+1.8); Met (+1.9); Cys (+2.5); Phe (+2.8); Leu (+3.8); Val (+4.2); and Ile (+4.5). Additionally, a value has also been given to various amino acids based on hydrophilicity, which may also be used as a criterion for substitution (U.S. Pat. No. 4,554,101). The hydrophilicity values for the common amino acids are: Trp (−3.4); Phe (−2.5); Tyr (−2.3); Ile (−1.8); Leu (−1.8); Val (−1.5); Met (−1.3); Cys (−1.0); Ala (−0.5); His (−0.5); Pro (−0.5+/−0.1); Thr (−0.4); Gly (0); Asn (+0.2); Gln (+0.2); Ser (+0.3); Asp (+3.0+/−0.1); Glu (+3.0+/−0.1); Arg (+3.0); and/or Lys (+3.0). In aspects wherein an amino acid may be conservatively substituted (i.e., exchanged) for an amino acid comprising a similar or same hydropathic index and/or hydrophilic value, the difference between the respective index and/or value may be generally within +/−2, within +/−1, and/or within +/−0.5. A biological functional equivalence may typically be maintained wherein an amino acid substituted (e.g., conservatively substituted). Thus, it is expected that isoleucine, for example, which has a hydropathic index of +4.5, can be substituted for valine (+4.2) or leucine (+3.8), and still obtain a proteinaceous molecule (e.g., a protein) having similar activity (e.g., a biologic activity). A lysine (−3.9) can be substituted for arginine (−4.5), and so on. These amino acid substitutions are generally based on the relative similarity of R-group substituents, for example, in terms of size, electrophilic character, charge, and the like. Although these are not the only such substitutions, the substitutions which take the foregoing characteristics into consideration, for example for a hydropathic index, include An alanine substituted with a Gly and/or a Ser; an arginine substituted with a Lys; an asparagine substituted with a Gln and/or a His; an aspartate substituted with a Glu; a cysteine substituted with a Ser; a glutamate substituted with an Asp; a glutamine substituted with an Asn; a glycine substituted with an Ala; a histidine substituted with an Asn and/or a Gln; an isoleucine substituted with a Leu and/or Val; a leucine substituted with an Ile and/or a Val; a lysine substituted with an Arg, a Gln, and/or a Glu; a methionine substituted with a Met, a Leu, a Tyr; a serine substituted with a Thr; a threonine substituted with a Ser; a tryptophan substituted with a Tyr; a tyrosine substituted with a Trp and/or a Phe; a valine substituted with a Ile and/or a Leu; or a combination thereof. In aspects wherein an amino acid may be non-conservatively substituted, the difference between the respective hydropathic index and/or hydrophilic value may be greater than +/−0.5, greater than +/−1, and/or greater than +/−2.

In certain embodiments, a proteinaceous molecule may possess a metal binding property, an anti-biological property, an anti-fouling property (e.g., an anti-biofouling property), or a combination thereof. For example, a proteinaceous sequence rich in both positively charged residue(s) and metal binding residue(s) is contemplated as possessing multiple activities, as such amino acid(s) may reversible bind a metal ion toxic to a fouling organism as well as possess an anti-biological activity in reduced concentration (e.g., an undetectable amount) of such a metal ion. In an example, it is contemplated that proteinaceous sequences rich in positively charged residue(s) such as an arginine, a lysine, and/or a histidine may possess an anti-biological property. In specific aspects, such an anti-biological property may be effective against a biological unit (e.g., a cell, a virus) that comprises a lipid bilayer (e.g., a cellular membrane). A multi-functional metal-binding, anti-biological, and/or anti-fouling sequence may be produced, for example, by substitution of a Xaa amino acid of a metal binding sequence with a positively charged residue in an anti-biological peptide and/or substitution of a defined residue of the metal binding sequence with a positively charged functional equivalent residue based upon hydropathic and/or hydrophilicity index similarity (i.e., within about +/−2 of a hydropathic and/or hydrophilicity index values of another amino acid). In another example, such a dual function sequence may be produced by substitution of a Xaa amino acid of an anti-biological peptide with a metal binding residue and/or substitution of a defined residue in an anti-biological peptide with a metal binding residue that is a functional equivalent based upon hydropathic and/or hydrophilicity index similarity. Further, a proteinaceous molecule may comprise one or more metal binding sequences and one or more anti-biological sequences.

A proteinaceous molecule may be constructed as retroinversopeptidomimetic of a proteinaceous sequence (e.g., a D-configuration, an L-configuration). For example, a proteinaceous composition (e.g., a metal binding sequence, an anti-fouling sequence, an anti-biological sequence) may comprise, for example, an L-amino acid, a D-amino acid, a cyclic amino acid, and/or a non-natural amino acid(e.g., a β-amino acid), a backbone where one or more amide moiety(s) are reduced into an isomer analogue which may be done through peptide chemical synthesis (Stemmer, C. et al., J. Biol. Chem. 274(9):5550-5556, 1999), or a combination thereof, and various substitutions may be selected from these types of amino acids. In another example, a mixture of different proteinaceous molecules may comprises one or more peptides comprising L amino acids; one or more peptides comprising D amino acids; and/or one or more peptides comprising both an L amino acid and an D-amino acid. In other embodiments, a D-amino acid may increase the stability of a proteinaceous molecule, such as making the proteinaceous molecule insensitive and/or less susceptible to an L-amino acid biodegradation pathway. In a specific example, an L-amino acid peptide may be stabilized by addition of a D-amino acid at one or both of the peptide termini.

In another example, a proteinaceous sequence may also be produced by a reverso sequence, wherein the order of amino acids are the same, but the N- and C-termini are reversed from any sequence described herein and/or in the sequence listing. For example, a C terminus His-Xaa-Xaa-Xaa N terminus sequence is the reverso sequence of an N-terminus His-Xaa-Xaa-Xaa C terminus sequence, and both are contemplated as being functionally equivalent of each other. A functionally equivalent of a proteinaceous sequence may also be produced by an inverso sequence, wherein one or more D-amino acids are substituted for L amino acids, or vice versa. A partly or fully inverso proteinaceous sequence may be produced by partial or full amino acid substitutions, respectively. A functionally equivalent of a proteinaceous sequence may also be produced by both retro conversion of the carboxyl-amino linkages and partial or full inverso substitution of amino acids, to produce a retroinversopeptidomimetic sequence. For example, a retroinversopeptidomimetic of SEQ ID No. (41) demonstrated inhibitory function, albeit less so than either the D- or L-configurations, against certain household fungi such as a Fusarium and an Aspergillus (Guichard, 1994). The chemical structure of such amino acids (which term is used herein to include imino acids), regardless of stereoisomeric configuration, may be based upon that of the naturally-occurring (e.g., a common) amino acid. Thus, a proteinaceous molecule may possess an activity (e.g., a metal binding activity, an anti-biological activity) in the form of one type of stereoisomer and/or as a mixed stereoisomeric composition.

For example, synthetic peptide combinational libraries (“SPCLs”) were evaluated for activity against fungal pathogens, including pathogens of plants as well as those of animals. The library was comprises of 52,128,400 six-residue peptides, each peptide being comprised of D-amino acids and having non-acetylated N-termini and amidated C-termini. As described in U.S. Pat. Nos. 6,020,312; 5,885,782; and 5,602,097, and patent application Ser. Nos. 10/884,355 and 11/368,086, a hexapeptide library comprised peptides with the first two amino acids in each peptide chain individually and specifically defined and with the last four amino acids comprising an equimolar mixtures of 20 amino acids. In some embodiments, an antibiotic composition(s) comprising equimolar mixture of peptides produced in a synthetic peptide combinatorial library have been derived and shown to have desirable antibiotic activity. In certain embodiments, these relatively variable compositions are based upon the sequences of one or more of the peptides disclosed in any of the U.S. Pat. Nos. 6,020,312; 5,885,782; and 5,602,097, and patent application Ser. Nos. 10/884,355 and 11/368,086. Using these techniques, selected pathogenic fungi, some pathogens of plants and others pathogens of animals, have been tested. These processes have, been shown to be effective where the fungal cell comprises a fungal cell selected from the group of fungi consisting of Fusarium and Aspergillus.

In some aspects, a proteinaceous molecule may comprise or be modified to comprises fewer cysteines and/or exclude cysteine(s) to reduce and/or prevent disulfide linkage and/or cross-linking problem that may occur in certain facets (e.g., a product). Since methods of production can control the chemical nature of the proteinaceous molecule thus produced, synthesis and purification (if desired) of a shorter sequence peptidic agent may be less problematic (e.g., a cysteine is eliminated, which amino acid's free sulfhydryl groups may cause unwanted cross linking).

In another example, a proteinaceous composition comprises proteinaceous molecule (e.g., a peptide, a peptide library) has not been completely defined at one or more amino acid position(s) (e.g., comprising one or more peptides of undefined and/or partly defined sequence), comprises a side chain that has not been de-blocked (i.e., comprises a blocked side chain), comprises a covalent attachment to the synthetic resin (e.g., has not been cleared from a synthetic resin) used to anchor the growing amino acid chain of a peptide, or a combination thereof (e.g., both blocked at a side chain and attached, such as covalently attached, to a resin).

A proteinaceous molecule may be chemically synthesized to incorporate a non-nature amino acid comprising an olefinic side chain, and the olefinic side chain of two or more amino acids may cross-link (e.g., an intra-proteinaceous molecule cross-link, an inter-proteinaceous molecules cross-link) to promote resistance to degradation such as biological (e.g., a protease) and/or chemical degradation (Walensky, L. D. et al., Science 305:1466-1470, 2004). A proteinaceous molecule comprising an olefinic side chain may also cross-link to a non-amino acid based polymer, such as a polymer comprising an olefinic monomer, using standard cross-linking chemistry for a vinyl moiety (e.g., free radical activated cross-linking)., etc, and substitutions may be selected from these types of amino acids.

A proteinaceous molecule may comprise a proteinaceous molecule longer or shorter than the wild-type amino acid sequence(s). For example, an enzyme comprising longer or shorter sequence(s) may be encompassed, insofar as it retains enzymatic activity. In some embodiments, a proteinaceous molecule may comprise one or more peptide and/or polypeptide sequence(s). In certain embodiments, a modification to a proteinaceous molecule may add and/or subtract one or two amino acids from a peptide and/or polypeptide sequence. In other embodiments, a change to a proteinaceous molecule may add and/or remove one or more peptide and/or polypeptide sequence(s). Often a peptide or a polypeptide sequence may be added or removed to confer or remove a specific property from the proteinaceous molecule, and numerous examples of such modifications to a proteinaceous molecule are described herein, particularly in reference to fusion proteins. For example, this signal sequence's amino acid sequence may be deleted by genetic modification in the DNA construction placed into Escherichia coli host cells to enhance its production.

Removal of one or more amino acids from a proteinaceous molecule's sequence may reduce or eliminate a detectable property such as enzymatic activity, binding activity, an anti-fouling activity, etc. However, a longer sequence, particularly a proteinaceous molecule, may consecutively and/or non-consecutively comprises and/or even repeats one or more sequences of a proteinaceous molecule (e.g., a repeated enzymatic sequence, a repeated anti-biological peptide sequence), including but not limited to those disclosed herein. Additionally, a fusion protein may be bioengineered to comprise a wild-type sequence and/or a functional equivalent of a proteinaceous molecule's sequence and an additional peptide and/or polypeptide sequence that confers a property and/or function.

A “fusion protein,” as used herein, comprises an expressed contiguous amino acid sequence comprising a proteinaceous molecule of interest (e.g., a metal binding sequence, an anti-fouling sequence, an anti-biological sequence, an enzyme) and one or more additional peptide and/or polypeptide sequence(s) [“fusion partner(s)]. A “fusion protein” may be a peptide or polypeptide, and does not necessarily comprise a “protein” as previously described. As used herein a “fusion partner” comprises a peptide or polypeptide operatively associated to the sequence of proteinaceous molecule (e.g., a peptide, a polypeptide) of interest (e.g., bioactivity to be conferred to a material formulation). The fusion partner sequence generally provides an useful additional property to the fusion protein, including but not limited to, enhanced expression, enhanced solubility, ease of detection, targeting the fusion protein to a particular location within and/or external to the host cell (e.g., a signal peptide); promoting the ease of purification and/or detection of the fusion protein (e.g., a tag, a fusion partner, a binding sequence); promoting the ease of removal of one or more additional sequences from the peptide and/or the polypeptide of interest (e.g., a protease cleavage site); separating one or more sequences of the fusion protein to allow improved activity and/or function of the sequence(s) (e.g., a linker sequence); and/or promoting retention of a proteinaceous composition in a material formulation (e.g., an immobilization agent sequence).

For example, a fusion partner may comprise a proteinaceous binding sequence for a ligand that may be used to purify the fusion protein from other cellular component(s) and/or material(s), and it is contemplated that such a binding sequence may also be used to immobilize (e.g., promote retention) of the fusion protein as part of (e.g., internally retained) a material formulation that comprise a ligand for the proteinaceous binding sequence. The proteinaceous sequence of interest may also possess one or more sequences and/or property(s) described for a fusion partner sequence (e.g., a metal binding sequence used to purify a fusion protein may also confer metal binding activity to a material formulation). Examples of a binding sequence include an enzyme such as a glutathione-S-transferase that may bind an affinity resin comprising glutathione, a chloramphenicol acetyltransferase that may bind a chloramphenicol; a polypeptide-binding protein such as a staphylococcal protein A and/or a streptococcal protein G that may bind a mammalian IgG's constant Fc region (e.g., an IgG sepharose resin); a calmodulin-binding domain that may bind an affinity resin comprising calmodulin in the presence of calcium; a carbohydrate-binding domain such as a maltose-binding protein, a starch-binding domain, and/or a cellulose-binding domain that may bind maltose (e.g., an affinity resin comprising maltose), starch, and/or cellulose, respectively; chitin-binding domain that may bind an affinity resin comprising chitin; a biotin-binding domain from avidin and/or streptavidin that may bind biotin (e.g., an affinity resin comprising biotin); an antigenic epitope (e.g., a recA, a Flag sequence) that promotes antigenic responses for the production of an antibody that may bind the antigenic epitope sequence; a charged amino acid rich polymer [e.g., a poly(Arg), a poly(Asp)] may be used to bind an ion (e.g., an ion exchange resin's bound ion) and/or an ionomer (e.g., a polyethyleneimine); a histidine rich polymer such as a poly(His) tag that may bind a cation (e.g., a cation of the surface of a metallic pigment, a binder comprising a metallic moiety); a hydrophobic amino acid rich polymer such as a poly(Phe) tag that may bind a hydrophobic material; a cysteine rich polymer such as a poly(Cys) sequence that may bind a thiol moiety comprising material; or a combination thereof (Ljungquist, C. et al., 1989). For example, an arginine rich peptide sequence (e.g., a poly-arginine), which is typically about 2 to about 10 amino acids long (e.g. about 5 amino acids, about 6 amino acids) may bind a cationic polymer (e.g. SP-Sephadex) and/or a negatively charged component of a material formulation such as an aluminosilicate (e.g. a mica). An arginine affinity tag will often disassociate with increasing salt concentration [“Proteins Labfax (Price, N.C., Ed.) BIOS scientific publishers Ltd., Oxford, UK, pp. 52-53, 1996; Unger, T. F., 1997]. In another example, a chitin-binding domain typically comprises an intein cleavage site sequence to allow the self-cleavage in the presence of thiols at reduced temperature to release the peptide and/or the polypeptide sequence of interest. A proteinaceous binding sequence generally has a reduced effect on a fusion protein's folding, particularly as a proteinaceous binding sequence becomes shorter (e.g. about two to about 100 amino acids) and/or the another proteinaceous sequence becomes longer [Proteins Labfax (Price, N.C., Ed.) BIOS scientific publishers Ltd., Oxford, UK, pp. 52-53, 1996]. Examples of a fusion partner that may be used to promote ease of purification includes a thioredoxin, a cellulose-binding domain, a calmodulin binding domain, an avidin, a protein A, a protein G, a glutathione-S-transferase, a chitin-binding domain, an elastin like polypeptide, a maltose-binding domain, or a combination thereof.

As used herein a “tag” comprises a peptide sequence (i.e., a peptide fusion partner) operatively associated to the sequence of another proteinaceous (e.g., a peptide) sequence. In an example, various fusion proteins comprise a proteinaceous tag binding sequence, often as a C-terminal sequence (“tail”), an N-terminal sequence (“tail”), and/or an internal sequence. Examples of a tag include a His-tag, a strep-tag, a flag-tag, a T7-tag, a S-tag, a HSV-tag, a polyarginine-tag, a polycysteine-tag, a polyaspartic acid-tag, a polyphenylalanine-tag, or a combination thereof. A His-tag may comprise about 6 to about 10 amino acids in length, and can be incorporated at the N-terminus, C-terminus, and/or within an amino acid sequence for use in detection and purification. A His tag binds affinity columns comprising nickel, and may be eluted using low pH conditions or with imidazole as a competitor. A strep-tag may comprise about 10 amino acids in length, and may be incorporated at the C-terminus. A strep-tag binds streptavidin or affinity resins that comprise streptavidin. A flag-tag may comprise about 8 amino acids in length, and may be incorporated at the N-terminus and/or the C-terminus of an amino acid sequence for use in purification. A T7-tag may comprise about 11 to about 16 amino acids in length, and may be incorporated at the N-terminus and/or within an amino acid sequence for use in purification. A S-tag may comprise about 15 amino acids in length, and may be incorporated at the N-terminus, C-terminus and/or within an amino acid sequence for use in detection and purification. A HSV-tag may comprise about 11 amino acids in length, and may be incorporated at the C-terminus of an amino acid sequence for use in purification. The HSV tag binds an anti-HSV antibody in purification procedures (Unger, T. F., 1997). A polyarginine-tag may comprise about 5 to about 15 amino acids in length, and may be incorporated at the C-terminus of an amino acid sequence for use in purification. A polycysteine-tag may comprise about 4 amino acids in length, and may be incorporated at the N-terminus of an amino acid sequence for use in purification. A polyaspartic acid-tag may comprise about 5 to about 16 amino acids in length, and may be incorporated at the C-terminus of an amino acid sequence for use in purification. A polyphenylalanine-tag may comprise about 11 amino acids in length, and may be incorporated at the N-terminus of an amino acid sequence for use in purification.

A proteinaceous molecule herein may comprise a cleavage site (e.g., a protease cleavage site, a chemical cleavage site). For example, in recombinant expression, a protease and/or chemical cleavage site may be included between a proteinaceous sequence (e.g., a metal binding sequence) and another sequence (e.g. another metal binding sequence) to allow cleavage to release the sequence(s), such as to convert a contiguous proteinaceous molecule comprising a plurality of peptide sequences (e.g., metal binding peptide sequences) into separate peptide molecules. For example, such a cleavable sequence may be inserted between metal binding sequence(s), such as replacing a series of undefined “Xaa” residues in a sequence comprising a plurality of metal binding motifs. In another example, a longer metal binding sequence may be cleaved into smaller metal binding motif(s) by incorporation of cleavage sequences as substitutes for Xaa rich sequences within the longer sequence. Defined amino acids of a metal binding sequence may also be part of a cleavable sequence. An example of a protease cleavage site includes a subtilisin cleavage site such as a Phe-Ala-His-Tyr-Xaa (SEQ ID No. 246) sequence, with cleavage between the Try and the Xaa; a protease 3C cleavage site such as an Glu-Thr-Leu-Phe-Gln-/Gly-Pro (SEQ ID No. 248) sequence and/or an Glu-Ala-Leu-Phe-Gln-/Gly-Pro (SEQ ID No. 249) sequence, with cleavage before the Gly; a PreScission™ cleavage site such as a thrombin cleavage site such as a Leu-Val-Pro-Arg-/Gly-Ser sequence (SEQ ID No. 244), with cleaved between the Arg and the Gly; a Factor Xa cleavage site such as an Ile-Glu-Gly-Arg-/Xaa (SEQ ID No. 245) sequence and/or an Ile-Asp-Gly-Arg-/Xaa (SEQ ID No. 303) sequence, with cleavage after the Arg; an enterokinase cleavage site such as an Asp-Asp-Asp-Asp-Lys-/Xaa (SEQ ID No. 247) sequence, with cleavage after the Lys (New England Biolabs, 240 County Road, Ipswich, Mass.); and/or a TEV protease cleavage site such as a Glu-Asn-Leu-Tyr-Phe-Gln-/Gly (SEQ ID No. 304) sequence, with cleavage between the Gln and the Gly (Invitrogen Biolabs) [“Protein Microarrays” (Schena, Mark Ed.) Jones and Bartlett Publishers 40 Tall Pine Drive Sudbury, Mass., pp. 271-272, 2005; “High Throughput Protein Expression and Purification Methods and Protocols,” (Doyle, S. A., Ed.) Humana Press, Walnut Creek, Calif., USA, pp. 12-13, 2009]. A chemical agent may be used to cleave a proteinaceous molecule. For example, hydroxylamine cleaves the peptide bond between an asparagine and glycine residues (Moks, T. et al. Biochemistry 26:5239-5244. 1987). In another example, cyanogen bromide may be used to cleave a peptide bond at the carboxyl side of a methionine residue (Itakura, K. et al. Science 198:1056-1063, 1977).

The side chains of amino acids comprise one or more moiety(s) with specific chemical and physical properties. Certain side chains contribute to a ligand binding property, a catalytic property, a stability property, a property related to environmental safety, or a combination thereof. In an example, various acidic, basic, hydrophobic, hydrophilic, and/or aromatic side chains present at or near a binding site of a proteinaceous structure may affect the affinity for a proteinaceous sequence for binding a ligand and/or a substrate for a catalytic reaction, based on the covalent, ionic, Van der Waal forces, hydrogen bond, hydrophilic, hydrophobic, and/or aromatic interactions at a binding site. As used herein, a residue may be “at or near” a residue and/or a group of residues when it is within about 15 Å, about 14 Å, about 13 Å, about 12 Å, about 11 Å, about 10 Å, about 9 Å, about 8 Å, about 7 Å, about 6 Å, about 5 Å, about 4 Å, about 3 Å, about 2 Å, and/or about 1 Å the residue or group of residues such as residues identified as contributing to the active site and/or the binding site of a proteinaceous molecule.

Identification of an amino acid whose chemical modification may likely change a property of a proteinaceous molecule may be accomplished using such methods as a chemical reaction, mutation, X-ray crystallography, nuclear magnetic resonance (“NMR”), computer based modeling, or a combination thereof. Selection of an amino acid on the basis of such information may then be used in the rational design of a mutant proteinaceous sequence that may possess an altered property (Bugg, C. E. et al., 1993; Cohen, A. A. and Shatzmiller, S. E., 1993; Hruby, V. J., 1993; Moore, G. J., 1994; Dean, P. M., 1994; Wiley, R. A. and Rich, D. H., 1993). Additionally, many primary, secondary, tertiary, and/or quaternary structures of proteinaceous molecules may be obtained using a public computerized database [e.g., the Protein Data Bank (PDB)].

A residue of a proteinaceous molecule that contributes to the property(s) of the proteinaceous molecule comprises chemically reactive moiety(s). Such a residue is often susceptible to chemical reaction that may alter (e.g., inhibit) the residue's ability to contribute to a property of the proteinaceous molecule. Thus, a chemical reaction may be used to identify one or more amino acids comprised within the proteinaceous molecule that may contribute to a property. An identified amino acid then may be subject to a modification such as an amino acid substitution and/or a chemical modification to produce a functional equivalent. Examples of amino acids that may be so chemically reacted include Arg, which may be reacted with butanedione; Arg and/or Lys, which may be reacted with phenylglyoxal; Asp and/or Glu, which may be reacted with carbodiimide and HCl; Asp and/or Glu, which may be reacted with N-ethyl-5-phenylisoxazolium-3′-sulfonate (“Woodward's reagent K”); Asp and/or Glu, which may be reacted with 1,3-dicyclohexyl carbodiimide; Asp and/or Glu, which may be reacted with 1-ethyl-3-β-dimethylaminopropyl)carbodiimide (“EDC”); Cys, which may be reacted with p-hydroxy mercuribenzoate; Cys, which may be reacted with dithiobisnitrobenzoate (“DTNB”); Cys, which may be reacted with iodoacetamide; His, which may be reacted with diethylpyrocarbonate (“DEPC”); His, which may be reacted with diazobenzenesulfonic acid (“DBS”); His, which may be reacted with 3,7-bis(dimethylamino)phenothiazin-5-ium chloride (“methylene blue”); Lys, which may be reacted with dimethylsuberimidate; Lys and/or Arg, which may be reacted with 2,4-dinitrofluorobenzene; Lys and/or Arg, which may be reacted with trinitrobenzene sulfonic acid (“TNBS”); Trp, which may be reacted with 2-hydroxy-5-nitrobenzyl bromide 1-ethyl-3(3-dimethylaminopropyl); Trp, which may be reacted with 2-acetoxy-5-nitrobenzyl chloride; Trp, which may be reacted with N-bromosucinimide; Tyr, which may be reacted with N-acetylimidazole (“NAI”); or a combination thereof (Hartleib, J. and Ruterjans, H., 2001b; Josse, D. et al., 1999; Josse, D. et al., 2001).

The modifications that have been made to various enzymes exemplify the types of functional equivalents that may be produced. Recombinant wild-type and mutant forms of the opd gene have been expressed, predominantly in Escherichia coli, for further characterization and analysis. Unless otherwise noted, the various OPH enzymes, whether wild-type or mutants, that act as functional equivalents were prepared using the OPH genes and encoded enzymes first isolated from Pseudomonas diminuta and Flavobacterium spp. OPH normally binds two atoms of Zn²⁺ per monomer when endogenously expressed. While binding a Zn²⁺, this enzyme may comprise a stable dimeric enzyme, with a thermal temperature of melting (“Tₘ”) of approximately 75° C. and a conformational stability of approximately 40 killocalorie per mole (“kcal/mol”) (Grimsley, J. K. et al., 1997). However, structural analogs have been made wherein a Co²⁺, a Fe²⁺, a Cu²⁺, a Mn²⁺, a Cd²⁺, and/or a Ni²⁺ are bound instead to produce enzymes with altered stability and rates of activity (Omburo, G. A. et al., 1992). For example, a Co²⁺ substituted OPH does possess a reduced conformational stability (˜22 kcal/mol). But this reduction in thermal stability may be offset by the improved catalytic activity of a Co²⁺ substituted OPH in degrading various OP compounds. For example, five-fold or greater rates of detoxification of sarin, soman, and VX were measured for a Co²⁺ substituted OPH relative to OPH binding Zn²⁺. A structural analog of an OPH sequence may be prepared comprising a Zn²⁺, a Co²⁺, a Fe²⁺, a Cu²⁺, a Mn²⁺, a Cd²⁺, a Ni²⁺, or a combination thereof. Generally, changes in the bound metal may be achieved by using cell growth media during cell expression of the enzyme wherein the concentration of a metal present may be defined, and/or removing the bound metal with a chelator (e.g., 1,10-phenanthroline; 8-hydroxyquinoline-5-sulfphonic acid; ethylenediaminetetraacetic acid) to produce an apo-enzyme, followed by reconstitution of a catalytically active enzyme by contact with a selected metal (Omburo, G. A. et al., 1992; Watkins, L. M. et al., 1997a; Watkins, L. M. et al., 1997b). A structural analog of an OPH sequence may be prepared to comprise one metal atom per monomer.

In an additional example, OPH structure analysis has been conducted using NMR (Omburo, G. A. et al., 1993). In a further example, the X-ray crystal structure for OPH has been determined (Benning, M. M. et al., 1994; Benning, M. M. et al., 1995; Vanhooke, J. L. et al., 1996), including the structure of the enzyme while binding a substrate, further identifying residues involved in substrate binding and catalytic activity (Benning, M. M. et al., 2000). From these structure evaluations, the amino acids His55, His57, His201, His230, Asp301, and the carbamylated lysine, Lys169, have been identified as coordinating the binding of the active site metal. Additionally, the positively charged amino acids His55, His57, His201, His230, His254, and His257 are counter-balanced by the negatively charged amino acids Asp232, Asp233, Asp235, Asp 253, Asp301, and the carbamylated lysine Lys169 at the active site area. A water molecule and amino acids His55, His57, Lys169, His201, His230, and Asp301 are thought to be involved in direct metal binding. The amino acid Asp301 may aid a nucleophilic attack by a bound hydroxide upon the phosphorus to promote cleavage of an OP compound, while the amino acid His354 may aid the transfer of a proton from the active site to the surrounding liquid in the latter stages of the reaction (Raushel, F. M., 2002). The amino acids His254 and His257 are not thought to comprise direct metal binding amino acids, but may comprise residues that interact (e.g., a hydrogen bond, a Van der Waal interaction) with each other and other active site residue(s), such as a residue that directly contact a substrate and/or bind a metal atom. In particular, amino acid His254 may interact with the amino acids His230, Asp232, Asp233, and Asp301. Amino acid His257 may comprise a participant in a hydrophobic substrate-binding pocket. The active site pocket comprises various hydrophobic amino acids, Trp131, Phe132, Leu271, Phe306, and Tyr309. These amino acids may aid the binding of a hydrophobic OP compound (Benning, M. M. et al., 1994; Benning, M. M. et al., 1995; Vanhooke, J. L. et al., 1996). Electrostatic interactions may occur between phosphoryl oxygen, when present, and the side chains of Trp131 and His201. Additionally, the side chains of amino acids Trp131, Phe132, and Phe306 are thought to be orientated toward the atom of the cleaved substrate's leaving group that was previously bonded to the phosphorus atom (Watkins, L. M. et al., 1997a).

Substrate binding subsites known as the small subsite, the large subsite, and the leaving group subsite have been identified (Benning, M. M. et al., 2000; Benning, M. M. et al., 1994; Benning, M. M. et al., 1995; Vanhooke, J. L. et al., 1996). The amino acids Gly60, Ile106, Leu303, and Ser308 are thought to comprise the small subsite. The amino acids Cys59 and Ser61 are near the small subsite, but with the side chains thought to be orientated away from the subsite. The amino acids His254, His257, Leu271, and Met317 are thought to comprise the large subsite. The amino acids Trp131, Phe132, Phe306, and Tyr309 are thought to comprise the leaving group subsite, though Leu271 may be considered part of this subsite as well (Watkins, L. M. et al., 1997a). Comparison of this opd product with the encoded sequence of the opdA gene from Agrobacterium radiobacter P230 revealed that the large subsite possessed generally larger residues that affected activity, specifically the amino acids Arg254, Tyr257, and Phe271 (Home, I. et al., 2002). Few electrostatic interactions are apparent from the X-ray crystal structure of the inhibitor bound by OPH, and hydrophobic interaction(s) and the size of the subsite(s) may affect substrate specificity, including steriospecificity for a stereoisomer, such as a specific enantiomer of an OP compound's chiral chemical moiety (Chen-Goodspeed, M. et al., 2001b).

Using the sequence and structural knowledge of OPH, numerous mutants of OPH comprising a sequence analog have been specifically produced to alter one or more properties relative to a substrate's cleavage rate (k_{cat}) and/or specificity (k_{cat}/Kₘ). Examples of OPH sequence analog mutants include H55C, H57C, C59A, G60A, S61A, I106A, I106G, W131A, W131F, W131K, F132A, F132H, F132Y, L136Y, L140Y, H201C, H230C, H254A, H254R, H254S, H257A, H257L, H257Y, L271A, L271Y, L303A, F306A, F306E, F306H, F306K, F306Y, S308A, S308G, Y309A, M317A, M317H, M317K, M317R, H55C/H57C, H55C/H201C, H55C/H230C, H57C/H201C, H57C/H230C, A80V/S365P, I106A/F132A, I106A/S308A, I106G/F132G, I106G/S308G, F132Y/F306H, F132H/F306H, F132H/F306Y, F132Y/F306Y, F132A/S308A, F132G/S308G, L182S/V310A, H201C/H230C, H254R/H257L, H55C/H57C/H201C, H55C/H57C/H230C, H55C/H201C/H230C, I106A/F132A/H257Y, I106A/F132A/H257W, I106G/F132G/S308G, L130M/H257Y/I274N, H257Y/I274N/S365P, H55C/H57C/H201C/H230C, I106G/F132G/H257Y/S308G, and/or A14T/A80V/L185R/H257Y/I274N (Li, W.-S. et al., 2001; Gopal, S. et al., 2000; Chen-Goodspeed, M. et al., 2001a; Chen-Goodspeed, M. et al., 2001b; Watkins, L. M. et al., 1997a; Watkins, L. M. et al., 1997b; diSioudi, B. et al., 1999; Cho, C. M.-H. et al., 2002; Shim, H. et al., 1996; Raushel, F. M., 2002; Wu, F. et al., 2000a; diSioudi, B. D. et al., 1999).

For example, the sequence and structural information has been used in production of mutants of OPH possessing cysteine substitutions at the metal binding histidines His55, His57, His201, and His230. OPH mutants H55C, H57C, H201C, H230C, H55C/H57C, H55C/H201C, H55C/H230C, H57C/H201C, H57C/H230C, H201C/H230C, H55C/H57C/H201C, H55C/H57C/H230C, H55C/H201C/H230C, H57C/H201C/H230C, and H55C/H57C/H201C/H230C were produced binding either a Zn²⁺; a Co²⁺ and/or a Cd²⁺. The H57C mutant had between 50% (i.e., binding a Cd²⁺, a Zn²⁺) and 200% (i.e., binding a Co²⁺) wild-type OPH activity for paraoxon cleavage. The H201C mutant had about 10% activity, the H230C mutant had less than 1% activity, and the H55C mutant bound one atom of a Co²⁺ and possessed little detectable activity, but may still be useful if possessing an useful property (e.g., enhanced stability) (Watkins, L. M., 1997b).

In an additional example, the sequence and structural information has been used in production of mutants of OPH possessing altered metal binding and/or bond-type cleavage properties. OPH mutants H254R, H257L, and H254R/H257L have been made to alter amino acids that are thought to interact with nearby metal-binding amino acids. These mutants also reduced the number of metal ions (i.e., Co²⁺, Zn²⁺) binding the enzyme dimer from four to two, while still retaining 5% to greater than 100% catalytic rates for the various substrates. These reduced metal mutants possess enhanced specificity for larger substrates such as NPPMP and demeton-S, and reduced specificity for the smaller substrate diisopropyl fluorophosphonate (diSioudi, B. et al., 1999). In a further example, the H254R mutant and the H257L mutant each demonstrated a greater than four-fold increase in catalytic activity and specificity against VX and its analog demeton S. The H257L mutant also demonstrated a five-fold enhanced specificity against soman and its analog NPPMP (diSioudi, B. D. et al., 1999).

In an example, specific mutants of OPH (i.e., a phosphotriesterase), were designed and produced to aid phosphodiester substrates to bind and be cleaved by OPH. These substrates either comprised a negative charge and/or a large amide moiety. A M317A mutant was created to enlarge the size of the large subsite, and M317H, M317K, and M317R mutants were created to incorporate a cationic group in the active site. The M317A mutant demonstrated a 200-fold cleavage rate enhancement in the presence of alkylamines, which were added to reduce the substrate's negative charge. The M317H, M317K, and M317R mutants demonstrated modest improvements in rate and/or specificity, including a 7-fold k_{cat}/Kₘ improvement for the M317K mutant (Shim, H. et al., 1998).

In a further example, the W131K, F132Y, F132H, F306Y, F306H, F306K, F306E, F132H/F306H, F132Y/F306Y, F132Y/F306H, and F132H/F306Y mutants were made to add and/or change the side chain of active site residues to form a hydrogen bond and/or donate a hydrogen to a cleaved substrate's leaving group, to enhance the rate of cleavage for certain substrates, such as phosphofluoridates. The F132Y, F132H, F306Y, F306H, F132H/F306H, F132Y/F306Y, F132Y/F306H, and F132H/F306Y mutants all demonstrated enhanced enzymatic cleavage rates, of about three- to ten-fold improvement, against the phosphonofluoridate, diisopropyl fluorophosphonate (Watkins, L. M. et al., 1997a).

In an additional example, OPH mutants W131F, F132Y, L136Y, L140Y, L271Y and H257L were designed to modify the active site size and placement of amino acid side chains to refine the structure of binding subsites to specifically fit the binding of a VX substrate. The refinement of the active site structure produced a 33% increase in cleavage activity against VX in the L136Y mutant (Gopal, S. et al., 2000).

Various mutants of OPH have been made to alter the steriospecificity, and in some cases, the rate of reaction, by substitutions in substrate binding subsites. For example, the C59A, G60A, S61A, I106A, W131A, F132A, H254A, H257A, L271A, L303A, F306A, S308A, Y309A, and M317A mutants of OPH have been produced to alter the size of various amino acids associated with the small subsite, the large subsite and the leaving group subsite, to alter enzyme activity and selectivity, including sterioselectivity, for various OP compounds. The G60A mutant reduced the size of the small subsite, and decreased both rate (k_{cat}) and specificity (k_{cat}/Kₐ) for Rₚ-enantiomers, thereby enhancing the overall specificity for some Sₚ-enantiomers to over 11,000:1. Mutants I106A and S308A, which enlarged the size of the small subsite, as well as mutant F132A, which enlarged the leaving group subsite, all increased the reaction rates for Rₚ-enantiomers and reduced the specificity for Sₚ-enantiomers (Chen-Goodspeed, M. et al., 2001a).

Additional mutants I106A/F132A, I106A/S308A, F132A/S308A, I106G, F132G, S308G, I106G/F132G, I106G/S308G, F132G/S308G, and I106G/F132G/S308G were produced to further enlarge the small subsite and leaving group subsite. These OPH mutants demonstrated enhanced selectivity for Rₚ-enantiomers. Mutants H254Y, H254F, H257Y, H257F, H257W, H257L, L271Y, L271F, L271W, M317Y, M317F, and M317W were produced to shrink the large subsite, with the H257Y mutant, for example, demonstrating a reduced selectivity for Sₚ-enantiomers (Chen-Goodspeed, M. et al., 2001b). Further mutants I106A/H257Y, F132A/H257Y, I106A/F132A/H257Y, I106A/H257Y/S308A, I106A/F132A/H257W, F132A/H257Y/S308A, I106G/H257Y, F132G/H257Y, I106G/F132G/H257Y, I106G/H257Y/S308G, and I106G/F132G/H257Y/S308G were made to simultaneously enlarge the small subsite and shrink the large subsite. Mutants such as H257Y, I106A/H257Y, I106G, I106A/F132A, and I106G/F132G/S308G were effective in altering steriospecificity for Sₚ:Rₚ enantiomer ratios of some substrates to less than 3:1 ratios. Mutants including F132A/H257Y, I106A/F132A/H257W, I106G/F132G/H257Y, and I106G/F132G/H257Y/S308G demonstrated a reversal of selectivity for Sₚ:Rₚ enantiomer ratios of some substrates to ratios from 3.6:1 to 460:1. In some cases, such a change in steriospecificity was produced by enhancing the rate of catalysis of a Rₚ enantiomer with little change on the rate of Sₚ enantiomer cleavage (Chen-Goodspeed, M. et al., 2001b; Wu, F. et al., 2000a).

Such alterations in sterioselectivity may enhance OPH performance against a specific OP compound that may comprise a target of detoxification, including a CWA. Enlargement of the small subsite by mutations that substitute the Ile106 and Phe132 residues with the less bulky amino acid alanine and/or reduction of the large subsite by a mutation that substitutes His257 with the bulkier amino acid phenylalanine increased catalytic rates for the Sₚ-isomer; and decreased the catalytic rates for the Rₚ-isomers of a sarin analog, thus resulting in a triple mutant, I106A/F132A/H257Y, with a reversed sterioselectivity such as a Sₚ:Rₚ preference of 30:1 for the isomers of the sarin analog. A mutant of OPH designated G60A has also been created with enhanced steriospecificity relative to specific analogs of enantiomers of sarin and soman (Li, W.-S. et al., 2001; Raushel, F. M., 2002). Of greater interest, these mutant forms of OPH have been directly assayed against sarin and soman nerve agents, and demonstrated enhanced detoxification rates for racemic mixtures of sarin or soman enantiomers. Wild-type OPH has a k_{cat} for sarin of 56 s⁻¹, while the I106A/F132A/H257Y mutant has k_{cat} for sarin of 1000 s⁻¹. Additionally, wild-type OPH has a k_{cat} for soman of 5 s⁻¹, while the G60A Mutant has k_{cat} for soman of 10 s⁻¹ (Li, W.-S. et al., 2001).

It is also possible to produce a mutant enzyme with an enhanced enzymatic property against a specific substrate by evolutionary selection and/or exchange of encoding DNA segments with related proteins rather than rational design. Such techniques may screen hundreds or thousands of mutants for enhanced cleavage rates against a specific substrate [see, for example, “Directed Enzyme Evolution: Screening and Selection Methods (Methods in Molecular Biology) (Arnold, F. H. and Georgiou, G) Humana Press, Totowa, N.J., 2003; Primrose, S. et al., “Principles of Gene Manipulation” pp. 301-303, 2001]. The mutants identified may possess substitutions at amino acids that have not been identified as directly comprising the active site, or its binding subsites, using techniques such as NMR, X-ray crystallography and computer structure analysis, but still contribute to activity for one or more substrates. For example, selection of OPH mutants based upon enhanced cleavage of methyl parathion identified the A80V/S365P, L182S/V310A, I274N, H257Y, H257Y/I274N/S365P, L130M/H257Y/I274N, and A14T/A80V/L185R/H257Y/I274N mutants as having enhanced activity. Amino acids Ile274 and Val310 are within 10 Å of the active site, though not originally identified as part of the active site from X-ray and computer structure analysis. However, mutants with substitutions at these amino acids demonstrated improved activity, with mutants comprising the I274N and H257Y substitutions particularly active against methyl parathion. Additionally, the mutant, A14T/A80V/L185R/H257Y/I274N, further comprising a L185R substitution, was active having a 25-fold improvement against methyl parathion (Cho, C. M.-H. et al., 2002).

In an example, a functional equivalent of OPH may be prepared that lacks the first 29-31 amino acids of the wild-type enzyme. The wild-type form of OPH endogenously or recombinantly expressed in Pseudomonas or Flavobacterium removes the first N-terminal 29 amino acids from the precursor protein to produce the mature, enzymatically active protein (Mulbry, W. and Karns, J., 1989; Serdar, C. M. et al., 1989). Recombinant expressed OPH in Gliocladium vixens apparently removes part or all of this sequence (Dave, K. I. et al., 1994b). Recombinant expressed OPH in Streptomyces lividans primarily has the first 29 or 30 amino acids removed during processing, with a few percent of the functional equivalents having the first 31 amino acids removed (Rowland, S. S. et al., 1992). Recombinant expressed OPH in Spodoptera frupperda cells has the first 30 amino acids removed during processing (Dave, K. I. et al., 1994a).

The 29 amino acid leader peptide sequence targets OPH enzyme to the cell membrane in Escherichia coli, and this sequence may be partly or fully removed during cellular processing (Dave, K. I. et al., 1994a; Miller, C. E., 1992; Serdar, C. M. et al., 1989; Mulbry, W. and Karns, J., 1989). The association of OPH comprising the leader peptide sequence with the cell membrane in Escherichia coli expression systems seems to be relatively weak, as brief 15 second sonication releases most of the activity into the extracellular environment (Dave, K. I. et al., 1994a). For example, recombinant OPH may be expressed without this leader peptide sequence to enhance enzyme stability and expression efficiency in Escherichia coli (Serdar, C. M., et al. 1989). In another example, recombinant expression efficiency in Pseudomonas putida for OPH was improved by retaining this sequence, indicating that different species of bacteria may have varying preferences for a signal sequence (Walker, A. W. and Keasling, J. D., 2002). However, the length of an enzymatic sequence may be readily modified to improve expression or other properties in a particular organism, or select a cell with a relatively good ability to express a biomolecule, in light of the present disclosures and methods in the art (see U.S. Pat. Nos. 6,469,145, 5,589,386 and 5,484,728).

In an example, recombinant OPH sequence-length mutants have been expressed wherein the first 33 amino acids of OPH have been removed, and a peptide sequence M-I-T-N-S added at the N-terminus (Omburo, G. A. et al., 1992; Mulbry, W. and Karns, J., 1989). Often removal of the 29 amino acid sequence may be used when expressing mutants of OPH comprising one or more amino acid substitutions such as the C59A, G60A, S61A, I106A, W131A, F132A, H254A, H257A, L271A, L303A, F306A, S308A, Y309A, M317A, I106A/F132A, I106A/S308A, F132A/S308A, I106G, F132G, S308G, I106G/F132G, I106G/S308G, F132G/S308G, I106G/F132G/S308G, H254Y, H254F, H257Y, H257F, H257W, H257L, L271Y, L271W, M317Y, M317F, M317W, I106A/H257Y, F132A/H257Y, I106A/F132A/H257Y, I106A/H257Y/S308A, I106A/F132A/H257W, F132A/H257Y/S308A, I106G/H257Y, F132G/H257Y, I106G/F132G/H257Y, I106G/H257Y/S308G, and I106G/F132G/H257Y/S308G mutants (Chen-Goodspeed, M. et al., 2001a). In a further example, LacZ-OPH fusion protein mutants lacking the 29 amino acid leader peptide sequence and comprising an amino acid substitution mutant such as W131F, F132Y, L136Y, L140Y, H257L, L271L, L271Y, F306A, or F306Y have been recombinantly expressed (Gopal, S. et al., 2000).

In an additional example, OPH mutants that comprise additional amino acid sequences are also known in the art. An OPH fusion protein lacking the 29 amino acid leader sequence and possessing an additional C-terminal flag octapeptide sequence was expressed and localized in the cytoplasm of Escherichia coli (Wang, J. et al., 2001). In another example, nucleic acids encoding truncated versions of the ice nucleation protein (“InaV”) from Pseudomonas syringae have been used to construct vectors that express OPH-InaV fusion proteins in Escherichia coli. The InaV sequences targeted and anchored the OPH-InaV fusion proteins to the cells' outer membrane (Shimazu, M. et al., 2001a; Wang, A. A. et al., 2002). In a further example, a vector encoding a similar fusion protein was expressed in Moraxella sp., and demonstrated a 70-fold improved OPH activity on the cell surface compared to Escherichia coli expression (Shimazu, M. et al., 2001b). In a further example, fusion proteins comprising the signal sequence and first nine amino acids of lipoprotein, a transmembrane domain of outer membrane protein A (“Lpp-OmpA”), and either a wild-type OPH sequence or an OPH truncation mutant lacking the first 29 amino acids has been expressed in Escherichia coli. These OPH-Lpp-OmpA fusion proteins were targeted and anchored to the Escherichia coli cell membrane, though the OPH truncation mutant had 5% to 10% the activity of the wild-type OPH sequence (Richins, R. D. et al., 1997; Kaneva, I. et al., 1998). In one example, a fusion protein comprising N-terminus to C-terminus, a (His)6 polyhistidine tag, a green fluorescent protein (“GFP”), an enterokinase recognition site, and an OPH sequence lacking the 29 amino acid leader sequence has been expressed within Escherichia coli cells (Wu, C.-F. et al., 2000b, Wu, C.-F. et al., 2002). A similar fusion protein a (His)6 polyhistidine tag, an enterokinase recognition site, and an OPH sequence lacking the 29 amino acid leader sequence has also been expressed within Escherichia coli cells (Wu, C.-F. et al., 2002). Additionally, variations of these GFP-OPH fusion proteins have been expressed within Escherichia coli cells where a second enterokinase recognition site was placed at the C-terminus of the OPH gene fragment sequence, followed by a second OPH gene fragment sequence (Wu, C.-F. et al., 2001b). The GFP sequence produced fluorescence that was proportional to both the quantity of the fusion protein, and the activity of the OPH sequence, providing a fluorescent assay of enzyme activity and stability in GFP-OPH fusion proteins (Wu, C.-F. et al., 2000b, Wu, C.-F. et al., 2002).

In a further example, a fusion protein comprising an elastin-like polypeptide (“ELP”) sequence, a polyglycine linker sequence, and an OPH sequence was expressed in Escherichia coli (Shimazu, M. et al., 2002). In an additional example, a cellulose-binding domain at the N-terminus of an OPH fusion protein lacking the 29 amino acid leader sequence, and a similar fusion protein wherein OPH possessed the leader sequence, where both predominantly excreted into the external medium as soluble proteins by recombinant expression in Escherichia coli (Richins, R. D. et al., 2000).

In another example of enzyme functional equivalents, various chemical modifications to the amino acid residues of the recombinantly expressed human paraoxonase have been used to identify specific residues including tryptophans, histidines, aspartic acids, and glutamic acids as functioning in enzymatic activity for the cleavage of phenylacetate, paraoxon, chlorpyrifosoxon. and diazoxon. Additionally, comparison to conserved residues in human, mouse, rabbit, rat dog, chicken, and turkey paraoxonase enzymes was used to further identify amino acids for the production of specific mutants. Site-directed mutagenesis was used to alter the enzymatic activity of human paraoxonase through conservative and non-conservative substitutions, and thus clarify the specific amino acids functioning in enzymatic activity. Specific paraoxonase mutants include the sequence analogs E32 Å, E48 Å, E52 Å, D53 Å, D88 Å, D107 Å, H114N, D121 Å, H133N, H154N, H160N, W193 Å, W193F, W201 Å, W201F, H242N, H245N, H250N, W253 Å, W253F, D273 Å, W280 Å, W280F, H284N, and/or H347N. The various paraoxonase mutants generally had different enzymatic properties. For example, W253 Å had a 2-fold greater k_{cat}; and W201F, W253 Å and W253F each had a 2 to 4 fold increase in k_{cat}, though W201F also had a lower substrate affinity. A non-conservative substitution mutant W280 Å had 1% wild-type paraoxonase activity, but the conservative substitution mutant W280F had similar activity as the wild-type paraoxonase (Josse, D. et al., 1999; Josse, D. et al., 2001).

In a further example of functional equivalents, various chemical modifications to the amino acid residues of the recombinantly expressed squid-type DFPase from Loligo vulgaris has been used to identify which specific types of residues of modified arginines, aspartates, cysteines, glutamates, histidines, lysines, and tyrosines, function in enzymatic activity for the cleavage of DFP. Modification of histidines generally reduced enzyme activity, and site-directed mutagenesis was used to clarify which specific histidines function in enzymatic activity. Specific squid-type DFPase mutants include the sequence analogs H181N, H224N, H274N, H219N, H248N, and/or H287N. The H287N mutant lost about 96% activity, and may act as a hydrogen acceptor in active site reactions. The H181N and H274N mutants lost between 15% and 19% activity, and are thought to help stabilize the enzyme. The H224N mutant gained about 14% activity, indicating that alterations to this residue may also affect activity (Hartleib, J. and Ruterjans, H., 2001b). In a further example of squid-type DFPase functional equivalents, recombinant squid-type DFPase sequence-length mutants have been expressed wherein a (His)6 tag sequence and a thrombin cleavage site has been added to the squid-type DFPase (Hartleib, J. and Ruterjans, H., 2001a). In an additional example, a polypeptide comprising amino acids 1-148 of squid-type DFPase has been admixed with a polypeptide comprising amino acids 149-314 of squid-type DFPase to produce an active enzyme (Hartleib, J. and Ruterjans, H., 2001a).

Further, a variety of modification(s) (e.g., a chemical modification) of the art can be made to a proteinaceous molecule (e.g., a peptide), particularly a modification that may confer, retain, and/or alter a property (e.g., a metal binding property, an anti-fouling property, an anti-biological activity). For example, some modifications may be used to increase the metal binding, anti-biological potency of a proteinaceous molecule. In another example, though a modification may reduce a metal binding activity of a proteinaceous molecule, such a reduction may still produce a proteinaceous molecule with suitable anti-fouling activity. Other modifications may facilitate handling of a peptide. Other modifications may alter a binding property. A proteinaceous molecule's functional moiety that may typically be modified include a hydroxyl, an amino, a guanidinium, a carboxyl, an amide, a phenol, an imidazol ring(s), and/or a sulfhydryl. Typical reactions of these moieties include, for example, acetylation of a hydroxyl group by an alkyl halide; esterification, amidation (e.g., carbodiimides or other catalyst mediated amidation), and/or reduction to an alcohol of a carboxyl moiety; acidic or basic condition deamidation of an asparagine and/or a glutamine; an acylation, an alkylation, an arylation, and/or an amidation reaction of an amino group such as the primary amino group of a proteinaceous molecule (e.g., a peptide) and/or the amino group of a lysine residue; halogenation and/or nitration of the phenolic moiety of a tyrosine; or a combination thereof. Examples where solubility of a proteinaceous molecule (e.g., a peptide) may be decreased include acylating a charged lysine residue and/or acetylating a carboxyl moiety of an aspartic acid and/or a glutamic acid. Additional examples of chemical modifications include, when applicable, a hydroxylation of a proline and/or a lysine; a phosphorylation of a hydroxyl group of a serine and/or a threonine; a methylation of an alpha-amino group of a lysine, an arginine and/or a histidine (Creighton, T. E., 1983); adding a detectable label such as a fluorescein isothiocyanate compound (“FITC”) to a lysine side chain and/or a terminal amine (Rogers, K. R. et al., 1999); covalent attachment of a poly ethylene glycol (Yang, Z. et al., 1995; Kim, C. et al., 1999; Yang, Z. et al., 1996); an acylatylation of an amino acid, particularly at the N-terminus; an amination of an amino acid, particularly at the C-terminus (Greene, T. W. and Wuts, P. G. M. “Productive Groups in Organic Synthesis,” Second Edition, pp. 309-315, John Wiley & Sons, Inc., USA, 1991); a deamidation of an asparagine or a glutamine to an aspartic acid or glutamic acid, respectively; a derivation of an amino acid by a sugar moiety, a lipid, a phosphate, and/or a farnysyl group; an aggregation (e.g., a dimerization) of a plurality of proteinaceous molecules, whether of identical sequence or varying sequences; an ionization of an amino acid into an acidic, basic or neutral salt form; an oxidation of an amino acid; or a combination thereof of any of the forgoing. Such modifications may produce an alteration in a property of a proteinaceous molecule and/or may aid in immobilization of a proteinaceous molecule. For example, a N-terminal glycosylation may enhance a proteinaceous molecule's stability (Powell, M. F. et al., 1993). In an additional example, substitution of a beta-amino acid isoserine for a serine may enhance the aminopeptidase resistance a proteinaceous molecule (Coller, B. S. et al., 1993). Techniques and materials for such modification of a proteinaceous molecule (e.g., a peptide) to other molecules described herein and/or of the art (e.g., the literature), may be used.

In an additional example, a biomolecular composition (e.g., a proteinaceous molecule) may be chemically linked and/or bonded (e.g., covalently linked, ionically associated) to a component (e.g., a polymer) of a material formulation (e.g., a plastic, a coating, a coating produced film) to incorporate a biomolecular composition into a material formulation. For example, that ability to link a proteinaceous molecule to a polymeric carrier may also be used for chemically linking or otherwise associating one or more anti-biological proteinaceous molecule (e.g., a metal binding peptide having an anti-fouling activity) to a polymeric material (e.g., a plastic fabric, a roofing material) which would otherwise be more susceptible to infestation, defacement and/or deterioration by a cell. Conventional techniques for linking the N- or C-terminus of a peptide to a long-chain polymer may be employed. For example, an anti-biological proteinaceous molecule may include additional amino acids on the linking end to facilitate linkage to the polymer (e.g., a PVC polymer). A polyvinyl chloride (“PVC”) is only one example of many types of a polymeric material (e.g., a plastic) that may be linked to a proteinaceous molecule (e.g., an antifungal peptide) in this manner.

9. Processing of Cells and Biomolecules

After production of a biomolecule by chemical synthesis, production in a living cell such as via endogenous expression and/or expression due to recombinant engineering, the cell and/or biomolecule may undergo one or more processing techniques to prepare a biomolecular composition. Such a biomolecular composition may comprise various additional cellular component(s) and/or chemical(s), though in some embodiments a biomolecule may be isolated and/or purified with only a small percentage (e.g., about 0.0000001% to about 20% or less; a trace amount) of additional biomolecule(s)/cellular components. For example, a biomolecular composition (e.g., a proteinaceous agent) comprises a substantially homogeneous biomolecule composition, and/or a mixture of biomolecules (e.g., a plurality of peptides). For example, a homogeneous peptide composition may comprise a single active peptide specie of a well-defined (e.g., most or all amino acids defined) sequence, though a minor amount (e.g., less than about 20% by moles) of impurity(s) [e.g., residual chemical(s) from chemical synthesis] may coexist with the peptide in the peptide composition so long as the impurity does not interfere with a desired property(s) of the active peptide (e.g., a growth inhibitory property). For example, a “crude cell preparation” comprises a desired biomolecule within and/or otherwise in contact with a cell (e.g., a sterilized cell, a permeabilized cell) and/or a cellular debris from the biomolecule's production. In certain aspects, the total content of desired biomolecule may range from about 0.0000001% to about 99.9999% of a crude cell preparation, by volume and/or dry weight, depending upon factors such as expression efficiency of the biomolecule in the cell and the amount of processing and/or purification techniques.

In certain embodiments, the biomolecular composition comprises cellular components, particularly a cell wall and/or a cell membrane material, to provide material that may be protective (e.g., provide a stabilizing effect for a desired biomolecule, protect a desired biomolecule from a chemical) of the biomolecule, enhances the particulate nature of the biomolecular composition, or a combination thereof. For example, a biomolecule encapsulated (e.g., partly encapsulated, fully encapsulated) by a cellular material such as a cell wall may be protected from a material formulation's component (e.g., a solvent, a binder, a polymer, a cross-linking agent, a reactive chemical such as a peroxide, an additive, etc.); a material formulation related chemical reaction (e.g., thermosetting reaction); a potentially damaging agent that a material formulation may contact (e.g., a chemical, a solvent, a detergent, etc.); or a combination thereof. A “whole cell material” or “whole cell particulate material,” which refers to cell-based particulate material resembling an intact living cell upon microscopic examination, in contrast to cell fragments of varying shape and size, and may comprise protective cellular components for a biomolecule. The whole cell particulate material comprises about 50% to about 100%, of a whole cell material. The percentage of whole cell material and cell fragments may be determined by any applicable technique in the art such as microscopic examination, centrifugation, etc, as well as any technique described for determining the properties of a pigment, an extender, and/or other particulate material either alone and/or comprised in a material formulation. A preparation of a cell-based particulate material may comprise a certain percentage of cell fragments, which comprise pieces of a cell wall, a cell membrane, and/or other cell components (e.g., an expressed biomolecule). In some aspects, cell fragments may be used as a cell-based particulate material. The cell fragment particulate material comprises about 50% to about 100%, of cell fragment material, and the cellular fragments may also be protective of a desired biomolecule.

Depending upon the type of processing used various cell components may be partly and/or fully removed from the cell to produce a biomolecular composition (e.g., a cell-based particulate material, an isolated and/or purified biomolecule). For example, a processing technique may comprise contacting a cell with a liquid (e.g., an organic liquid) to dissolve a cell component(s). Removal of the solvent may thereby remove (“extract”) the dissolved cell component(s) from the particulate matter. However, a large biomolecule/cell fragment material (e.g., greater than about 1,000 kDa molecular mass), particularly a polymer comprised as part of a cell wall (e.g., a peptidoglycan, a teichoic acid, a lipopolysacharide) may be resistant to extraction and thus be retained as a component of the particulate matter. A chemical moiety of the large biomolecule at the interface of the particulate matter and the external environment may chemically react with, for example, a component of a material formulation, and such a reaction may be used in the chemical cross-linking of biomolecular composition (e.g., a cell-based particulate material) to a component of a material formulation (e.g., a binder in a thermosetting coating).

One or more processing techniques that may be used in preparing, isolating, and/or purifying a biomolecular composition (e.g., a biomolecule). For example, a processing technique may comprise sterilizing a biomolecular composition. Sterilizing (“inactivating”) kills living matter (e.g., a cell, a virus), while attenuation reduces the virulence of a living matter. A sterilizing and/or attenuating technique may be used as continued post expression growth of a cell, a virus, and/or a contaminating organism may detrimentally affect a material formulation in some embodiments.

In certain embodiments, it contemplated that sterilization and/or attenuation may be accomplished by contact with biologically detrimental component of such a material formulation such as a solvent and/or chemically reactive component (e.g., a thermosetting binder, a cross linking agent). Sterilizing and/or attenuation of a biomolecular composition and/or material formulation (e.g., a cell-based particulate material) comprising such a material may be accomplished by any method known in the art. Examples of sterilizing and/or attenuating may include contacting the biomolecular composition with a toxin, irradiating (e.g., ionizing irradiation, infrared irradiation, ultra-violet irradiation, particle irradiation, microwave irradiation, etc), heating the living matter above a temperature suitable for life (e.g., 100° C. in many cases, more for an extremophile), or a combination thereof. However, in alternative embodiments, a partly sterilized, partly attenuated, a non-sterilized and/or attenuated biomolecular composition (e.g., a cell-based particulate material) may be suitable for a temporary material formulation (e.g., a polymeric material with a relatively reduced service life, a temporary coating).

A processing technique may comprise concentrating a biomolecular composition. As used herein, “concentrating” refers to any process reducing the volume of a composition, an article, etc. Concentrating a biomolecular composition (e.g., cell-based particulate material) may be by any method known in the art, including, for example, washing, filtrating, a gravitational force, a gravimetric force, or a combination thereof. An example of a gravimetric force comprises the force exerted during centrifugation. After desired biomolecule(s) (e.g., cell based particulate materials) are localized to the bottom of a centrifugation devise, the media may be removed via such techniques as decanting, aspiration, etc.

In additional embodiments, the biomolecular composition may be dried. Such a drying technique may remove an undesired liquid, such as from a cell-based particulate material. Examples of drying include freeze-drying, spray drying, lyophilizing, or a combination thereof. In some aspects, a cryoprotectant may be added to the biomolecular composition during a drying technique (e.g., lyophilizing). In some aspects, a biomolecular composition (e.g., a particulate material) comprise a form (e.g., a powder) sufficiently liquid free (“dry”) that it may be suitable for convenient storage at ambient and/or other temperature conditions without desiccation.

An application of physical force (e.g., grinding, milling, shearing) may enhance the particulate nature of the material, and/or the average particle size may be reduced to a desired range, including the conversion of cell(s) into disrupted cell(s) and/or cell debris. Such a physical force may produce a powder form, such as a power of a cell-based particulate material. Physical force may also be used in processing techniques dealing with a purified and/or a semi-purified biomolecule (e.g., an enzyme, such as a powdered enzyme).

A biomolecule (e.g., an undesired biomolecule, a desired biomolecule) may be removed by extraction of a biomolecular composition (e.g., a cell-based particulate material). For example, a lipid and/or an aqueous component of a cell-based particulate material may be partly or fully removed by extraction with appropriate solvents. Such extraction may be used to dry the cell-based particulate material by removal of liquid (e.g., water, lipids), remove of a biotoxin, sterilize/attenuate living material in the composition, disrupt and/or permeabilize a cell, alter the physical and/or chemical characteristics of the cell-external environment interface, or a combination thereof.

A purification technique may comprise resuspending a biomolecular composition comprising a biomolecule (e.g., a desired enzyme). For example, the biomolecular composition may be prepared by suspending the biomolecular composition in a liquid component (e.g., a solvent, glycerol), a cryopreservative (“cryoprotector”), a xeroprotectant, and/or a biomolecule stabilizer, prior to adding the biomolecular composition to the coating. The resuspended material to be into a form suitable for storage, further processing, and/or addition to a material formulation.

In some embodiments, a processing technique may comprise maintaining a biomolecular composition (e.g., a composition comprising an enzyme) at a temperature at or less than the optimum temperature for the activity of a living organism and/or a biomolecule (e.g., a proteinaceous biomolecule) that may detrimentally affect a proteinaceous molecule. For example, temperatures at or less than about 37° C. are contemplated in such aspects, during processing of materials derived from a human cell.

In some aspects, a biomolecular composition comprises a cell based particulate material wherein the cell membrane and/or the cell wall has been altered through a permeabilizing process, a disruption process, or a combination thereof. Permeabilization and/or disruption may promote the separation of cells, reduce the average particle size of the material, allow greater access to a biomolecule in a cell (e.g., to promote ease of extraction), or a combination thereof. A permeabilizing process may include contacting a cell (e.g., a cell based particulate material) with a permeabilizing agent such as DMSO, ethylenediaminetetraacetic acid (“EDTA”), tributyl phosphate, or a combination thereof. A permeabilizing technique may increase the mass transport of a substance (e.g., a ligand) into the interior of a cell (e.g., a cell based particulate material) where, for example a binding interaction with a biomolecule may occur, such as an enzyme localized inside the cell (e.g., a cell based particulate material) catalyzes a chemical reaction with the substance. (Martinez, M. B. et al., 1996; Martinez, M. B. et al., 2001; Hung, S.-C. and Liao, J. C., 1996), or a ligand binding a proteinaceous molecule (e.g., a peptide, a polypeptide). Cell permeabilizing using EDTA has been described (Leduc, M. et al., 1985).

A cell based material may be disrupted by any method known in the art, including, for example, a chemical method, a mechanical method, a biological method, or a combination thereof. Examples of a chemical cell disruption method include suspension in a liquid component (e.g., a solvent) for certain cellular components. In specific facets, such a solvent may comprise an organic solvent (e.g., acetone), a volatile solvent, or a combination thereof. In a particular facet, a cell may be disrupted by acetone (Wild, J. R. et al., 1986; Albizo, J. M. and White, W. E., 1986). In certain facets, the cells are disrupted in a volatile solvent for ease in evaporation. Examples of a mechanical cell disruption method include pressure (e.g., processing through a French press), sonication, mechanical shearing, or a combination thereof. An example of a pressure cell disruption method includes processing through a French press. Examples of a biological cell disruption method include contacting the cell with one or more proteins and/or polypeptides that are known to possess such disrupting activity including a porin and/or an enzyme such as a lysozyme, as well as contact/cell infection with a virus that weakens, damages, and/or permeabilizes a cell membrane, a cell wall, or a combination thereof. In another example, a cell-based particulate material comprising cell(s) and/or cellular component(s) may be homogenized, sheared, undergo one or more freeze thaw cycles, be subjected to enzymatic and/chemical digestion of a cellular material (e.g., a cell wall, a sugar, etc.), undergo extraction with a liquid component (e.g., an organic solvent, an aqueous solvent), etc., to weaken interactions between the cellular material(s). A processing technique may comprise sonicating a composition. Other disrupting and/or drying may be done by freeze-drying with a reduced and/or absent cryoprotector (e.g., a sugar).

A biomolecule such as a proteinaceous molecule may be further purified and/or isolated using any technique in the art (e.g., size fractionation, affinity chromatography, binding separation on a column, etc). Although synthetically obtained anti-biological proteinaceous molecules (i.e., peptides, polypeptides and proteins) that are identified and produced as described above may be used, it may be also possible to employ suitable naturally occurring anti-biological proteinaceous molecules, and microbes that produce such agents, as additives in a material formulation (e.g., a paint. a coating). The use of naturally produced anti-biological products isolated and/or purified in commercial quantity from microorganisms may also be done. Large-scale cell culture of the anti-biological agent-producing microorganism may be done for the purification of the anti-biological product. In many instances, the cultural isolate responsible for the production of the anti-biological agent may be batch cultured. Furthermore, purification strategies may be conducted to purify the active product to a level of homogeneity. A naturally derived anti-biological agent has the potential for co-purification of unwanted microbial byproducts, especially byproducts which may be undesirably toxic. In many cases, these factors lead to higher production costs, such as during large-scale isolation of anti-biological products. Purifications may be conducted where racemized mixtures are possible where only a single stereoisomer may be active, or where disulfide linkages are possible between peptide monomers. In some aspects, when desirable naturally occurring anti-biological proteins or polypeptides are isolated, for example, and their amino acid sequences may be partially or fully identified, synthesis of the native molecule or portions thereof, may be problematic due to specific disulfide bond formation, high histidine requirements, and so forth. Nonetheless, natural sources provide additional sequences as coating additives.

For example, a biomolecular composition such as a proteinaceous molecule (e.g., an antifungal peptide sequence identified as described herein) may be grown in suitable cell(s) (e.g., a bacterial cell, an insect cell) employing recombinant techniques and materials described herein and/or of the art, using DNA encoding the proteinaceous molecule's sequence (e.g., encoding an antifungal peptide's sequence described herein) which may be used instead of and/or in combination with a previous DNA sequence. For example, an expression vector may comprise a DNA sequence encoding SEQ ID No. 1 in the correct orientation and reading frame with respect to the promoter sequence to allow translation of the DNA encoding the SEQ ID No. 1. Examples of such cloning and expression of an exemplary gene and DNAs are described herein and in the art. Such a proteinaceous molecule, whether synthetically and/or recombinantly produced, may comprise one or more other sequences (e.g., extracellular and/or intracellular signal sequence(s) to target a proteinaceous molecule, restriction enzyme site(s), ion and/or metal binding sites such as a His-Tag), for ease of processing, preparation, and/or to alter and/or confer an additional property. For example, a plurality of peptide sequence(s), which may comprise multiple copies of the same and/or different sequences, may be produced. One or more restriction enzyme site(s) may expressed between selected sequence(s), to allow cleavage into smaller proteinaceous molecules (e.g., cleavage into smaller peptide sequences). A metal binding site such as a His-tag may be added for ease of purification and/or to confer a metal binding property. Thus, a peptide sequence may be included as part of a polypeptide by incorporation of one or more copies of peptide sequence(s), additional sequences (e.g., His-tags, restriction enzyme sites). Further, one or more peptide sequence(s) and/or one or more such additional sequences may be added to the C-terminus and/or the N-terminus of another proteinaceous sequence (e.g., an enzyme). For example, an enzyme (e.g., an anti-biological enzyme, an esterase) may be modified to comprise an anti-biological peptide sequence, a restriction enzyme site, and/or a metal binding domain (e.g., a His-Tag), with the additional proteinaceous sequence(s) added at the N-terminus, the C-terminus, or a combination thereof

10. Modifications a Biomolecular Composition for Retention in a Material Formulation

A material formulation (e.g., a marine coating, a pipeline coating) may be subject to contact with a liquid (e.g., water) that may elute a biomolecular composition (e.g., a proteinaceous molecule) and/or a component associated with the biomolecular composition (e.g., a metal ion ligand of a proteinaceous molecule). Various technique(s) and/or modification(s) may be used to promote retention (e.g., modify the disassociation constant Kd, modify the association constant Ka, etc) of a biomolecular composition (e.g., a proteinaceous molecule), a ligand (e.g., a metal ion) bound to a biomolecular composition, an immobilization agent of a biomolecular composition, or a combination thereof, as part of a material formulation. Immobilization refers to attachment (i.e., by covalent attachment, “linking,” “tethering,” “conjugation”) of a biomolecular composition such as a proteinaceous molecule (e.g., an enzyme, a peptide, a polypeptide) to another molecule (“immobilization agent,” “carrier”), often to promote retention in a material formulation and/or alter a property of the biomolecular composition. Immobilization may also refer to cross-linking to a like molecule, such as cross-linking an enzyme (e.g., a CLEC) to another enzyme (e.g., the same type of enzyme), which results in an increase in size of the linked molecules that may retard loss from a material formulation.

An immobilization agent may be one suitable for use in a permanent, a semi-permanent, and/or a temporary material formulation (e.g., a permanent surface coating application, a semi-permanent coating, a non-film forming coating, a temporary coating). In some embodiments, a biomolecular composition may comprise an immobilization agent (e.g., a microsphere, a liposome, a soluble carrier, an insoluble carrier) to promote handling (e.g., dispersion) of the biomolecular composition in part (e.g., a saline solution, a buffer, a solvent) and/or all of a material formulation and/or promote localization to a part of a material formulation (e.g., at or near a surface layer). For example, a microsphere may be effectively utilized with a proteinaceous composition in order to deliver the composition to a selected site of activity (e.g., at or near the surface of a material formulation). Any technique and/or material for immobilizing a biomolecular composition (e.g., a proteinaceous molecule) to an immobilization agent described herein and/or of the art (e.g., the literature), may be used.

An example of an immobilization agent for a biomolecule's immobilization include a reverse micelle, a zeolite, a Celite Hyflo Supercel, an anion exchange resin, a Celite® (diatomaceous earth), a polyurethane foam particle, a macroporous polypropylene Accurel® EP 100, a macroporous packing particulate, a macroporous anionic resin bead, a polypropylene membrane, an acrylic membrane, a nylon membrane, a cellulose ester membrane, a polyvinylidene difuoride membrane, a filter paper, a Teflon membrane, a ceramic membrane, a polyamide, a cellulose hollow fibre, a resin, a polypropylene membrane pretreated with a blocked copolymer, an immunoglobin (e.g., an antibody binding a biomolecular composition, an immobilization via enzyme-linked immunosorbent assay) such as a single chain antibody that may bind a biomolecular composition (e.g., a peptide, a polypeptide), an agarose, an ion-exchange resin, and/or a sol-gel (In “Engineering of/with Lipases” (F. Xavier Malcata., Ed.) pp. 298, 408, 409,414, 422, 447, 448, 451, 461, 494, 501, 516, 546, 549, 1996; U.S. Pat. No. 4,939,090; Lopez, M. et al., 1998; “Methods in non-aqueous enzymology” (Gupta, M. N., Ed.) pp. 41-51, 63-65, 2000]. Examples of an immobilization agent that is typically soluble include certain polymer(s) (e.g., a polyethyleneglycol, a polyvinylpyrrolidone). Examples of an immobilization agent that is typically insoluble include sand, a silicate, and/or certain polymer(s) (e.g., a polystyrene, a cellulosic polymer, a polyvinylchloride). Examples of reactive moieties of a proteinaceous molecule that may be used to chemically bind a proteinaceous molecule to an immobilization agent (e.g., bind to a solid support immobilization agent) include a lysine amino moiety, an aspartate carboxyl moiety, a glutamate carboxyl moiety, though other chemically reactive moiety(s) described herein or known in the art may be used.

In many embodiments, an immobilization agent may be selected to comprise a chemical and/or a physical characteristic which does not significantly interfere with the activity of a biomolecular (e.g., a peptide) composition and/or the immobilization agent, though in other embodiments, a property such ligand (e.g., substrate) selectivity and/or binding property(s); anti-biological activity; stability (e.g., thermal stability; solubility; pH and temperature optimums; kinetic properties such as Kₘ; etc. may be altered by immobilization. For example, a proteinaceous molecule immobilized to a typical component of a material formulation acting as an immobilization agent within a material formulation may have limited conformational changes in the presence of a solvent that result in loss of activity, prevent aggregation of a proteinaceous molecule (e.g., an enzyme), improve a proteinaceous molecule's resistance to proteolytic digestion by limiting conformational change(s) and/or exposure of cleavage site(s), to increase the surface area of a proteinaceous molecule to contact with a ligand (e.g., an enzyme's substrate, a metal binding sequence's ligand), or a combination thereof [In “Engineering of/with Lipases” (F. Xavier Malcata., Ed.) pp. 457-458, 1996; “Methods in non-aqueous enzymology” (Gupta, M. N., Ed.) p. 37, 2000]. In a further example, immobilization of a proteinaceous molecule may be used to improve stability against oxidation (e.g., autooxidation); reduce denaturation upon contact with a solvent, a solute, and/or a shear force; reduce self digestion; prevent loss of a proteinaceous molecule by dissolving, suspension, etc into a liquid component (e.g., water, a solvent) and being washed away; and providing an increased concentration of a proteinaceous molecule in a local area for highest yield of an proteinaceous molecule's activity (e.g., binding a metal ligand, anti-biological activity, enzymatic catalysis). For certain embodiments, a material formulation comprising an immobilized biomolecular composition may possess extended bioactivity (e.g., activity retained for an additional week, month, year, etc.) relative to a like material formulation comprising a biomolecular composition with a reduced amount of immobilization (e.g., not immobilized).

An example of immobilization includes, for example, absorption, ionic binding, covalent attachment, cross-linking, entrapment into a gel, entrapment into a membrane compartment, encapsulation (e.g., microencapsulation), or a combination thereof (Kurt Faber, “Biotransformations in Organic Chemistry, a Textbook, Third Edition.” pp. 345-356, 1997). Absorption (i.e., non-covalent binding) to a component of a material formulation may be used, for example, to attach a biomolecular composition onto a material where it may be held by a non-covalent (e.g., hydrogen bonding, Van der Waals forces) interaction. An example of absorption would be a coating comprising a nitrocellulose polymer absorbedly binding a proteinaceous molecule. Examples of a material that may be used for absorption of a proteinaceous molecule (e.g., an enzyme) include a woodchip, an activated charcoal, an aluminum oxide, a diatomaceous earth (e.g., Celite), a cellulose material, a controlled pore glass, a siliconized glass bead, or a combination thereof. Ionic binding refers to an ionic interaction promoting binding of molecules (e.g., a biomolecule to an ionic immobilization carrier), such as occurs in an ion exchange resin, such as a cation (e.g., carboxymethyl cellulose, Amberlite IRA) resin, an anion (e.g., sephadex, diethyl-aminoethylcellulose) resin, or a combination thereof.

Covalent bonding immobilization generally involves chemical reaction at an amino acid residue's amino moiety (e.g., lysine's epsilon amino group), phenolic moiety, sulfhydryl moiety, hydroxyl moiety, carboxy moiety, or a combination thereof, usually with a spacer chemical (e.g., a cross-linking agent) that may be used to bind to a proteinaceous molecule to an immobilization agent. Examples of an covalent bonding immobilization agent includes porous glass via a spacer (e.g., an aminoalkylethoxy-chlorosilane, an aminoalkyl-chlorosilane); a polysaccharide polymer carrier (e.g., agarose, chitin, cellulose, dextran, starch) via a cyanogen bromide reaction; a synthetic co-polymer (e.g., polyvinyl acetate) via an epichlorohydrin activation reaction; an epoxy-activate resin; a cation exchange resin activated to covalently bond by acid chloride conversion of a carboxylic acid, or a combination thereof. For example, a surface and/or a component of a material formulation (e.g. a polystyrene, a nylon, a glass, a silica, an agarose, a polypropylene modified with a polyphenylene, a polyacrylamide, an amino silane, a mercaptosilane, a glycol) comprising a reactable moiety such as an anime, epoxide, and/or aldehyde moiety, may form a covalent bond with a proteinaceous molecule's reactive moiety [“Protein Microarrays” (Schena, Mark Ed.) Jones and Bartlett Publishers 40 Tall Pine Drive Sudbury, Mass., pp. 142-147, 162, 2005]. In another example, a proteinaceous molecule may be covalently bonded to a polymer such as a polyethylene glycol (“PEG”) comprising an ester and/or an aldehyde moiety. In a specific example, a succinimidyl propionate monomethoxy PEG ester moiety may be reacted with both N-terminal and side chain amino group of a proteinaceous molecule, while the aldehyde moiety of a succinimidyl butyraldehyde-mPEG preferentially reacted, in the presence of sodium cyanoborohydride at acidic pH, with an N-terminus amino group of a proteinaceous molecule. Increasing size of the PEG bonded to the proteinaceous molecule (i.e., a peptide), such as 5 kDa over 2 kDa PEG, enhanced resistance to proteolytic degradation. In another example, a 6-maleimido caproic acyl N-hydroxy succinimide ester (“MCS”; Sigma, 15 Fleetwood Court, Ronkonkoma, N.Y.) bifunctional cross-linker may be used to link a proteinaceous molecule comprising a sulfhydro moiety (e.g., a cysteine) to a PEG molecule comprising an amine (e.g., a monomethoxypolyethylene glycol amine) (He, X.-H. et al., Life Science 64(14):1163-1175, 1999). A polymer such as PEG may also reduce chemical degradation of a proteinaceous molecule (Diago, M. et al. Aliment Pharmacol Ther 26:1131-1138, 2007). It is contemplated that a polymer (e.g., a PEG) that may be covalently bound to a proteinaceous molecule may range from about 0.5 kDa to about 5,000,000 kDa or more in average molecular weight.

In particular embodiments, a biomolecular composition such as a proteinaceous molecule may comprise a carbohydrate moiety (e.g., a sugar, a galactose) such as due to post-translational modification during biologically based production. A carbohydrate moiety may be oxidized (e.g. enzyme oxidation, periodate oxidation) to produce the aldehyde moiety that may be covalently bonded by reaction with a hydrazide moiety of a material formulation component (e.g., a polyacrylamide). In another example, an agarose polymer may become able to covalently bond to an amino moiety (e.g., a proteinaceous molecule's amino moiety) upon activation by contact with NaIO4 [“Protein Microarrays” (Schena, Mark Ed.) Jones and Bartlett Publishers 40 Tall Pine Drive Sudbury, Mass., pp. 142-147, 162, 2005].

In certain embodiments, a chemical cross-linker may be used to increase molecular space between a biomolecular composition (e.g., a proteinaceous molecule) and a reactive moiety of a component of a material formulation. For example, a silane (e.g., an alkylsilane) may covalently bind a material (e.g., a glass) comprising a hydroxyl moiety, and the silane may also comprise a reactive moiety (e.g., an aldehyde, an amino moiety, an epoxy moiety) that may be used to covalently bind another molecule (e.g., a proteinaceous molecule). In another example, a 6-maleimido caproic acyl N-hydroxy succinimide ester (“MCS”; Sigma, 15 Fleetwood Court, Ronkonkoma, N.Y.) bifunctional cross-linker may be used to link a proteinaceous molecule comprising a sulfhydro moiety (e.g., a cysteine) to a proteinaceous molecule comprising an amine (e.g., an N-terminal amine, a lysine side chain amine, an arginine side chain amine, etc.) (Lee, A. C. J. et al., Molecular Immunology, 17:749-756, 1980). A silane may be reacted with a cross-linking agent to produce a silane derivative comprising a linked cross-linker (e.g. a heterobifunctional cross-linker) comprising a reactive moiety (e.g., a thiol, an aldehyde, an amino moiety) [“Protein Microarrays” (Schena, Mark Ed.) Jones and Bartlett Publishers 40 Tall Pine Drive Sudbury, Mass., pp. 261, 2005]. Additional examples of a cross-linking agent that may be used to bond one or more proteinaceous molecule(s) and/or material formulation component(s) include a 1,1-bis(diazoacetyl)-2-phenylethane; a glutaraldehyde; a N-hydroxysuccinimide ester; a 3,3′-dithiobis (succinimidyl-propionate); a bis-N-maleimido-1,8-octanel; or a combination thereof.

A cross-linking enzyme may comprise an enzyme interconnect to a like and/or a different enzyme, via a bifunctional cross-linking agent (e.g., a glutaraldehyde, dimethyl adipimidate, dimethyl suberimidate and hexamethylenediisocyanate), sometimes with larger molecule such as a proteinaceous molecule (e.g., a “filler protein”) (e.g., an albumin) separating the enzyme(s) molecule(s). This technique may be adapted to other biomolecules(s) (e.g., a proteinaceous molecule, a peptide, a polypeptide, an antibody, a receptor, etc.), and may be used to modify the size of a component. For example, one or more anti-biological peptide(s) may be cross-linked to each other and/or another material formulation (e.g., a coating) component. In certain embodiments, a proteinaceous molecule such as an enzyme may be in the form of a crystal. In other aspects, one or more enzyme crystals may be cross-linked to from a CLEC (Hoskin, F. C. G. et al., 1999; Lalonde, J. J. et al., 1995; Persichetti, R. A., 1996). In certain embodiments, such a physical spacer such as a cross-linking agent (e.g., a bifunctional chemical cross-linking agent) and/or an additional proteinaceous sequence may be used to separate proteinaceous sequences of the same and/or different function, such as a metal binding proteinaceous sequence and another proteinaceous sequence, to promote greater conformation flexibility and/or binding availability of the sequence(s).

Gel entrapment includes incorporation of a biomolecular composition (e.g., an enzyme) and/or a biological cell into a gel matrix (e.g., an alginate, a carragenan gel, a polyacrylamide gel, or a combination thereof) that may be formed into various shapes (Karube, I. et al., 1985; Qureshi, N. et al., 1985; Umemura, I. et al., 1984; Fukui, S. and Tanaka, A. 1984; Mori, T. et al., 1972; Martinek, K. et al., 1977; Kurt Faber, “Biotransformations in Organic Chemistry, a Textbook, Third Edition.” pp. 350-352, 1997).

Membrane entrapment refers to restricting the space a biomolecular composition (e.g., an enzyme) functions in by being placed in a compartment, often imitating the separation of a biomolecule (e.g., an enzyme) that occurs inside a living cell (e.g., localization of an enzyme inside an organelle). An examples of membrane entrapment composition that may act as an immobilization agent include a micelle, a reversed micelle, a vesicle (e.g., a liposome), a synthetic membrane (e.g., a polyamide, a polyethersulfone) with a pore size smaller than the sequestered biomolecule (e.g., a membrane enclosed enzymatic catalysis or “MEEC”).

In further embodiments, a proteinaceous molecule may comprise a binding sequence for ligand that may be used for immobilizing (e.g., promoting retention) of another proteinaceous sequence (e.g., a metal binding proteinaceous sequence, an anti-biological proteinaceous sequence). The proteinaceous binding sequence may be a contiguous sequence with another proteinaceous sequence (e.g., a fusion protein), be a separate proteinaceous molecule, and/or function as a cross-linking agent. In another example, a proteinaceous molecule comprising a metal binding sequence may be immobilized in a material formulation comprising a metal (e.g., a metal cation, a metal pigment). For example, a histidine rich tag typically binds Ni²⁺ immobilized by chelation with a metal ion chelator (e.g. a polyvalent metal ion chelator) such as a nitrilotriacetic acid (“NTA”), a NTA derivative [e.g., a N,N-bis(carboxymethyl) lysine], an iminodiacetic acid (“IDA”), a macrocycle triazacyclononane, and/or a Co²⁺-carboxymethylaspartate. For example, N,N-bis(carboxymethyl) lysine comprises an amine moiety capable of reacting with a carboxyl moiety to form a covalent amide linkage, and such a reaction may be used to bind a chelator to a support component (i.e., acting as an immobilization agent) of a material formulation. Another example of a support component comprises a paramagnetic particle [e.g., a MagneHis Ni-Particle (Promega)] that is that binds a metal binding sequence (e.g., a histidine rich tag) [“High Throughput Protein Expression and Purification Methods and Protocols,” (Doyle, S. A., Ed.) Humana Press, Walnut Creek, Calif., USA, pp. 129-132, 2009]. An additional example of an immobilization agent comprises a polymer (e.g. a polyether, a polyethylene glycol) comprising a moiety (e.g. a hydroxyl moiety) that binds a chelating agent and/or the metal ion [“Functional Protein Microarrays In Drug Discovery,” (Pradki, P. F., Ed.) CRC Press Taylor & Frances Group 6000 Broken Sound Parkway NW, Suite 300 Boca Raton, Fla., pp. 62-64, 2007]. Such a metal binding component of a material formulation may be used to bind a metal, and thereby, for example, bind a proteinaceous sequence that also binds the metal. In some embodiments, a proteinaceous molecule comprising a plurality of metal binding sequences that may bind both a solid support (e.g., a solid support immobilization agent comprising a metal ion) that may be a component of a material formulation and a free metal ion.

A biomolecular composition (e.g., a metal binding peptide, an enzyme) may be encapsulated (e.g., microencapsulated, such as for use in a material formulation) using a microencapsulation technique. Such encapsulation may enhance and/or confer the particulate nature of the biomolecular composition; provide protection to the biomolecular composition; stabilize a biomolecular composition; increase the average particle size to a desired range; allow slow and/or controlled release from the encapsulating material of a component of a biomolecular composition such as a cellular component (e.g., a biomolecule) and/or an additional encapsulated material (e.g., a chemical preservative/pesticide, an isolated biomolecule, etc.); alter surface charge, hydrophobicity, hydrophilicity, solubility and/or dispensability of a biomolecular composition (e.g., a biomolecular particulate material) and/or an additional encapsulated material; or a combination thereof. For example, an encapsulating agent (e.g., an encapsulating membrane) may provide protection to a proteinaceous molecule from peptidase(s), protease(s), and/or other peptide bond and/or side chain modifying substance. Examples of microencapsulation (e.g., microsphere) compositions and techniques are described in, for example, Wang, H. T. et al., 1991; and U.S. Pat. Nos. 4,324,683, 4,839,046, 4,988,623, 5,026,650, 5,153,131, 6,485,983, 5,627,021 5,827,531; 6,103,271; 6,387,399, and 6,020,312. Examples of a microencapsulating material includes a gelatin, a hydrogenated vegetable oil, a maltodextrin, a polyurea, a sucrose, an acacia, an amino resin, an ethylcellulose, a polyester, or a combination thereof. In some facets, an encapsulating material (e.g., a polymer) swells, dissolves, and/or degrades upon contact with a liquid component, a chemical, a biomolecule (e.g., a proteinaceous molecule, an enzyme), the environment, or a combination thereof. For example, a polyvinyl alcohol, which comprises a water soluble polymer, may be used to encapsulate a peptide antifungal agent for incorporation into a bathroom caulk to allow greater release of the peptide/ease of contact with a microorganism, upon contact of the caulk with moisture/water during the normal use of the caulk. A poly(D,L-lactide-co-glycolide) (“PLGA”) and poly(D,L-lactide) (“PLA”) micro-sphere (Sandostatin LAR depot, Novartis Pharma, Basel, Switzerland; Boehringer Ingelheim, Binger Str. 173, 55216 Ingelheim, Germany) have been used to encapsulate proteinaceous molecules, and may release the proteinaceous molecule upon environmental (e.g., biological) degradation of the microsphere's polymer (Diago, M. et al. Aliment Pharmacol Ther 26:1131-1138, 2007). In another example, a polyester microsphere encapsulating agent may be used to encapsulate and stabilize a biomolecular composition (e.g., a proteinaceous molecule) in a material formulation (e.g., a paint, a coating) during storage (e.g., multi-pack storage; in can storage), or to provide for prolonged, gradual release of the biomolecular composition after it is dispersed in a material formulation during use (e.g., a paint film covering a surface).

11. Incorporation of a Biomolecular Composition into a Material Formulation

A biomolecular composition (e.g., a proteinaceous molecule), a substrate for an enzyme, a ligand for the biomolecular composition (e.g., a metal ion that binds a metal binding sequence), an added material that may affect the activity and/or function of a biomolecular composition (e.g., an enzyme inhibitor, a cofactor, a buffer, etc.), etc., may be incorporated (e.g., embedded) within a material formulation via various methods described herein and/or as know in the art. These methods include, for example, direct addition to a prepared material formulation; incorporation as a component during preparation of a de novo material formulation, post preparation absorption of a component, or a combination thereof; and may be used a substitute for, or in combination with, the other techniques described herein for processing (e.g., encapsulation) and incorporation of a component into a material formulation. For example, in some embodiments, little or no formulation modification, other than the inclusion of a presently described biomolecular composition (e.g., a proteinaceous molecule) may be conducted to obtain enhancement of a metal binding, anti-biological and/or anti-fouling properties of a material formulation (e.g., a coating). For example, a coating (e.g., a paint) comprising a metal binding proteinaceous molecule additive may retain an metal binding activity after being admixed with the coating composition, and may confer metal binding and/or anti-fouling activity after application of the coating composition to a surface (e.g., after formation of a marine paint film). In another example, incorporation of a component such as a biomolecular composition (e.g., a metal binding proteinaceous molecule) may be conducted using electric charge, such as by contact of a material formulation with a liquid comprising an electrically charged component, and using electrophoresis to promote movement of the additional component on and/or into the material formulation.

A biomolecular composition may function as an additional component to a material formulation [e.g., added to a previous material formulation such as a commercially available product], and/or may substitute for all and or part of one or more component(s) of a material formulation (e.g., an anti-biological proteinaceous molecule substitution of some or all of a non-proteinaceous preservative) during preparation of the material formulation. For example, a material formulation comprising such a biomolecular composition may be free and/or comprise a reduced content of component(s) (e.g., a chemical, an additive) that are toxic a non-target organism (e.g., a humans, certain animals, certain plants, etc.) and/or that fail to comply with applicable environmental safety rule and/or guideline. In some aspects, a biomolecular composition may work in combination with and/or synergistically with a component of a material formulation (e.g., a metal binding peptide with a metallic pigment; an anti-biological enzyme and/or an anti-biological peptide combined with a preservative, a co-biocide, etc.). In some embodiments, a plurality of different biomolecular compositions(s) may work in combination with and/or synergistically with each other and/or one or more non-biomolecular component(s) of a material formulation.

In some embodiments, a material formulation (e.g., a coating) may comprise an insoluble particulate material. For example, a pigment, an extender, certain types of rheology modifiers, certain types of dispersants, or a combination thereof are the major sources of particulate material(s) in a coating. A cell-based particulate material generally may also be a source of particulate material in a material formulation. Any technique used in the preparation of a material formulation (e.g., a coating) comprising a pigment, an extender and/or any other form of particulate material described herein and/or in the art may be used in the preparation of a material formulation comprising a particulate biomolecular composition (e.g., a cell-based particulate material). For example, incorporation of a particulate material (e.g., a pigment), an assay for determining a rheological property and/or a related property (e.g., viscosity, flow, molecular weight, component concentration, particle size, particle shape, particle surface area, particle spread, dispersion, flocculation, solubility, oil absorption values, CPVC, hiding power, corrosion resistance, wet abrasion resistance, stain resistance, optical properties, porosity, surface tension, volatility, settling, leveling, sagging, slumping, draining, floating, flooding, cratering, foaming, splattering) of a coating component and/or a coating (e.g., pigment, binder, vehicle, surfactant, dispersant, paint) and procedures for determining such properties, as well as procedures for large scale (e.g., industrial) coating preparation (e.g., wetting, pigment dispersion into a vehicle, milling, letdown) are described in, for example, in Patton, T. C. “Paint Flow and Pigment Dispersion, A Rheological Approach to Coating and Ink Technology,” 1979, and may be applied in the incorporation of a biomolecular composition having a particulate property or nature.

Detection of the incorporation of biomolecular composition into a material formulation may be conducted by any assay of the art for detection of the presence of a particular biomolecule, such as to insure the adequate incorporation and/or retention of a biomolecular composition in a material formulation. For example, a proteinaceous molecule's incorporation into a material formulation may be monitored (e.g., detected) by use of dye (e.g., a fluorophore) that may stain the proteinaceous molecule to demonstrate the presence (e.g., amount) of the proteinaceous molecule in a material formulation. An example of a dye that may stain a proteinaceous molecule includes a succinimidyl ester cyanine dye, a tetrafluorophenyl ester dye, a sulfonyl chloride (e.g., a Texas Red sulfonyl chloride dye), an isothiocyanate (e.g., a fluorescein isothiocyanate, a tetramethylrhodamine isothiocyanate), a maleimide, an iodoacetamide, a carbodiimide, a hydrazide, a photo-reactive cross-linking agent [e.g., a sulfosuccinimidyl 2-(7-azido-4-methylcoumarin-3-acetamide], or a combination thereof [“Functional Protein Microarrays In Drug Discovery,” (Pradki, P. F., Ed.) CRC Press Taylor & Frances Group 6000 Broken Sound Parkway NW, Suite 300 Boca Raton, Fla., pp. 156-160, 2007].

The incorporation method selected may influence biomolecule's activity (e.g., binding activity, enzymatic activity). A material formulation may undergo a chemical reaction and/or comprise a component that may partly or fully damage, inhibit, and/or inactivate an active biomolecule. For example, a surface treatment such as a coating (e.g., a polyurethane) may cure by a chemical reaction. In some embodiments, the biomolecular composition may be incorporated after the bulk of a chemical reaction in a material formulation has occurred. The bulk of these reactions typically occur during typically material preparation, are referred to as “body time,” “curing,” “cure time,” etc, with some residual reactions occurring after cure that may be not considered significant to the potential detrimental influence on a biomolecular composition. Incorporation of the biomolecular composition or other component after part or the majority of this main cure time may serve to protect the biomolecular composition from these reactions. These cure times are typically know (e.g., described in manufacturer's instruction) and/or readily determined by standard assays for a material and/or an enzyme properties. In some embodiments, the biomolecular composition may be incorporated after about 0%, to about 100% of the cure time has passed. For example, a peptide may be incorporated by admixing after about 80% or more of a body time as passed for a polyurethane coating. In another example, a biomolecular composition may be incorporated post-cure (e.g., after about 90% curing has occurred) for a thermosetting material formulation. In another embodiment, a biomolecular composition may be incorporated during post-cure processing. In other embodiments, a biomolecular composition may be incorporated after about 100% of the cure time has passed. A multi-pack embodiment of a material formulation may be used to allow such timing of incorporation during preparation and use of the material formulation. Additionally, a biomolecular composition may comprise a protective material (e.g., an immobilization agent, additional biomolecules found in a cell-based preparation of a desired biomolecule) to protect a desired biomolecule from damage by a chemical reaction and/or a component of a material formulation, protect the desired biomolecule from damage during normal use (e.g., environmental damage, washings, etc) of a material formulation, or a combination thereof

12. Coatings Comprising a Biomolecular Composition

An example of a material formulation is a coating, such as a marine coating, an architectural coating, an industrial coating, and/or a specification coating. One or more of the biomolecular compositions (e.g., a metal binding proteinaceous molecule, an anti-biological proteinaceous molecule, an enzyme such as an OP degrading enzyme), may be prepared with a coating (e.g., a base paint). A coating may be any suitable commercially available product, a wide variety of which are known in the art, and/or may be custom formulated (i.e., de novo formulated for use with the biomolecular composition) and/or blended using any combination of various naturally-occurring and synthetic components and additives that are known in the art such as those described in U.S. patent application Ser. No. 10/655,345 filed Sep. 4, 2003 or U.S. patent application Ser. No. 10/792,516 filed on Mar. 3, 2004, which are hereby expressly incorporated herein by reference in their entirety.

A coating generally comprises one or more component(s) such as a binder, a liquid component, a colorizing agent, and/or one or more additive(s). A “binder” refers to the primary material in a coating capable of undergoing film formation. Film formation which refers to a physical and/or a chemical change of the binder to produce a film. Often, a binder converts into a film through a polymerization reaction (e.g., a thermosetting binder), and/or comprises a polymer that undergoes film formation via a physical process (e.g., a thermoplastic binder), such as loss of a volatile component from a coating. Examples of a binder include an oil-based binder (e.g., an oil, an alkyd resin, an oleoresinous binder, an fatty acid epoxy ester); a polyester resin; a modified cellulose; a polyamide; an amidoamine; an amino resin; an urethane; a phenolic resin; an epoxy resin; a polyhydroxyether; an acrylic resin; a polyvinyl binder; a rubber resin; a bituminous; a polysulfide and/or a silicone.

In many embodiments, a coating may comprise a liquid component (e.g., a solvent, a thinner, a diluents, a plasticizer, water). A liquid component comprises a chemical composition in a liquid state, and typically is added to a coating formulation to improve a rheological property for ease of application, alter the period of time that thermoplastic film formation occurs, alter an optical property (e.g., color, gloss) of a film, alter a physical property of a coating (e.g., reduce flammability) and/or a film (e.g., increase flexibility), or a combination thereof. A coating often comprises a volatile liquid component such as a volatile organic compound (“VOC”), water, or a combination thereof, which may be lost during film formation. Organic compounds that may be used as a liquid component include a hydrocarbon; an oxygenated solvent; a chlorinated hydrocarbon, a nitrated hydrocarbon, and/or an other organic liquid. In certain aspects, a coating comprises a waterborne coating and/or a solvent based coating.

Usually a coating (e.g., a paint) comprises a colorant (“colorizing agent”), which refers to a composition that confers an optical property to a material formulation such as coating and/or film. Examples of a colorant include a pigment, an extender, a dye, or a combination thereof. A pigment comprises a composition that is insoluble in the other component(s) of a coating, and further confers an optical property, confers a property affecting the application of the coating (e.g., a rheological property), confers a performance property (e.g., a corrosion resistance property, a magnetic property, a camouflage property) to a coating, reduces the cost of the coating, or a combination thereof. An extender is usually a less expensive type of pigment that may act as an opacifying agent and/or bulk material. A dye comprises a composition that is soluble in the other component(s) of a coating, and further confers a color property to the coating.

A coating often comprises an additive, which refers to a composition incorporated into a coating to reduce and/or prevent the development of a physical, chemical, and/or aesthetic defect in the coating and/or film; confer some additional desired property to a coating and/or film; or a combination thereof. Examples of an additive include a buffer (e.g., ammonium bicarbonate, a monobasic buffer, a dibasicphosphate buffer, Trizma base, a zwitterionic buffer); a catalyst (e.g., a drier, an acid, a base, a urethane catalyst); a coalescing agent; a corrosion inhibitor; a cryopreservative; a defoamer; a dehydrator; a dispersant; a drier; a filler; a film-formation promoter; a flame/fire retardant; a flatting agent; a flow control agent; a gloss aid; a leveling agent; a light stabilizer; a light stabilizer; a marproofing agent; a matting agent; a neutralizing agent; a pH indicator; a preservative; a rheology modifier; a silicone additive; a slip agent; a surfactant; a viscosity control agent; a wetting agent; a xeroprotectant; an accelerator; an adhesion promoter; an anti-floating agent; an anti-flooding agent; an anti-foaming agent; an anti-insect additive; an antioxidant; an anti-skinning agent; an electrical additive; and/or an emulsifier. The content for an individual coating additive in a coating generally comprises about 0.0001% to about 20.0% (e.g., between about 0.0001% and about 10.0%).

a. Marine Coatings Comprising a Biomolecular Composition

A material formulation may comprise a marine coating (e.g., a marine coating having an anti-fouling property). A marine coating is used on a surface that contacts water and/or a surface that comprises part of a structure continually near water (e.g., a ship, a dock, a drilling platform for fossil fuels, etc). For embodiments wherein a surface contacts water, the type of marine coating may be selected to resist fouling, corrosion, or a combination thereof. Other properties that are often used in a marine coating include chemical resistance, impact resistance, fire resistance, abrasion resistance, friction resistance, acoustic camouflage, electromagnetic camouflage, or a combination thereof. Marine coatings and their use are known in the art (see, for example, in “Paint and Surface Coatings: Theory and Practice,” 2^{nd} Edition, pp. 529-549, 1999; in “Paints, Coatings and Solvents,” 2^{nd} Edition, pp. 252-258, 1998; in “Organic Coatings: Science and Technology, Volume 1: Film Formation, Components, and Appearance,” 2^{nd} Edition, pp. 138, 317-318).

Fouling may damage a material (e.g., a surface, a film), and many marine coating(s) are formulated with an anti-fouling agent (e.g., an anti-fouling preservative), an anti-corrosion property (e.g., an anti-corrosion pigment), or a combination thereof, as such damage often leads to corrosion of a metal surface. In other embodiments, a material formulation comprising a metal binding proteinaceous molecule may possess an anti-fouling property, an anti-biological property, a metal binding property (e.g., binding a metal cation), an enhanced adherence for a metal surface, or a combination thereof. In some embodiments, a material formulation comprising a metal binding proteinaceous molecule may reversible and/or regeneratively bind a metal atom (e.g., an ion). In other embodiments, a marine coating may comprise an anti-fouling enzyme, such as an OP compound degrading enzyme. In further embodiments, a marine coating may comprise a plurality of biomolecular compositions such as those described herein.

Various binders may be used in a marine coating to achieve the properties suitable for a marine environment. For example, an oleoresinous binder generally may be used in a marine coating, a clear varnish such as a lacquer, as well as in applications as a primer, an undercoat, or a combination thereof. An oleoresinous binder may be prepared from heating a resin and an oil. Examples of a resin typically used in the preparation of an oleoresinous binder include resins obtained from a biological source (e.g., a wood resin, a bitumen resin); a fossil source (e.g., a copal resin, a Kauri gum resin, a rosin resin, a shellac resin); a synthetic source (e.g., a rosin derivative resin, a phenolic resin, an epoxy resin); or a combination thereof. An example of an oil typically used in the preparation of an oleoresinous binder includes a vegetable oil, particularly an oil comprising a polyunsaturated fatty acid such as a tung, a linseed, or a combination thereof. The type of resin and oil used may identify an oleoresinous binder such as a copal-tung oleoresinous binder, a rosin-linseed oleoresinous binder, etc. An epoxy ester resin may be selected for use as a marine coating, an industrial maintenance coating, a floor topcoat, as a substitute for an alkyd, or a combination thereof. A fatty acid epoxide ester resin comprises an ester of an epoxide resin and a fatty acid, which may be used to produce an ambient cure coating that undergoes film formation by an oxidative reaction as an oil-based coating. In certain facets, an epoxy coating may be cured by fatty acid oxidation rather than an epoxide moiety and/or a hydroxyl moiety cross-linking reaction(s). In certain embodiments, an epoxy resin may be selected with an epoxy equivalent weight of about 800 to about 1000. A short, a medium, and a long oil epoxide ester resin comprise about 30% to about 50%, about 50% to about 70%, or about 70% to about 90% fatty acid esterification, respectively, with similar, though sometimes improved, properties relative to an analogous alkyd. An epoxide ester resin produced film may be reduced in chemical resistance than a film produced by an epoxy and a curing agent comprising an amine.

A polyamine-epoxy coating may be used as a marine coating, an industrial coating (e.g., an industrial maintenance coating), or a combination thereof. A polyamide (“fatty nitrogen compound,” “fatty nitrogen product”) comprises a reaction product of a polyamine and a dimerized and/or a trimerized fatty acid. In typical embodiments, a polyamide comprises an oligomer. An amide resin comprises a terminal amine moiety capable of cross-linking with an epoxy moiety, and a polyamide binder may be combined with an epoxide binder. In other aspects, a polyamide may be considered an additive (e.g., a curing agent, a hardening agent, a coreactant) of an epoxide coating. A polyamide-epoxide coating may be applied to a surface such as, for example, a wood, a masonry, a metal (e.g., a steel), or a combination thereof. However, in some embodiments, a surface may be thoroughly cleaned prior to application to promote adhesion. Such surface preparation in the art may be used, and include, for example, removal of rust, a degraded film, a grease, etc. A polyamide-epoxy coating may comprise a solvent-borne coating. Examples of a solvent for a polyamide include an alcohol, an aromatic hydrocarbon, a glycol ether, a ketone, or a combination thereof. In certain embodiments, a polyamide-epoxy coating may comprise a two-pack coating, wherein a coating component(s) comprising the polyamide resin may be stored in one container, and a coating component(s) comprising the epoxy resin may be stored in a second container. Such a two-pack coating may be admixed immediately before application, as the stoichiometric mix ratio of resin may be formulated to promote a rapid cure. However, in other embodiments, a polyamide-epoxy coating may comprise a single container coating. Such a solvent-borne polyamine-epoxy coating may be formulated for a storage life of a year or more. An aluminum and/or a stainless steel container may be suitable, though a carbon steel container may alter coating and/or film color. However, such a coating typically undergoes film formation in stages, wherein the liquid component may be physically lost by evaporation while thermosetting produces a physically durable film in about 8 to about 10 hours, a chemically resistant film in about three to about four days, and final cross-linking completed in about three weeks. In some embodiments, a polyamine-epoxy coating may undergo chalking upon exterior weathering.

An ambient cure epoxide may be selected for a marine coating, an industrial coating (e.g., an industrial maintenance coating), an aircraft primer, a pipeline coating, a high performance architectural coating, or a combination thereof. In certain embodiments, a curing agent suitable for curing at ambient conditions comprises an amine moiety such as a polyamine adduct, which comprises an epoxy resin modified to comprise an amine moiety, a polyamide, a ketimine, an aliphatic amine, or a combination thereof. Examples of an aliphatic amine include an ethylene diamine (“EDA”), a diethylene triamine (“DETA”), a triethylene tetraamine (“TETA”), or a combination thereof. Selection of a polyamine adduct generally produces a film with excellent solvent resistance, corrosion resistance, acid resistance, flexibility, impact resistance, or a combination thereof. Selection of a polyamide generally produces a film with improved adhesion, particularly to a moist and/or poorly prepared surface, good solvent resistance, excellent corrosion resistance, good acid resistance, improved flexibility retention, improved impact resistance retention, or a combination thereof. A ketimine comprises a reaction product of a primary amine and a ketone, and produces a coating and/or a film with similar properties as a polyamine and/or an amine adduct. However, the pot life may be longer with a ketimine, and moisture (e.g., atmospheric humidity) activates this cure agent. Examples of an epoxide selected for curing at ambient conditions include a low mass epoxide resin with a n value from about 0 to about 2.0. In certain embodiments, an epoxy resin may be selected with an epoxy equivalent weight of about 182 to about 1750. In specific aspects, the greater then value of an epoxide resin, the longer the pot life in a two-pack coating, the greater the coating leveling property, the lower the film solvent resistance, the lower the film chemical resistance, the greater the film flexibility, or a combination thereof. In certain aspects, an ambient curing epoxide coating comprises a two-pack coating, wherein the epoxide resin may be in one container and the curing agent in a second container. In typical aspects, the pot life upon mixing the coating components may comprise about two hours to about two days.

A coating comprising a chlorinated rubber resins may be used, for example, on surfaces that contact a gaseous, a liquid and/or a solid external environments. Examples of such uses include a marine coating (e.g., a marine vehicle, a swimming pool), a coating for an architectural coating (e.g., a masonry coating), a traffic marker coating, a metal primer, a metal topcoat, or a combination thereof. In general embodiments, a rubber resin comprises a chlorinated rubber resin, wherein a rubber isolated from a biological source has been chemically modified by reaction with chlorine to produce a resin comprising about 65% to about 68% chlorine by weight. A chlorinated rubber resins generally are in a molecular weight range of about 3.5 kDa to about 20 kDa. A chlorinated rubber coating may comprise another binder, such as, for example, an acrylic resin, an alkyd resin, a bituminous resin, or a combination thereof. In specific aspects, a chlorinated rubber resin comprises about 10% to about 50%, by weight, of the binder when in combination with an acrylic resin, an alkyd resin, or a combination thereof. In general embodiments, a chlorinated rubber coating comprises a solvent-borne coating. In certain aspects, a chlorinated rubber coating comprises a liquid component, such as, for example, a solvent, a diluent, a thinner, a plasticizer, or a combination thereof. A thermoplastic coating may comprise a chlorinated rubber coating. To reduce the T_{g} of a film produced from a chlorinated rubber resin, the liquid component generally comprises a plasticizer. In certain aspects, a chlorinated rubber coating comprises about 30% to about 40%, by weight, of plasticizer. In certain facets, a plasticizer may be selected for water resistance (e.g., hydrolysis resistance) such as a bisphenoxyethylformal. In certain facets, a chlorinated rubber coating comprises a light stabilizer, an epoxy resin, an epoxy plasticizer (e.g., epoxidized soybean oil), or a combination thereof, to chemically stabilize a chlorinated resin, coating and/or a film. In other embodiments, a chlorinated rubber coating comprises a pigment, an extender, or a combination thereof. In particular aspects, the pigment comprises a corrosion resistant pigment. A chlorinated rubber film are generally has good chemical resistance (e.g., acid resistance, alkali resistance), water resistance, or a combination thereof.

In some aspects, a binder for a marine coating may comprise a water sensitive binder, such as a dissolvable coating (e.g., a paint) that may release a component such as a biomolecular composition (e.g., an anti-fouling, an anti-biological agent). In other embodiments, to achieve the various properties of a marine coating, often a multicoat system may be used. In other aspects, for metal surfaces, a primer referred to as a blast primer may be applied to the surface within seconds of blast cleaning. Examples of a blast primer include a polyvinyl butyral (“PVB”) and phenolic resin coating; a two-pack epoxy coating; and/or a two-pack zinc and ethyl silicate coating. A marine metal surface undercoat and/or a topcoat may comprise an alkyd coating, a bitumen coating, a polyvinyl coating, or a combination thereof.

Additional various marine material formulation(s) (e.g., a marine anti-fouling coating such as a paint) and anti-fouling agents (e.g., an metal, a metal compound, a metal pigment) have been described (see, for example, Almeida, E. et al., Progress in Organic Coatings 59:2-20, 2007; and Yebra, D. M. et al., Progress in Organic Coatings 50:75-104, 2004), and may be used in conjunction with a biomolecular composition described herein (e.g., a metal binding proteinaceous molecule, an anti-fouling enzyme). An example is a marine coating (e.g., a top coat) to reduce fouling which may be part of a coating system that may comprise an anti-corrosive coating (e.g., an primer, an undercoat, a cathodic protective coating) to reduce corrosion on a metal surface (e.g., a steel plate, a steel profile) that frequently contacts water, particularly those immersed in an aqueous environment for extended periods of time. A surface that does not typically corrode (e.g., wood) but frequently contacts water may be coated with an anti-fouling coating, though the anti-fouling coating may be part of a multicoat system, such as a primer and/or undercoat to promote adhesion of the anti-fouling coating to the surface. For example, a marine coating (e.g., an anti-fouling coating, an anticorrosion coating, a multicoat system) may be used on the below surface part of a ship (e.g., the ship bottom), an area periodically immersed in water (e.g., a boottop area), and/or an area less frequently contacted with water (e.g., a splash area, a top side) to protect against corrosion and/or fouling. In another example, a low friction coating (e.g., a smooth topcoat) may be a marine coating to reduce attachment of a drag inducing marine organism (e.g., a fouling plant, a fouling animal) to an aquatic vessel's bottom surface(s), and reduce fuel consumption.

A marine surface may be protected with a surface treatment (e.g., a coating) of varying compositions and properties due to the differing contact frequencies to water and/or a fouling material. For example, a superstructure or side surface may be coated with one or more coats of epoxy (e.g., a pure epoxy, a modified epoxy), an aliphatic polyurethane, an acrylic/aliphatic polyurethane, and/or a polysiloxane/epoxy, to confer properties such as anticorrosive protection (e.g., antirust stain), UV resistance, washability and suitable aesthetic appearance. In another example, a vessel's deck may be have one or more surfaces treatments (e.g., a multi-surface treatment system) applied such as a coating comprising zinc silicate, a two pack epoxy/polyurethane, a high build primer, an elastomer, a topcoat with a non-slip property (e.g., comprise an aggregate of silica, aluminum oxide), to confer properties such as non-slip, washability, anticorrosive, UV resistance, and/or a suitable aesthetic appearance. A surface of a tank (e.g., a ballast tank) may be, for example, coated with one or more layers of a modified epoxy, pure epoxy (e.g., a pure epoxy comprising an aluminum pigment), a solvent free epoxy, a waterborne asphaltic emulsion and/or an acrylic reinforced with cement, to confer water (e.g., corrosion) resistance and/or fouling resistance. A surface for contact with cargo (e.g., a hydrocarbon material, petroleum) may comprise one or more coats not necessarily to convey an anti-fouling property but chemical cargo resistance property, and such a coating may comprise, for example, a polyamine-cured epoxy (e.g., a solvent free epoxy, a high-solids epoxy) and/or an epoxy-cyclosilicone coating. A metal surface that is typically underwater and/or in a boottop area often comprise an anticorrosive primer (e.g., a primer comprising coal tar, often an epoxy; a two-pack epoxy, a polyurethane) which may comprise a fibre (e.g., glass) for enhanced water vapour resistance and/or mechanical strength; an undercoat often to aid in adhesion of a topcoat; and/or an anti-fouling topcoat. Such an anti-fouling surface treatment (e.g., a coating) may be applied, for example, at a shipyard, a factory, and/or dry-dock to surfaces that may be assembled into an marine object such as device, apparatus, machine, etc. (e.g., a buoy, an aquatic vessel, a dock) and/or directly applied to the surface of a completed marine object, using techniques of the art (e.g., metal surface blasting of a part and coating with a shopprimer, assembly, additional coating application).

Often an anti-fouling surface treatment (e.g., a self-polishing coating) is designed to allow leaching of an anti-fouling agent from the coating, creating pores in the coating, and may also be designed for concurrent release of binder to ease of release of the anti-fouling agent as the surface of the coating is eroded. The salinity (i.e., the concentration of all dissolved salts including NaCl) of water may influence the rate of anti-fouling agent release via an ion exchange reaction. The surface salinity of sea water is about 3.3 to 3.8 wt. % (e.g., about 3.5 wt. %). Major ions in solution in “open sea” water include about 19.37 g/kg chloride; 10.77 g/kg sodium; 2.71 g/kg SO₄ (sulfate); 1.30 g/kg magnesium; 0.409 g/kg calcium; 0.338 g/kg potassium; 0.065 g/kg bromide; 0.026 g/kg H₃BO₃ (boric acid); and 0.010 g/kg strontium; with some trace organic compounds. Sea water salinity of total salts is about 3.5 wt. %. A higher temperature may also enhance anti-fouling agent and/or binder release, while, for example, an increased pH value enhances the solubility of rosin in a coating comprising rosin. Ocean water temperatures vary from about 28° C. on the Equator to about −2° C. at a polar region (e.g., about 10° C. to about 18° C. in temperate zones). The pH of surface ocean waters is typically about 8.0 to about 8.3. Oxygen concentration of sea water is about 0 to 0.8 vol. %, and oxidation may partly precipitate copper anti-fouling agents, such as in a rosin based coating.

To reduce fouling on a wooden surface (e.g., a wooden boat), a marine surface treatment that may be used includes: a wax; tar; asphalt; arsenic and/or sulphur admixed with oil; pitch (i.e., tar) admixed with a scraping of slime and/or alga; a resin, tallow, and/or oil admixed with pitch; pitch admixed with animal hair; and/or cement admixed with powdered iron, and optionally a copper and/or an arsenic compound; as well as an outer metal cladding surface and/or nails (e.g., copper, lead).

For a marine metal surface, a metal primer (e.g., a shellac, a varnish) are often used in a multicoat system, as a metallic pigment in a traditional anti-fouling topcoat often promotes corrosion. For example, a separating undercoat (e.g., a varnish, a shellac) were sometimes used to reduce a copper anti-fouling agent's corrosion of a surface comprising iron. A coating comprising a binder such as linseed oil, shellac varnish, tar resins (e.g., coal tar), and/or idem, have been used with an anti-fouling agent, such as an elemental pigment (e.g., an oxide, a sulphate) including an arsenic, a copper, an iron, a zinc, a tin, a titanium, and/or a mercury pigment. A liquid component of such coating's often comprised naphtha, benzene, and/or turpentine oil. In some cases a metallic soap (“hot plastic”) comprising an anti-fouling agent (e.g., copper sulphate, red mercury oxide, zinc dust, Indian red, zinc oxide) has been used as an anti-fouling surface treatment, with a gum shellac, coal tar, and/or rosin and a solvent liquid component (e.g., turpentine, pine tar oil, alcohol) used in a related formulation. In other instances, a metallic (e.g., cadmium, zinc) coating has been used as an anti-fouling coating.

A “soluble matrix coating” refers to a type of marine (e.g., anti-fouling) coating generally comprising a rosin type binder rich in an acidic moiety capable of reacting with an ion (e.g., sodium, potassium) and also typically comprises an anti-fouling agent. Often a plasticizer and/or an additional binder may be added to moderate a rosin's binder's brittleness and/or rate of dissolving in water. A soluble matrix coating typically has properties such as: sensitivity to oxygen due to double bonds at the acid moieties, so that a minimal time in dry-dock vs. immersion in water is common during service life; a service life of up to about 15 months; a reduced mechanical strength property; a thin film formation/application; suitability for use over a bituminous type primer; and a relatively low ability to be loaded with a biosoluble substance; and an anti-fouling property more effective in a higher speed vessel relative to a stationary surface.

A “controlled depletion coating” (e.g., a controlled depletion paint also known as a “CDP”), also referred to as an ablative/erodible coating, generally is similar to a soluble matrix coating, but comprises a soluble, physically curing binder whose release (e.g., erosion, dissolution) upon contact with water is regulated by a resin such as a polymeric synthetic organic resin (e.g., a partial or full substitute for rosin). These coatings produce pores as the anti-fouling agent(s) releases, but generally comprise a reduced (e.g., absent) amount of TBT. A CDP typically comprises a copper oxide anti-fouling agent, and may further comprise a co-biocide. Examples of a commercially produced CDP include Sea Tender 10/12/15 and TFA 10/30 (Chugoku Marine Paints, Ltd.; Tokyo Club Building, 2-6, Kasumigaseki 3-chome, Chiyoda-ku, Tokyo, 100-0013, Japan); Interspeed®340 (International Paint LLC, 6001 Antoine Drive, Houston, Tex. 77091); New Crest (Kansai Paint Co., Ltd., 6-14, Imabashi 2-chome, Chuo-ku, Osaka 541-8523, Japan); and Optima 2.30-2.36 (Transocean Coatings Sp. z o.o., 41-800 Zabrze, ul. Pawliczka 25). A CDP typically has a service life up to about 3 years, has a reduced self-polishing property relative to a self-polishing coating, and/or is often applied to a smaller vessel (e.g., a recreational boat).

An “insoluble matrix coating” (“hard anti-fouling coating,” “contact leaching coating,” “continuous contact coating”) generally refers to a relatively water insoluble anti-fouling coating comprising a high molecular mass binder (e.g., an acrylic binder, an epoxy binder, a chlorinated rubber binder, a vinyl binder). An insoluble matrix coating's properties often include: a polymer matrix that resists being polished/eroded in water; enhanced mechanical strength; oxidation resistance; photodegradation resistance; an ability to load a relatively large amount of an anti-fouling agent; pore creation in the coating as an anti-fouling agent is released, and/or a service life of up to about 1 to 2 years.

A “self polishing coating” (e.g., a self-polishing tributyl tin coating, a tin-free self-polishing system) refers to a coating that typically has properties such a smooth surface, particularly upon abrasion by moving water. A self polishing coating generally releases an anti-fouling agent at a relatively constant rate during service life (e.g., about 5 years). In some instance, a self polishing coating comprising fibres (e.g., about 2 to about 10 μm thick; about 50 to about 100 μm in length) and a binder such as a silylate, and acrylate, and/or a methacrylate, may be formulated with a higher solids content for better regulation of the self-polishing property.

A “self-polishing” (“SP”) tributyl tin (“TBT”) [e.g., a bis-oxide TBT (“TBTO”); a fluoride TBT (“TBTF”)] anti-fouling coating generally comprises a water soluble acrylic binder (e.g., a methyl methyacrylate and methacrylate copolymer) that binds TBT via ester linkages, and may comprise another anti-fouling agent (e.g., a pigment, a ZnO, a copper oxide). Hydrophobic regions of polymer bound TBT reduce pore creation as the additional anti-fouling agent diffuses from the coating. The TBT is also released by hydrolysis of the ester linkage in alkaline conditions. As the top layers of the coating's binder becomes brittle and erodes, reducing the surface friction (“self-polish”) and promotes further release anti-fouling agent(s) from deeper regions of the coating. A SP-TBT coating generally produces a more steady rate of anti-fouling activity over the coating's service life (e.g., about 5 years). The binder's monomer composition may be formulated to undergo differing rates of self-polishing, with slower or non-moving surfaces having a faster rate of coating degradation to enhance the anti-fouling effect. A self-polishing tributyl tin coating often possesses mechanical strength and durability, and TBT is relatively non-corrosive on an aluminum and/or a steel surface.

A “tin-free self-polishing system” (“TF-SPC”) is similar to a self-polishing tributyl tin anti-fouling coating but with a reduced (e.g., absent) tin component. A TF-SPC generally comprises an acrylic binder with moieties (“pendent groups”) added to the main polymer chain(s) that may bind an anti-fouling agent that typically comprises copper, zinc, and/or silicon (e.g., a copper acrylate coating). Acrylate (e.g., alkyl acrylate) and methacrylate (e.g., methoxy elthyl acrylate) monomers may be used in such a polymer. The bound anti-fouling agent is released possibly by an ion exchange reaction with sodium in sea water and/or a hydrolysis reaction upon contact with water. For copper, a release rate of about 10 g/cm² per day typically may produce anti-fouling activity. A co-biocide may also be a coating component. A reduced content of a rosin generally enhances the coating's photostability. Service life for a TF-SPC may be between about 3 to about 5 years. Examples of a commercially produced TF-SPC includes an ABC3 series (PPG Protective & Marine Coatings; One PPG Place, Pittsburgh, Pa. 15272); a Sea Granprix series, some of which have comprised a copper acrylate binder, a zinc acrylate binder, and/or a silyl acrylate binder (e.g., a methyl methacrylate and tributylsilyl methacrylate and/or tripropylsilyl methacrylate copolymer), as well as a possibly a chlorinated paraffin copolymer to increase peeling or cracking resistance, and/or co-biocides (Chugoku Marine Paints, Ltd.; Tokyo Club Building, 2-6, Kasumigaseki 3-chome, Chiyoda-ku, Tokyo, 100-0013, Japan); a Globoic Series coating, which has comprised a synthetic rosin (“colophony”) binder for oxidation resistance and bind zinc carboxylate for ion exchange release/hydrolysis and/or a rosin binder, mineral fibers to reinforce mechanical properties of the binder, copper pyrithione and/or co-biocide(s), a thixotropic agent (e.g., benonite), a flexibilizer (e.g., a vinyl resin, an oil resin, an ethyl acrylate, acrylamide-based terpolymer) and a plasticizer [Hempel (USA) Inc., 600 Conroe Park North Dr., Texas 77303]; an Intersmooth Ecoflex SPC, which has comprised an acrylic binder and a copper salt (International Paint LLC, 6001 Antoine Drive, Houston, Tex. 77091); and SeaQuantum, which comprises a silyl acrylate polymer and possibly rosin derivatives (Jotun Paints Inc., P.O. Box 159, 9203 Highway 23, Belle Chasse, La. 70037).

An “anti-fouling marine hybrid coating” comprising compositional and function features of a CDP and a TF-SPC is also available. Examples of a commercially available TF-SPC/CDP hybrid coating include Interswift 655 (International Paint LLC, 6001 Antoine Drive, Houston, Tex. 77091) and a Combic Series coating [Hempel (USA) Inc., 600 Conroe Park North Dr., Texas 77303].

An anti-fouling coatings may also be categorized as “tin-free” coating(s) (e.g., a tin free system), which generally refer to anti-fouling coatings with reduced tin concentration, such as a controlled depletion system and/or a TF-SPC.

A “biocide free anti-fouling coating” (e.g., a non-stick, fouling-release coating, a low fiction coating) refers to a reduced concentration (e.g., absence) of one or more biocides in the coating relative to other anti-fouling coating, though it is contemplated that such a coating may be modified to incorporate any one or more anti-fouling agent(s) (e.g., a peptide) described herein or as would be know in the art. A “non-stick, fouling-release coating” (e.g., a silicon rubber finish, a Teflon surface) generally possesses a low-coefficient of friction and/or smooth surface to reduce adhesion of a fouling molecule and/or organism. For example, a slime may undergo sloughing, which refers to removal of a slime due to hydrodynamic force, from a surface treatment such as a non-stick, fouling release coating. A non-stick, fouling-release coating generally possesses a non-polar (i.e., hydrophobic) and/or low friction property to inhibit the initial attachment of a polar adhesive fouling biomolecule and/or a marine fouling organism. Motion through water and/or a cleaning technique such as a water jet may remove a loosely attached fouling biofilm. A non-stick, fouling-release coating tend to work better for surfaces in contact with water moving at about 6 knots or faster (e.g., 30 knots or more), such as a high speed vessel. For example, barnacle attachment may be reduced at about 7 knots or more, algae at 18 knots or more, and slime films at 30 knots or more (Ryle, M., 1999). A non-stick, fouling-release coating may be part of a multicoat system (e.g., a surface adhesive primer, an undercoat, a biocide free topcoat) to promote adhesion to a surface (e.g., a steel surface). For example, a basecoat for adhesion both to the substrate and to the generally non-adhesive topcoat may be applied, with adhesion to the topcoat promoted by a chemical bonding reaction between the two coating layers. A non-stick anti-fouling coating is often compatible for use on aluminum surfaces.

Often a binder for a non-stick coating is selected to possess a relatively flexible polymer backbone, moieties that can move to the molecular surface and provide a low free surface energy that promotes low friction, and provide a relatively smooth molecular surface to reduce infiltration of a fouling film component. Examples of a non-stick, fouling-release coating (e.g., a topcoat) include those comprising a fluoropolymer [e.g., an epoxy fluorinated polymer, a polyurethane fluorinated polymer, a fluoridated poly(ethylene), a fluoridated poly(propylene), a poly(tetrafluoroethylene)], and/or a silicone polymer [e.g., a poly(dimethylsiloxane), a poly(3,3,3-trifluoromethyl)(methyl)siloxane]. A fluoropolymer binder may comprise, for example a perfluoroalkyl moiety (e.g., CF₃) that become cross-linked at the surface to reduce additional interactions with a fouling adhesive. In comparison, a silicone [e.g., a poly(dimethylsiloxane)] coating has low surface energy and polymer moieties that are mobile enough to reduce successful adhesive interactions with a fouling biofilm. A silicone coating may also comprise a liquid component (e.g., an oil) to promote this molecular scale motion, though the coating may become brittle and less fouling resistant as the liquid component is eluted from the coating, producing a service life of up to about 2 years.

An additional example of a non-stick, fouling-release coating (e.g., two coat system, a multi-coat system) include a primer (e.g., a polybutadiene coating, a urethane coating) for surface adhesion properties, and a non-stick, fouling-release topcoat (e.g., a silicone coating, a hydrocarbon coating) to reduce a fouling biofilm's adhesion. Further examples of a non-stick, fouling-release coating include a polyurethane-silicone-hydrocarbon coating system comprising a polyurethane basecoat; a 1,2-polybutadiene-silicone coating; a silicone-urethane coating (e.g., a paint, a clear coat); a urethane-hydrocarbon coating system (e.g., two coats) comprising a polyurethane basecoat having unreacted isocyantate moieties and a hydroxy-functional hydrocarbon topcoat, so that the hydroxyl moieties react with the isocyanate moieties for adhesion between the coating layers; and a two pack urethane coating comprising a silicone oil and an extender pigment (i.e., hydrophobic silica) to absorb and gradually release the silicone oil to promote retention of coating properties (e.g., cracking resistance, delamination resistance) (Adkins, J. D. et al., Progress in Organic Coatings, 29:1-5, 1996).

Further marine material formulations (e.g., coatings) have been described as well that may be combined with the biomolecular composition(s) describe herein. For example, U.S. Pat. No. 2,989,407 describes a rosin and blown fish oil binder based coating comprising an anti-fouling agent such as cuprous oxide, cuprous sulfide, and/or copper pigment at about 27% to about 35% (by weight and/or volume) and/or mercuric oxide at about 0.8% to about 5.3%. U.S. Pat. No. 2,602,752 describes a rosin and wax coating comprising anti-fouling cuprous oxide at about 32.4% to about 37.9% and/or copper linoleate at about 13.6% to about 24%. U.S. Pat. No. 2,579,610 describes a phenol formaldehyde based coating comprising an anti-fouling lead acetate at about 4.5% to about 21%. U.S. Pat. No. 3,219,505 anti-fouling copper particles (e.g. copper pigment). U.S. Pat. No. 3,154,460 describes a polyester and/or epoxy based coating comprising an anti-fouling such as copper, arsenic, or mercury. U.S. Pat. No. 3,033,809 describes a polyisobutylene elastomer coating and an anti-fouling copper pigment. U.S. Pat. No. 3,332,789 describes a rosin based coating comprising an anti-fouling copper pigment (e.g. cuprous oxide) and about 16.4% and dichlorodiphenyldichloroethane about 4.1%. U.S. Pat. No. 2,533,744 describes an anti-fouling cuprous lower alkyl mercaptan salt (e.g., a cuprous isopropyl mercaptan, a cuprous methyl mercaptan, a cuprous sec-butyl mercaptan, a cuprous ethyl mercaptan, a cuprous propyl mercaptan). U.S. Pat. No. 3,065,087 describes an anti-fouling organocopper compound (e.g., copper ethyl acetoacetate, copper o-benzoylbenzoate, copper 2,4-dinitrophenolate, copper pentachloropheoxyacetate, copper pentachlorophenoxyacetate, copper 4,6-dinitro-o-cresolate). U.S. Pat. No. 3,111,456 describes an anti-fouling copper naphthenate, crystal violet, malachite green oxalate, tributyltin oxide, a tributyltin fatty acid salt, a cellosolve, a chlordan, a dieldrin, and a toxaphine. U.S. Pat. No. 2,738,283 describes an anti-fouling cupric hydroxide. U.S. Pat. Nos. 2,476,372 and 2,583,545 describes an anti-fouling metallic copper, zinc oxide, metallic zinc, cuprous oxide, and mercuric oxide. U.S. Pat. No. 3,100,719 describes an anti-fouling pigment prepared as silica covered in copper oxide. U.S. Pat. No. 2,514,868 describes an anti-fouling pigment prepared from precipitated copper and/or cement copper. U.S. Pat. No. 2,420,540 describes an anti-fouling cupreous powder. U.S. Pat. No. 2,690,399 describes an anti-fouling black cupreous powder. U.S. Pat. No. 3,476,577 describes anti-fouling borate glass and cupric oxide granules. U.S. Pat. No. 3,579,533 describes an anti-fouling phenyl mercury maleate. U.S. Pat. No. 2,434,291 describes an anti-fouling phyenyl mercury borate. U.S. Pat. No. 2,423,044 describes an anti-fouling aryl polymercury naphthenate. U.S. Pat. No. 2,389,229 describes an anti-fouling aromatic murcurial. U.S. Pat. No. 2,525,155 describes an anti-fouling murcuric salt of n-propyl or n-butyl. U.S. Pat. No. 2,521,720 describes an anti-fouling perthiocyanic acid organo-metal salt. U.S. Pat. No. 3,214,281 describes an anti-fouling 5-hydro-10-substituted phenarsazine organo arsenic compound. U.S. Pat. No. 3,337,352 describes a coating (e.g., a paint) comprising about 1% to about 35% of an anti-fouling triphenarsazine chloride. U.S. Pat. No. 3,041,188 describes a coating (e.g., a paint) comprising an anti-fouling 5-hydro-10-fluorophenyarsazine and derivatives of about 10% to about 30%. U.S. Pat. No. 3,234,032 describes a marine coating comprising about 2.5% to about 40% of an anti-fouling bis(tri-n-butyltin) sulfide. U.S. Pat. Nos. 3,211,680 and 3,236,793 describes a coating (e.g., a varnish, a vinyl acetate-vinyl chloride copolymer paint) comprising an anti-fouling tributyltin toluenesulfonate, tributyltin isonicotinate, bis(tributyltin) terephthalate, tetrakis(tributyltin) pyromellitate, and/or an aliphatic dicarboxylic acid bis(tributyltin) ester. U.S. Pat. No. 2,970,923 describes a marine paint comprising an anti-fouling triphenyltin chloride of about 6% to about 18%. U.S. Pat. No. 3,268,347 describes an anti-fouling paint comprising about 20% of an anti-fouling 2,5-dimercapto-1,3,4-thiadiazole organic tin salt. U.S. Pat. No. 3,625,966 describes a coating (e.g., a paint) comprising an anti-fouling oxobenzothiazine acetic acid trihydrocarbyltin salt. U.S. Pat. No. 3,684,752 describes a coating comprising about 30% to about 40% an anti-fouling high molecular weight organic tin. U.S. Pat. No. 3,615,744 describes a coating (e.g., a paint) comprising a combination of an anti-fouling organic tin, a copper compound, and 2-amino-3-chloro-1,4-naphthoquinone. U.S. Pat. No. 3,227,563 describes an anti-fouling chlorinated methanobenzene and an organotin. U.S. Pat. No. 3,287,210 describes an antimicrobial triphenyl antimony compound. U.S. Pat. No. 2,580,025 describes a pipeline bitumen enamel comprising an anti-fouling antimony oxide and a barium carbonate. U.S. Pat. No. 3,197,314 describes an anti-fouling organobismuth compound. U.S. Pat. No. 3,623,896 describes an anti-fouling terephthalic acid heavy metal (e.g., manganese, cobalt, copper) salt. U.S. Pat. No. 3,266,913 describes an anti-fouling metal (e.g., a mercury, lead, copper, zinc) salt of aspartic and glutamic acid. U.S. Pat. No. 3,350,211 describes a marine anti-fouling coating comprising about 15% to about 47% of an anti-fouling 2-thiazolyl benzimidazole complex comprising zinc, lead, or mercury. U.S. Pat. No. 3,211,679 describes a coating (e.g., a marine paint) comprising about 25% or more of an anti-fouling triphenylborane-amine complex. U.S. Pat. No. 3,597,440 describes an anti-fouling isoperthiocyanic acid salt. U.S. Pat. No. 3,677,777 describes a paint comprising an anti-fouling 2-(N,N-diethylthiocarbamoylthio)-5-nitrothiazol and/or 2-(N,N-dimethylthiocarbamoylthio)-5-nitrothiazol. U.S. Pat. No. 3,557,281 describes an anti-fouling dithiooxamide and related compounds. U.S. Pat. No. 3,347,686 describes an anti-fouling N-(deca-chloro-3-hydroxypentacyclo-decyl-3) amide. U.S. Pat. No. 2,978,338 describes an anti-fouling thiotetrahydrophthalimide. U.S. Pat. No. 3,265,567 describes an anti-fouling chlorophenyl methylcaramate (e.g., a 5-tert-butyl-2-chlorophenyl methyl-carbamate). U.S. Pat. No. 3,652,496 describes an anti-fouling biacetyl dihydrazone. U.S. Pat. No. 3,279,984 describes an anti-fouling 1-bromo-3-nitrobenzene. An anti-fouling agent component of a material formulation, such as those described above, typically may comprise between about 1% to about 70% of a material formulation (e.g., about 10% to about 70%). Additionally, U.S. Pat. Nos. 3,081,175 and 3,326,174 describes liquid jets and liquid washing agents for reducing fouling on a ship's exterior [“Anti-fouling Marine Coatings” Williams, A. Noyes, Data Corporation, Park Ridge, N.J. 1973].

Various pigments may be used in a marine and/or an anti-fouling coating, and some may provide a metal ion to a metal binding proteinaceous molecule. For example, a modified zinc phosphate, such as, for example, an aluminum zinc phosphate, a basic zinc phosphate hydrate, a zinc silicophosphate hydrate, a basic zinc molybdenum phosphate, or a combination thereof may confer improved corrosion resistance for a salt water embodiment. A zinc phosphate may be less selected for a marine coating for salt water embodiments. A zinc molybdate, a zinc phosphate, a zinc hydroxy phosphite, or a combination thereof may confer a white color. These zinc pigments function by reducing an anodic process, though a zinc hydroxy phosphite may form corrosion resistant soap in an oleoresinous-coating. A basic zinc molybdate may be selected for an alkyd-coating, an epoxide-coating, an epoxy ester-coating, a polyester-coating, a solvent-borne coating, or a combination thereof. A basic zinc molybdate-phosphate may be similar to a basic zinc molybdate, though it may provide improved corrosion resistance for a rusted steel surface. A basic calcium zinc molybdate may be selected for a water-borne coating, a two-pack polyurethane coating, a two-pack epoxy coating, or a combination thereof. A combination of a basic calcium zinc molybdate and a zinc phosphate may confer an improved adhesion property to a surface comprising an iron, and may be selected for a water-borne coating and/or a solvent-borne coating. A zinc phosphate may be selected for an alkyd coating, a water-reducible coating, a coating cured by an acid and baking, or a combination thereof. A zinc hydroxy phosphite may be selected for a solvent-borne coating.

A silicate pigment such as a barium borosilicate, a calcium borosilicate, a strontium borosilicate, a zinc borosilicate, a calcium barium phosphosilicate, a calcium strontium phosphosilicate, a calcium strontium zinc phosphosilicate, or a combination thereof, typically acts through inhibiting an anodic and/or a cathodic process, as well as forming a corrosion resistant soap in an oleoresinous-coating. A grade I and/or a grade III calcium borosilicate may be selected for a medium oil alkyd-coating, a long oil alkyd, an epoxy ester-coating, a solvent-borne coating, an architectural coating, an industrial coating, or a combination thereof, but may be less selected for a marine coating, an epoxide-coating, a water-borne coating, or a combination thereof. A calcium barium phosphosilicate grade I pigment may be selected for a solvent-borne epoxy-coating, to confer an anti-settling property to a primer comprising zinc, or a combination thereof. A calcium barium phosphosilicate grade II pigment may be selected for a water-borne coating, an alkyd-coating, or a combination thereof. A calcium strontium phosphosilicate may be selected for a water-borne acrylic lacquer, a water-borne sealant, or a combination thereof. In aspects wherein a water-borne acrylic lacquer comprises a calcium strontium phosphosilicate, about a 1:1 ratio of a zinc phosphate pigment may be included. A calcium strontium zinc phosphosilicate may be selected for an alkyd-coating, an epoxide coating, a coating cured by a catalyst and baking, a water-borne coating, or a combination thereof.

Specific procedures for determining the purity/properties of a marine coating, an anti-fouling coating, and/or a coating component thereof (e.g., a cuprous oxide, a copper powder, an organotin) under marine conditions (e.g., submergence, water based erosion, seawater biofouling resistance, barnacle adhesion resistance) and/or a marine film have been described, for example, in “ASTM Book of Standards, Volume 06.02, Paint—Products and Applications; Protective Coatings; Pipeline Coatings,” D3623-78a, D4938-89, D4939-89, D5108-90, D5479-94, D6442-99, D6632-01, D4940-98, and D5618-94, 2002; and “ASTM Book of Standards, Volume 06.03, Paint—Pigments, Drying Oils, Polymers, Resins, Naval Stores, Cellulosic Esters, and Ink Vehicles,” D912-81 and D964-65, 2002.

b. Additional Coatings Comprising a Biomolecular Composition

Various types of coatings may comprise a biomolecular composition, including an architectural coating, an industrial coating, or a specification coating. An architectural coating (“trade sale coating,” “building coating,” “decorative coating,” “house coating”) comprises a coating suitable to coat surface materials commonly found as part of buildings and/or associated objects (e.g., furniture). Examples of a surface an architectural coating may be applied to include, a plaster surface, a wood surface, a metal surface, a composite particle board surface, a plastic surface, a coated surface (e.g., a painted surface), a masonry surface, a floor, a wall, a ceiling, a roof, or a combination thereof. Additionally, an architectural coating may be applied to an interior surface, an exterior surface, or a combination thereof. An interior coating generally possesses properties such as minimal odor (e.g., no odor, very low VOC), good blocking resistance, print resistance, good washability (e.g., wet abrasion resistance), or a combination thereof. An exterior coating may be selected to possess good weathering properties. Examples of coating type commonly used as an architectural coating include an acrylic-coating, an alkyd-coating, a vinyl-coating, a urethane-coating, or a combination thereof. In certain aspects, a urethane-coating may be applied to a piece of furniture. In other facets, an epoxy-coating, a urethane-coating, or a combination thereof, may be applied to a floor. In some embodiments, an architectural coating comprises a multicoat system. In certain aspects, an architectural coating comprises a high performance architectural coating (“HIPAC”). A HIPAC produces a film with a combination of good abrasion resistance, staining resistance, chemical resistance, detergent resistance, and mildew resistance. Examples of binders suitable for producing a HIPAC include a two-pack epoxide, a two-pack urethane, and/or a moisture cured urethane. In general embodiments, an architectural coating comprises a liquid component, an additive, or a combination thereof. In other aspects, an architectural coating comprises a pigment. In some aspects, such an architectural coating may be formulated to comprise a reduced amount or lack a toxic coating component. Examples of a toxic coating component include a heavy metal (e.g., lead), a formaldehyde, a nonyl phenol ethoxylate surfactant, a crystalline silicate, or a combination thereof. An example of an architectural coating includes those formulated for use on a wood surface (“wood coating,” “architectural wood coating”); a masonry surface (“masonry coating,” “architectural masonry coating”) such as a stone, a brick, a tile, a cement-based material (e.g., concrete, a mortar); as well as an artist coating formulated for a decorative purpose, or a combination thereof.

An industrial coating is a coating applied to a surface of a manufactured product in a factory setting. An industrial coating typically undergoes film formation to produce a film with a protective and/or aesthetic purpose. Examples of coating types that are commonly used for an industrial coating include an epoxy-coating, a urethane-coating, alkyd-coating, a vinyl-coating, chlorinated rubber-coating, or a combination thereof. Examples of a surface commonly coated by an industrial coating include metal (e.g., aluminum, zinc, copper, an alloy, etc); glass; plastic; cement; wood; paper; or a combination thereof. An industrial coating may be storage stable for 12 months or more, applied at ambient conditions, applied using a hand-held applicator, undergo film formation at ambient conditions, or a combination thereof. However, an industrial coating often does not meet one or more of these characteristics previously described as preferred for an architectural coating. For example, an industrial coating may have a storage stability of only days, weeks, or months, as due to a more rapid use rate in coating factory prepared items. An industrial coating may be applied and/or undergo film formation at baking conditions. An industrial coating may be applied using techniques such as, for example, spraying by a robot, anodizing, electroplating, and/or laminating of a coating and/or film onto a surface. In some embodiments, an industrial coating undergoes film formation by irradiating the coating with non-visible light electromagnetic radiation and/or particle radiation such as UV radiation, infrared radiation, electron-beam radiation, or a combination thereof. An example of an industrial coating includes a marine coating, a coating used on an automotive vehicle (“automotive coating”); and/or a coating used on a container (“can coating”) such as a metal container (e.g., an aluminum container, a steel container) for containing a material such as a food, a chemical, etc.

A specification coating may be formulated by selection of coating components to fulfill a set of requirement(s) (e.g., particular properties) prescribed by a consumer. An example of a specification coating by customer type includes a military specified coating, a Federal agency specified coating (e.g., Department of Transportation), a state specified coating, or a combination thereof. Additional examples of a specification coating includes a chemical agent resistant coating, a camouflage coating, a pipeline coating, a traffic marker coating, an aircraft coating, or a nuclear power plant coating. A pipeline coating, such as, for example, a coating for a metal pipeline used to convey a fossil fuel, often possesses corrosion resistance. A traffic marker coating is typically used to visibly convey information on a surface usually subjected to weathering and abrasion (e.g., a pavement). An aircraft coating protects and/or decorates a surface (e.g., metal, plastic) of an aircraft, and often may be selected for excellent weathering properties, heat and/or cold resistance (e.g., about −54° C. to about 177° C.). A nuclear power plant coating generally possesses particular properties (e.g., gamma radiation resistance, chemical resistance) suitable for use in such an environment. A camouflage coating is generally formulated with materials (e.g., pigments) that reduce the detection (e.g., by visible light, by a non-visible light such as infrared radiation) of a coated surface from the surrounding environment.

13. Empirically Determining the Properties of Coatings and/or Films

A coating and/or a film may be prepared by varying the ranges and/or combinations of coating component(s) including a biomolecular composition described herein, to achieve a desired/suitable set of property(s) for a particular use (e.g., surface, environment, etc), and such coating and/or component selection and preparation may be done in light of the present disclosures. For example, a variety of assays are available to measure various properties of a coating, a coating application, and/or a film to determine the degree of suitability of a coating composition for use in a particular use (see, for example, in “Hess's Paint Film Defects: Their Causes and Cure,” 1979). In a further example, the physical properties (e.g., purity, density, solubility, volume solids and/or specific gravity, rheology, viscometry, and particle size) of the resulting a liquid paint and/or other coating product (e.g., on comprising a biomolecular composition), can be assessed using standard techniques of the art and/or as described in PAINT AND COATING TESTING MANUAL, 14^{th} ed. of the Gardner-Sward Handbook, J. V. Koleske, Editor (1995), American Society for Testing and Materials (ASTM), Ann Arbor, Mich., and applicable published ASTM assay methods. Any other suitable assay method of the art may be employed for assessing physical properties of the paint or coating mixture comprising an above-described biomolecular composition (e.g., an enzyme, an antifungal peptide additive, etc.).

14. Combinations of Biomolecular Compositions and Chemical Preservatives and Antimicrobial Agents

A material formulation such as a surface treatment (e.g., a coating) and/or a polymeric material (e.g., a plastic) may comprise an anti-biological agent to reduce and/or prevent the deterioration of the material formulation by an organism (e.g., a biological cell, a virus) such as a microorganism. An organism may, for example, infest, survive upon, survive within, grow on the surface, and/or grow within, an inanimate object. An “inanimate object” refers to a structure and/or object other than a living cell (e.g., a living organism), such a surface and/or a coated surface. In some embodiments, a target cell and/or a target virus may be capable of infesting an inanimate object (e.g., a ship, a building, a piece of furniture, a wall, a coated surface, a material formulation, etc). For example, a microorganism may be considered a contaminant capable damaging a material formulation (e.g., a film, a coating) to the point of no longer being of suitable usefulness in a given embodiment. In another example, an undesirable growth of a microorganism is generally more prevalent in a water-borne coating, as the solvent component of a solvent borne-coating usually acts as a preservative. In further example, a film of a terrestrial coating is generally susceptible to such damage by growth of a microorganism after loss of a solvent (e.g., evaporation) during film formation. In an additional example, various bacteria (e.g., Bacillus spp.) and fungi produce spores, which are cells that are relatively durable to unfavorable conditions (e.g., cold, heat, dehydration, a biocide) and may persist in a terrestrial coating and/or film for months or years prior to germinating into a damaging colony of cells.

In certain embodiments, a biomolecular composition (e.g., a proteinaceous molecule, a microorganism based particulate material, an enzyme), may be used as a purposefully added material formulation component as an anti-biological agent. The amount of an anti-biological agent added to a material formulation comprising a biomolecular composition may be increased relative to a typical content of a similar material formulation lacking such an added biomolecular composition. In certain aspects, the amount of an anti-biological agent may be increased about 1.01 to about 10-fold or more, the amount of an example of an anti-biological agent content described herein or used in the art, in light of the present disclosures. In some embodiments, the concentration of any individual anti-biological agent (e.g., an anti-biological biomolecular composition) comprises about 0.000000001% to about 20% (e.g., about 0.000000001% to about 4%) or more, of a material formulation. In some aspects, an improved (e.g., additive, synergistic) effect may occur between a plurality of anti-biological agents wherein one comprises a biomolecular composition, so that the concentration of one or more components of the anti-biological agent may be reduced relative to the component's use alone or in a combination comprising fewer components.

Anti-biological activity (e.g., growth inhibition, biocidal activity) of an anti-biological agent can provide and/or facilitate disinfection, decontamination and/or sanitization of an material and/or an object (e.g., an inanimate object, a building material), which refer to the process of reducing the number of cell(s) and/or viruses to levels that no longer pose a threat (e.g., a threat of damage to a property, a detrimental threat of damage to a surface, a threat to the health of a desired organism such as human). For example, use of a bioactive anti-biological agent can be accompanied by removal (e.g., manual removal, machine aided removal) of a biological entity and/or a material contaminated with a biological entity, and use of the bioactive anti-biological agent may promote ease of removal of the biological entity.

A biomolecular composition (e.g., an anti-biological proteinaceous molecule) may be used alone or combined with any other anti-biological agent described herein and/or known in the art, such as an anti-fouling agent typically used with an aqueous surface treatment (e.g., a marine coating, a pipeline coating), a preservative (e.g., a chemical biocide, a chemical biostatic) typically used in a surface treatment (e.g., a terrestrial surface treatment), an antimicrobial agent (e.g., a chemical biocide, a chemical biostatic) typically used in a polymeric material (e.g., a plastic, an elastomer, an adhesive, a sealant, etc), and/or an anti-biological technique (e.g., cleaning, disinfection) (see for example, Baldridge, G. D. et al, 2005; Hancock, R. E. W. and Scott, M. G., 2000). A biomolecular composition and/or the additional anti-biological agent may reduce and/or prevent such growth of a contaminating organism. Some such combinations of anti-biological biomolecular compositions and/or combinations with another anti-biological agent may provide an advantage such as a broader range of activity against various biological entities (e.g., a bacteria, an alga, a fungus, etc.), a synergistic anti-biological (e.g., preservative) effect, a longer duration of effect, or a combination thereof. For example, one or more anti-biological proteinaceous molecule(s) may be used in combination with and/or as a substitute for one or more existing anti-biological agent(s) (e.g., a preservative, an antimicrobial agent, an anti-fouling agent, a fungicide, a fungistatic, a bactericide, an algacide, etc.) identified herein and/or in the art. In another example, a coating material typically contains a polymeric substances such as casein, acrylic, polyvinyl and/or carbon polymer(s) (e.g., binders) which may serve as nutrient(s) for a cell (e.g., a fungal cell) that may support the growth of, for example, a cell on a paint film and/or a coated surface, inside a can of a liquid coating (e.g., a paint) during storage. A proteinaceous molecule may be added to such a coating material, for example, to render the coating composition resistant to a cell's infestation and growth, provide a coated surface with a sustainable anti-biological (e.g., anti-fouling) activity that protects the recipient surface from biodegradation and/or fouling, and/or provides organism resistance (e.g., fouling organism resistance) to the coating composition.

Examples of an anti-biological agent (e.g., a preservative) that an anti-biological proteinaceous molecule (e.g., an anti-fouling proteinaceous molecule) may substitute for and/or be combined with include, but are not limited to those non-proteinaceous anti-biological (e.g., antimicrobial) compounds (i.e., biocides, bactericides, fungicides, algaecides, mildewcides, etc.) which have been shown to be of utility and are currently available and approved for use in the U.S./NAFTA, Europe, and the Asia Pacific region, and numerous examples are described herein for use with a material formulation such as a surface treatment (e.g., a coating), etc. In a particular example, an anti-biological peptidic agent comprises ProteCoat® (Reactive Surfaces, Ltd.; also described in U.S. Pat. Nos. 6,020,312; 5,885,782; and 5,602,097, and patent application Ser. Nos. 10/884,355 and 11/368,086). For example, certain peptides contemplated for use (e.g., ProteCoat®; Reactive Surfaces, Ltd.) as described herein have been shown to involve synergy between the peptides (e.g., antifungal peptides) and non-peptide antifungal agents that may be useful in controlling growth of a Fusarium, a Rhizoctonia, a Ceratocystis, a Pythium, a Mycosphaerella, an Aspergillus and/or a Candida genera of fungi. In particular, synergistic combinations have been described and successfully used to inhibit the growth of an Aspergillus fumigatus and an A. paraciticus, and also a Fusarium oxysporum with respect to agricultural applications. These and other additive or synergistic combination(s) of biomolecular (e.g., proteinaceous) anti-biological agent(s) described herein and a non-biomolecular (e.g., a non-proteinaceous) anti-biological agent(s) may be useful as, for example, a component (e.g., an additive) in a material formulation (e.g., a paint, a coating) to confer an anti-biological activity (e.g., an anti-fouling activity) such as for deterring, preventing, and/or treating an infestation of a biological entity. In another example, an additional anti-biological agent comprises a non-proteinaceous antimicrobial agent, a non-amino based antimicrobial agent, a compounded peptide anti-biological agent, an enzyme-based anti-biological agent, or a combination thereof, such as those described in U.S. patent application Ser. No. 11/865,514 filed Oct. 1, 2007, incorporated by reference.

Various metal and/or organic compounds have been used as anti-fouling agent(s). An organometallic compound comprising an organic radical, a metal, and a halide (e.g., organoarsenic, organomercury, organotin such as tributyltin, organolead) has been used as an anti-fouling agent, often in combination with a copper compound. India red is sometimes used anti-fouling agent. For example, an anti-fouling organometallic coating (e.g., a paint) may comprise a polymeric binder (e.g., an acrylic ester) and organotin. Other anti-fouling agents that may be used include copper (I) thiocyanate, zinc (II) oxide, titanium (IV) oxide, iron (III) oxide, cuprous bromide, and/or cuprous cyanide. Oxygenated water may chemically react with a metal based anti-fouling agent (e.g., a Cu₂O pigment) to produce a toxic cation (e.g., Cu′) that possesses much of the anti-fouling activity. A free copper ion, for example, is generally more toxic than bound copper (e.g., chelated copper). Copper compounds are generally more effective against a microorganism, while an organometallic compound are often more toxic to a macro-organism such as a crustacean, a mollusk, and/or an oyster. In another example, an anti-fouling coating comprising a binder such as colophony, an acrylic resin, a vinyl resin, a chlorinated rubber polymer, an acrylic polymer (e.g., a methyl meta-acrylate) and an anti-fouling agent (e.g., an anti-fouling pigment), sometimes comprises another anti-fouling preservative (e.g., a co-preservative, a co-biocide, a reinforcing anti-fouling preservative). For example, Cu₂O pigment generally further comprising an organic co-biocide (e.g. a thiocarbamate, a dithiocarbamate such as Maneb, Zineb, Ziram, Thiram), such as in the case for a desire to reduce fouling against a copper resistant alga (e.g. Enteromorpha spp.). Examples of a reinforcing anti-fouling agent includes a polyvalent metal (e.g., copper, zinc) pyrithione salt that acts as a biocide (e.g., algaecide, fungicide, bactericide) generally with limited aqueous solubility; 2-methylthio-4-tert-butylamino-6-cyclopropylamino-s-triazine (“Irgarol 1051”), a herbicide which disperses in an aqueous medium; 3-(3,4-dichlorophenyl)-1,1-dimethylurea (“Diuron”), a water soluble urea-based herbicide that acts as a photosynthesis inhibitor, and hydrolyzes in a strong acid or a strong alkaline condition; a 2-(thiocyanatomethylthio)-1,3-benzothiazole 9 (“TCMTB”), a fungicide; 4,5-dichloro-2-n-octyl-3(2H)-isothiazolone (“Sea-Nine 211”), which has antimicrobial activity; 4,5,dichloro-2-n-octyl-4-isothiazolin-3-one (“Kathon 5287”); 2,4,5,6-tetrachloroisophthalonitrile (“Chlorothalonil”), a fungicide; N-dimethyl-N-phenylsulphamide (“Dichlofluanid”), a fungicide; bis(dimethylthiocarbamoyl)disulphide (“Thiram”); zinc bis(dimethylthiocarbamates) (“Ziram”), a fungicide; a zinc complex of 2-mercaptopyridine-1-oxide (“zinc pyrithione”), a fungicide and bactericide; manganese ethylene bisdithiocarbamate (“Maneb”); zinc ethylene bisdithiocarbamate (“Zineb”), a fungicide; benzmethylamide, fluorofolpet, polyphase, pyridone-triphenylborane, TCMS, TCMTB, tolyfluanid, or a combination thereof.

It is contemplated that a material isolated and/or purified from an organism may be used as an anti-fouling agent and/or a more generic anti-biological agent. For example, a marine organism may secrete a secondary metabolite that reduces fouling upon them. A slime film and/or a scraping of an alga admixed with pitch may have anti-fouling compound such as a secondary metabolite with an anti-fouling activity. It is contemplated that such a secondary metabolite may be used as an anti-fouling agent in other types of surface treatment(s). A secondary metabolite may possibly act as an anti-fouling agent by altering a biofouling organism's metabolism; catalytically degrade fouling biofilm adhesives; possessing biocidal activity; increasing negative chemotaxis; inhibiting a biofouling organism and/or fouling biofilm component's attachment; inhibiting a biofouling organism's growth; inhibiting a biofouling organism's metamorphosis; repelling a biofouling organism; or a combination thereof. An example of a secondary metabolite that is contemplated for use herein includes a terpenoid, a steroid, a zosteric acid, a fatty acid, an amino acid, a heterocyclic compound such as a furan and/or a lactone, an acetogenin, an alkaloid, and/or a polyphenol. Additionally, it is contemplated that a proteolytic enzyme may also be included in a surface treatment as an anti-fouling agent to cleave a protein (e.g., an adhesive protein used by a barnacle). For example, an aquatic animal, such as a whale that possesses a low fouling surface, may have a glycoprotein, a hydrated jelly that possesses a pore size smaller than that of many biofouling organisms, and/or a surface rich in hydrolytic enzyme(s), and an isolated gel and/or such a hydrolytic enzyme may be incorporated into a surface treatment as an anti-fouling agent.

Other anti-fouling agent(s) may include a radioisotope (e.g., ⁶⁰Co, ²⁰⁴Tl, ⁹¹Y, ⁹⁵Tc, ⁹⁹Tc) and/or UV irradiation to irradiate a fouling organism; an electrical agent and/or technique at a surface such as a conductive coating comprising a plastified chlorinated rubber polymer, coal suspension and graphite that is capable functioning as an anode for the production of biocidal chlorine via water electrolysis; a surface treatment having an electrical charge to promote production of ozone, ammonia, bromine, copper ions, oxygen, and/or platinum compounds with anti-fouling properties; toxic direct electron transfer from a surface acting as an electrode (e.g., a chloroprene carbon sheet, a surface treatment comprising a ferrocene derivative) to a fouling organism; or an organic (e.g., polyaniline) coating including a metallic anti-fouling agent, wherein a current released the metals as anti-fouling agents into the aqueous medium as well as offering corrosion resistance. An adhesive comprising electrostatically charged fibres has also been used to reduce hard biofouling accumulation. Acoustic vibrations, such as can be delivered via a piezoelectric coating, have been used to reduce fouling, as described in the art.

Examples of a preservative, which is an anti-biological agent commonly used in a coating (though an anti-biological agent such as a preservative may be adapted for use in other material formulations), include a biocide, which reduces and/or prevents the growth of an organism by killing the organism (e.g., a microorganism, a spore), a biostatic, which reduces and/or prevents the growth of an organism (e.g., a microorganism, a spore) but generally does not necessarily kill the organism, or a combination thereof (e.g., a combination of the effects). For example, a “fungicide” comprises a biocidal substance used to kill a specific microbial group, the fungi; while a “fungistatic” denotes a substance that prevents fungal microorganism from growing and/or reproducing, but do not result in substantial killing. Examples of a biocide include, for example, a microbiocide, a germicide, a herbicide, a bactericide, a fungicide, an algacide, a mildewcide, a molluskicide, a slimicide, a viricide, or a combination thereof. Examples of a biostatic include, for example, a microbiostatic, a germistatic, a herbistatic, a bacteristatic, a fungusstatic, an algastatic, a mildewstatic, a molluskistatic, a slimistatic, a viristatic, or a combination thereof. An anti-biological agent may possess a biocidal and/or biostatic activity.

In certain embodiments, a preservative may comprise an in-can preservative, an in-film preservative, or a combination thereof. An in-can preservative refers to a composition that reduces and/or prevents the growth of a microorganism prior to film formation. Addition of an in-can preservative during a water-borne coating production typically occurs with the introduction of water to a coating composition. Typically, an in-can preservative may be added to a coating composition for function during coating preparation, storage, or a combination thereof. An in-film preservative refers to a composition that reduces or prevents the growth of a microorganism after film formation. In many embodiments, an in-film preservative comprises the same chemical as an in-can preservative, but added to a coating composition at a higher (e.g., about two-fold or more) concentration for continuing activity after film formation.

Examples of a preservative used in a coating include a metal compound (e.g., an organo-metal compound) biocide, an organic biocide, or a combination thereof. Examples of a metal compound biocide include a barium metaborate (CAS No. 13701-59-2), which may function as a fungicide and/or a bactericide; a copper(II) 8-quinolinolate (CAS No. 10380-28-6), which may function as a fungicide; a phenylmercuric acetate (CAS No. 62-38-4), a tributyltin oxide (CAS No. 56-35-9), which may be less often selected for use against Gram-negative bacteria; a tributyltin benzoate (CAS No. 4342-36-3), which may function as a fungicide and a bactericide; a tributyltin salicylate (CAS No. 4342-30-7), which may function as a fungicide; a zinc pyrithione (“zinc 2-pyridinethiol-N-oxide”; CAS No. 13463-41-7), which may function as a fungicide; a zinc oxide (CAS No. 1314-β-2), which may function as a fungusstatic, a fungicide and/or an algacide; a combination of zinc-dimethyldithiocarbamate (CAS No. 137-30-4) and a zinc 2-mercaptobenzothiazole (CAS No. 155-04-4), which acts as a fungicide; a zinc pyrithione (CAS No. 13463-41-7), which may function as a fungicide; a metal soap; or a combination thereof. Examples of a metal comprised in a metal soap biocide include a copper, a mercury, a tin, a zinc, or a combination thereof. Examples of an organic acid comprised in a metal soap biocide include a butyl oxide, a laurate, a naphthenate, an octoate, a phenyl acetate, a phenyl oleate, or a combination thereof.

An example of an organic biocide that acts as an algacide includes a 2-methylthio-4-tert-butylamino-6-cyclopropylamino-s-triazine (CAS No. 28159-98-0). Examples of an organic biocide that acts as a bactericide include a combination of a 4,4-dimethyl-oxazolidine (CAS No. 51200-87-4) and a 3,4,4-trimethyloxazolidine (CAS No. 75673-43-7); a 5-hydroxy-methyl-1-aza-3,7-dioxabicyclo (3.3.0.) octane (CAS No. 59720-42-2); a 2(hydroxymethyl)-aminoethanol (CAS No. 34375-28-5); a 2-(hydroxymethyl)-amino-2-methyl-1-propanol (CAS No. 52299-20-4); a hexahydro-1,3,5-triethyl-s-triazine (CAS No. 108-74-7); a 1-β-chloroallyl)-3,5,7-triaza-1-azonia-adamantane chloride (CAS No. 51229-78-8); a 1-methyl-3,5,7-triaza-1-azonia-adamantane chloride (CAS No. 76902-90-4); a p-chloro-m-cresol (CAS No. 59-50-7); an alkylamine hydrochloride; a 6-acetoxy-2,4-dimethyl-1,3-dioxane (CAS No. 828-00-2); a 5-chloro-2-methyl-4-isothiazolin-3-one (CAS No. 26172-55-4); a 2-methyl-4-isothiazolin-3-one (CAS No. 2682-20-4); a 1,3-bis(hydroxymethyl)-5,5-dimethylhydantoin (CAS No. 6440-58-0); a hydroxymethyl-5,5-dimethylhydantoin (CAS No. 27636-82-4); or a combination thereof. Examples of an organic biocide that acts as a fungicide include a parabens; a 2-(4-thiazolyl)benzimidazole (CAS No. 148-79-8); a N-trichloromethyl-thio-4-cyclohexene-1,2-dicarboximide (CAS No. 133-06-2); a 2-n-octyl-4-isothiazoline-3-one (CAS No. 26530-20-1); a 2,4,5,6-tetrachloro-isophthalonitrile (CAS No. 1897-45-6); a 3-iodo-2-propynyl butyl carbamate (CAS No. 55406-53-6); a N-(trichloromethyl-thio)phthalimide (CAS No. 133-07-3); a tetrachloroisophthalonitrile (CAS No. 1897-45-6); a potassium N-hydroxy-methyl-N-methyl-dithiocarbamate (CAS No. 51026-28-9); a sodium 2-pyridinethiol-1-oxide (CAS No. 15922-78-8); or a combination thereof. Examples of a parbens include a butyl parahydroxybenzoate (CAS No. 94-26-8); an ethyl parahydroxybenzoate (CAS No. 120-47-8); a methyl parahydroxybenzoate (CAS No. 99-76-3); a propyl parahydroxybenzoate (CAS No. 94-β-3); or a combination thereof. Examples of an organic biocide that acts as a bactericide and fungicide include a 2-mercaptobenzo-thiazole (CAS No. 149-30-4); a combination of a 5-chloro-2-methyl-3(2H)-isothiazoline (CAS No. 26172-55-4) and a 2-methyl-3(2H)-isothiazolone (CAS No. 2682-20-4); a combination of a 4-(2-nitrobutyl)-morpholine (CAS No. 2224-44-4) and a 4,4′-(2-ethylnitrotrimethylene dimorpholine (CAS No. 1854-23-5); a tetra-hydro-3,5-di-methyl -2H-1,3,5-thiadiazine-2-thione (CAS No. 533-74-4); a potassium dimethyldithiocarbamate (CAS No. 128-03-0); or a combination thereof. An example of an organic biocide that acts as an algacide and fungicide includes a diiodomethyl-p-tolysulfone (CAS No. 20018-09-1). Examples of an organic biocide that acts as an algacide, a bactericide and a fungicide include a glutaraldehyde (CAS No. 111-30-8); a methylenebis(thiocyanate) (CAS No. 6317-18-6); a 1,2-dibromo-2,4-dicyanobutane (CAS No. 35691-65-7); a 1,2-benzisothiazoline-3-one (“1,2-benzisothiazolinone”; CAS No. 2634-33-5); a 2-(thiocyanomethyl-thio)benzothiazole (CAS No. 21564-17-0); or a combination thereof. An example of an organic biocide that acts as an algacide, a bactericide, a fungicide and a molluskicide includes a 2-(thiocyanomethyl-thio)benzothiozole (CAS No. 21564-17-0) and/or a methylene bis(thiocyanate) (CAS No. 6317-18-6).

In some embodiments, an antifungal agent (e.g., a fungicide, a fungusstatic) may comprise a copper (II) 8-quinolinolate (CAS No. 10380-28-6); a zinc oxide (CAS No. 1314-β-2); a zinc-dimethyl dithiocarbamate (CAS No. 137-30-4); a 2-mercaptobenzothiazole, zinc salt (CAS No. 155-04-4); a barium metaborate (CAS No. 13701-59-2); a tributyl tin benzoate (CAS No. 4342-36-3); a bis tributyl tin salicylate (CAS No. 22330-14-9), a tributyl tin oxide (CAS No. 56-35-9); a parabens: ethyl parahydroxybenzoate (CAS No. 120-47-8), a propyl parahydroxybenzoate (CAS No. 94-β-3); a methyl parahydroxybenzoate (CAS No. 99-76-3); a butyl parahydroxybenzoate (CAS No. 94-26-8); a methylenebis(thiocyanate) (CAS No. 6317-18-6); a 1,2-benzisothiazoline-3-one (CAS No. 2634-33-5); a 2-mercaptobenzo-thiazole (CAS No. 149-30-4); a 5-chloro-2-methyl-3(2H)-isothiazolone (CAS No. 57373-19-0); a 2-methyl-3(2H)-isothiazolone (CAS No. 57373-20-3); a zinc 2-pyridinethiol-N-oxide (CAS No. 13463-41-7); a tetra-hydro-3,5-di-methyl-2H-1,3,5-thiadiazine-2-thione (CAS No. 533-74-4); a N-trichloromethyl-thio-4-cyclohexene-1,2-dicarboximide (CAS No. 133-06-2); a 2-n-octyl-4-isothiazoline-3-one (CAS No. 26530-20-1); a 2,4,5,6-tetrachloro-isophthalonitrile (CAS No. 1897-45-6); a 3-iodo-2-propynyl butylcarbamate (CAS No. 55406-53-6); a diiodomethyl-p-tolylsulfone (CAS No. 20018-09-1); a N-(trichloromethyl-thio)phthalimide (CAS No. 133-07-3); a potassium N-hydroxy-methyl-N-methyl-dithiocarbamate (CAS No. 51026-28-9); a sodium 2-pyridinethiol-1-oxide (CAS No. 15922-78-8); a 2-(thiocyanomethylthio) benzothiazole (CAS No. 21564-17-0); a 2-4(-thiazolyl) benzimidazole (CAS No. 148-79-8); or a combination thereof [see, for example, V. M. King, “Bactericides, Fungicides, and Algicides,” Ch. 29, pp. 261-267; and D. L. Campbell, “Biological Deterioration of Paint Films,” Ch. 54, pp. 654-661; both in PAINT AND COATING TESTING MANUAL, 14^{th} ed. of the Gardner-Sward Handbook, J. V. Koleske, Editor (1995), American Society for Testing and Materials, Ann Arbor, Mich.]. Additional biological products that may possess antifungal activity are described in the background discussion of U.S. Pat. Nos. 6,020,312; 5,602,097; and 5,885,782. U.S. Pat. No. 5,882,731 (Owens) describes a number of common and proprietary chemical mildewcide-comprising products that have been investigated as additives for water-based latex mixtures.

In certain embodiments an environmental law or regulation may encourage the selection of an organic biocide such as a benzisothiazolinone derivative. An example of a benzisothiazolinone derivative comprises a Busan™ 1264 (Buckman Laboratories, Inc.), a Proxel™ GXL (BIT), a Proxel™ TN (BIT/Triazine), a Proxel™ XL2 (BIT), a Proxel™ BD20 (BIT) and a Proxel™ BZ (BIT/ZPT) (Avecia Inc.), a Preventol® VP OC 3068 (Bayer Corporation), and/or a Mergal® K1ON (Troy Corp.) which comprises a 1,2-benzisothiazoline-3-one (CAS No. 2634-33-5). In the case of a Busan™ 1264, the primary use may be function as a bactericide and/or a fungicide at about 0.03% to about 0.5% in a water-borne coating, though a Busan™ may be used as a wood and/or a packaging preservative (e.g., a biocide, a mold inhibitor, a bactericide). A Proxel™ TN comprises a 1,2-benzisothiazoline-3-one (CAS No. 2634-33-5) and a hexahydro-1,3,5-tris(2-hydroxyethyl)-s-triazine (“triazine”; CAS No. 4719-04-4), a Proxel™ GXL, a Proxel™ XL2 and a Proxel™ BD20 comprises a 1,2-benzisothiazoline-3-one (CAS No. 2634-33-5), a Proxel™ BZ comprises a 1,2-benzisothiazoline-3-one (CAS No. 2634-33-5) and a zinc pyrithione (CAS No. 13463-41-7), and are typically used in an industrial coating and/or a water-based coating as a bactericide and/or a fungicide. A Mergal® K10N comprises a 1,2-benzisothiazoline-3-one (CAS No. 2634-33-5), and may be used in a water-borne coating as a bactericide and/or a fungicide.

Often, a preservative comprises a proprietary commercial formulation and/or a compound sold under a tradename. Examples include an organic biocide under the tradename Nuosept® (International Specialty Products, “ISP”), which are typically used in a water-borne coating, often as an antimicrobial agent. Specific examples of a Nuosept® biocide include a Nuosept® 95, which comprises a mixture of bicyclic oxazolidines, and may be added to about 0.2% to about 0.3% concentration to a coating; a Nuosept® 145, which comprises an amine reaction product, and may be added to about 0.2% to about 0.3% concentration to a coating; a Nuosept® 166, which comprises a 4,4-dimethyloxazolidine (CAS No. 51200-87-4), and may be added to about 0.2% to about 0.3% concentration to a basic pH water-borne coating; or a combination thereof. A further example comprises a Nuocide® (International Specialty Products) biocide(s), which are typically used fungicide(s) and/or algaecide(s). Examples of a Nuocide® biocide comprises Nuocide® 960, which comprises about 96% tetrachloroisophthalonitrile (CAS No. 1897-45-6), and may be used at about 0.5% to about 1.2% in a water-borne and/or a solvent-borne coating as a fungicide; a Nuocide® 2010, which comprises a chlorothalonil (CAS No. 1897-45-6) and an IPBC (CAS No. 55406-53-6) at about 30%, and may be used at about 0.5% to about 2.5% in a coating as a fungicide and/or an algacide; a Nuocide® 1051 and a Nuocide® 1071, each which comprises about 96% N-cyclopropyl-N-(1-dimethylethyl)-6-(methylthio)-1,3,5-triazine-2,4-diamine (CAS No. 28159-98-0), and may be used as an algacide in an anti-fouling coating at about 1.0% to about 6.0% or a water-based coating at about 0.05% to about 0.2%, respectively; and a Nuocide® 2002, which comprises a chlorothalonil (CAS No. 1897-45-6) and a triazine compound at about 30%, and may be used at about 0.5% to about 2.5% in a coating and/or a film as a fungicide and/or an algacide; or a combination thereof.

An additional example of a tradename biocide for a coating includes a Vancide® (R. T. Vanderbilt Company, Inc.). Examples of a Vancide® biocide include a Vancide® TH, which comprises a hexahydro-1,3,5-triethyl-s-triazine (CAS No. 108-74-7), and may be used in a water-borne coating; a Vancide® 89, which comprises a N-trichloromethylthio-4-cyclohexene-1,2-dicarboximide (CAS No. 133-06-2) and related compounds such as a captan (CAS No. 133-06-2), and may be used as a fungicide in a coating; or a combination thereof. A bactericide and/or a fungicide for a coating, particularly a water-borne coating, comprises a Dowicil™ (Dow Chemical Company). Examples of a Dowicil™ biocide include a Dowicil™ QK-20, which comprises a 2,2-dibromo-3-nitrilopropionamide (CAS No. 10222-01-2), and may be used as a bactericide at about 100 ppm to about 2000 ppm in a coating; a Dowicil™ 75, which comprises a 1-β-chloroallyl)-3,5,7-triaza-1-azoniaadamantane chloride (CAS No. 51229-78-8), and may be used as a bactericide at about 500 ppm to about 1500 ppm in a coating; a Dowicil™ 96, which comprises a 7-ethyl bicyclooxazolidine (CAS No. 7747-35-5), and may be used as a bactericide at about 1000 ppm to about 2500 ppm in a coating; a Bioban™ CS-1135, which comprises a 4,4-dimethyloxazolidine (CAS No. 51200-87-4), and may be used as a bactericide at about 100 ppm to about 500 ppm in a coating, or a combination thereof the forgoing. An additional example of a tradename preservative (e.g., a biocide) for a coating includes a Kathon® (Rohm and Haas Company). An example of a Kathon® biocide includes a Kathon® LX, which typically comprises a 5-chloro-2-methyl-4-isothiazolin-3-one (CAS no 26172-55-4) and a 2-methyl-4-isothiazolin-3-one (CAS no 2682-20-4) at about 1.5%, and may be added from about 0.05% to about 0.15% in a coating. Examples of tradename fungicide and/or an algacide include those described for a Fungustrol® (International Specialty Products), which typically may be used as fungicide(s), and a Biotrend® (International Specialty Products), which often is used as biocide(s); and are often formulated for a solvent-borne and/or a water-borne coating, an in-can and/or a film preservation. An example comprises a Fungustrol® 158, which comprises about 15% tributyltin benzoate (CAS No. 4342-36-3) and about 21.2% alkylamine hydrochlorides, and may be used at about 0.35% to about 0.75% in a water-borne coating for in-can and/or a film preservation. An additional example comprises a Fungustrol® 11, which comprises a N-(trichloromethylthio) phthalimide (CAS No. 133-07-3), and may be used at about 0.5% to about 1.0% as a fungicide for solvent-borne coating. A further example comprises a Fungustrol® 400, which comprises about 98% a 3-iodo-2-propynl N-butyl carbamate (“IPBC”) (Cas No. 55406-53-6), and may be used at about 0.15% to about 0.45% as a fungicide for a water-borne and/or a solvent-borne coating.

In additional facets, the preservative comprises 1-β-chloroallyl)-3,5,7-triaza-1-azoniaadamantane chloride; 1,2-benzisothiazoline-3-one; 1,2-dibromo-2,4-dicyanobutane; 1,3-bis(hydroxymethyl)-5,5-dimethylhydantoin; 1-methyl -3,5,7-triaza-1-azonia-adamantane chloride; 2-bromo-2-nitropropane-1, 3-diol; 2-(4-thiazolyl)benzimidazol e; 2-(hydroxymethyl)-amino-2-methyl-1-propanol; 2(hydroxymethyl)-aminoethanol; 2,2-dibromo-3-nitrilopropionamide; 2,4,5,6-tetrachloro-isophthalonitrile; 2-mercaptobenzo-thiazole; 2-methyl-4-isothiazolin-3-one; 2-n-octyl-4-isothiazoline-3-one; 3-iodo-2-propynl N-butyl carbamate; 4,5-dichloro-2-N-octyl-3 (2H)-isothiazolone; 4,4-dimethyloxazolidine; 5-chloro-2-methyl -4-isothiazolin-3-one; 5-hydroxy-methyl-1-aza-3,7-dioxabicylco (3.3.0.) octane; 6-acetoxy-2,4-dimethyl-1,3-dioxane; 7-ethyl bicyclooxazolidine; a combination of 1,2-benzisothiazoline-3-one and hexahydro-1,3,5-tris(2-hydroxyethyl)-s-triazine; a combination of 1,2-benzisothiazoline-3-one and zinc pyrithione; a combination of 2-(thiocyanomethyl-thio)benzothiozole and methylene bis(thiocyanate); a combination of 4-(2-nitrobutyl)-morpholine and 4,4′-(2-ethylnitrotrimethylene) dimorpholine; a combination of 4,4-dimethyl-oxazolidine and 3,4,4-trimethyloxazolidine; a combination of 5-chloro-2-methyl -4-isothiazolin-3-one and 2-methyl -4-isothiazolin-3-one; a combination of carbendazim and 3-iodo-2-propynl N-butyl carbamate; a combination of carbendazim, 3-iodo-2-propynl N-butyl carbamate and diuron; a combination of chlorothalonil and 3-iodo-2-propynl N-butyl carbamate; a combination of chlorothalonil and a triazine compound; a combination of tributyltin benzoate and alkylamine hydrochlorides; a combination of zinc-dimethyldithiocarbamate and zinc 2-mercaptobenzothiazole; a copper soap; a metal soap; a mercury soap; a mixture of bicyclic oxazolidines; a tin soap; an alkylamine hydrochloride; an amine reaction product; barium metaborate; butyl parahydroxybenzoate; carbendazim; copper(II) 8-quinolinolate; diiodomethyl-p-tolysulfone; dithio-2,2-bis(benzmethylamide); diuron; ethyl parahydroxybenzoate; glutaraldehyde; hexahydro-1,3,5-triethyl-s-triazine; hexahydro-1,3,5-tri s(2-hydroxyethyl)-s-triazine; hydroxymethyl-5,5-dimethylhydantoin; methyl parahydroxybenzoate; N-butyl-1,2-benzisothiazolin-3-one; N-(trichloromethylthio) phthalimide; N-cyclopropyl-N-(1-dimethylethyl)-6-(methylthio)-1,3,5-triazine-2,4-diamine; N-trichloromethyl-thio-4-cyclohexene-1,2-dicarboximide; p-chloro-m-cresol; phenoxyethanol; phenylmercuric acetate; poly(hexamethylene biguanide) hydrochloride; potassium dimethyldithiocarbamate; potassium N-hydroxy-methyl -N-methyl -dithiocarbamate; propyl parahydroxybenzoate; sodium 2-pyridinethiol-1-oxide; tetra-hydro-3,5-di-methyl-2H-1,3,5-thiadiazine-2-thione; tributyltin benzoate; tributyltin oxide; tributyltin salicylate; zinc pyrithione; sodium pyrithione; copper pyrithione; zinc oxide; a zinc soap; or a combination thereof.

Further examples of a tradename preservative (e.g., a biocide) for a coating includes various Omadine® and/or Triadine® product(s) (Arch chemicals, Inc.), a Densil™ P, Densil™ C404 (e.g., a chlorthalonil), a Densil™ DN (BUBIT), a Densil™ DG20 and a Vantocil™ D3 (Avecia Inc.), a Polyphase® 678, a Polyphase® 663, a Polyphase® CST, a Polyphase® 641, a Troysan® 680 (Troy Corp.), a Rocima® 550 (i.e., a preservative), a Rocima® 607 (i.e., a preservative), a Rozone® 2000 (i.e., a dry film fungicide), and a Skane™ M-8 (i.e., a dry film fungicide; Rohm and Haas Company) and a Myacide™ GDA, a Myacide™ GA 15, a Myacide™ Ga 26, a Myacide™ 45, a Myacide™ AS Technical, a Myacide™ AS 2, a Myacide™ AS 30, a Myacide™ AS 15, a Protectol™ PE, a Daomet™ Technical and/or a Myacide™ HT Technical (BASF Corp.). A zinc Omadine® (“zinc pyrithione”; CAS No. 13463-41-7) may function as a fungicide and/or an algacide typically used as an in-film preservative and/or an anti-fouling preservative; a sodium Omadine® (“sodium pyrithione”; CAS No. 3811-73-2) may be used as a fungicide and/or an algacide in-film preservative; a copper Omadine® (“copper pyrithione”; CAS No. 14915-37-8) may be used as a fungicide and/or an algacide in-film preservative and/or an anti-fouling preservative; a Triadine® 174 (“triazine,” “1,3,5-triazine-(2H,4H,6H)-triethanol”; “hexahydro-1,3,5-tris(2-hydroxyethyl)-s-triazine”; Cas No. 4719-04-4) may function as a bacteria biostatic and/or a bactericide typically used in a water-borne coating; an omacide IPBC (“Iodopropynyl-butyl carbomate”) may function as a fungicide; a Densil™ P comprises a dithio-2,2-bis(benzmethylamide) (CAS No. 2527-58-4) and may be used in an industrial coating, a water-based coating and/or a film as a fungicide and/or a bactericide; a Densil™ C404 comprises a 2,4,5,6-tetrachloroisophthalonitrile (“chlorothalonil”; CAS No. 1897-45-6) and may be used as a fungicide; a Densil™ DN and a Densil™ DG20 comprise a N-butyl-1,2-benzisothiazolin-3-one (CAS No. 4299-07-4), and each may be used as a fungicide; a Vantocil™ D3 comprises a poly(hexamethylene biguanide) hydrochloride (“PHMB”; CAS No. 27083-27-8) and may function as a microbiocide; a Polyphase® 678 comprises carbendazim (CAS No. 10605-21-7) and a 3-iodo-2-propynyl butyl carbamate (CAS No. 55406-53-6), and may be used as an antimicrobial biocide for an exterior coating and/or a surface treatment; a Polyphase® 663 comprises a 3-iodo-2-propynyl butyl carbamate (CAS No. 55406-53-6), a carbendazim (CAS No. 10605-21-7) and a diuron (CAS No. 330-54-1) and may be used as a fungicide and/or an algacide in an exterior coating; a Rocima® 550 comprises a 2-methyl-4-isothiazolin-3-one (CAS No. 2682-20-4), and may be used as a bactericide and/or a fungicide for a water-borne coating; a Rozone® 2000 comprises a 4,5-dichloro-2-N-octyl-3(2H)-isothiazolone (CAS No. 64359-81-5) and may be used as a microbiocide for a latex coating; a Skane™ M-8 comprises a 2-Octyl-4-isothiazolin-3-one (CAS No. 26530-20-1), and may be used as an in-film fungicide; a Myacide™ GDA Technical (50% Glutaraldehyde), a Myacide™ GA 15, a Myacide™ Ga 26 and a Myacide™ 45 each comprise a glutaraldehyde solution (CAS No. 111-30-8), and are typically used as an algacide, a bactericide, and/or a fungicide; a Myacide™ AS Technical (Bronopol, solid), a Myacide™ AS 2, Myacide™ AS 30, a Myacide™ AS 15 each comprise a 2-bromo-2-nitropropane-1,3-diol solution (“bronopol”; Cas No. 52-51-7) and are typically used as an algacide; a Protectol™ PE comprises a phenoxyethanol liquid (CAS No. 122-99-6) and may be used as a microbiocide and/or a fungicide; a Dazomet™ Technical comprises a 3,5-dimethyl-2H-1,3,5-thiadiazinane-2-thione solid (“dazomet”; CAS No. 533-74-4) and may be used as a microbiocide and/or a fungicide; a Myacide™ HT Technical comprises a 1,3,5-tris-(2-hydroxyethyl)-1,3,5-hexahydrotriazine liquid (“Triazine,” CAS No. 4719-04-4) and may be used as a microbiocide and/or a fungicide. Additional examples of tradename preservatives (all from Cognis Corp., Ambler, Pa.) includes a Nopcocide® N400, which comprises a Cholorthalonil-40% solution; a Nopcocide® N-98, which comprises a Chlorothalonil-100%; a Nopcocide® β-20, which comprises an IPBC-20% solution; a Nopcocide® β-40, which comprises an IPBC-40% solution; a Nopcocide® β-100, which comprises an IPBC-100% active; or a combination thereof.

An anti-microbial agent typically comprises a biocide (e.g., a fungicide, a bactericide, a herbicide a mildewcide, an algacide, a viricide, a germicide, a microbiocide, a slimicide) and/or a biostatic (e.g., a fungusstatic, a bacteristatic, a mildewstatic, an algastatic, a viristatic, a herbistatic, a germistatic, a microbiostatic, a slimistatic) to inhibit the growth of an organism such as a bacteria, a fungus, a mildew, an alga, a virus, a microorganism, or a combination thereof, on and/or within a material formulation such as a polymeric material (e.g., a plastic). An anti-microbial agent within a polymeric material typically diffuses and/or travels to the surface of the polymeric material during normal service life to provide a more continuous activity at the surface in reducing microbial grow. Often an anti-microbial agent comprises a carrier such as a liquid component (e.g., a solvent, a plasticizer), a resin, or a combination thereof. Specific examples of a carrier typically used as an anti-microbial agent carrier includes plasticizer (e.g., a diisodecyl phthalate, an epoxidized soybean oil), an oil, or a combination thereof. Examples of an anti-microbial agent commonly used in a polymeric material includes 2-n-octy-4-ixothiazonin-3-1; 10,10-oxybisphenoxarsine (“OBPA”); zinc 2-pyrodinethanol-1-oxide (“zinc-omadine”), trichlorophenoloxyphenol (“trislosan”), or a combination thereof, though a preservative used in a coating as well as an anti-microbial peptide are contemplated for use as an anti-microbial agent in a polymeric material, and such an anti-microbial agent may be used either alone or in combination with another anti-microbial agent in any composition, article, method, machine, etc. described herein in light of the present disclosures. An antimicrobial agent generally comprises about 0.000001% to about 1% of a polymeric material, and about 2% to about 10% of and anti-microbial agent and a carrier mixture, respectively, though given the inclusion of a biomolecular composition as part of a polymeric material and other compositions described herein, the content of an antimicrobial agent may be increased from about 0.000001% to about 10% or more. An antimicrobial agent often acts as a deodorant by reducing the growth of odor producing microorganism, particularly in a fiber (e.g., a textile) and/or a polymeric film application for packaging of food and/or trash.

In some aspects, an anti-biological biomolecular composition and/or a material formulation comprising an anti-biological biomolecular composition may be combined with an anti-biological technique and/or agent such as a wash material, or treated with such an anti-biological technique and/or wash material. For example, in some aspects, an anti-biological agent and/or an anti-biological technique comprises a detergent (e.g., a nonionic detergent, a zwitterionic detergent, an ionic detergent), such as CHAPS (zwitterionic), a Triton X series detergent (nonionic), and/or a SDS (ionic); a basic protein such as a protamine; a cationic polysaccharide such as chitosan; a metal ion chelator such as EDTA; or a combination thereof, all of which have may have effectiveness against a lipid cellular membrane, and may be incorporated into a material formulation and/or used in a washing composition (e.g., a washing solution, a washing suspension, a washing emulsion) applied to a material formulation. For example, a material formulation comprising an anti-biological peptide (e.g., a metal binding peptide with an anti-fouling activity) and an anti-biological enzyme may be washed with a commercial washing solution that may also comprise an anti-biological proteinaceous molecule. In another example, an anti-fouling agent may also include a material and/or a technique such as desiccation, disinfection (e.g., heat disinfection), and/or cleaning (e.g., contact with a cleaning agent, scrubbing, wiping, etc), etc used to reduce (e.g., remove, attenuate, sterilize) fouling. Such an anti-fouling agent may be applied while a surface is in contact with water (e.g., under the waterline) or dry (e.g., while in dry-dock). Often biofouling may be reduced (e.g., a slime reduced, a biofouling organism reduced) by sanitizing, which refers to cleaning and disinfection. An example of a chemical cleaning agent used to reduce fouling includes an oxidant (e.g., ozone, hydrogen peroxide, chlorine, peracetic acid), a surfactant (e.g., a tenside), an alkaline, an enzyme, a biodispersant (e.g., a polyethylene glycol), or a combination thereof. An example of a disinfectant used to reduce biofouling (e.g., reduce a biofouling organism, reduce a slime) include a chloramine (e.g., a monochloramine); chlorine; a potassium permanganate; a chlorine dioxide; ozone; ultra violet light; ionization, which in this context refers to ionizing a metal such as copper and/or silver with an electric current in a liquid (e.g., an aqueous fluid); an alkali (e.g., a pH increase); an acid (e.g., a pH decrease); or a combination thereof. An example of a mechanical cleaning agent includes sonication, brushing, rinsing (e.g., rinsing with a fluid, air, etc), or a combination thereof [Industrial Biofouling Detection, Prevention and Control (Walker, J., Surman, S., Jass, J. Eds.) John Wiley & Sons, LTD West Sussex UK, 2000]. Of course, these anti-fouling materials and techniques may be adapted for use in other material formulations such as a terrestrial material formulation.

Of course, an anti-biological agent (e.g., an anti-biological agent, an anti-fouling agent, an enzyme, a peptide, a preservative) may be combined with another biomolecular composition (e.g., an enzyme, a cell based particulate material), for the purpose to confer an additional property (e.g., a catalytic activity, a binding property) other than one related to anti-biological and/or anti-fouling function. Examples of another biomolecular composition include an enzyme such as a lipolytic enzyme, though some lipolytic enzymes may have anti-biological and/or anti-fouling activity; a phosphoric triester hydrolase; a sulfuric ester hydrolase; a peptidase, some of which may have an anti-biological and/or anti-fouling activity; a peroxidase, or a combination thereof. Alternatively, in several embodiments, a biomolecular composition may be used with little or no anti-biological and/or anti-fouling function. For example, a material formation may comprise a combination of active enzymes with little or no active anti-fouling, and/or anti-biological enzyme present.

15. Detection of a Biomolecular Composition's Activity

In general embodiments, a material formulation comprising a biomolecular composition comprises a desired biomolecule (e.g., a metal binding proteinaceous molecule, an enzyme, a peptide), whether endogenously or recombinantly produced, that may alter and/or confer a desired property to the material formulation (e.g., a polymeric material, a surface treatment, a filler). Such a property conferred to a material formulation by incorporation of an active biomolecule may be determined using a standard procedure for material formulation described herein or in the art, in light of the present disclosures.

For example, a property such as metal binding activity, anti-biological activity, anti-fouling activity, and/or enzymatic activity of a proteinaceous molecule may be detected using various techniques, and numerous (e.g., millions or more) sequences may be screened at once using techniques of the art. For example, a random and/or a non-random peptide library may be expressed using a bacterial phage, and then screened for such a property. In a particular example, thousands of random and/or non-random peptide sequences may be expressed as part of a bacteriophage protein that comprises part of a virion's proteinaceous coat that may contact and bind another molecule (e.g., an antibody, a ligand, a metal ion) (Scott, J. K. and Smith, G. P., Science 249:386-390, 1990). In another example, a metal ion (e.g. a Zn, a Cd) may be immobilized to a chelating Sepharose gel and phages expressing a peptide library contacted with the gel. The non-binding phages may be eluted away to identify peptide(s) that bind a metal ion (Mejáre, M. et al. 1998). In a further example, a radioactive isotope of a metal ion may be contacted with a proteinaceous molecule and excess isotope washed away, followed by detection (e.g., autoradiography) of the bound isotope to identify a proteinaceous molecule with a metal binding property (Regan, L. and Clarke, N. D., Biochemistry, 29:10878-10883, 1990).

The various assays described herein and/or in the art in light of the present disclosure, may be used to determine the biomolecule's activity (e.g., a metal binding property, an anti-biological property) as part of a material formulation (e.g., a coating, a film, etc.). For example, an assay may be used to determine the anti-fouling activity of a proteinaceous molecule (e.g., a metal binding proteinaceous molecule), such as measuring the anti-fouling property of a material formulation comprising the proteinaceous molecule. Such an assay may be used to measure a change (e.g., a reduction) in biofouling due to the presence of a proteinaceous molecule by comparing biofouling (e.g., a fouling coat comprising organic molecules produce by a fouling organism and/or a fouling living cell) between a material formulation comprising a proteinaceous molecule to a like material formulation comprising a different (e.g., reduced) amount of the proteinaceous molecule. For example, biofouling may be detected by standard techniques such as cell (e.g., microorganism) culturing from a sample of a material formulation; counting cells; using a stain (e.g., an acridine orange; 4′,6-diamidino-2-phenylindole; 5-cyano-2,3-ditolyl tetrazolium chloride) to promote visualization of a cell; cell detection with an antibody; cell detection (e.g., PCR, in situ hybridization) with a molecular probe (e.g., a nucleic acid, PCR, etc.); microscopy (e.g., light microscopy, electron microscopy); or a combination thereof [Industrial Biofouling Detection, Prevention and Control (Walker, J., Surman, S., Jass, J. Eds.) John Wiley & Sons, LTD West Sussex UK, 2000].

In an additional example of an assay for an anti-biological activity, a poor and/or a low microorganism/biological resistance rating for a coating may be denoted as a colony recovery/growth rating of 2 to 4, a discoloration/disfigurement rating of 0 to 5, a fouling resistance (“F.R.”) or anti-fouling film (“A.F”) rating of 0 to 70, and observed growth (e.g., fungal growth) on specimens of 2 to 4, respectively, as described in “ASTM Book of Standards, Volume 06.01, Paint—Tests for Chemical, Physical, and Optical Properties; Appearance,” D3274-95, D2574-00, D3273-00, D5589-97 and D5590-00, 2002; and in “ASTM Book of Standards, Volume 06.02, Paint—Products and Applications; Protective Coatings; Pipeline Coatings,” D3623-78a, 2002. An additional example of a standard microorganism/biological resistance assay may be described in “ASTM Book of Standards, Volume 06.01, Paint—Tests for Chemical, Physical, and Optical Properties; Appearance,” D4610-98 and D3456-86, 2002; in “ASTM Book of Standards, Volume 06.02, Paint—Products and Applications; Protective Coatings; Pipeline Coatings,” D4938-89, D4939-89, D5108-90, D5479-94, D6442-99, D6632-01, D4940-98 and D5618-94, 2002; and “ASTM Book of Standards, Volume 06.03, Paint—Pigments, Drying Oils, Polymers, Resins, Naval Stores, Cellulosic Esters, and Ink Vehicles,” D912-81 and D964-65, 2002.

In a further example, determination of whether damage to a coating and/or a film may be due to a microorganism (e.g., a film algal defacement, a film fungal defacement), as well as the efficacy of addition of an anti-biological agent (e.g., an anti-biological proteinaceous molecule, a preservative, an antimicrobial agent) to a coating and/or a film composition in reducing microbial damage to a coating and/or a film, may be empirically determined [see, for example, Flick, E. W. “Handbook of Paint Raw Materials, Second Edition,” 263-285 and 879-998, 1989; in “Paint and Coating Testing Manual, Fourteenth Edition of the Gardner-Sward Handbook,” (Koleske, J. V. Ed.), pp 261-267 and 654-661, 1995; in “Paint and Surface Coatings, Theory and Practice, Second Edition,” (Lambourne, R. and Strivens, T. A., Eds.), pp. 193-194, 371-382 and 543-547, 1999; Wicks, Jr., Z. W., Jones, F. N., Pappas, S. P. “Organic Coatings, Science and Technology, Volume 1: Film Formation, Components, and Appearance,” pp. 318-320, 1992; Wicks, Jr., Z. W., Jones, F. N., Pappas, S. P. “Organic Coatings, Science and Technology, Volume 2: Applications, Properties and Performance,” pp. 145, 309, 319-323 and 340-341, 1992; in “Paints, Coatings and Solvents, Second, Completely Revised Edition,” (Stoye, D. and Freitag, W., Eds.) pp 6, 127 and 165, 1998; In “Waterborne Coatings and Additives,” 202-216, 1995; in “Handbook of Coatings Additives,” pp. 177-224, 1987; and in “PCI Paints & Coatings Industry,” pp. 56, 58, 60, 62, 64, 66-68, 70, 72 and 74, July 2003]. In conducting such tests, microorganisms such as, for example, Gram-negative Eubacteria including Alcaligenes faecalis (ATCC No. 8750), Pseudomonas aeruginosa (ATCC Nos. 10145 and 15442), Pseudomonas fluorescens (ATCC No. 13525), Enterobacter aerogenes (ATCC No. 13048), Escherichia coli (ATCC No. 11229), Proteus vulgaris (ATCC No. 8427), Oscillatoria sp. (ATCC No. 29135), and Calothrix sp. (ATCC No. 27914); Gram-positive Eubacteria including Bacillus subtilis (ATCC No. 27328), Brevibacterium ammoniagenes (ATCC No. 6871), and Staphylococcus aureus (ATCC No. 6538); filamentous fungi including Aspergillus oryzae (ATCC No. 10196), Aspergillus flavus (ATCC No. 9643), Aspergillus niger (ATCC Nos. 9642 and 6275), Aureobasidium pullulans (ATCC No. 9348), Penicillium sp. (ATCC No. 12667), Penicillium citrinum (ATCC No. 9849), Penicillium funiculosum (ATCC No. 9644), Cladosporium cladosporoides (ATCC No. 16022), Trichoderma viride (ATCC No. 9645), Ulocladium atrum (ATCC No. 52426), Alternaria alternate (ATCC No. 52170), and Stachybotrys chartarum (ATCC No. 16026); yeast including Candida albicans (ATCC No. 11651); and Protista including Chlorella sp. (ATCC No. 7516), Chlorella vulgaris (ATCC No. 11468), Chlorella pyrenoidosa (UTEX No. 1230), Chlorococcum oleofaciens (UTEX No. 105), Ulothrix acuminata (UTEX No. 739), Ulothrix gigas (ATCC No. 30443), Scenedesmus quadricauda (ATCC No. 11460), Trentepohlia aurea (UTEX No. 429), and Trentepohlia odorata (CCAP No. 483/4); have been used as positive control contaminants of a coating.

Additional methods for assaying and/or selecting an antibiological agent (e.g., an antibiotic composition) for a material formulation are described in U.S. Pat. Nos. 6,020,312; 5,885,782; and 5,602,097, and patent application Ser. Nos. 10/884,355 and 11/368,086, such as, for example, contacting a material formulation (e.g., a coating) comprising an anti-biological proteinaceous molecule (e.g., a peptide) with a biological entity, and measuring growth over time relative to a like material formulation comprising less or no selected anti-biological proteinaceous molecule content. For example, a fungal cell may be used in assaying and/or screening for an antifungal composition (e.g., a peptide library), may comprise a fungal organism known to, or suspected of, infesting a vulnerable material(s) and/or surface(s) (e.g., a construction material). Such methods may be used to assay and/or screen, for example, antifungal activity against a wide variety of fungus genera and species, such as in the case of selecting a composition comprising a broad-spectrum antifungal activity. Similar methods may be used to identify, for example, a particular proteinaceous composition(s) (e.g., a peptide, a plurality of peptides) that target specific fungus genera or species. Examples of such a fungal cell often used in such an assay include members of the genera Stachybotrys (e.g., Stachybotrys chartarum), Aspergillus species (sp.), Penicillium sp., Fusarium sp., Alternaria dianthicola, Aureobasidium pullulans (aka Pullularia pullulans), Phoma pigmentivora and Cladosporium sp, though an assay may be adapted for other cell(s). An example of an assay for evaluating an antifungal agent is described by the American Society for Testing and Materials (ASTM) in D-5590-94. In another example, a proteinaceous molecule may be effective (e.g., inhibit growth, treat infestation, etc.) against a biological entity from a genera and/or a species of, for example, an Alternaria (e.g., an Alternaria dianthicola), an Aspergillus [(e.g., an Aspergillus species (sp.), an Aspergillus fumigatus, an Aspergillus Parasiticus], an Aureobasidium (e.g., an Aureobasidium pullulans a.k.a. a Pullularia pullulans), a Candida; a Ceratocystis (e.g., a Ceratocystis Fagacearum), a Cladosporium (e.g., a Cladosporium sp.), a Fusarium (e.g., a Fusarium sp., a Fusarium oxysporum, a Fusariam Sambucinum), a Magaporthe (e.g., a Magaporthe Aspergillus nidulans), a Mycosphaerella, a Penicillium (e.g., a Penicillium sp.), a Phoma (e.g., a Phoma pigmentivora), a Pphiostoma (e.g., a Pphiostoma ulmi), a Pythium (e.g., a Pythium ultimum, a Rhizoctonia (e.g., Rhizoctonia Solani), a Stachybotrys (e.g., a Stachybotrys chartarum), or a combination thereof. Cell and/or viral culture conditions may be modified appropriately to provide favorable growth and proliferation conditions, using the techniques of the art, and to assay and/or screen for activity against a target cell (e.g., a bacterium, an alga, etc.) and/or a virus. Any suitable peptide/polypeptide/protein screening method in the art may be used to identify an anti-biological proteinaceous molecule for an assay as active anti-biological agent in a material formulation (e.g., a paint, a coating material, a biomolecular composition). For example, an in vitro method to determine bioactivity of a peptide, such as a peptide from a synthetic peptide combinational library, may be used (Furka, A., et al., 1991; Houghten, R. A., et al., 1991; Houghten, R. A., et al., 1992).

In a further example, use of a preservative in a coating, is known in the art, and all such materials and techniques for using a preservative in a coating may be applied in various embodiments(see, for example, Flick, E. W. “Handbook of Paint Raw Materials, Second Edition,” 263-285 and 879-998, 1989; in “Paint and Coating Testing Manual, Fourteenth Edition of the Gardner-Sward Handbook,” (Koleske, J. V. Ed.), pp 261-267 and 654-661, 1995; in “Paint and Surface Coatings, Theory and Practice, Second Edition,” (Lambourne, R. and Strivens, T. A., Eds.), pp. 193-194, 371-382 and 543-547, 1999; Wicks, Jr., Z. W., Jones, F. N., Pappas, S. P. “Organic Coatings, Science and Technology, Volume 1: Film Formation, Components, and Appearance,” pp. 318-320, 1992; Wicks, Jr., Z. W., Jones, F. N., Pappas, S. P. “Organic Coatings, Science and Technology, Volume 2: Applications, Properties and Performance,” pp. 145, 309, 319-323 and 340-341, 1992; in “Paints, Coatings and Solvents, Second, Completely Revised Edition,” (Stoye, D. and Freitag, W., Eds.) pp 6, 127 and 165, 1998; and in “Handbook of Coatings Additives”, pp. 177-224, 1987). Such an assay may be used to determine infestation of a coating by a microorganism (e.g., a bacteria, a fungus, an alga), and the activity of an anti-biological composition in reducing such infestation. Examples of a bacteria commonly found to contaminate a terrestrial coating and/or a film include a Pseudomonas spp., an Aerobacter spp., an Enterobacter spp., a Flavobacterium spp. (e.g., a Flavobacterium marinum), a Bacillus spp., or a combination thereof. Examples of a fungus commonly found to contaminate a terrestrial coating and/or a film include an Aureobasidium pullulans, an Alternaria dianthicola, a Phoma pigmentivora, or a combination thereof. Examples of an alga commonly found to contaminate a terrestrial coating and/or a film include an Oscillotoria sp., a Scytonema sp., a Protoccoccus sp., or a combination thereof. Techniques for determining microbial contamination of a coating and/or a coating component have been described (see, for example, “ASTM Book of Standards, Volume 06.01, Paint—Tests for Chemical, Physical, and Optical Properties; Appearance,” D5588-97, 2002).

In another example, any assay described herein or in the art in light of the present disclosures may be used to determine the bioactivity resistance wherein an enzyme retains detectable enzymatic activity. Additionally, in certain aspects, it is contemplated that a material formulation comprising an biomolecule such as an enzyme may lose part or all of a detectable, desirable bioactivity during the period of time of contact with standard assay condition, but regain part or all of the enzymatic bioactivity after return to non-assay conditions. An example of this process is the thermal denaturation of an enzyme at an elevated temperature range into a configuration with lowered or absent bioactivity, followed by refolding of an enzyme, upon return to a more suitable temperature range for the enzyme, into a configuration possessing part or all of the enzymatic bioactivity detectable prior to contact with the elevated temperature. In another example, an enzyme may demonstrate such an increase in bioactivity upon removal of a solvent, a chemical, etc.

In some embodiments, an enzyme identified as having a desirable enzymatic property for one or more target substrates may be selected for incorporation into a material formulation. The determination of an enzymatic property may be conducted using any technique described herein or in the art, in light of the present disclosures. For example, the determination of the rate of cleavage of a substrate, with or without a competitive or non-competitive enzyme inhibitor, can be utilized in determining the enzymatic properties of an enzyme, such as Vₘₐₓ, Kₘ, K_{cat}/Kₘ and the like, using analytical techniques such as Lineweaver-Burke analysis, Bronsted plots, etc Brockerhoff, Hans and Jensen, Robert G. “Lipolytic Enzymes”, pp 10-24, 1974; Dumas, D. P. et al., 1989a; Dumas, D. P. et al., 1989b; Dumas, D. P. et al., 1990; Caldwell, S. R. and Raushel, F. M., 1991c; Donarski, W. J. et al., 1989; Raveh, L. et al., 1992; Shim, H. et al., 1998; Watkins, L. M. et al., 1997a; diSioudi, B. et al., 1999; Hill, C. M., 2000; Hartleib, J. and Ruterjans, H., 2001b; Lineweaver, H. and Burke, D., 1934; Segel, I. H., 1975). Such analysis may be used to identify an enzyme with a specifically enzymatic property for one or more substrates, given that use of an assay for an enzyme's activity may be incorporated with identification of a proteinaceous molecule as having enzymatic activity.

For example, lipolytic enzymes and phosphoric triester hydrolases have demonstrated the ability to degrade a wide variety of lipids and OP compounds, respectively. Methods for measuring the ability of an enzyme to degrade a lipid or an OP compound are described herein as well as in the art. Any such technique may be utilized to determine enzymatic activity of a composition for a particular lipid or an OP compound. For example, techniques for measuring the enzymatic degradation for various lipids comprising an ester and/or other hydrolysable moiety, including a triglyceride such as a triolein, an olive oil, and/or a tributyrin; a chromogenic substrate such as 4-methylumbelliferone, and/or a 4-methylumbelliferone; and/or a radioactively labeled glycerol ester substrate, such as a glycerol [³H]oleic acid esters; may be used (see, for example, Brockerhoff, Hans and Jensen, Robert G. “Lipolytic Enzymes.” pp-25-34, 1974). To measure a lipolytic enzyme's activity against a substrate, a molecular monolayer of a lipid substrate may be used to control variables such as pressure, charge potential, density, interfacial characteristics, enzyme binding, and/or the effects of an inhibitor, in measuring lipolytic enzyme kinetics [see for example, Gargouri, Y. et al., 1989; Melo, E. P. et al., 1995; In “Methods and Molecular Biology, Volume 109 Lipase and Phospholipase Protocols.” (Mark Doolittle and Karen Reue, Eds.), pp 279-302, 1999].

Techniques for measuring the kinetics of enzymatic degradation for various OP-compounds in the art may be used. In one example, the cleavage rate of a phosphonothiolate OP substrate comprising a β-S bond can be measured using a method known as the Ellman reaction. Such substrates may produce a β-S bond cleavage product comprising a free thiol group, which can chemically react with the Ellman's reagent, 5,5′-dithio-bis-2-nitrobenzoic acid (“DTNB”). This reaction produces a 5′-thiol-2-nitrobenzoate anion with a maximum absorbency at 412 nm. β-S cleavage can be determined by the appearance of the free thiol group, measured using a spectrophotometer (Rastogi, V. H. et al., 1997; Gopal, S. et al., 2000; diSioudi, B. et al., 1999; Watkins, L. M. et al., 1997a; Hoskin, F. C. G. et al., 1995; Chae, M. Y. et al., 1994; Ellman, G. L. et al., 1961). In an additional example, the cleavage of an OP substrate can be measured by detecting the production of a cleavage product comprising a released ion. In a further example, the cleavage of a phosphonofluoridate can be measured by the release of cleavage product comprising a fluoride ion (F⁻) using a fluoride ion specific electrode and a pH/mV meter (Hartleib, J. and Ruterjans, H., 2001a; Gopal, S. et al., 2000; diSioudi, B. et al., 1999; Watkins, L. M. et al., 1997a; DeFrank, J. and Cheng, T., 1991; Dumas, D. P. et al., 1990; Dumas, D. P. et al., 1989a). In another example, the cleavage of a phosphonocyanate can be measured by the release of a cleavage product comprising a cyanide ion (CN⁻) using a cyanide selective electrode with a pH meter (Raveh, L. et al., 1992). The cleavage of DEPP can be measured at 280 nm, using a spectrophotometer to detect a phenol cleavage product (Watkins, L. M. et al., 1997a; Hong, S.-B. and Raushel, F. M., 1996). In a further example, various phosphodiesters (e.g., an ethyl-4-nitrophenyl phosphate) have been made to evaluate OPH cleavage rates, and their cleavage measured at 280 nm by the production of a substituted phenol cleavage product (Shim, H. et al., 1998). In a further example, a paraoxon is often used to measure OPH activity, because it is both rapidly hydrolyzed by the enzyme and produces a visible cleavage product. To determine kinetic properties, the production of paraoxon's cleavage product, p-nitrophenol, may be measured with a spectrophotometer at 400 nm and/or 420 nm (Dumas, D. P. et al., 1990; Kuo, J. M. and Raushel, F. M., 1994; Watkins, L. M. et al., 1997a; Gopal, S. et al., 2000). In an additional example, a NPPMP cleavage can also be measured by the release of a p-nitrophenol as a cleavage product (diSioudi, B. et al., 1999).

16. Multipacks/Kits

For a purpose such as ease of production, a material formulation (e.g., an antifungal paint, a coating product comprising an antifungal proteinaceous molecule) may be provided to a consumer as a single premixed formulation. In some embodiments, the components of a material formulation may be stored separately prior to combining for use. For example, for a purpose such as to optimize the initial activity (e.g., the activity of a biomolecular composition component) and/or extend the useful lifetime of the material formulation and/or the activity of a biomolecular composition, a biomolecular composition may be packaged separately from the material formulation (e.g., a paint, a coating) into which the biomolecular composition (e.g., an anti-fouling proteinaceous molecule) may be added/incorporated. Thus, in certain embodiments, one or more components (e.g., a biomolecular composition, a polymer, an additive such as a substrate, a ligand, a protective material, etc.), of a material formulation may be stored separately (e.g., a kit of components) prior to combining. The components may be stored in two or more containers (“pot”) (e.g., about 2 to about 20 containers) in a multipack kit. In certain embodiments, a material formulation (e.g., a coating comprising a biomolecular composition) comprises a multi-pack material formulation, such as a two-pack material formulation (“two-pack kit”), a three-pack material formulation, four-pack material formulation, five-pack material formulation, or more wherein the material formulation components are stored in separate containers. Separate storage may reduce, for example, microorganism growth in a component (e.g., a coating component), damage to the biomolecular composition by a component (e.g., a coating component), increase the storage life (“pot life”) of material formulation (e.g., a coating), reduce the amount of a component such as a preservative in a material formulation (e.g., a coating), allow separate and/or sequential incorporation of a component into a material formulation (e.g., addition of a component post-cure, addition of a component during service life), or a combination thereof. In certain aspects, about 0.000001% to about 100%, including all intermediate ranges and combinations thereof, of one component of a material formulation (e.g., a biomolecular composition, an antifungal composition) may be stored in a separate container from another component of a material formulation. For example, a material formulation may be in the form of a precursor material (e.g., a thermosetting resin that cures into solid plastic) in a container, and a container comprising a biomolecular composition and/or a ligand for the biomaterial composition, to be combined (e.g., admixed, etc.) with the precursor material for use (e.g., application of a surface treatment to a surface).

### SPECIFIC EXAMPLES

The general effectiveness of various embodiments is demonstrated in the following Examples. Some methods for preparing compositions are illustrated. Starting materials are made according to procedures known in the art or as illustrated herein. The following Examples are provided so that the embodiments might be more fully understood. These Examples are illustrative only and should not be construed as limiting in any way, as other material formulations such as a polymeric material, a surface treatment (e.g., a different paint formulation), and/or a filler, comprising different biomolecular compositions (e.g., a different purified or partly purified enzyme, a different cell-based particulate material comprising an enzyme, a peptide, a polypeptide) may be prepared.

Example 1—This Example describes a screening method for identifying an anti-biological activity of a peptide library.

The screening method for a coating generally includes: creating a synthetic peptide combinatorial library using known methods and materials; testing a battery of biological entities that are known to, or suspected of, infesting a material formulation (e.g., a building material, an object) having a biological-infestation susceptible surface with aliquots of the synthetic peptide library, wherein each aliquot comprises an equimolar mixture of peptides in which at least one of the terminal amino acid residues (e.g., C-terminal residues) are defined and which residues are in common for each peptide in the mixture; admixing said aliquots with a coating typically used on such building material and coating a surface with the admixture; allowing an appropriate period of time for growth of the biological entity under suitable culture conditions; comparing the growth of the treated biological entity(s) with untreated control biological entity(s); identifying which of the aliquots demonstrate an anti-biological activity [e.g., reduced the growth of the biological entity(s)]; and, optionally, assessing the relative growth inhibitory activity of each aliquot compared to that of other aliquots (e.g., comparing IC₅₀ data). Of course, such a screening method may be adapted for other material formulations than a coating, may be used assay for anti-biological activity in (e.g., on the surface of, within) a material formulation, and may be used to assay other proteinaceous molecules than those of a peptide library.

In the above-referenced U.S. Pat. Nos. 6,020,312; 5,885,782; and 5,602,097 an iterative process was used to identify active peptide sequences with broad spectrum anti-biological (e.g., antifungal) activity. A representative method employs a hexapeptide library with the first two amino acids in each peptide chain individually and specifically defined and with the last four amino acids consisting of equimolar mixtures of 20 amino acids. Four hundred (400) (20²) different peptide mixtures each comprising 130,321 (19⁴)(cysteine was eliminated) individual hexamers were evaluated. In such a peptide mixture, the final concentration for each peptide was 9.38 ng/ml, in a mixture comprised of 1.5 mg (peptide mix)/ml solution. This mixture profile assumed that an average peptide has a molecular weight of 785. This concentration was sufficient to permit testing for anti-biological activity. Both D- and L-amino acid comprising peptides may be constructed and tested to identify peptide compositions that can inhibit or kill biological entity(s) that can grow on the surfaces of inanimate objects. Peptide compositions comprising substantially homogeneous peptide compositions, as well as mixtures of peptides derived from amino acids that are between 3 to 25 residues in length (a length readily accomplished using standard peptide synthesis procedures), especially six residues in length, are disclosed in U.S. Pat. Nos. 6,020,312; 5,885,782; and 5,602,097. An example of an anti-biological peptide that inhibits or kills one or more fungus that infests and grows on the surfaces of inanimate objects comprises a hexapeptide having the amino acid sequence Phe Arg Leu Lys Phe His (SEQ ID No. 41).

Another example of a method for selecting antibiotic compositions includes first creating a synthetic peptide combinatorial library as described herein. Next, as further described in detail herein, a step of contacting a battery of fungal cells with aliquots of the synthetic peptide combinatorial library, each of which aliquots represents an equimolar mixture of peptides in which at least the two C-terminal amino acid residues are defined and which residues are in common for each peptide in said mixture may be accomplished. After allowing an appropriate period for growth, a next step may be accomplished in which the growth of the battery of fungal cells as compared to untreated control cells may be measured. Lastly, a determination may be made of which of the aliquots reduces (e.g., reduces to the relatively greatest extent) the growth of fungal cells in a coating overall in the battery of fungal cells. Of course, the same method may be carried out in which each of the aliquots represents an equimolar mixture of peptides in which at least three, four, five or more C-terminal amino acid residues are defined (depending upon the overall length of the ultimate peptide in the SPCL). Typically, such increasingly defined aliquots may be sequentially tested to select the succeeding candidate peptides for testing. Thus, an additional step in the method entails utilizing the determination of which of the aliquots reduces the growth of fungal cells in a coating overall in said battery of fungal cells to select which aliquots to next test of a synthetic peptide combinatorial library where at least one additional C-terminal amino acid residue may be defined.

Example 2—This Example describes methods of identifying an anti-biological biomolecular composition (e.g., a proteinaceous molecule).

The testing methods described in U.S. Pat. Nos. 6,020,312; 5,885,782; and 5,602,097 may be employed to screen one or more peptidic agent(s) (e.g., a peptide library) for anti-biological activity against a wide variety of genera and species. The methods may be modified to screen against organisms that are known to, or suspected of, infesting, for example, construction materials or other vulnerable materials and surfaces. In some embodiments, examples of cells used for screening the peptide library include members of the fungal genera Stachybotrys (especially Stachybotrys chartarum), Aspergillus species (sp.), Penicillium sp., Fusarium sp., Alternaria dianthicola, Aureobasidium pullulans (aka Pullularia pullulans), Phoma pigmentivora and Cladosporium sp. Cell culture conditions may also be modified appropriately to provide favorable growth and proliferation conditions, using techniques of the art. The above-mentioned methods may be used to identify one or more peptidic agent(s) [e.g., a peptide(s), group(s) of peptide(s)] demonstrating broad-spectrum anti-biological activity. Similar methods may be used to identify one or more peptidic agent(s) [e.g., a peptide(s), group(s) of peptide(s)] that target specific genera or species. For example, certain of the peptides of particular usefulness in the coatings, as disclosed in U.S. Pat. Nos. 6,020,312; 5,602,097; and 8,885,782, exhibit variable abilities to inhibit fungal growth as adjudged by the minimal inhibitory concentrations (MIC mg/ml) and/or the concentrations to inhibit growth of fifty percent of a population of fungal spores (IC₅₀ mg/ml). MICs may range depending upon peptide additive and target organism from about 3 to about 300 mg/ml, while IC₅₀'s may range depending upon peptide additive and target organisms from about 2 to about 100 mg/ml. Target organisms susceptible to these amounts include Fusarium oxysporum, Fusariam Sambucinum, Rhizoctonia Solani, Ceratocystis Fagacearum, Pphiostoma ulmi, Pythium ultimum, Magaporthe Aspergillus nidulans, Aspergillus fumigatus, and Aspergillus Parasiticus. Alternatively, any other suitable peptide/polypeptide/protein screening method could be used instead to identify anti-biological peptide candidates for testing as active anti-biological agents in a material formulation (e.g., a paint, a coating).

The mode of action of anti-biological peptides, polypeptides and/or proteins, by which they exert their anti-biological effect(s) (e.g., inhibitory effect, bioicidal activity), can be varied. For example, certain peptides may operate to destabilize fungal cell membranes, while the modes of action of others could include disruptions of macromolecular synthesis or metabolism. While the modes of action of some antifungal peptides have been determined (see, e.g., Fiedler, H. P., et al. 1982. J Chem. Technol. Biotechnol. 32:271-280; Isono, K. and S. Suzuki. 1979. Heterocycles 13:333-351), mechanisms which explain their modes of action and specificity have typically not yet been determined. Initial studies to elucidate antifungal mode of action of peptides involves a physical examination of mycelia and cells to determine if the peptides can perturb membrane functions responsible for osmotic balance, as has been observed for other peptides (Zasloff, M. 1987. Proc. Natl. Acad. Sci. USA 84:5449-5453). Disruption of appressorium formation may also be the mechanism by which some peptides inhibit fungal growth (see e.g., published U.S. patent application Ser. No. 10/601,207, expressly incorporated herein by reference in its entirety). For the purposes of preparing and using anti-biological peptides, polypeptides and/or proteins as active anti-biological agents in a material formulation (e.g., a paint, a coating), the mechanism by which the anti-biological effect may be exerted on a biological entity (e.g., one or more cells) may, in some embodiments, may not be understood.

Example 3—This Example describes coating formulations comprising a metal binding and/or an anti-biological biomolecular composition.

A paint or coating composition may comprise an anti-biological biomolecular composition (i.e., one or more peptides, polypeptides or proteins) such as, for example, as described herein (e.g., a metal binding proteinaceous composition, an anti-fouling proteinaceous composition). For example, an anti-biological proteinaceous molecule may be used as a partial or complete substitute (“replacement”) for another biocide and/or biostatic typically used in a biological entity prone composition. It is contemplated that about 0.0001% to about 100%, including all intermediate ranges and combinations thereof, of a conventional anti-biological agent component in a coating formulation may be substituted by an anti-biological biomolecular composition. For example, the concentration of anti-biological proteinaceous molecule may exceed 100%, by weight or volume, of the non-proteinaceous anti-biological component (e.g., a bioicide, a bioistatic) being replaced. In another example a conventional non-proteinaceous anti-biological component may be replaced with an anti-biological proteinaceous molecule equivalent to 0.001% to 500% (by weight, or by volume), including all intermediate ranges and combinations thereof, of the substituted anti-biological component. For example, to produce a coating with similar biological resistance properties as a non-substituted formulation, it may require that 20% (e.g., 0.2 kg) of a chemical bioicide may be replaced by 10% (e.g., 0.1 kg) of an anti-biological proteinaceous molecule. In another exemplary formulation, to produce a coating with similar biological resistance as a non-substituted formulation, it may require replacing 70% of a chemical bioicide (e.g., 0.7 kg) with the equivalent of 127% (e.g., 1.27 kg) of anti-biological proteinaceous molecule. The various assays described herein, or as would be known in the art in light of the present disclosure, may be used to determine the biological resistance properties of a material formulation (e.g., a coating, a film) produced by direct addition of an anti-biological biomolecular composition and/or substitution of some or all of a non-biomolecular or chemical anti-biological component by an anti-biological biomolecular composition. Such additives may be directly admixed with the coating, applied as a primer coating, applied as an overcoat, or any combination of these application techniques.

It is contemplated that any previously described formulation of a coating composition may be modified to incorporate a biomolecular composition. Examples of described coating compositions include over 200 industrial water-borne coating formulations (e.g., air dry coatings, air dry or force air dry coatings, anti-skid of non-slip coatings, bake dry coatings, clear coatings, coil coatings, concrete coatings, dipping enamels, lacquers, primers, protective coatings, spray enamels, traffic and airfield coatings) described in “Industrial water-based paint formulations,” 1988, over 550 architectural water-borne coating formulations (e.g., exterior paints, exterior enamels, exterior coatings, interior paints, interior enamels, interior coatings, exterior/interior paints, exterior/interior enamels, exterior/interior primers, exterior/interior stains), described in “Water-based trade paint formulations,” 1988, the over 400 solvent borne coating formulations (e.g., exterior paints, exterior enamels, exterior coatings, exterior sealers, exterior fillers, exterior primers, interior paints, interior enamels, interior coatings, interior primers, exterior/interior paints, exterior/interior enamels, exterior/interior coatings, exterior/interior varnishes) described in “Solvent-based paint formulations,” 1977; and the over 1500 prepaint specialties and/or surface tolerant coatings (e.g., fillers, sealers, rust preventives, galvanizers, caulks, grouts, glazes, phosphatizers, corrosion inhibitors, neutralizers, graffiti removers, floor surfacers) described in Prepaint Specialties and Surface Tolerant Coatings, by Ernest W. Flick, Noyes Publications, 1991.

From these representative formulations, it will be readily appreciated that a wide variety of coating compositions (e.g., paints) may be improved by addition of a biomolecular composition. Some of these include industrial water-borne coating formulations (e.g., air dry coatings, air dry or force air dry coatings, anti-skid of non-slip coatings, bake dry coatings, clear coatings, coil coatings, concrete coatings, dipping enamels, lacquers, primers, protective coatings, spray enamels, traffic and airfield coatings); architectural water-borne coating formulations (e.g., exterior paints, exterior enamels, exterior coatings, interior paints, interior enamels, interior coatings, exterior/interior paints, exterior/interior enamels, exterior/interior primers, and exterior/interior stains); solvent borne coating formulations (e.g., exterior paints, exterior enamels, exterior coatings, exterior sealers, exterior fillers, exterior primers, interior paints, interior enamels, interior coatings, interior primers, exterior/interior paints, exterior/interior enamels, exterior/interior coatings, and exterior/interior varnishes); and prepaint specialties and/or surface tolerant coatings (e.g., fillers, sealers, rust preventives, galvanizers, caulks, grouts, glazes, phosphatizers, corrosion inhibitors, neutralizers, graffiti removers and floor surfacers).

For example, an anti-biological and/or metal binding coating (e.g, a paint) comprising a biomolecular composition may then be tested and used as described elsewhere herein, or the product may be employed for any other suitable purpose as would be recognized in the art in light of this disclosure. For instance, the physical properties (e.g., purity, density, solubility, volume solids and/or specific gravity, rheology, viscometry, and particle size) of the resulting anti-biological liquid paint or other coating product, can be assessed using standard techniques that are known in the art and/or as described in PAINT AND COATING TESTING MANUAL, 14^{th} ed. of the Gardner-Sward Handbook, J. V. Koleske, Editor (1995), American Society for Testing and Materials (ASTM), Ann Arbor, Mich., and applicable published ASTM test methods. Alternatively, any other suitable testing method of the art in light of the present disclosures may be employed for assessing physical properties of the coating mixture comprising an above-described biomolecular composition.

Example 4—This Example describes latex paints with an anti-biological peptidic agent.

Both the interior latex (Olympic Premium, flat, ultra white, 72001) and acrylic paints (Sherwin Williams DTM, primer/finish, white, B66W1; 136-1500) appeared to be toxic to both Fusarium and Aspergillus. Therefore, eight individual wells (48-well microtito plate) of each paint type were extracted on a daily basis with 1 ml of phosphate buffer for 5 days (1-4 & 6) and then allowed the plates were allowed to dry before running the assay. Each well comprised 16 ul of respective paint.

Extract testing: The extract from two wells each of the two paints for each day was evaluated for toxicity by mixing the extract 1:1 with 2× medium and inoculating with spores (10⁴) of Aspergillus or Fusarium. The extracts had no affect on growth of either test fungus.

Well testing: The extracted and non-extracted wells for each of the paints were tested with a range of inoculum levels in growth medium using the two different fungi. For Fusarium the range was 10¹-10⁴ and for Aspergillus 10²-10⁵. Well Testing of Acrylic Paint Plates: Both Fusarium and Aspergillus grew in all extracted wells at all inoculum levels. Only Aspergillus grew in non-extracted wells at the 10⁵ level and not at lower levels indicative of an inherent biocidal capability. Well Testing of Latex Paint Plates: Fusarium grew in the extracted wells only at the 10⁴ inoculum level but not at 10¹-10³. Aspergillus grew in all extracted wells showing an inoculum level effect. No growth was observed for either Fusarium or Aspergillus in non-extracted wells.

Conclusion: Extraction of the toxic factor(s) found in both paints was possible. However, it appeared that it may be less extractable from the latex paint.

Evaluation of peptidic agent activity in presence of acrylic and latex paints: It was established that it was possible to extract both acrylic and latex paints dried in a 48-well format to make them non-toxic to the test microorganisms—Fusarium and Aspergillus. Using that information an assay was designed to determine the effect the paint has on peptidic agent activity against two test organisms.

Assay design: Coat 48-well plastic plates with 16μ1 of acrylic or latex paint. Dry for two days under hood. Extract designated wells with 1-ml phosphate buffer changing the buffer on a daily basis for 7 days. Control wells were not extracted to confirm paint toxicity. Add 20μ1 of peptidic agent series in duplicate to designated dry paint coated wells. Peptide, SEQ ID No. 41, series were added in a two-fold dilution series to wells and allowed to dry. The concentration of peptide added ranged from 200 μg/20μ1 to 1.5 μg/20μ1. Inoculated paint-coated plates as follows: Extracted control wells received 180μ1 of medium+20μ1 of spore suspension (10⁴ spores/20 μl of medium). Inoculum was either Fusarium or Aspergillus in each case. Non-extracted control wells received 180 μl of medium+20 μl of spore suspension (10⁴ spores/20 μl of medium). Extract wells with dried peptide series received 180 μl of medium+20 μl of spore suspension (10⁴ spores/20 μl of medium). In duplicate. Extract wells that did not have dried peptide series received 160 μl of medium+20 μl of spore suspension (10⁴/20 μl of medium)+20 μl peptide series as above. In duplicate. Plates were observed for growth over a 5-day period.

Growth and peptide controls: Used sterile non-paint coated 48 well plastic plates

Growth control wells for each test fungus received 180 μl of medium+20 μl of spore suspension (10⁴ spores/20 μl of medium). Peptide activity controls received 160 μl of medium+20 μl of spore suspension (10⁴ spores/20 μl of medium)+20 μl peptide series as above. Peptide series were added in a two-fold dilution series to wells and range from 200 μg/20 μl to 1.5 μg/20μ1. Therefore, the range of peptide tested was 200 μg/200 μl or 1.0 μg/μ1 (1000 μg/ml) to 0.0075 μg/μ1 (7.5 μg/ml). Uninoculated medium served as blank for absorbance readings taken at 24, 48, 72, 96 and 120h.

Results: Unextracted wells comprising either latex or acrylic paint inhibited growth of both Fusarium and Aspergillus. Extracted wells comprising either latex or acrylic paint allowed growth of both Fusarium and Aspergillus. The calculated MIC for Fusarium in peptide activity control assays was 15.62 μg/ml. For Aspergillus the calculated MIC was 61.4 μg/ml. For extracted acrylic-coated plates the following results were obtained. Controls as stated in above. For Fusarium with dried peptide, inhibition was seen at 1000 and 500 μg/ml after 5 days. Spores exposed to liquid peptide added to dry paint wells were inhibited at 1000, 500 and 250 μg/ml after 4 days, and 1000 and 500 μg/ml after 5 days. For Aspergillus with dried peptide, inhibition was seen at 1000 μg/ml after 5 days. Spores exposed to liquid peptide added to dry paint wells were inhibited at 1000 and 500 μg/ml after 5 days.

For extracted latex-coated plates the following results were obtained. Controls as stated above.

For Fusarium with dried peptide, inhibition was seen at 1000 μg/ml after 5 days. Spores exposed to liquid peptide added to dry paint wells were inhibited at 1000 μg/ml after 5 days. For Aspergillus with dried peptide, inhibition was seen at 1000 μg/ml after 5 days. Spores exposed to liquid peptide added to dry paint wells were inhibited at 1000 μg/ml after 5 days.

Example 5—This Example describes the combined use of an anti-biological biomolecular composition and another anti-biological agent.

A material formulation (e.g, a paint composition) comprising one or more conventional anti-biological substance may be modified by addition of one or more of the anti-biological proteinaceous molecules(s) (e.g., an anti-fouling peptide) described herein. For example, the anti-biological proteinaceous molecule may comprise one or more “pure” anti-biological peptides of defined sequence, and/or it may include a peptide library aliquot comprising a mixture of peptides in which at least two (e.g., three, four) of the N-terminal amino acid residues are defined (as in SEQ ID Nos. 1-24). In another example, if the proteinaceous molecule comprises a mixture of metal binding peptides, at least one may have anti-biological activity.

Combining a non-proteinaceous anti-biological agent with one or more anti-biological proteinaceous molecule may provide anti-biological activity above that seen with either the proteinaceous molecule) or the non-proteinaceous molecule alone. The expected additive inhibitory activity of the combination may be calculated by summing the inhibition levels of each component alone. The combination may be assayed on the test organism to derive an observed additive inhibition. If the observed additive inhibition is greater than that of the expected additive inhibition, synergy is exhibited. More specifically, a synergistic combination of an anti-biological proteinaceous molecule (e.g., an aliquot of a peptide library comprising at least one anti-biological peptide), occurs when two or more anti-biological (e.g., cell growth-inhibitory) substances distinct from the proteinaceous molecule are observed to be more inhibitory to the growth of an evaluated organism than the sum of the inhibitory activities of the individual components alone.

An example of an assay method for determining additive or synergistic combinations comprises first creating a synthetic peptide combinatorial library. Each aliquot of the library represents an equimolar mixture of peptides in which at least the two C-terminal amino acid residues are defined. Using the testing methods described in one or more of U.S. Pat. Nos. 6,020,312, 5,885,782, and 5,602,097 it is possible to determine for each such aliquot of the synthetic peptide combinatorial library, a calculated concentration at which it may inhibit a target organism in a coating. Next, the aliquot of the synthetic peptide combinatorial library may be mixed with at least one non-peptide anti-biological compound to create a test mixture. As with the peptide component of the mixture, the baseline ability of the non-peptide anti-biological substance to inhibit the assay's organism may be determined initially. Next, the assay's organism may be contacted with the mixture being assayed, and the inhibition of growth of the organism may be measured as compared to at least one untreated control. More controls may be used, such as a control for each individual component of the mixture. Similarly, where there are more than two components being assayed, the number of controls to be used may be increased in a manner of the art of growth inhibition testing. From the separate assay results for the proteinaceous and non-proteinaceous molecules the expected additive anti-biological effect (e.g., inhibition of growth) may be determined using standard techniques. For example, after the growth inhibition assays are complete for the combination of proteinaceous and non-proteinaceous molecules, the actual or observed effect on the inhibition of growth may be determined. The expected additive effect and the observed effect are then compared to determine whether a synergistic inhibition of growth of the organism has occurred. The methods used to detect synergy may utilize non-peptide antimicrobial agents in combination with the inhibitory proteinaceous molecule described herein.

As described herein, an anti-biological proteinaceous molecule may be used in combination with one or more existing anti-biological agents (e.g., a biocide, a biostatic) identified herein or in the art. It is expected that some such combinations of the anti-biological proteinaceous molecule with another anti-biological agent may provide, for example, a broader range of activity against various organisms, a synergistic anti-biological (e.g, a preservative) effect, and/or a longer duration of effect.

Another example combination includes an anti-biological proteinaceous molecule and a preservative and/or antimicrobial agent that acts against non-fungal organisms (e.g., a bactericide, an algacide), as it is contemplated that many fungal prone material formulations and/or surfaces coated with a surface treatment are also susceptible to damage by a variety of organisms. Examples of these preservatives and antimicrobial agents are described herein.

Example 6—This Example describes the combined use of an anti-biological biomolecular composition and/or an OP degrading enzyme.

A multifunctional material formulation (e.g., surface treatment) may combine an anti-biological property with the ability to degrade an organophosphorus compound. An enzyme that functions to degrade an organophosphorus compound is contemplated as having an anti-biological (e.g., anti-fouling) activity. Examples of such a composition may be in the form of a coating, a paint, a non-film forming coating, an elastomer, an adhesive, an sealant, a material applied to a textile, a polymeric material, or a wax, and may be modified by addition of one or more anti-biological biomolecular composition(s) (e.g., a metal binding peptide having an anti-fouling activity) selected as described herein and an organophosphorus compound detoxifying agent such as an OP degrading enzyme or cellular material comprising such activity.

Example 7—This Example describes adhesives, sealants, and elastomers comprising a metal binding and/or an anti-biological biomolecular composition.

The metal binding and/or anti-biological biomolecular composition(s) (e.g., a metal binding peptide having an anti-fouling activity) described herein are expected to be additionally useful for coating or mixing into adhesive(s) and sealant(s) such as a grout and/or a caulk, such as those that are in frequent contact with, or constantly exposed to biological entity (e.g., fungal) nutrients and/or moisture. Examples of adhesives and sealants (e.g., caulks, acrylics, elastomers, phenolic resin, epoxy, polyurethane, anaerobic and structural acrylic, high-temperature polymers, water-based industrial type adhesives, water-based paper and packaging adhesives, water-based coatings, hot melt adhesives, hot melt coatings for paper and plastic, epoxy adhesives, plastisol compounds, construction adhesives, flocking adhesives, industrial adhesives, general purpose adhesives, pressure sensitive adhesives, sealants, mastics, urethanes) for various surfaces (e.g., metal, plastic, textile, paper), and techniques of preparation and assays for properties, have been described in Skeist, I., ed., Handbook of Adhesives, 3rd Ed., Van Nostrand Reinhold, New York, 1990; Satriana, M. J. Hot Melt Adhesives: Manufacture and Applications, Noyes Data Corporation, New Jersey, 1974; Petrie, E. M., Handbook of Adhesives and Sealants, McGraw-Hill, New York, 2000; Hartshorn, S. R., ed., Structural Adhesives-Chemistry and Technology. Plenum Press, New York, 1986; Flick, E. W., Adhesive and Sealant Compound Formulations, 2nd Ed., Noyes Publications, New Jersey, 1984; Flick, E., Handbook of Raw Adhesives 2nd Ed., Noyes Publications, New Jersey, 1989; Flick, E., Handbook of Raw Adhesives, Noyes Publications, New Jersey, 1982; Dunning, H. R., Pressure Sensitive Adhesives-Formulations and Technology, 2nd Ed., Noyes Data Corporation, New Jersey, 1977; and Flick, E. W., Construction and Structural Adhesives and Sealants, Noyes Publications, New Jersey, 1988. For example, an adhesive, sealant or elastomer composition comprising one or more conventional anti-biological substance(s) may be modified by addition of and/or substitution by one or more of the anti-biological proteinaceous molecule(s) described in herein. Examples of adhesive include a thermoplastic adhesive, a thermoset adhesive, an elastomeric adhesive, an alloy adhesive, a non-polymeric adhesive, or a combination thereof. Examples of an adhesive includes a cellulosic adhesive, a cyanoacrylate adhesive, a dextrin adhesive, an ethylene-vinyl acetate copolymer adhesive, a melamine formaldehyde adhesive, a natural rubber adhesive, a neoprene/phenolic adhesive, a neoprene rubber adhesive, a nitrile rubber adhesive, a nitrile/phenolic adhesive, a phenolic adhesive, a phenol/resorcinol formaldehyde adhesive, a phenoxy adhesive, a polyamide adhesive, a polybenzimidazole adhesive, a polyethylene adhesive, a polyester adhesive, a polyimide adhesive, a polyisobutylene adhesive, a polysulfide adhesive, a polyurethane adhesive, a polyvinyl acetal adhesive, a polyvinyl acetal/phenolic adhesive, a polyvinyl acetate adhesive, a polyvinyl alcohol adhesive, a reclaimed rubber adhesive, a resorcinol adhesive, a silicone adhesive, a styrenic TPE adhesive, a styrene butadiene adhesive, a vinyl phenolic adhesive, a vinyl vinylidene adhesive, an acrylic acid diester adhesive, an epoxy adhesive, an epoxy/phenolic adhesive, an epoxy/polysulfide adhesive, a urea formaldehyde adhesive, a urea formaldehyde/melamine formaldehyde adhesive, a urea formaldehyde/phenol resorcinol adhesive, or a combination thereof. Examples of a thermosetting adhesive comprise an acrylic adhesive, an acrylic acid diester adhesive, a cyanoacrylate adhesive, a cyanate ester adhesive, an epoxy adhesive, a melamine formaldehyde adhesive, a phenolic adhesive, a polybenzimidazole adhesive, a polyester adhesive, a polyimide adhesive, a polyurethane adhesive, a resorcinol adhesive, a urea formaldehyde adhesive, or a combination thereof. Examples of a thermoplastic adhesive comprise an acrylic adhesive, an ethylene-vinyl acetate copolymer adhesive, a carbohydrate adhesive (e.g., a dextrin adhesive, a starch adhesive), a cellulosic adhesive (e.g., a cellulose acetate adhesive, cellulose acetate butyrate adhesive, cellulose nitrate adhesive), a polyethylene adhesive, a phenoxy adhesive, a polyamide adhesive, a polyvinyl acetal adhesive, a polyvinyl acetate adhesive, a polyvinyl alcohol adhesive, a protein adhesive (e.g., an animal adhesive, a soybean adhesive, a blood adhesive, a fish adhesive, a casein adhesive), a vinyl vinylidene adhesive, or a combination thereof. Examples of an elastomeric adhesive comprise a butyl rubber adhesive, a natural rubber adhesive, a neoprene rubber adhesive, a nitrile rubber adhesive, a polyisobutylene adhesive, a polysulfide adhesive, a reclaimed rubber adhesive, a silicone adhesive, a styrenic TPE adhesive, a styrene butadiene adhesive, or a combination thereof. Examples of an alloy adhesive comprise an epoxy/polyamide adhesive, an epoxy/phenolic adhesive, an epoxy/polysulfide adhesive, a neoprene/phenolic adhesive, a nitrile/phenolic adhesive, a phenol/resorcinol formaldehyde adhesive, a polyvinyl acetal/phenolic adhesive, a vinyl/phenolic adhesive, a urea formaldehyde/phenol resorcinol adhesive, a urea formaldehyde/melamine formaldehyde adhesive, or a combination thereof. Examples of a non-polymeric adhesive include a mucilage adhesive.

It is contemplated that a biomolecular composition may also be incorporated into an elastomer. An elastomer may comprise a polymer that can undergo large, but reversible, deformations upon a relatively low physical stress. It is contemplated that an elastomer composition may incorporate a biomolecular composition, such as by preparation with the biomolecular composition and/or direct addition such as by a multi-pack composition. Elastomers (e.g., tire rubbers, polyurethane elastomers, polymers ending in an anionic diene, segmented polyerethane-urea copolymers, diene triblock polymers with styrene-alpha-methylstyrene copolymer end blocks, poly(p-methylstyrene-b-p-methylstyrene), polydimethylsiloxane-vinyl monomer block polymers, chemically modified natural rubber, polymers from hydrogenated polydienes, polyacrylic elastomers, polybutadienes, trans-polyisoprene, polyisobutene, cis-1,4-polybutadiene, polyolefin thermoplastic elastomers, block polymers, polyester thermoplastic elastomer, thermoplastic polyurethane elastomers) and techniques of elastomer synthesis and elastomer property analysis have been described, for example, in Walker, B. M., ed., Handbook of Thermoplastic Elastomers, Van Nostrand Reinhold Co., New York, 1979; Holden, G., ed., et. al., Thermoplastic Elastomers, 2n^{d} Ed., Hanser Publishers, Verlag, 1996. An example of an elastomer includes a thermoplastic elastomer, a melt processable rubber (“NPR”), a synthetic rubber (“SR”), a natural rubber (“NR”), a non-polymeric elastomer, or a combination thereof. In some embodiments, the elastomer comprises a thermoplastic elastomer, a melt processable rubber, a synthetic rubber, a natural rubber, a propylene oxide elastomer, an ethylene-isoprene elastomer, an ethylene-vinyl acetate elastomer, a non-polymeric elastomer, or a combination thereof. In other embodiments, the composition comprises an adhesive, a sealant, or a combination thereof.

Example 8—This Example describes a textile finish comprising a metal binding and/or an anti-biological biomolecular composition.

A metal binding and/or an anti-biological biomolecular composition (e.g., a metal binding peptide having an anti-fouling activity) may also be incorporated into a material applied to a textile, such as, for example, a textile finish. Textile finishes (e.g., soil-resistant finishes, stain-resistant finishes) and related materials for application to a textile are described, for example, in Johnson, K., ANTISTATIC COMPOSITIONS FOR TEXTILES AND PLASTICS, Noyes Data Corporation, New Jersey, 1976; Rouette, H. K., ENCYCLOPEDIA OF TEXTILE FINISHING, Springer, Verlag, 2001; TEXTILE FINISHING CHEMICALS: AN INDUSTRIAL GUIDE, by Ernest W. Flick, Noyes Publications, 1990; and HANDBOOK OF FIBER FINISH TECHNOLOGY, by Philip E. Slade, Marcel Dekker, 1998. One type of water repellent and/or oil repellent textile finish comprises Scotchguard™ (3M Corporate Headquarters, Maplewood, Minn., U.S.A.). For example, a textile finish comprising one or more conventional anti-biological substance(s) may be modified by addition of and/or substitution by one or more of the anti-biological proteinaceous molecule(s) described in herein.

Example 9—This Example describes a polymeric material comprising a metal binding and/or an anti-biological biomolecular composition.

It is contemplated that a biomolecular composition (e.g., an enzyme, a proteinaceous sequence, a metal binding peptide, an anti-biological peptide) may also be incorporated into a polymeric material such as a plastic (e.g., a thermoplastic, a thermoset). A polymeric material may comprise a plurality of polymers (“polymer blends”), an ionomer, a thermoplastic polymer, a thermoset polymer, or an elastomer. A thermoplastic comprises a thermoplastic polymer, while a thermoset plastic comprises a thermosetting polymer. A thermoplastic polymeric material may, for example, comprise a biodegradable polymer, a cellulosic polymer, a fluoropolymer, a polyether, a polyamide, a polyacrylonitrile, a polyamide-imide, a polyarylate, a polybenzimidazole, a polybutylene, a polycarbonate, a thermoplastic polyester, a polyetherimide, a polyethylene, a polyimide, a polyketone, an acrylic, a polymethylpentene, a polyphenylene oxide, a polyarylene sulphide, a polypropylene, a polyurethane, a polystyrene, a polysulfone resin, a polyterpene, a polyvinyl acetal, a polyvinyl acetate, a thermoplastic vinyl ester, a polyvinyl ether, a polyvinyl carbazole, a polyvinyl chloride, a polyvinylidene chloride, a polyimidazopyrrolone, a polyacrolein, a polyvinylpyridine, a polyvinylamide, a polyurea, a polyquinoxaline, or a combination thereof. A thermoplastic polymer may comprise an environmentally degradable polymers (e.g., a biodegradable polymer), a natural polymer, a photodegradable polymer, a synthetic biodegradable polymer (e.g., a poly(alkylene oxalate)s, a polyamino acid, a pseudo-polyamino acid, a polyanhydride, a polycaprolactone, a polycyanoacrylate, a polydioxanone, a polyglycolide, poly(hexamethylene-co-trans-1,4-cyclohexane dimethylene oxalate), a polyhydroxybutyrate, a polyhydroxyvalerate, a polylactide, a poly(ortho ester), a poly (p-dioxanone), a polyphosphazene, a poly(propylene fumarate), a polyvinyl alcohol), a biological degradable polymer (e.g., a collagen, a fibrinogen/fibrin, a gelatin, a polysaccharide), a cellulosic polymer (e.g., cellulose acetate, a cellulose acetate butyrate, a cellulose acetate propionate, a cellulose methylcellulose, a methylcellulose, a cellulosehydroxyethyl, an ethylcellulose, a hydroxypropylcellulose), a fluoropolymer, an ethylene chlorotrifluoroethylene, an ethylene tetrafluoroethylene, a fluoridated ethylene propylene, a polyvinylidene fluoride, a polychlorotrifluoroethylene, a polytetrafluoroethylene, a polyvinyl fluoride), a polyoxymethylene, a polyamide, an aromatic polyamide, a polyacrylonitrile, a polyamide-imide, a polyarylate, a polybenzimidazole, a polybutylene, a polycarbonate, a polyester (e.g., a liquid crystal polyester polycarbonate, a polybutylene terephthalate, a polycyclohexylenedimethylene terephthalate, a poly(ethylene terephthalate)), a polyetherimide, polyethylene (e.g., a very low-density polyethylene, a low-density polyethylene, a linear low-density polyethylene, a medium-density polyethylene, a high-density polyethylene, an ultrahigh molecular weight polyethylene, a chlorinated polyethylene, a chlorosulfonated polyethylene, a phosphorylated polyethylene, an ethylene-acrylic acid copolymer, an ethylene-methyl acrylate copolymer, an ethylene-ethyl acrylate copolymer, an ethylene-n-butyl acrylate copolymer, an ethylene-vinyl acetate copolymer, an ethylene-vinyl alcohol copolymer), a polyimide, a polyketone, a poly(methylmethacrylate), a polymethylpentene, a polyphenylene oxide, a polyphenol sulfide, a polyphthalamide, a polypropylene, a polyurethane, a polystyrene (e.g., styrene-acrylonitrile copolymer, a styrene-butadiene copolymer, an acrylonitrile butadiene styrene terpolymer, an acrylonitrile-chlorinated polyethylene-styrene terpolymer, an acrylic styrene acrylonitrile terpolymer), a polysulfone resin (e.g., a polysulfone, a polyaryl sulfone, a polyether sulfone), a polyvinyl chloride (e.g., a chlorinated polyvinyl chloride), a polyvinylidene chloride, or a combination thereof. A thermoset polymeric material may comprise, for example, an alkyd resin, an allyl resin, an amino resin, a bismaleimide resin, a cyanate ester resin, an epoxy resin, a furane resin, a phenolic resin, a thermosetting polyester resin, a polyimide resin, a polyurethane resin, a silicone resin, a vinyl ester resin, a casein, or a combination thereof. Polymeric materials often comprise an additive, such as a filler, a plasticizer, a lubricant, a flame retarder, a colorant, a blowing agent, an anti-aging additive, a cross-linking agent, etc. or a combination thereof. Polymeric materials and methods of preparation of preparing a polymeric material and assays for a polymeric material's properties have been described, for example, “Handbook of Plastics, Elastomers, & Composites Fourth Edition” (Harper, C. A. Ed.) McGraw-Hill Companies, Inc, New York, 2002; and Tadmor, Z. and Costas, G. G. “Principles of Polymer Processing Second Edition,” John Wiley & Sons, Inc. Hoboken, N.J., 2006.

Example 10—This Example demonstrates the ability of a lysozyme to survive the incorporation process into a coating, demonstrates lysozyme hydrolytic activity in a coating environment, and demonstrates the ability of lysozyme to survive in can conditions for 48 hours. A Sherwin-Williams Acrylic Latex paint was used.

Materials, reagents and equipment used are shown in the tables below.

TABLE 4
Materials and Reagents
0.1M potassium phosphate buffer, pH 6.4
Micrococcus lysodeikticus (Worthington Biochemicals, #8736)
Sherwin-Williams Acrylic Latex paint
Lysozyme (chicken egg white) (Sigma Product #L 6876,
CAS 12650-88-3)
15 mL plastic test tubes

TABLE 5
Equipment
Paint spreader (1-8 mil)
Polypropylene blocks
Lightnin Labmaster Mixer
Rotator shaker
Pipettes and Pipetteman
Klett-Sumerson Colorimeter (Filter D35: 540 nm)

The reagents prepared included a Micrococcus cell suspension comprising 9 mg M. lysodeikticus in 25 mL sodium phosphate buffer, and a lysozyme solution comprising a 5 mg/mL stock solution. The paint formulations used are shown in the table below.

TABLE 6
Paint Preparation
Sherwin-Williams Acrylic Latex Control (no additive)
Sherwin-Williams Acrylic Latex with 1 mg/mL lysozyme

The paint was mixed with a glass stirring rod and a paint mixer. Each film was immediately drawn onto polypropylene surfaces with a thickness of 8 mil. Cure time for the Sherwin-Williams was 72 hrs. To demonstrate in can durability, the Sherwin-Williams Acrylic Latex comprising lysozyme wet paint was sealed and shelf stored at ambient temperature. After 48 hrs in can, films were drawn onto polypropylene surfaces with a thickness of 8 mils and were allowed to cure 72 hrs prior to assay. Coupons were generated as free films from the polypropylene surface. Films were generated in three sizes: 2 cm²: 1 cm by 2 cm; 4 cm²: 1 cm by 4 cm; or 6 cm²: 1 cm by 6 cm.

For qualitative assessment, individual films were placed into labeled 15 mL tubes. Films of each size (2, 4 and 6 cm²) were evaluated in triplicate. In addition to a control paint with no additive, two other controls were utilized, a positive control and a negative control. The positive control comprised: lysozyme in buffer added to each of three 15 mL tubes in concentrations approximating the amount of lysozyme in the films (i.e., 40 μg, 80 μg, and 120 μg). Each amount was assayed in triplicate. The negative control comprised: 5 mL of 0.36 mg/mL M. lysodeikticus cell suspension pipetted into a single 15 mL tube. 5 mL 0.36 mg/mL Micrococcus lysodeikticus cell suspension was added to all reaction tubes to begin the reaction. The tubes were placed on a rocker at ambient conditions for approximately 22 hours. Where possible, the films were removed from the suspension and determine opacity using the Klett-Summerson Colorimeter (turbidity unit: Klett Unit or KU).

Particulate matter in the samples interfered with quantitation; photographs of each set of 2 cm² paint films and controls following 22 hour contact to M. lysodeikticus cell suspension were taken, and observations recorded in the Tables below.

TABLE 7
Qualitative Observations (visual assessments)

| | Lysozyme | Film Size | |
|---|---|---|---|
| Sample¹ | (μg) | (cm²) | Clarity |
| Suspension/Solution | | | |
| Controls | | | |
| M. lysodeikticus | — | — | Translucent |
| Lysozyme | 40 | — | Transparent² |
| | 80 | — | Transparent |
| | 120 | — | Transparent |
| Control Films | | | |
| S-W | | 2, 4, 6 | Translucent |
| Films Comprising | | | |
| Lysozyme | | | |
| S-W | | 2, 4, 6 | Transparent |

¹Each evaluation was performed in triplicate
²Thinned in opacity, with some suspended particulate matter

The strips comprising lysozyme of all three sizes of coupons cleared the M. lysodeikticus suspension, indicating that the lysozyme maintains activity in the coating environment. Cleared suspensions (lysozyme comprising coupons and controls) comprised large particles which interfere with the quantitation of the cleared suspensions. The particulate matter was less detectable in the 2 cm² set comprising lysozyme, so this size coupon was used for the quantitative demonstrations.

TABLE 8
Quantiative Assessment of Lysozyme In-Film Activity (2 cm² film, 4
hr time point, 3 independent assays, each performed in triplicate.)

| | | Replicate 1 | | Replicate 2 | | Replicate 3 | |
|---|---|---|---|---|---|---|---|
| | In can | | Cell | | Cell | | Cell |
| Formulation | (hrs) | KU | lysis | KU | lysis | KU | lysis |
| Suspension Controls | | | | | | | |
| M. lysodeikticus | | 81.5 | 0.0% | 101 | 0% | | |
| Lysozyme | | 17 | | 27 | | | |
| S-W Acrylic Latex | | | | | | | |
| Control Films | | | | | | | |
| | — | 75 | 18% | 74 | 19% | 71 | 22% |
| | — | 79 | 13% | 82 | 10% | 76 | 17% |
| | — | 83 | 9% | 81 | 11% | 73 | 20% |
| Films Comprising | | | | | | | |
| Lysozyme | | | | | | | |
| | — | 8 | 91% | 20 | 78% | 11 | 88% |
| | — | 13 | 86% | 11 | 88% | 15 | 84% |
| | — | 13 | 86% | 5 | 95% | 0 | 100% |
| Control Films | 48 hrs | 82 | 10% | 65 | 29% | 68 | 25% |
| Films Comprising | 48 hrs | 36 | 61% | 26 | 72% | 37 | 59% |
| Lysozyme | | | | | | | |

KU = Klett Units, measure of turbidity at 540 nm.

A lysozyme in Sherwin-Williams Acrylic Latex was able to lyse about 88% of the M. lysodeikticus culture over 4 hours, relative to the control which exhibited about a 15% drop in opacity. After in-can shelving for 48 hrs (i.e., the lysozyme was mixed into the Sherwin-Williams Acrylic Latex, capped and shelved for 48 hrs prior to drawing down the films), the lysozyme remained active, lysing about 64% of the M. lysodeikticus culture relative to the about 21% lysis exhibited by the control panels.

Example 11—This Example demonstrates the retention of lysozyme vs. loss due to leaching in a paint film in a saturated condition at 1, 2 and 24 hours after submersion.

Materials, reagents and equipment used are shown in the Tables below.

TABLE 9
Materials and Reagents
0.1M potassium phosphate buffer, pH 6.4
Micrococcus lysodeikticus (Worthington Biochemicals, #8736)
Lysozyme (chicken egg white) (Sigma Product #L 6876, CAS 12650-88-3)
Sherwin-Williams Acrylic Latex paint
15 mL plastic test tubes

TABLE 10
Equipment
Paint spreader (1-8 mil)
Polypropylene blocks
Lightnin Labmaster Mixer
Rotator shaker
Pipetter and tips
Klett-Sumerson Colorimeter (Filter D35: 540 nm)

The reagents prepared included a Micrococcus cell suspension comprising 9 mg M. lysodeikticus in 25 mL sodium phosphate buffer, and a lysozyme solution comprising a 5 mg/mL stock solution.

The paint formulations that were prepared included a Sherwin-Williams Acrylic Latex Control (no additive), and a Sherwin-Williams Acrylic Latex comprising 1 mg/mL lysozyme. Each paint was mixed with a glass stirring rod and a paint mixer. Each film was immediately drawn onto polypropylene surfaces with a thickness of 8 mil. Cure time was 120 hrs. The Sherwin-Williams Acrylic Latex comprising a lysozyme wet paint was sealed and shelf stored at ambient temperature. After 48 hrs in can storage, films were drawn onto polypropylene surfaces with a thickness of 8 mils and were allowed to cure 72 hrs prior to assay. Materials for assay were generated from the polypropylene surface as a 2 cm′ (1×2 cm) free film.

The assay procedure included placing individual films into labeled 15 mL tubes. 24 hours prior to addition of Micrococcus lysodeikticus cell suspension, 5 mL KPO₄ buffer was added to the 24-hour control and coupon comprising a lysozyme tube, as well as one tube comprising 41 lysozyme solution (positive control) and one tube comprising 5 mL of the M. lysodeikticus cell suspension (negative control). These tubes were placed on the shaker for 24 hrs.

2 hours prior to addition of M. lysodeikticus, 5 mL potassium phosphate buffer was added to the 2-hour control and lysozyme tubes each comprising a coupon, as well as one tube comprising 41 μg lysozyme solution (positive control) and one tube comprising 5 mL of the M. lysodeikticus cell suspension (negative control). These tubes were placed on the shaker for 2 hrs.

1 hour prior to addition of M. lysodeikticus cell suspension, 5 mL potassium phosphate buffer was added to 1-hour control and coupon comprising a lysozyme tubes, as well as one tube comprising 41 μg lysozyme solution (positive control) and one tube comprising 5 mL of the M. lysodeikticus cell suspension (negative control). These tubes were placed on the shaker for one hour.

The paint coupons were then transferred from each tube to a second reaction tube. 5 mL of the M. lysodeikticus cell suspension was added to both film and KPO₄ buffer incubation buffer. The tubes were placed on the rotating shaker horizontally and shaken for approximately 4 hours, at which time each tube was measured in a Klett-Summerson Photoelectric Colorimeter to determine opacity.

TABLE 11
Assessment of lysis and enzyme leaching (free film) after 1, 2 and
24 hr, relative to the internal control (i.e., the no lysozyme films).

| | | Replicate 1 | | Replicate 2 | | Replicate 3 | | Average | |
|---|---|---|---|---|---|---|---|---|---|
| | | | Cell | | Cell | | Cell | | Cell |
| Formula- | Time | | lysis | | lysis | | lysis | | Lysis |
| tion | (hrs) | KU | (dKU) | KU | (dKU) | KU | (dKU) | KU | (dKU) |
| KPO₄ | | | | | | | | | |
| Buffer | | | | | | | | | |
| Control | 1 hr | 110 | 0% | 90 | 0% | 104 | 0% | 101 | 0% |
| Lysozyme | 1 hr | 62 | 39% | 42 | 59% | 52 | 49% | 52 | 49% |
| Control | 2 hr | 92 | 0% | 102 | 0% | 106 | 0% | 100 | 0% |
| Lysozyme | 2 hr | 74 | 26% | 65 | 35% | 65 | 35% | 68 | 32% |
| Control | 24 hr | 95 | 0% | 95 | 0% | 92 | 0% | 94 | 0% |
| Lysozyme | 24 hr | 80 | 15% | 62 | 34% | 55 | 41% | 66 | 30% |
| Film | | | | | | | | | |
| Control | 1 hr | 64 | 0% | 54 | 0% | 38 | 0% | 52 | 0% |
| Lysozyme | 1 hr | 3 | 94% | 40 | 23% | 4 | 92% | 16 | 81% |
| Control | 2 hr | 63 | 0% | 73 | 0% | 72 | 0% | 69 | 0% |
| Lysozyme | 2 hr | 10 | 86% | 23 | 67% | 45 | 35% | 26 | 54% |
| Control | 24 hr | 65 | 0% | 65 | 0% | 68 | 0% | 66 | 0% |
| Lysozyme | 24 hr | 30 | 55% | 52 | 21% | 52 | 21% | 45 | 32% |

KU = Klett Unit, measure of turbidity at 540 nm

At the three time points assayed, lysozyme leached out of films that comprised a lysozyme. The ability of the films comprising a lysozyme to lyse M. lysodeikticus was inversely related to the time the coupon was submerged. Over the first 2 hrs the films lost approximately 21%±3% of the lytic activity per hour. This loss decreased substantially over the following 22 hrs, with the loss slowing to approximately 3% per hour. After 24 hours of liquid submersion, approximately one-third of the activity of a coupon comprising a lysozyme was retained. Though reduction of activity due to leaching may continue, activity may also be permanently retained in the films. The total percentage lysis by coupon and buffer pairs decreased with increasing leaching time.

Example 12—This Example demonstrates the surface efficacy of paint films comprising a lysozyme in actively lyse M. lysodeikticus in a minimally hydrated environment.

Materials, reagents and equipment used are shown in the tables below.

TABLE 12
Materials and Reagents
0.1M potassium phosphate buffer, pH 6.4
Micrococcus lysodeikticus (Worthington Biochemicals, #8736)
Lysozyme (chicken egg white) (Sigma Product #L 6876, CAS 12650-88-3)
Sherwin-Williams Acrylic Latex paint
15 mL plastic test tubes

TABLE 13
Equipment
Paint spreader (1-8 mil)
Polypropylene blocks
Lightnin Labmaster Mixer
Rotator shaker
Pipetter and tips
Klett-Sumerson Colorimeter (Filter D35: 540 nm)

The reagents prepared included a Micrococcus cell suspension comprising 9 mg Micrococcus lysodeikticus in 25 mL sodium phosphate buffer, and a lysozyme solution comprising a 5 mg/mL stock solution.

The paint formulations prepared for the assay included a Sherwin-Williams Acrylic Latex Control (no additive), and a Sherwin-Williams Acrylic Latex with 1 mg/mL lysozyme. Each paint was mixed with a glass stirring rod and a paint mixer. Each film was immediately drawn onto polypropylene surfaces with a thickness of 8 mil. Cure time was 72 hrs. Assay materials were generated from the polypropylene surface as a 2 cm′ (1×2 cm) free film.

The assay procedure included placing individual coupons into separate Petri dishes. Each set of control coupons and coupons comprising a lysozyme was assayed in triplicate. Two controls were set up for this experiment: a M. lysodeikticus suspension control comprising 90 μL 20 mg/mL M. lysodeikticus cell suspension that was pipetted into a petri dish; and a 1 mg/mL lysozyme control comprising 40.64 μL 1 mg/mL lysozyme solution (an amount approximately equal to the amount of lysozyme in the 2 cm² coupon comprising a lysozyme) that was pipetted into a petri dish. M. lysodeikticus cell suspension was distributed onto the surface of each individual coupon in a minimal volume (90 Petri dishes were kept on a flat surface. After 4 hours, KPO₄ buffer was added to all samples to recover the unlysed portion of the M. lysodeikticus cell suspension. The suspension was removed from each dish with a pipette and placed into individual test tubes. Each suspension was read in the Klett-Summerson Photoelectric Colorimeter, using potassium phosphate buffer as a control.

TABLE 14
Surface Efficacy of Films comprising lysozyme in a low hydration
environment.

| | Replicate 1 | | Replicate 2 | | Replicate 3 | | Average | |
|---|---|---|---|---|---|---|---|---|
| | | Cell | | Cell | | Cell | | Cell |
| Formulation | KU | lysis | KU | lysis | KU | lysis | KU | Lysis |
| Suspension/ | | | | | | | | |
| Solution Controls | | | | | | | | |
| M. lysodeikticus | 80 | | | | | | | |
| Lysozyme | 10 | | | | | | | |
| S-W Acrylic Latex | | | | | | | | |
| Control Films | 75 | 6% | 70 | 13% | 78 | 3% | 74 | 7% |
| Lysozyme Films | 35 | 56% | 19 | 76% | 31 | 61% | 28 | 65% |

KU = Klett units, measure of turbidity at 540 nm.

The paint comprising a lysozyme contacted with 0.18 mg of a M. lysodeikticus suspension for 4 hours lysed 65%±10% of the Micrococcus cells, compared to only 7%±5% of cells lysed by the paint controls. This demonstrated that lysozyme can function in the low water (i.e., a minimally hydrated) environment of a coating. It is contemplated that a biological assay including a spray application of an assay organism would also demonstrate biostatic and/or biocidal activity.

Example 13—This Example demonstrates the ability of a chymotrypsin to survive the incorporation process into a coating and demonstrates chymotrypsin activity in a coating environment.

A chymotrypsin free film assay was used for determining the activity of chymotrypsin, as measured by ester hydrolysis (esterase) activity of a p-nitrophenyl acetate substrate, in free-films using a plate reader. A functioning vent hood was used for the assay when appropriate for material handling. A Sherwin-Williams Acrylic Latex paint was used. Equipment and reagents that were used are shown in the tables below.

TABLE 15
Equipment
Plate Reader
2 ml microtubes

TABLE 16
Reagents
α-Chymotrypsin from bovine pancreas, Type II (Sigma Cat# C4129)
4-Nitrophenyl acetate, MW 181.15 (Sigma Cat# N8130)
Trizma base (Sigma Cat# T1503)

Sample preparation included: 14.5 mM p-nitrophenyl acetate (66 mg/25 ml) in isopropyl alcohol, and 200 mM TRIS; pH 7.1 (adjust to pH 7.1 with HCl).

The paint formulations that were prepared included a Sherwin-Williams Acrylic Latex control (no additive), and a Sherwin-Williams Acrylic Latex comprising 200 mg/mL α-Chymotrypsin. Each paint was mixed with a glass stirring rod and a paint mixer. Each film was immediately drawn onto polypropylene surfaces with a thickness of 8 mil. Cure time was 24 days. Materials for assay were generated from the polypropylene surface as 1 cm², 2 cm² and 3 cm² free films.

The plate reader assay comprised: cutting free films into appropriate size pieces; adding 600 μL ddH₂O into a 2 ml microtube; then adding 750 μL 200 mM TRIS to each microtube; adding 150 μL of 14.5 mM p-nitrophenyl acetate to each tube; and taking the 0 time sample, then adding the free film to the tube (control sample is free film with no chymotrypsin).

The analysis included: taking out 100 μl and reading the absorbance at 405 nm, at the appropriate time points; and determining the initial rate slope by plotting absorbance vs. time to calculate chymotrypsin activity.

TABLE 17A
Absorbance at 405 nm Chymotrypsin in Sherwin-Williams
Acrylic Latex

| Time | Blank | | | 3 cm X 1 cm Control | | |
|---|---|---|---|---|---|---|
| 0 | 0.0480 | 0.0429 | 0.0446 | 0.0480 | 0.0429 | 0.0446 |
| 15 | 0.0482 | 0.0489 | 0.0479 | 0.0518 | 0.0541 | 0.0541 |
| 30 | 0.0571 | 0.0558 | 0.0555 | 0.0596 | 0.0612 | 0.0609 |
| 45 | 0.0608 | 0.0617 | 0.0617 | 0.0679 | 0.0709 | 0.0690 |
| 60 | 0.0683 | 0.0690 | 0.0679 | 0.0773 | 0.0826 | 0.0781 |
| Slope | 0.0004 | 0.0004 | 0.0004 | 0.0005 | 0.0006 | 0.0005 |

TABLE 17B
Absorbance at 405 nm Chymotrypsin in Sherwin-Williams
Acrylic Latex

| Time | 3 cm X 1 cm Enzyme | | | 2 cm X 1 cm Enzyme | | |
|---|---|---|---|---|---|---|
| 0 | 0.0480 | 0.0429 | 0.0446 | 0.0480 | 0.0429 | 0.0446 |
| 15 | 0.2364 | 0.2356 | 0.2347 | 0.1690 | 0.1801 | 0.1749 |
| 30 | 0.4504 | 0.4375 | 0.4208 | 0.3040 | 0.3149 | 0.3172 |
| 45 | 0.6395 | 0.6267 | 0.6441 | 0.4348 | 0.4579 | 0.4474 |
| 60 | 0.8358 | 0.7957 | 0.7970 | 0.5682 | 0.5942 | 0.5930 |
| Slope | 0.0132 | 0.0126 | 0.0128 | 0.0087 | 0.0092 | 0.0091 |

TABLE 17C
Absorbance at 405 nm Chymotrypsin in Sherwin-Williams Acrylic Latex

| Time | 1 cm × 1 cm Enzyme | | |
|---|---|---|---|
| 0 | 0.0480 | 0.0429 | 0.0446 |
| 15 | 0.1156 | 0.1155 | 0.1164 |
| 30 | 0.1886 | 0.1932 | 0.1872 |
| 45 | 0.2688 | 0.2745 | 0.2684 |
| 60 | 0.3427 | 0.3479 | 0.3578 |
| Slope | 0.0050 | 0.0051 | 0.0052 |

TABLE 18A
Absorbance Averages Chymotrypsin in Sherwin-Williams Acrylic Latex

| | Absorbance Average | | | | |
|---|---|---|---|---|---|
| | | | Chymo- | Chymo- | Chymo- |
| | | Control | trypsin | trypsin | trypsin |
| Time | Blank | 3 cm² | 3 cm² | 2 cm² | 1 cm² |
| 0 | 0.0452 | 0.0452 | 0.0452 | 0.0452 | 0.0452 |
| 15 | 0.0483 | 0.0533 | 0.2356 | 0.1747 | 0.1158 |
| 30 | 0.0561 | 0.0606 | 0.4362 | 0.3120 | 0.1897 |
| 45 | 0.0614 | 0.0693 | 0.6368 | 0.4467 | 0.2706 |
| 60 | 0.0684 | 0.0793 | 0.8095 | 0.5851 | 0.3495 |

TABLE 18B
Absorbance Averages Standard Deviations
Chymotrypsin in Sherwin-Williams Acrylic Latex

| | AbsorbanceStandard Deviation | | | | |
|---|---|---|---|---|---|
| | | | Chymo- | Chymo- | Chymo- |
| | | Control | trypsin | trypsin | trypsin |
| Time | Blank | 3 cm² | 3 cm² | 2 cm² | 1 cm² |
| 0 | 0.0026 | 0.0026 | 0.0026 | 0.0026 | 0.0026 |
| 15 | 0.0005 | 0.0013 | 0.0009 | 0.0056 | 0.0005 |
| 30 | 0.0009 | 0.0009 | 0.0148 | 0.0071 | 0.0031 |
| 45 | 0.0005 | 0.0015 | 0.0090 | 0.0116 | 0.0034 |
| 60 | 0.0006 | 0.0029 | 0.0228 | 0.0147 | 0.0077 |

TABLE 19
Absorbance vs. Time Slope

| | Slope | U | U | U |
|---|---|---|---|---|
| Sample | (A/min) | (umol/min) | Average | Deviation |
| Blank | 0.0004 | 0.0776 | 0.09 | 0.01 |
| | 0.0004 | 0.0949 | | |
| | 0.0004 | 0.0881 | | |
| Control | 0.0005 | 0.1090 | 0.12 | 0.02 |
| 3 cm² | 0.0006 | 0.1404 | | |
| | 0.0005 | 0.1195 | | |
| Chymotrypsin | 0.0132 | 2.8876 | 2.82 | 0.06 |
| 3 cm² | 0.0126 | 2.7679 | | |
| | 0.0128 | 2.7935 | | |
| Chymotrypsin | 0.0087 | 1.9062 | 1.97 | 0.06 |
| 2 cm² | 0.0092 | 2.0145 | | |
| | 0.0091 | 1.9983 | | |
| Chymotrypsin | 0.0050 | 1.0837 | 1.11 | 0.03 |
| 1 cm² | 0.0051 | 1.1222 | | |
| | 0.0052 | 1.1359 | | |

A chymotrypsin in Sherwin-Williams Acrylic Latex was able to hydrolyze the model substrate at rate 20× faster than the control. The test coupons demonstrate a dose response which corresponds to a hydrolytic capacity of 0.86 umol/min/cm², as formulated in this demonstration.

Quality control included reading and become familiar with the operating instructions for equipment used in the analysis. Operating instructions and preventive maintenance records were placed near the relevant equipment, and kept in a labeled central binder in the work area. Working solutions which are out of date or prepared incorrectly were disposed of and not used.

Safety procedures and precautions included wearing a full length laboratory coat; and not eating, drinking, smoking, use of tobacco products or application of cosmetics near the procedure. Consumables and disposable items that come in contact with or are used in conjunction with samples disposal were in the proper hazard containers. This includes, but is not limited to, pipette tips, bench-top absorbent paper, diapers, kimwipes, test tubes, etc. Biohazard containers were considered full when their contents reach three-quarters of the way to the top of the bag or box. Bench-top biohazard bags were placed into a large biohazard burn box when full. Biohazard containers were not filled to overflowing. Biohazard bags were disposed of by closing with autoclave tape, and autoclaving immediately. Spills and spatters were immediately cleaned from durable surfaces by applying 70% ethanol (for bacteriological spills) to the spill, followed by wiping or blotting. All equipment used in sample analyses were wiped down on a daily basis or whenever tests were performed. Absorbent pads were placed under samples when useful. Hands were washed with antibacterial soap before exiting the room, when a test was finished, and before the end of the day. The Material Safety Data Sheet (“MSDS”) applicable to each chemical was read. MSDS documents have been prominently posted in the laboratory. During a fire alarm during laboratory operations, evacuation procedures were followed. Nitrile protective gloves were worn whenever handling organophosphates. All organophosphate waste was disposed of properly.

Example 14—This Example demonstrates the ability of a cellulase to survive the incorporation process into a coating and demonstrates cellulase activity in a coating environment. A Glidden Latex paint was used. A plate reader was used to assay a free-film comprising a cellulase for the enzyme's activity.

Equipment and reagents that were used are shown in the table below.

TABLE 20
Equipment and Reagents
Equipment
Plate Reader
Reagents
Sodium Acetate (Sigma Cat# S8625)
4-Nitrophenyl β-D-cellobioside (Sigma Cat# N5759)
Cellulase (TCI Cat# C0057)
Sodium Hydroxide

Sample preparation included: 14.5 mM 4-Nitrophenyl β-D-cellobioside in ddH₂O; 50 mM sodium acetate buffer; pH 5.0 (adjust to pH 5.0 with HCl); and 2 N NaOH in ddH₂O.

The plate reader assay comprised: placing free films into 2 ml microtubes; add 1.2 ml 50 mM sodium acetate buffer, 0.15 ml 14.5 mM 4-Nitrophenyl β-D-cellobioside and 0.15 ml ddH₂O, in the 2 ml microtube; placing tubes on rocker; taking out 100 μl from the tubes into a 96-well plate at desired time points; adding 200 μl of 2 N NaOH and reading the absorbance at 405 nm; and determining the initial rate slope by plotting absorbance vs. time to calculate cellulase activity.

The paint formulations that were prepared included a Sherwin-Williams Acrylic Latex control (no additive), and a Sherwin-Williams Acrylic Latex comprising 100 g/gal, 200 g/gal and 300 g/gal cellulase. Each paint was mixed with a glass stirring rod and a paint mixer. Each film was immediately drawn onto polypropylene surfaces with a thickness of 8 mil. Cure time was 24 hrs. Materials for assay were generated from the polypropylene surface as a 3 cm² free film.

TABLE 21A
Glidden Latex Cellulase Free Films - Dose Response - pNP
Absorbance at 405 nm

| Time | | | | | | | |
|---|---|---|---|---|---|---|---|
| (min) | Blank | Control | | | 100 g/gal | | |
| 0 | 0.0600 | 0.0600 | 0.0600 | 0.0600 | 0.0600 | 0.0600 | 0.0600 |
| 30 | 0.0496 | 0.0588 | 0.0488 | 0.0476 | 0.0744 | 0.0753 | 0.0716 |
| 60 | 0.0496 | 0.0605 | 0.0505 | 0.0532 | 0.0975 | 0.1158 | 0.1007 |
| 120 | 0.0507 | 0.0519 | 0.0522 | 0.0514 | 0.1691 | 0.1823 | 0.1672 |
| 180 | 0.0550 | 0.0643 | 0.0583 | 0.0511 | 0.2351 | 0.2312 | 0.2073 |
| 240 | 0.0512 | 0.0614 | 0.0518 | 0.0548 | 0.2876 | 0.2919 | 0.2720 |
| 300 | 0.0491 | 0.0574 | 0.0601 | 0.0575 | 0.3187 | 0.3123 | 0.3083 |
| 360 | 0.0528 | 0.0680 | 0.0540 | 0.0655 | 0.3322 | 0.3215 | 0.3309 |
| Slope | −0.0001 | −0.0001 | 0.0000 | 0.0000 | 0.0009 | 0.0011 | 0.0009 |
| (A/ | | | | | | | |
| min) | | | | | | | |

TABLE 21B
Glidden Latex Cellulase Free Films - Dose Response - pNP
Absorbance at 405 nm

| Time (min) | 200 g/gal | | | 300 g/gal | | |
|---|---|---|---|---|---|---|
| 0 | 0.0600 | 0.0600 | 0.0600 | 0.0600 | 0.0600 | 0.0600 |
| 30 | 0.0986 | 0.0866 | 0.0927 | 0.1207 | 0.1170 | 0.1146 |
| 60 | 0.1387 | 0.1341 | 0.1432 | 0.1637 | 0.1711 | 0.1670 |
| 120 | 0.2285 | 0.2219 | 0.2364 | 0.2864 | 0.2685 | 0.2965 |
| 180 | 0.2891 | 0.2740 | 0.3071 | 0.3304 | 0.3262 | 0.3833 |
| 240 | 0.3174 | 0.3281 | 0.3270 | 0.3543 | 0.3638 | 0.4118 |
| 300 | 0.3449 | 0.3467 | 0.3511 | 0.3759 | 0.3891 | 0.4051 |
| 360 | 0.3714 | 0.3588 | 0.3632 | 0.3808 | 0.3964 | 0.3651 |
| Slope (A/min) | 0.0014 | 0.0014 | 0.0015 | 0.0019 | 0.0017 | 0.0020 |

TABLE 22A
Glidden Latex Cellulase Free Films—Dose
Response—pNP Absorbance at 405 nm Averages

| | Average | | | | |
|---|---|---|---|---|---|
| Time | | | 100 | 200 | 300 |
| (min) | Blank | Control | g/gal | g/gal | g/gal |
| 0 | 0.0600 | 0.0600 | 0.0600 | 0.0600 | 0.0600 |
| 30 | 0.0496 | 0.0517 | 0.0738 | 0.0926 | 0.1189 |
| 60 | 0.0496 | 0.0547 | 0.1047 | 0.1387 | 0.1674 |
| 120 | 0.0507 | 0.0518 | 0.1729 | 0.2289 | 0.2775 |
| 180 | 0.0550 | 0.0579 | 0.2245 | 0.2901 | 0.3283 |
| 240 | 0.0512 | 0.0560 | 0.2838 | 0.3242 | 0.3591 |
| 300 | 0.0491 | 0.0583 | 0.3131 | 0.3476 | 0.3825 |
| 360 | 0.0528 | 0.0625 | 0.3282 | 0.3645 | 0.3886 |

TABLE 22B
Glidden Latex Cellulase Free Films—Dose
Response—pNP Absorbance at 405 nm Averages' Deviations

| | Deviation | | | |
|---|---|---|---|---|
| Time | | 100 | 200 | 300 |
| (min) | Control | g/gal | g/gal | g/gal |
| 0 | 0.0000 | 0.0000 | 0.0000 | 0.0000 |
| 30 | 0.0061 | 0.0019 | 0.0060 | 0.0026 |
| 60 | 0.0052 | 0.0098 | 0.0046 | 0.0052 |
| 120 | 0.0004 | 0.0082 | 0.0073 | 0.0127 |
| 180 | 0.0066 | 0.0151 | 0.0166 | 0.0030 |
| 240 | 0.0049 | 0.0105 | 0.0059 | 0.0067 |
| 300 | 0.0015 | 0.0052 | 0.0032 | 0.0093 |
| 360 | 0.0075 | 0.0058 | 0.0064 | 0.0110 |

A cellulase in a Glidden Latex was able to hydrolyze the model substrate at a rate approximately 100× faster than the control. Quality control and safety procedures were as described in Example 13.

Example 15—This Example demonstrates preparation of technical papers coated with a latex coating comprising an antimicrobial enzyme additive, an antimicrobial peptide additive, or a combination thereof. The additives may be embedded in the coating.

The antimicrobial enzyme additive comprised lysozyme, and the antimicrobial peptide additive comprised ProteCoat® (Reactive Surfaces, Ltd.; also described in U.S. patent application Ser. Nos. 10/884,355; 11/368,086; and Ser. No. 11/865,514, each incorporated by reference). Materials that were used are shown in the tables below.

TABLE 23
Materials
30 mM Potassium Phosphate Buffer, was prepared by weighing out
416 mg of potassium phosphate into 2 × 50 mL conical tubes, and
adding 50 mL of water to each tube.
Micrococcus lysodeikticus (Worthington Biochemicals, #8736), was
prepared by weighing out 18 mg of Micrococcus into a single 50 mL
conical tube, adding KP0₄ buffer to 50 mLs, and mixing by inversion.
Lysozyme from chicken egg white (Sigma Product #L 6876; CAS no.
12650-88-3), was prepared by weighing out 1 g, 0.5 g and 0.1 g
lysozyme into 3 × 2 mL eppendorf tubes.
Dilute Acetic Acid Solution was prepared by measuring 1 mL of
glacial acetic acid into 11 mLs of water into a 15 mL conical tube,
and adding 50 μl of the dilute acetic acid to 1 mL of water.
ProteCoat ® was used at 125 mg ProteCoat ® per g coating,
dispensed as 250 mg ProteCoat ®, and resuspended in 2 mL dilute
acetic acid solution as appropriate.
5 × 15 mL conical tubes, glass stir rod
P1000 and P200 Pipetteman and Tips
5 × 15 mL conical tubes

Paint formulations comprising enzyme were prepared as follows: 1 g lysozyme per 100 g coating; 0.5 g lysozyme per 100 g coating; 0.1 9 lysozyme per 100 g coating; and a negative control (no additive). Paint formulations comprising a peptide additive were prepared as follows: 125 mg ProteCoat® per 1 g coating; 250 mg ProteCoat® per 1 g coating; 375 mg ProteCoat® per 1 g coating; or a negative control (no additive). Paint formulations comprising peptide and lysozyme were prepared as follows: 375 mg ProteCoat® per 1 g lysozyme (1 g) coating; 250 mg ProteCoat® per 1 g lysozyme (0.5 g) coating; 375 mg ProteCoat® per 1 g lysozyme (0.1 g) coating, and a negative Control (no additive). All paint formulations were mixed well. The paper was cut into quarters, coatings drawn onto paper surfaces with a spreader, and wet weight determined. The coated paper was dried at about 37.8° C. for approximately 10 min, and dry weight determined.

A single coating material and one paper stock was evaluated. The paper comprised celluosic fibers typically used in technical paper applications, and had an acrylic latex coating added to the fibers.

TABLE 24
Coating dry components added to paper

| Ingredient | % Dry Weight |
|---|---|
| Kaolin Clay | About 0.000000001% to about 90% |
| (filler/pigment) | |
| Titanium Dioxide | About 0.000000001% to about 90% |
| (pigment) | |
| Calcium Carbonate | About 0.000000001% to about 90% |
| (filler/pigment) | |
| Acrylic Latex | About 0.000000001% to about 80% |
| (Binder) | |

To prepare the antimicrobial paper (“AM-Paper”), the antimicrobial additives were formulated for each coating on percentage dry weight to standardize the coating for comparison. The antimicrobial additives are listed in the table below.

TABLE 25
Formulation details for antimicrobial papers

| Anti- | Desig- | Additive | Final Dry | Additive |
|---|---|---|---|---|
| microbial | nation | Formulation | Weight (gsm) | (%) |
| Control | | | 17.6 | None |
| | | | 21 | None |
| Enzymatic | | | | |
| | A | Powder | 21.9 | 0.2% |
| | B | Powder | 19.4 | 1% |
| | C | Powder | 23.2 | 2% |
| | D | Suspension | 23 | 0.2% |
| | E | Suspension | 23 | 1% |
| | F | Suspension | 20.7 | 2% |
| ProteCoat ® | | | | |
| | G | Suspension | 18.6 | 1% |
| | H | Powder | 23.9 | 2.5% |
| | I | Suspension | 20.6 | 0.5% |
| | J | Powder | 20.9 | 1.25% |
| | K | Powder | 20.9 | 0.25% |
| | L | Powder | 20.7 | 0.75% |
| Enzyme + | | | | |
| ProteCoat ® | | | | |
| | | Powder | 22.5 | 2% + 0.5% |
| | | Powder | 21.9 | 1% + 0.25% |

The antimicrobial additives were weighed out, added to pre-weighed coating suspensions and mixed by hand for 10 to 20 minutes. After mixing, the coating was applied by draw down, in which approximately 3-5 mLs of coating was applied along one 8.5″ edge of an 8.5″×11″ pre-weighed paper, and then spread evenly over the surface of the paper with a calibrated rod by drawing the rod down the full length of the paper. The coated paper was then placed into a 100° C. oven for 10 to 15 minutes to dry. After drying, the coated paper was weighed to determine the amount of coating on each sheet.

To conduct an assay to qualitatively assess antimicrobial activity, a paper strip of approximately 1 cm×5 cm was cut from the control and each antimicrobial paper. 5 mL of the M lysodeikticus suspension was poured into each of 4×15 mL conical tubes. The prepared strip was dropped into the suspension, and mixed occasionally by inversion. Clearing changes were observed.

Example 16—This Example demonstrates and provides a standard spectrophotometric assay procedure for lysozyme activity in a plate reader.

Equipment and reagents that were used are shown in the table below.

TABLE 26
Equipment and Reagents

Equipment
Thermo Multiskan Ascent Plate Reader
96-well assay plates
Multi-channels and single-channel pipettes and tips
Reagents
Tris(hydroxymethyl)aminomethane hydrochloride (Tris-HCI): [Sigma, cat #
T3253, Molecular Formula: NH₂C(CH₂OH)₃•HCI, Molecular Weight:
157.60, CAS Number 1185-53-1, pKa (25° C.) 8.1]
Micrococcus Iysodeikticus cell (Worthington Biochemicals, cat #8736)
Lysozyme: chicken egg white, Sigma cat #L6876; 50,000 U/mg; CAS
12650-88-3; molecular weight: 14.3 kD; solubility (H₂O) 10 mg/mL;
stability-1 month at 2-8° C. Standard: 25 μI of a 500,000 units (10 mg)/
mL (10 mM Tris-HCI) will typically lyse E. coli from >1 mL of culture
media cell pellet resuspended in 350 μl buffer (10 mM Tris HCI, pH 8.0,
with 0.1M NaCI, 1 mM EDTA, and 5% [w/v] Triton X-100). Typical
incubation conditions for lysis are 30 min at 37° C.

Micrococcus lysodeikticus cell suspension was made by adding 9 mg Micrococcus lysodeikticus to 25 mL 10 mM Tris-HCl, pH 8.0 and mixing well. Lysozyme solution was prepared by adding 10 mg lysozyme in 1 mL 10 mM Tris-HCl, pH 8.0, and mixing well. Reaction buffer was 10 mM Tris-HCl, pH 8.0, with an alternative reaction buffer being 0.1 M KPO₄ pH 6.4.

A standard curve of the M. lysodeikticus was prepared. The lysozyme stock solution was diluted with the reaction buffer to create the following series: 10 mg/mL (undiluted); 5.0 mg/mL; 2.5 mg/mL; 1 mg/mL; 0.5 mg/mL; 0.1 mg/mL; 0.05 mg/mL; 0.01 mg/mL; 0.005 mg/mL; 0.001 mg/mL; 0.0005 mg/mL; 0.0001 mg/mL, and 0 mg/mL. The controls included 3 replicates of 194 μL M. lysodeikticus cell suspension plus 6 μL buffer; and 3 replicates of 200 μL buffer.

Analysis of samples included determining activity by monitoring the clearing of the cell suspension at 570 nm and determining the best fit to a standard curve. For a 200 μL assay, 180 μL M. lysodeikticus in reaction buffer was added to each well 1 to 12 of 3 rows. The reaction was started by adding 20 μL of each lysozyme dilution to each well in the triplicate series. The plate was immediately placed into the reader, and the changes in absorbance at 570 nm (OD₅₇₀) recorded. The number of reads may be 10-20 with second intervals. The plate reader's velocity table contained data for reaction rate in mOD/min. This assay can be scaled by increasing each suspension proportionately (e.g., a 2 mL reaction is used for material strip analysis).

Analysis of the data included calculating the initial velocities for the recorded slopes: [mOD₅₄₀/min]/[slope standard curve (mOD/mg M. lysodeikticus]/[Iysozyme].

TABLE 27
Assay Standardization

| | |
|---|---|
| Coupon Size | None |
| Test Organism | Micrococcus lysodeikticus |
| Contamination level | 2.5 × 10⁸ cells/mL |
| Assay Time | 4 hr |

TABLE 28
Standardization of Assay

| [Lysozyme], (μg/mL)^{a} | OD₅₇₀ | % Lysis |
|---|---|---|
| 0 | 0.3 | 0.00 |
| 0.78 | 0.26 | 13.33 |
| 1.56 | 0.07 | 76.67 |
| 3.13 | 0.02 | 93.33 |
| 6.25 | 0.005 | 98.33 |
| 12.5 | 0.005 | 98.33 |
| 25 | 0.011 | 96.33 |
| 50 | 0.065 | 78.33 |

^{a}μg/mL = ppm

The M. lysodeikticus assay as described can detect lytic activity down to the fractional to low ppm range. The rate of lysis, in suspension, is 32% (about 8.0×10⁷ cells) of the M. lysodeikticus suspension per μg lysozyme.

Example 17—This Example demonstrates a spectrophotometric assay for antimicrobial paper with a lytic additive. Lysozyme was used as the lytic additive.

Equipment and reagents that were used are shown in the table below.

TABLE 29
Equipment and Reagents

Equipment
Spectrophotometer (Thermo Multiskan Ascent Plate Reader)
Cuvettes (96-well assay plates)
Multi-channels and single-channel pipettes and tips
Reagents
Tris(hydroxymethyl)aminomethane hydrochloride (Tris-HCI): [Sigma,
cat # T3253, Molecular Formula: NH₂C(CH₂OH)₃•HCI, Molecular
Weight: 157.60, CAS Number 1185-53-1, pKa (25° C.) 8.1]
Micrococcus Iysodeikticus cell (Worthington Biochemicals, cat #8736)
Lysozyme: chicken egg white, Sigma cat #L6876; 50,000 U/mg; CAS
12650-88-3; molecular weight: 14.3 kD; solubility (H₂O) 10 mg/mL;
stability-1 month at 2-8° C. Standard: 25 μI of a 500,000 units (10 mg)/
mL (10 mM Tris-HCI) will typically lyse E. coli from >1 mL of culture
media cell pellet resuspended in 350 μl buffer (10 mM Tris HCI, pH 8.0,
with 0.1M NaCI, 1 mM EDTA, and 5% [w/v] Triton X-100). Typical
incubation conditions for lysis are 30 min at 37° C.

Micrococcus lysodeikticus cell suspension was made by adding 9 mg M. lysodeikticus to 25 mL 10 mM Tris-HCl, pH 8.0 and mixing well. Lysozyme solution was prepared by adding 10 mg lysozyme in 1 mL 10 mM Tris-HCl, pH 8.0, and mixing well. Reaction buffer was 10 mM Tris-HCl, pH 8.0, with an alternative reaction buffer being 0.1 M KP0₄ pH 6.4. Antimicrobial paper coated with a coating comprising lysozyme and control paper was prepared in accordance with Example 15.

A standard curve of the M. lysodeikticus was prepared. The lysozyme stock solution was diluted with the reaction buffer to create the following series: 10 mg/mL (undiluted); 5.0 mg/mL; 2.5 mg/mL; 1 mg/mL; 0.5 mg/mL; 0.1 mg/mL; 0.05 mg/mL; 0.01 mg/mL; 0.005 mg/mL; 0.001 mg/mL; 0.0005 mg/mL; 0.0001 mg/mL and 0 mg/ml. The controls included 3 replicates of 194 μL M. lysodeikticus cell suspension plus 6 μL buffer; and 3 replicates of 200 μL buffer. Pipet tips used fitted the pipette (e.g., multichannel pipettes). The liquid level was correct in the tips, as air bubbles, etc may alter volume. Quality control and safety procedures were as described in Example 13.

Antimicrobial paper was cut into appropriately sized strips from both the antimicrobial and control paper. For a 5 mL assay in a 15 mL tube, standard sizes included 5×10 mm, 5×20 mm, and 5×40 mm. These strips could be combined to provide a desired step series.

Analysis of samples included determining activity by monitoring the clearing of the cell suspension at OD₅₇₀ and determining the best fit to a standard curve. For a 5 mL assay, M. lysodeikticus was added in reaction buffer to an OD₆₀₀ of 0.5. The reaction was started with the addition of the stripes. The tubes were immediately placed at 28° C. for a designated time (e.g., 4 hr and 24 hr). The absorbance at 570 nm was recorded.

Analysis of the data included calculating the initial velocities for the recorded slopes: [OD₆₀₀ min]/[slope standard curve (OD/mg M. lysodeikticus]/[Iysozyme]

Example 18—This Example demonstrates a biological assay for antimicrobial activity of paper strips comprising an antimicrobial enzyme additive against a microorganism.

The antimicrobial enzyme additive comprised lysozyme, the microorganism used was vegetative, gram-positive M. lysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 30
Equipment and Reagents

Equipment:
Petri Plates
Reagents:
Nutrient Yeast Extract (NBY) NBY Soft Agar
Lysozyme: chicken egg white, Sigma cat #L6876; 50,000 U/mg; CAS
12650-88-3; molecular weight: 14.3 kD; solubility (H₂O) 10 mg/mL;
stability-1 month at 2-8° C. Standard: 25 μI of a 500,000 units (10 mg)/
mL (10 mM Tris-HCI) will typically lyse E. coli from >1 mL of culture
media cell pellet resuspended in 350 μl buffer (10 mM Tris HCI, pH 8.0,
with 0.1M NaCI, 1 mM EDTA, and 5% [w/v] Triton X-100). Typical
incubation conditions for lysis are 30 min at 37° C.

Micrococcus lysodeikticus cell suspension was made by adding 9 mg Micrococcus lysodeikticus to NBY and mixing well, with OD₆₀₀ about 0.5. Antimicrobial paper coated with a latex coating comprising lysozyme and control paper was prepared in accordance with Example 15.

The assay includes cutting appropriated sized strips of both antimicrobial and control papers (e.g., a. 10×10 mm, 20×20 mm, 40×40 mm, or 50×50 mm). 100 μL of the prepared M. lysodeikticus suspension was transferred to 15 mL tube containing 5 mL NBY Soft Agar, held molten at 55° C., and mixed well. Pipet tips used fitted the pipette (e.g., multichannel pipettes). The liquid level was correct in the tips, as air bubbles, etc may alter volume. The mixture was immediately poured over a prepared sterile agar plate, rotating the dish to completely cover the agar with the M. lysodeikticus overlay. The dish was covered and allowed to solidify on level surface. The prepared antimicrobial paper(s) were placed (face down) on the soft agar overlay. Coupon(s) up to 20×20 mm were able to be paired with a control on a single petri dish. The dishes were left at 28° C. overnight, and visually evaluated for a zone of clearance around the antimicrobial coupon(s) relative to the control. Quality control and safety procedures were as described in Example 13.

Example 19—This Example demonstrates a biological assay for the antimicrobial activity of a paper strip comprising ProteCoat® against fungal spores.

The assay was adapted from ASTM 02020-92, Method A, Standard Test for Mildew (Fungus) Resistance of Paper and Paperboard (Reapproved 2003). Equipment and reagents that were used are shown in the table below.

TABLE 31
Equipment and reagents

Equipment:
Petri Plates
Incubator
Autoclave
Preval Sprayer
Reagents:
Nutrient Yeast Extract (NBY)
NBY Soft Agar
Micrococcus Iysodeikticus cell (Worthington Biochemicals, cat #8736)
ProteCoat ® was used at 125 mg ProteCoat ® per g coating, dispensed
as 250 mg ProteCoat ®, and resuspended in 2 mL dilute acetic acid
solution as appropriate.

Fusarium oxysporium spores were prepared by maintaining cultures of Fusarium oxysporum f sp. lycoperici race 1 (RM-1)[FOLRM-1 on Potato Dextrose Agar (PDA) slants. Microconidia of the Fusarium oxysporum f sp. lycoperici, were obtained by isolating a small portion of an actively growing culture from a PDA plate and transferring to 50 ml a mineral salts medium FLC (Esposito and Fletcher, 1961). The culture was incubated with shaking (125 rpm) at 25° C. After 960 h the fungal slurry consisting of mycelia and microconidia were strained twice through eight layers of sterile cheese cloth to obtain a microconidial suspension. The microcondial suspension was then calibrated with a hemacytometer. All fungal inocula were tested for the absence of contaminating bacteria before their use in experiments. Antimicrobial paper coated with a latex coating comprising ProteCoat® and control paper was prepared in accordance with Example 15.

The assay procedure included: cutting appropriated sized strips of both antimicrobial and control papers (e.g., 40×40 mm or 50×50 mm); centering the strips on a sterile Potato Dextrose Agar plate, treated side up; diluting spores to 2×10³ per mL Potato Dextrose broth; transferring to a calibrated preval sprayer (i.e., dispense 50 μL per single pump action); dispersing spores in a hood onto the agar and paper surface with a single pump action (delivers approximately 100 spores to the area); covering and leaving at ambient conditions; and observing growth over several days, though time of assay will depend on organism. Pipet tips fitted the pipette (e.g., multichannel pipettes). The liquid level was correct in the tips, as air bubbles, etc may alter volume. Quality control and safety procedures were as described in Example 13.

Example 20—This Example demonstrates a paper coating comprising an antimicrobial enzyme additive. The antimicrobial enzyme comprised a lysozyme.

Assay standardization and data are shown in the following tables.

TABLE 32
Assay Enzymatic Additive-Lysozyme

| | |
|---|---|
| Example Techniques Used | Example 15 and 17 |
| Coupon Size | Variable, 200-600 mm² |
| Paper Age | 3 months |
| Test Organism | Micrococcus lysodeikticus |
| Contamination level | 2.5 × 10⁸ cells/mL |
| Assay Time | 4 and 24 hrs |

TABLE 33A
Test Strips and Data

| Paper | Paper coupon (mm × | | |
|---|---|---|---|
| Type | mm) | Area (mm²) | [lysozyme], μg |
| 0 | | 0 | |
| 0.2% | 5 × 40 | 200 | 8.76 |
| 1.0% | 5 × 40 | 200 | 38.80 |
| 2.0% | 5 × 40 | 200 | 92.80 |
| 2.0% | 5 × 40 + 5 × 10 | 250 | 116.00 |
| 2.0% | 5 × 40 + 5 × 20 | 300 | 139.20 |
| 2.0% | 5 × 40 + 5 × 40 | 400 | 185.00 |
| 2.0% | 5 × 40 + 5 × 40 + 5 × 10 | 450 | 208.80 |
| 2.0% | 5 × 40 + 5 × 40 + 5 × 20 | 500 | 232.00 |
| 2.0% | 5 × 40 + 5 × 40 + 5 × 40 | 600 | 278.40 |

TABLE 33B
Antimicrobial Strips and Data

| Paper | Paper coupon (mm × | 4 hrs | | 24 hrs | |
|---|---|---|---|---|---|
| Type | mm) | OD₅₇₀ | % Lysis | OD₅₇₀ | % Lysis |
| 0 | | 0.305 | 0.00 | 0.27 | 0.00 |
| 0.2% | 5 × 40 | 0.301 | 1.31 | 0.275 | −1.85 |
| 1.0% | 5 × 40 | 0.277 | 9.18 | 0.2 | 25.93 |
| 2.0% | 5 × 40 | 0.172 | 43.61 | 0.0015 | 99.44 |
| 2.0% | 5 × 40 + 5 × 10 | 0.099 | 67.54 | 0.001 | 99.63 |
| 2.0% | 5 × 40 + 5 × 20 | 0.136 | 55.41 | 0.0025 | 99.07 |
| 2.0% | 5 × 40 + 5 × 40 | 0.017 | 94.43 | 0.005 | 99.81 |
| 2.0% | 5 × 40 + 5 × 40 + 5 × 10 | 0.023 | 92.46 | 0.001 | 99.63 |
| 2.0% | 5 × 40 + 5 × 40 + 5 × 20 | 0.024 | 92.13 | 0.001 | 99.63 |
| 2.0% | 5 × 40 + 5 × 40 + 5 × 40 | 0.015 | 95.08 | 0.0015 | 99.44 |

The rate of lysis upon contact with a coupon cut from antimicrobial treated paper, is approximately 0.5% (1.35×10⁷ cells) per μg lysozyme. This corresponds to a reduction in activity, per μg of lysozyme, of approximately 65% over that observed in suspension. Treated papers of identical size with antimicrobial loadings of 0.2%, 1.0% and 2.0%, demonstrated antimicrobial function. The antimicrobial concentration on a per unit of area for those loadings, is provided in the following table.

TABLE 34
Antimicrobial concentration per unit area

| | | | Lysozyme | | |
|---|---|---|---|---|---|
| Paper | Coating (gsm) | % lysozyme | g/m² | μg/m² | μg/mm² |
| A | 21.9 | 0.2% | 0.0438 | 4.38 × 10⁻⁸ | 0.0438 |
| B | 19.4 | 1.0% | 0.194 | 1.94 × 10⁻⁷ | 0.194 |
| C | 23.2 | 2.0% | 0.464 | 4.64 × 10⁻⁷ | 0.464 |

Example 21—This Example qualitatively demonstrates an antimicrobial enzyme additive combined with an antimicrobial peptide additive to provide antimicrobial functionality to a paper coating formulation.

An adaptation of ASTM 02020-92 was used as the assay to demonstrate the growth of a microorganism in a petri dish was inhibited by contact with the treated paper. The antimicrobial enzyme additive comprised lysozyme, and the antimicrobial peptide additive comprised ProteCoat® Reactive Surfaces, Ltd.; also described in U.S. patent application Ser. Nos. 10/884,355; 11/368,086; and Ser. No. 11/865,514, each incorporated by reference).

The spectrophotometric lysozyme assay uses Micrococcus lysodeikticus bacterial cells as a substrate, and measures the change in the turbidity of the cell suspension as described in Example 11 and Example 12. The efficacy of an antimicrobial peptide (e.g., ProteCoat™) may be monitored biologically. Though the contemplated mechanism of action for an antimicrobial or anti-fouling peptide is similar, i.e. disruption of the structural components of the microbial cell, the cell wall may remain relatively intact. As an antifungal or antimicrobial peptide's biocidal or biostatic activity inhibits the cell, the cell may not lyse for detection of a change in turbidity. Biological assay conditions are shown in the table below.

TABLE 35
Enzymatic Additive-Lysozyme (Qualitative)

| Example Techniques Used | Example 13 |
|---|---|
| Coupon Size | 100 mm² |
| Paper Age | 3 months |
| Test Organism | Micrococcus lysodeikticus |
| Growth Conditions | 28° C. |

A zone of clearing was seen around the antimicrobial paper in contact with a petri dish covered by M. lysodeikticus, whereas the control paper had no such zone. The coupon of paper was about half the size of the smallest coupons in the quantitative M. lysodeikticus assay, yet growth inhibition was seen.

Assay conditions for Fusarium oxysporum is shown at the table below.

TABLE 36
Enzymatic Additive-ProteCoat ® (Qualitative)

| Example Techniques Used | Example 14 |
|---|---|
| Coupon Size | 40 × 40 mm |
| Paper Age | 3 months |
| Test Organism | Fusarium oxysporum |
| Contamination level | 100 spore, aerosol delivery |
| Growth Conditions | Ambient |

Overgrowth of both test and control ProteCoat paper by the fungus, Fusarium oxysporium, was observed. The developmental state of the mycelium on the antimicrobial paper was retarded over that seen in the control paper, indicative of biostatic, and possibly biocide activity.

Example 22—This Example demonstrates synergism between an antimicrobial enzyme additive combined with an antimicrobial peptide additive in a coating applied to papers, and to demonstrate antimicrobial activity of a paper comprising the antimicrobial peptide.

The antimicrobial enzyme additive comprised lysozyme, and the antimicrobial peptide additive comprised ProteCoat® (Reactive Surfaces, Ltd.; also described in U.S. patent application Ser. Nos. 10/884,355; 11/368,086; and Ser. No. 11/865,514, each incorporated by reference). Assay conditions are shown at the tables below.

TABLE 37
Enzymatic Additive-2% Lysozyme +
0.5% ProteCoat ® (Titration Assay)

| Example Techniques Used | Example 19 |
|---|---|
| Coupon Size | Variable, 0-400 mm² |
| Paper Age | 3 months |
| Test Organism | Micrococcus lysodeikticus |
| Contamination level | 2.5 × 10⁸ cells/mL |
| Assay Time | 3 and 20 hrs |

TABLE 38A
Activity in Treated Papers

| | | Area | Lysozyme | | ProteCoat ® | |
|---|---|---|---|---|---|---|
| Paper | Strips (mm × mm) | (mm²) | mg | μg/mL | mg | μg/mL |
| 2% | | 0 | 0 | | | |
| Lysozyme | 5 × 5 | 25 | 11.60 | 2.90 | 0.00 | 0.00 |
| | 5 × 10 | 50 | 23.20 | 5.80 | 0.00 | 0.00 |
| | 5 × 20 | 100 | 46.40 | 11.60 | 0.00 | 0.00 |
| | 5 × 40 | 200 | 92.80 | 23.20 | 0.00 | 0.00 |
| | 5 × 40 + 5 × 5 | 225 | 104.40 | 26.10 | 0.00 | 0.00 |
| | 5 × 40 + 5 × 10 | 250 | 116.00 | 29.00 | 0.00 | 0.00 |
| | 5 × 40 + 5 × 20 | 300 | 139.20 | 34.80 | 0.00 | 0.00 |
| | 5 × 40 + 5 × 40 | 400 | 185.60 | 46.40 | 0.00 | 0.00 |
| 2% | | 0 | | | | |
| Lysozyme + | 5 × 5 | 25 | 11.60 | 2.90 | 2.90 | 0.73 |
| 0.5% | 5 × 10 | 50 | 23.20 | 5.80 | 5.80 | 1.45 |
| ProteCoat ® | 5 × 20 | 100 | 46.40 | 11.60 | 11.60 | 2.90 |
| | 5 × 40 | 200 | 92.80 | 23.20 | 23.20 | 5.80 |
| | 5 × 40 + 5 × 5 | 225 | 104.40 | 26.10 | 26.10 | 6.53 |
| | 5 × 40 + 5 × 10 | 250 | 116.00 | 29.00 | 29.00 | 7.25 |
| | 5 × 40 + 5 × 20 | 300 | 139.20 | 34.80 | 34.80 | 8.70 |
| | 5 × 40 + 5 × 40 | 400 | 185.60 | 46.40 | 46.40 | 11.60 |

TABLE 38B
Activity in Treated Papers

| | Strips | Area | 3 hrs | | 20 hrs | |
|---|---|---|---|---|---|---|
| Paper | (mm × mm) | (mm²) | OD₆₀₀ | % Lysis | OD₆₀₀ | % Lysis |
| 2% | | 0 | 0.266 | 0.00 | 0.258 | 0.00 |
| Lysozyme | 5 × 5 | 25 | 0.259 | 2.63 | 0.25 | 3.10 |
| | 5 × 10 | 50 | 0.259 | 2.63 | 0.23 | 10.85 |
| | 5 × 20 | 100 | 0.256 | 3.76 | 0.145 | 43.80 |
| | 5 × 40 | 200 | 0.228 | 14.29 | 0.038 | 85.27 |
| | 5 × 40 + 5 × 5 | 225 | 0.199 | 25.19 | 0.019 | 92.64 |
| | 5 × 40 + 5 × 10 | 250 | 0.148 | 44.36 | 0.011 | 95.74 |
| | 5 × 40 + 5 × 20 | 300 | 0.177 | 33.46 | 0.013 | 94.96 |
| | 5 × 40 + 5 × 40 | 400 | 0.09 | 66.17 | 0.012 | 95.35 |
| 2% | | 0 | 0.266 | 0.00 | 0.258 | 0.00 |
| Lysozyme + | 5 × 5 | 25 | 0.255 | 4.14 | 0.23 | 10.85 |
| 0.5% | 5 × 10 | 50 | 0.248 | 6.77 | 0.057 | 77.91 |
| ProteCoat ® | 5 × 20 | 100 | 0.237 | 10.90 | 0.016 | 93.80 |
| | 5 × 40 | 200 | 0.195 | 26.69 | 0.012 | 95.35 |
| | 5 × 40 + 5 × 5 | 225 | 0.199 | 25.19 | 0.012 | 95.35 |
| | 5 × 40 + 5 × 10 | 250 | 0.15 | 43.61 | 0.012 | 95.35 |
| | 5 × 40 + 5 × 20 | 300 | 0.124 | 53.38 | 0.01 | 96.12 |
| | 5 × 40 + 5 × 40 | 400 | 0.031 | 88.35 | 0.012 | 95.35 |

The concentration of lysozyme in the papers corresponded to between 2 and 50 ppm, whereas ProteCoat® was between 0.5 and 12 ppm. The comparison of lysis between the 2% lysozyme paper, and the combined paper which contained 2% lysozyme and 0.5% ProteCoat® indicates synergism between the additives. For example, the 100 mm² coupon size exhibited 44% lysis, whereas the combined paper exhibited 93%. This is an observed/expected (93/44+0) of 2.1, indicative of significant synergism. To further demonstrate this activity, the assay was repeated by titrating the 2% lysozyme paper with individual swaths of 2.5% ProteCoat® paper. 5×10, 5×20, and 5×40 mm² lysozyme paper strips with increasing amount of Protecoat® paper were added to tubes in 4 ml total volume 2.5×10⁸ Micrococcus cells/ml. The assay conditions are shown at the tables below.

TABLE 39
Enzymatic Additive-2% Lysozyme & 2.5% ProteCoat ® (Titration)

| Example Techniques Used | Example 19 |
|---|---|
| Coupon Size | Variable |
| Lysozyme | 0-200 mm² |
| ProteCoat ® | 0-200 mm² |
| Paper Age | 3 months |
| Test Organism | Micrococcus lysodeikticus |
| Contamination level | 2.5 × 10⁸ cells/mL |
| Assay Time | 4 and 22 hrs |

TABLE 40
Activity of Protecoat ® paper with 50, 100 and 200 mm² Lysozyme paper
against Micrococcus lysodeikticus

| | | | Square | | |
|---|---|---|---|---|---|
| | | Square | area | | |
| | Strips | area (mm²) | (mm²) | [lysozyme] | [Protecoat ®] |
| Paper | (mm × mm) | Lysozyme | Protecoat ® | (ug/ml) | (ug/ml) |
| Control | 0 | 0 | 0 | 0 (0) | 0 (0) |
| 2% | 5 × 10 | 50 | 0 | 23.2 (5.8) | 0 (0) |
| Lysozyme | | | | | |
| 2.5% | 5 × 5 | 50 | 25 | 23.2 (5.8) | 15 (3.75) |
| Protecoat ® | 5 × 10 | 50 | 50 | 23.2 (5.8) | 30 (7.5) |
| | 5 × 20 | 50 | 100 | 23.2 (5.8) | 60 (15) |
| | 5 × 40 | 50 | 200 | 23.2 (5.8) | 120 (30) |
| | 5 × 40 × 2 | 50 | 400 | 23.2 (5.8) | 240 (60) |
| Control | 0 | 0 | 0 | 0 (0) | 0 (0) |
| 2% | 5 × 20 | 100 | 0 | 46.4 (11.6) | 0 (0) |
| Lysozyme | | | | | |
| 2.5% | 5 × 5 | 100 | 25 | 46.4 (11.6) | 15 (3.75) |
| Protecoat ® | 5 × 10 | 100 | 50 | 46.4 (11.6) | 30 (7.5) |
| | 5 × 20 | 100 | 100 | 46.4 (11.6) | 60 (15) |
| | 5 × 40 | 100 | 200 | 46.4 (11.6) | 120 (30) |
| | 5 × 40 × 2 | 100 | 400 | 46.4 (11.6) | 240 (60) |
| 2% | 5 × 40 | 200 | 0 | 92.8 (23.2) | 0 (0) |
| Lysozyme | | | | | |
| 2.5% | 5 × 5 | 200 | 25 | 92.8 (23.2) | 15 (3.75) |
| Protecoat ® | 5 × 10 | 200 | 50 | 92.8 (23.2) | 30 (7.5) |
| | 5 × 20 | 200 | 100 | 92.8 (23.2) | 60 (15) |
| | 5 × 40 | 200 | 200 | 92.8 (23.2) | 120 (30) |
| | 5 × 40 × 2 | 200 | 400 | 92.8 (23.2) | 240 (60) |

An example of a calculation for the lysozyme content in 2% lysozyme paper was: 23.2×2% g/m²=0.464 g/m²=0.464 μg/mm². An example of a calculation for the Protecoat® content in 2.5% Protecoat® paper was: 23.9×2.5% g/m²=0.60 g/m²=0.60 μg/mm².

TABLE 41
Activity of Protecoat ® paper with 50, 100 and 200 mm² Lysozyme paper
against Micrococcus lysodeikticus

| | Strips | 4 hrs | | 23 hrs | |
|---|---|---|---|---|---|
| Paper | (mm × mm) | OD₆₀₀ | % Lysis | OD₆₀₀ | % Lysis |
| Control | 0 | 0.278 | 0 | 0.276 | 0 |
| 2% | 5 × 10 | 0.269 | 3.24 | 0.206 | 25.36 |
| Lysozyme | | | | | |
| 2.5% | 5 × 5 | 0.264 | 5.04 | 0.235 | 14.86 |
| Protecoat ® | 5 × 10 | 0.268 | 3.60 | 0.213 | 22.83 |
| | 5 × 20 | 0.269 | 3.24 | 0.197 | 28.62 |
| | 5 × 40 | 0.266 | 4.32 | 0.172 | 37.68 |
| | 5 × 40 × 2 | 0.24 | 13.67 | 0.027 | 90.22 |
| Control | 0 | 0.254 | 0 | 0.229 | 0 |
| 2% | 5 × 20 | 0.224 | 11.81 | 0.026 | 88.65 |
| Lysozyme | | | | | |
| 2.5% | 5 × 5 | 0.22 | 13.39 | 0.023 | 89.96 |
| Protecoat ® | 5 × 10 | 0.204 | 19.69 | 0.013 | 94.32 |
| | 5 × 20 | 0.212 | 16.54 | 0.019 | 91.70 |
| | 5 × 40 | 0.178 | 29.92 | 0.014 | 93.89 |
| | 5 × 40 × 2 | 0.194 | 23.62 | 0.027 | 88.21 |
| 2% | 5 × 40 | 0.203 | 20.08 | 0.019 | 91.70 |
| Lysozyme | | | | | |
| 2.5% | 5 × 5 | 0.181 | 28.74 | 0.009 | 96.07 |
| Protecoat ® | 5 × 10 | 0.175 | 31.10 | 0.01 | 95.63 |
| | 5 × 20 | 0.165 | 35.04 | 0.012 | 94.76 |
| | 5 × 40 | 0.128 | 49.61 | 0.012 | 94.76 |
| | 5 × 40 × 2 | 0.145 | 42.91 | 0.019 | 91.70 |

TABLE 42A
% Lysis (relative to control without Protecoat ® added)
at given time

| | 4 hr | | |
|---|---|---|---|
| Square Area | 50 mm² | 100 mm² | 200 mm² |
| (mm²) of | Lysozyme | Lysozyme | Lysozyme |
| Protecoat ® paper | paper | paper | paper |
| 0 | 3.24 | 11.81 | 20.08 |
| 25 | 5.04 | 13.39 | 28.74 |
| 50 | 3.60 | 19.69 | 31.10 |
| 100 | 3.24 | 16.54 | 35.04 |
| 200 | 4.32 | 29.92 | 49.61 |
| 400 | 13.67 | 23.62 | 42.91 |

TABLE 42B
% Lysis (relative to control without Protecoat ® added)
at given time

| | 22 hr | | |
|---|---|---|---|
| Square Area | 50 mm² | 100 mm² | 200 mm² |
| (mm²) of | Lysozyme | Lysozyme | Lysozyme |
| Protecoat ® paper | paper | paper | paper |
| 0 | 25.36 | 88.65 | 91.70 |
| 25 | 14.86 | 89.96 | 96.07 |
| 50 | 22.83 | 94.32 | 95.63 |
| 100 | 28.62 | 91.70 | 94.76 |
| 200 | 37.68 | 93.89 | 94.76 |
| 400 | 90.22 | 88.21 | 91.70 |

The assay was repeated by titrating the 2% lysozyme paper with individual swaths of 2.5% ProteCoat® paper. Lysozyme in technical papers added to an assay at concentrations greater than 10 ppm exhibited antimicrobial activity in the M. lysodeikticus assay. Lysozyme at approximately 5 ppm in the assay did not exhibit significant antimicrobial activity over the course of the assay (20 hrs). The addition of ProteCoat® papers, with between 3 and 60 ppm ProteCoat® to the assay significantly enhanced the lytic activity of lysozyme, or possibly the reverse. This was also true with the 5 ppm lysozyme, in which the lytic activity was doubled by the addition of between 3 and 60 ppm ProteCoat® to the assay. The peptide additive may be enhancing the activity of the enzyme, or the enzyme enhancing the activity of the peptide, or both, to produce these results.

Example 23—This Example demonstrates a spectrophotometric assay for an antimicrobial coating with a lytic additive. The lytic additive comprised a lysozyme.

The antimicrobial coatings were created using acrylic latex, commercially available paints. Equipment and reagents that were used are shown in the table below.

TABLE 43
Equipment and Reagents

Equipment
Spectrophotometer (Thermo Multiskan Ascent Plate Reader)
Cuvettes (96-well assay plates)
Multi-channels and single-channel pipettes and tips
Reagents
Tris(hydroxymethyl)aminomethane hydrochloride (Tris-HCI): [Sigma, cat #T3253, Molecular
Formula: NH₂C(CH₂OH)₃ · HCI, Molecular Weight: 157.60, CAS Number 1185-53-1, pKa
(25° C.) 8.1]
Micrococcus lysodeikticus cell (Worthington Biochemicals, cat #8736)
Lysozyme: chicken egg white {Sigma cat #L6876; 50,000 U/mg; CAS 12650-88-3; molecular
weight: 14.3 kD; solubility (H₂O) 10 mg/mL; stability-1 month at 2-8° C.. Standard: 25 μI of
a 500,000 units (10 mg)/mL (10 mM Tris-HCI) will typically lyse E. coli from >1 mL of culture
media cell pellet resuspended in 350 μl buffer (10 mM Tris HCI, pH 8.0, with 0.1M NaCI, 1
mM EDTA, and 5% [w/v] Triton X-100). Typical incubation conditions for lysis are 30 min at
37° C.)

A Micrococcus lysodeikticus cell suspension was made by adding 1.5 mg Micrococcus lysodeikticus to 1 mL 10 mM Tris pH 8.0 and mixing well. A lysozyme solution was prepared by adding 10 mg lysozyme in 1 mL ddH₂₀, and mixing well.

The lysozyme stock solution was mixed into Sherwin Williams Acrylic (SW) or Glidden latex paint (1 part water:7 part paint). 4 mil, 6 mil, and 8 mil free films were created from Sherwin Williams paint comprising a lysozyme, a Glidden paint comprising a lysozyme, and controls for both. The plate controls included 3 replicates of 50 μL M. lysodeikticus cell suspension plus 50 μL buffer; and 3 replicates of 100 μL buffer. Pipet tips used fitted the pipette (e.g., multichannel pipettes). The liquid level was correct in the tips, as air bubbles, etc may alter volume. Quality control and safety procedures were as described in Example 13.

The antimicrobial films were cut into appropriately sized strips from both the antimicrobial and control coating. For a 5 mL assay in a 15 mL tube, standard size was 1×1 cm.

Analysis of samples included determining activity by monitoring the clearing of the cell suspension at OD₄₀₅ and determining the best fit to a standard curve. The reaction was started with the addition of 5 ml of the M. lysodeikticus stock. The tubes were immediately placed on a rocker for 3 hr; 100 μl samples were taken at 3 hr, and the absorbance at 405 nm was recorded.

TABLE 44
Sample Lysis Averages and Deviations

| | | Avg. % | |
|---|---|---|---|
| | | Lysis at | Standard |
| Sample | | 3 hr | Deviation |
| SW Control | 4 mils | 11.1057 | 0.5752 |
| | 6 mils | 12.2932 | 0.3812 |
| | 8 mils | 12.2802 | 0.5752 |
| SW Lysozyme | 4 mils | 65.0651 | 1.3638 |
| | 6 mils | 74.5744 | 3.8272 |
| | 8 mils | 84.2325 | 4.1432 |
| Glidden Control | 4 mils | 4.8514 | 0.4912 |
| | 6 mils | 5.1005 | 0.0569 |
| | 8 mils | 5.1749 | 0.6266 |
| Glidden | 4 mils | 18.3760 | 0.5846 |
| Lysozyme | 6 mils | 23.1840 | 3.6201 |
| | 8 mils | 29.1666 | 1.9095 |

Analysis of the data included calculating the initial velocities for the recorded slopes: [OD₄₀₅ min]/[slope standard curve (OD/mg M. lysodeikticus]/[Iysozyme].

Example 24—This Example demonstrates a biological assay for antimicrobial activity of coatings comprising an antimicrobial enzyme additive against a microorganism.

The antimicrobial enzyme additive comprised lysozyme, the microorganism used comprised vegetative, gram-positive M. lysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 45
Equipment and Reagents

Equipment:
Petri Plates
Reagents:
Luria Broth Agar (LBA)
Lysozyme: chicken egg white, Sigma cat #L6876; 50,000 U/mg; CAS 12650-88-3;
molecular weight: 14.3 kD; solubility (H₂O) 10 mg/mL; stability-1 month at 2-8° C..
Standard: 25 μI of a 500,000 units (10 mg)/mL (10 mM Tris-HCI) will typically lyse E. coli
from >1 mL of culture media cell pellet resuspended in 350 μl buffer (10 mM Tris HCI, pH
8.0, with 0.1M NaCI, 1 mM EDTA, and 5% [w/v] Triton X-100). Typical incubation
conditions for lysis are 30 min at 37° C..

A Micrococcus lysodeikticus cell suspension was made by adding 1.5 mg M. lysodeikticus to 10 mM Tris, pH 8.0, and mixing well. A lawn of M. lysodeikticus was generated by spreading 200 μl of this suspension onto a LBA plate, using a glass spreading rod. An antimicrobial latex coating comprising lysozyme and a control film was prepared in accordance with Example 23.

The assay includes cutting appropriated sized strips of both antimicrobial and control latex films (e.g., a 1×1 cm). In triplicate the free films are carefully placed onto the surface of the petri dishes spaced out equally. This procedure was repeated for each of the paint film types/thicknesses.

The paint films comprising a lysozyme were active in lysing M. lysodeikticus, producing circular zones of clearing. The difference in Zone of Clearing Diameter between the different thicknesses of film was deemed negligible.

TABLE 46
Diameter (cm) of Zones of Clearing

| Sample | 4 mils | 6 mils | 8 mils |
|---|---|---|---|
| Glidden Lysozyme | 2.8 | 2.8 | 2.8 |
| | 2.8 | 2.9 | 2.8 |
| | 2.7 | 2.9 | 2.9 |
| Glidden Control | 0 | 0 | 0 |
| | 0 | 0 | 0 |
| | 0 | 0 | 0 |
| Sherwin Williams Lysozyme | 2.1 | 1.9 | 2.2 |
| | 2.1 | 1.9 | 1.9 |
| | 2 | 2 | 1.8 |
| Sherwin Williams Lysozyme | 0 | 0 | 0 |
| | 0 | 0 | 0 |
| | 0 | 0 | 0 |

Example 25—This Example demonstrates a qualitative biological assay for survivability of an antimicrobial latex coating comprising an antimicrobial enzyme additive against a microorganism.

The antimicrobial enzyme additive comprised lysozyme, the microorganism used comprised vegetative, gram-positive M. lysodeikticus. The assay was adapted from ASTM 02020-92, Method A, Standard Test for Mildew (Fungus) Resistance of Paper and Paperboard (Reapproved 2003). Equipment and reagents that were used are shown in the table below.

TABLE 47
Equipment and Reagents

Equipment:
Petri Plates
Reagents:
Luria Broth Agar (LBA)
Lysozyme: chicken egg white, Sigma cat #L6876; 50,000 U/mg; CAS 12650-88-3;
molecular weight: 14.3 kD; solubility (H₂O) 10 mg/mL; stability-1 month at 2-8° C..
Standard: 25 μI of a 500,000 units (10 mg)/mL (10 mM Tris-HCI) will typically lyse
E. coli from >1 mL of culture media cell pellet resuspended in 35 μl buffer (10 mM
Tris HCI, pH 8.0, with 0.1M NaCI, 1 mM EDTA, and 5% [w/v] Triton X-100). Typical
incubation conditions for lysis are 30 min at 37° C..

A Micrococcus lysodeikticus cell suspension was made by adding 1.5 mg M. lysodeikticus to 10 mM Tris, pH 8.0, and mixing well. A lawn of M. lysodeikticus was generated by spreading 200 μl of this suspension onto a LBA plate, using a glass spreading rod.

The paint formulations that were prepared included a Sherwin-Williams Acrylic Latex or a Glidden Acrylic Latex as controls (no additive), and both a Sherwin-Williams Acrylic Latex or a Glidden Acrylic Latex comprising 10 mg/mL Lysozyme (ddH₂O). Each paint was made by adding 1 part additive to 7 parts paint, and then mixed with a glass stirring rod and a paint mixer. Each film was immediately drawn onto polypropylene surfaces with a thickness of 4 mil, 6 mil, and 8 mil. Cure time was 24 days. Materials for assay were generated from the polypropylene surface as 1 cm² free films.

The assay includes cutting appropriately sized strips of both antimicrobial and control latex films (e.g., a 1×1 cm). In triplicate the free films were carefully placed onto the surface of the petri dishes spaced out equally. This procedure was repeated for each of the paint film types/thicknesses.

After 24 hrs incubation, the diameter of the zones of clearing was measured for each film. Using sterile tweezer, the films were removed and transfer to a new LBA plate spread with M. lysodeikticus in the same orientation as the plates the films were removed from. Repeat the procedure of measuring the zones of clearing through transfer to a new plate every day for 5 days.

TABLE 48
Average Diameter (cm) of Zones of Clearing

| | | | Stand- | | Stand- | | Stand- |
|---|---|---|---|---|---|---|---|
| | | | ard | | ard | | ard |
| | | | Devia- | | Devia- | | Devia- |
| | | 4 mils | tion | 6 mils | tion | 8 mils | tion |
| Day 1 | Glidden | N/A | N/A | N/A | N/A | 0 | 0 |
| | Control | | | | | | |
| | Glidden | 2.5667 | 0.0577 | 2.5333 | 0.0577 | 2.7000 | 0.0000 |
| | Lysozyme | | | | | | |
| Day 2 | Glidden | N/A | N/A | N/A | N/A | 0 | 0 |
| | Control | | | | | | |
| | Glidden | 2.0000 | 0.0000 | 2.0000 | 0.0000 | 2.2000 | 0.0000 |
| | Lysozyme | | | | | | |
| Day 3 | Glidden | N/A | N/A | N/A | N/A | 0 | 0 |
| | Control | | | | | | |
| | Glidden | 1.4667 | 0.0577 | 1.6667 | 0.0577 | 1.9000 | 0.0000 |
| | Lysozyme | | | | | | |
| Day 4 | Glidden | N/A | N/A | N/A | N/A | 0 | 0 |
| | Control | | | | | | |
| | Glidden | 1.4333 | 0.1155 | 1.5667 | 0.0577 | 1.8000 | 0.0000 |
| | Lysozyme | | | | | | |
| Day 5 | Glidden | N/A | N/A | N/A | N/A | 0 | 0 |
| | Control | | | | | | |
| | Glidden | 1.2667 | 0.0577 | 1.4500 | 0.0707 | 1.6333 | 0.0577 |
| | Lysozyme | | | | | | |

¹N/A in this chart just means not available/ not applicable.

There were no 4 mil or 6 mil controls tested due to a limited LBA plate supply, though 8 mil control films were tested. The standard deviations for the 8 mil controls to 0, because all 3 controls produced a 0 cm zone of clearing in each case.

The paint films comprising lysozyme were active in lysing M. lysodeikticus, producing circular zones of clearing, for five cycles of contaminant control. The difference in Zone of Clearing Diameter between the different thicknesses of each film appeared negligible.

Example 26—To provide a description that is both concise and clear, various examples of ranges have been identified herein. Any range cited herein includes any and all sub-ranges and specific values within the cited range, this example provides specific numeric values for use within any cited range that may be used for an integer, intermediate range(s), subrange(s), combinations of range(s) and individual value(s) within a cited range, including in the claims. Examples of specific values (e.g., %, kDa, ° C., μm, kg/L, Ku) that can be within a cited range include 0.000001, 0.000002, 0.000003, 0.000004, 0.000005, 0.000006, 0.000007, 0.000008, 0.000009, 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.10, 99.20, 99.30, 99.40, 99.50, 99.60, 99.70, 99.80, 99.90, 99.91, 99.92, 99.93, 99.94, 99.95, 99.96, 99.97, 99.98, 99.99, 99.999, 99.9999, 99.99999, 99.999999, 99.9999999, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5250, 5500, 5750, 6000, 6250, 6500, 6750, 7000, 7250, 7500, 7750, 8000, 8250, 8500, 8750, 9000, 9250, 9500, 9750, 10,000, 25,000, 50,000, 75,000, 100,000, 250,000, 500,000, 1,000,000, or more. Additional examples of the use of this definition to specify sub-ranges are given herein. For example, a cited range of 25,000 to 100,000 would include specific values of 50,000 and/or 75,000, as well as sub-ranges such as 25,000 to 50,000, 25,000 to 75,000, 50,000 to 100,000, 50,000 to 75,000, and/or 75,000 to 100,000. In another example, the range 875 to 1200 would include values such as 910, 930, etc. as well as sub-ranges such as 940 to 950, 890 to 1150, etc.

In embodiments wherein a value or range is denoted in exponent form, both the integer and the exponent values are included. For example, a range of 1.0×10⁻¹⁷ to 2.5×10⁻⁷, would include a description for a sub-range such as 1.24×10⁻¹⁷ to 8.7×10⁻¹¹. However, general sub-ranges for each type of unit (e.g., %, kDa, ° C., μm, kg/L, Ku) are contemplated, as the values typically found within a particular type of unit are of a sub-range of the integers described above. For example, integers typically found within a cited percentage range, as applicable, include 0.000001% to 100%. Examples of values that can be within a cited molecular mass range in kilo Daltons (“kDa”) as applicable for many coating components include 0.50 kDa to 110 kDa. Examples of values that can be within a cited temperature range in degrees Celsius (“° C.”) as may be applicable in the arts of a polymeric material, a surface treatment (e.g., a coating), and/or a filler include −10° C. to 500° C. Examples of values that can be within a thickness range in micrometers (“μm”) as may be applicable to coating and/or film thickness upon a surface include 1 μm to 2000 μm. Examples of values that can be within a cited density range in kilograms per liter (“kg/L”) as may be applicable in the arts of a material formulation include 0.50 kg/L to 20 kDa. Examples of values that can be within a cited shear rate range in Krebs Units (“Ku”), as may be applicable in the arts of a material formulation, include 20 Ku to 300 Ku.

Example 27—This Example is directed to the assay for active phosphoric triester hydrolase expression in cells.

Routine analysis of parathion hydrolysis in whole cells is accomplished by suspending cultures in 10 milli-Molar (“mM”) Tris hydrocholoride at pH 8.0 comprising 1.0 mM sodium EDTA (“TE buffer”). Cell-free extracts are assayed using sonicated extracts in 0.5 milliLiters (“ml”) of TE buffer. The suspended cells or cell extracts are incubated with 10 microLiters (“μl”) of substrate, specifically 100 μg of parathion in 10% methanol, and p-nitrophenol production is monitored at a wavelength of 400 nm. To induce the opd gene under lac control, 1.0 μmol of isopropyl-β-D-thiogalactopyranoside (Sigma) per ml is added to the culture media.

Example 28—This Example is directed to the preparation of an enzyme powder. In a typical preparation, a single colony of bacteria that expresses the opd gene is selected and cultured in a rich media. After growth to saturation, the cells are concentrated by centrifugation at 7000 rotations per minute (“rpm”) for 10 minutes for example. The cell pellet is then resuspended in a volatile organic solvent such as acetone one or two times to desiccate the cells and to remove a substantial portion of the water contained in the cell pellet. The pellet may then be ground or milled to a powder form. The powder may be frozen or stored at ambient conditions for future use, or may be added immediately to a surface coating formulation. Additionally, the powder may be freeze dried, combined with a cryoprotectant (e.g., cryopreservative), or a combination thereof.

Example 29—This Example is directed to the formation of an OPH powder and latex coating. In an example of use of the powder prepared as described in Example 28, 3 mg of the milled powder was added to 3 ml of 50% glycerol. The suspension was then added to 100 ml of Olympic® premium interior flat latex paint (Olympic®, One PPG Place, Pittsburgh, Pa. 15272 USA). This paint with biomolecular composition was then used to demonstrate the activity of the paint biomolecular composition in hydrolysis of a pesticide or a nerve agent analog.

Example 30—This Example demonstrates, in a first set of assays, a paint product as prepared in Example 29 was applied to a hard, metal surface. The surface used in the present Example was a non-galvanized steel surface that was cleaned through being degreased, and pretreated with a primer coat. A control surface was painted with the identical paint with no biomolecular composition. Paraoxon, an organophosphorus nerve gas analog was used as an indicator of enzyme activity. Paraoxon, which is colorless, is degraded to form p-nitrophenol, which is yellow in color, plus diethyl phosphate, thus giving a visual indication of enzyme activity. In multiple assays, the surface with control paint remained white, indicating no production of p-nitrophenol, and the surface painted with the paint and biomolecular composition turned yellow within minutes, indicating an active OPH enzyme in the paint. This demonstration has shown that the surface remains active for more than 65 days, which was the maximum duration of the protocol.

Example 31—This Example demonstrates anti-fouling activity of a cell-based particulate material comprising an organophosphorus hydrolase in a marine coating that was superior to the activity of a different purified control enzyme (i.e., acetylase—subtilisin). A marine coating comprising an anti-biological peptide was also evaluated for anti-fouling activity.

To evaluate the anti-fouling properties of various enzymes and peptides, a marine coating (i.e., an emulsion blend of water dispersed resin system) had either: an cell-based particulate material comprising an organophosphorus hydrolase (EC 3.1.8.1) similar to that described for Example 28; an acetylase—subtilisin, which is a non-specific alkaline protease secreted by bacteria of the genus Bacillus (Novozymes); or a peptide preparation (ProteCoat™, comprising SEQ ID no. 40); blended into the coating sample. The final concentration of a cell-based particulate material comprising an organophosphorus hydrolase or the peptide preparation in the coating samples was about 3% to about 5% weight of these anti-biological agents. The concentration of the acetylase—subtilisin in the coating sample was unknown. A Ross blender was used to disperse the cell-based particulate material comprising an organophosphorus hydrolase into the coating resin sample. A marine coating sample not comprising any enzymatic or peptidic anti-biological agent was used as a control. The pot life time was about 45 to about 60 min prior to application. Each coating sample was applied at room temperature to a different steel surface previously coated with an epoxy undercoat. The cure time for each coating to tack was 20 min, cure time to tack free was 2 hr, and cure time to usable service was between about 24h (fast) to 48h (normal). The dry film thickness was about 5 to about 6 mils for each coating sample.

The coated steel surfaces were statically and continuously submerged in Atlantic Ocean's waters, during summer, for about 1 month. Coating properties such as adhesion and hardness was not altered by incorporation of the anti-biological agents into the coating samples. No corrosion was detected in the steel surfaces for any coating sample.

All coating samples were fouled. The coating comprising an cell-based particulate material comprising an organophosphorus hydrolase, however, was easily cleaned from the surface relative to the other coating samples. Specifically, removal of fouling from the coating sample comprising the cell-based particulate material comprising an organophosphorus hydrolase did not require mechanical scrapping, unlike the other coating samples. The organophosphorus hydrolase bioadditive reduced and/or prevented adherence of the fouling organisms and fouling film. Barnacles that did attach were unable to glue themselves to the surface sufficiently to require mechanical removal methods. Some grasses may have attached to the surface of some coated surface when free of barnacles.

The marine coating used was a 2K system, that is, a 2-pack coating where separate coating components are admixed shortly before application. The addition of an additional enzyme or peptide to the coating effectively made the coating a 3K (i.e., 3 pack) system. It is contemplated that such a multi-pack system comprising a container with an anti-fouling bioadditive will retain greater bioactivity during pot-life storage, as the bioadditive is not contacted with other potentially damaging coating components prior to preparation for use. It is further contemplated that anti-fouling activity for a coating comprising an cell-based particulate material comprising an OP compound degrading enzyme, cell debris from a microorganism typically used to produce the OP compound degrading enzyme (e.g., Escherichia coli), and/or a purified OP compound degrading enzyme, may also possess such anti-biological and/or anti-fouling activity, possibly for an extended period of time (e.g., about 1, 2, 3, 4, or 5 years or more). It is also contemplated that combinations of such anti-biological enzyme and/or peptide with each other and/or another anti-biological agent (e.g., a preservative, a co-biocide, an anti-fouling agent, an anti-microbial agent) may produce additive and/or synergistic anti-biological (e.g., anti-fouling activity) in a coating.

Example 32—This Example demonstrates immobilization of streptavidin in a coating. The streptavidin is for promoting the retention of a biotin labeled hexahistidine metal binding sequence in the coating.

Streptavidin was added to a Joncryl® 74 coating, so that the streptavidin would function as an anchor for biotin labeled hexahistidine in the coating.

The equipment used included polypropylene sheet(s), a drawdown bar, and a balance. The reagents used included: streptavidin (Cat #PRO-283, Lot #1209STREP01 from ProSpec.com; ProSpec, Bonsal American, 8201 Arrowridge Blvd., Charlotte, N.C. 28273, USA), Joncryl® 74 Å (48.5% Solids; BASF Corporation, 8310 16^{th} Street; P.O. Box 902; Sturtevant, Wis., 53177, USA) and 25% aqueous glutaraldehyde. The Joncryl® 74 Å coating was a soft film forming acrylic polymer emulsion, with approximate properties of: a 8.1 pH; a 700 cps viscosity; a 1.03 g/cm³ density at 25° C.; a −16° C. Tg; a 50 acid number (“NV”); a >200,000 molecular weight (“Mw”); a freeze/thaw stability property; a minimum film forming temperature of <5° C.; a 1.0 total wt. % VOC; grease (i.e., ink and overprint varnish formulations) resistance, water resistance, rub resistance, and a high slide angle coating property suitable for use on folding carton and multiwall bag applications.

Joncryl® 74 Å neat coating samples were drawn down onto two separate polypropylene sheets (i.e., two 5 mil draws per sheet with an uncoated section of polypropylene between each draw). The films were allowed to cure overnight at room temperature.

Streptavidin was rehydrated using 2 mL sterile water to make a final concentration of 5 mg/mL. 10 mL Joncryl® 74 Å (4.80 g solids) were blended with 1 mL (5 mg) of the streptavidin, and 120 μL of 25% aqueous glutaraldehyde added for a final cross-linker concentration of 0.5%. The blended overcoating was allowed to react for 5 min. The overcoat material was drawn at 3 mil thickness in three separate (i.e., independent) locations across the 5 mil supporting undercoat on a polypropylene sheet as well as the uncoated section of the polypropylene sheet. For a control, 5 mL Joncryl® 74 Å (2.4 g solids) was blended with 500 μL (2.5 mg) streptavidin without glutaraldehyde. Three separate control resin films were similarly drawn at 3 mil thickness across the undercoat layer of another polypropylene sheet as well as the uncoated section of the polypropylene sheet. The overcoats were cured at room temperature overnight. The undercoat and overcoat coated sections, as well as the overcoat only sections, of the polypropylene sheets were sectioned for analysis. It is contemplated that the thinner film of the overcoat only sections may result in less noise and/or lower sensitivity in an absorbance dependant assay.

The resin system with the glutaraldehyde incorporated turned yellow within 5 min of blending. The coating was drawn over the control films quickly at 3 mils. No color changes were observed with the glutaraldehyde free streptavidin-Joncryl blend. It is contemplated that that the aldehydes likely reacted with the coating polymer amines creating chromatic aberrations. The aldehyde functional groups may have reacted with the polymer and the proteins to crosslink them to the coating material. There is no data to indicate that the color change was due to the aldehyde covalently linking with the protein.

Example 33—This Example demonstrates Biotin labeled histidine (“HH-Biotin,” “HH-Bio oligopeptide”) incubation onto a coating comprising streptavidin (“streptavidin coating”).

Upper coatings of Joncryl® 74 Å comprising immobilized streptavidin as previously described was used to bind Biotin labeled histidine (“HH-Biotin,” “HH-Bio oligopeptide”) tag.

The equipment used included a balance, a pipette, and a 40° C. shaker plate. The reagents used included a His6-Bio-0240 (Lot #10-9712-17687, 93.5%; structure His-His-His-His-His-His-OCO₂-OCO₂-(K-Ahx-biotin)-amide; 21st Century Biochemicals, 33 Locke Drive, Marlboro, Mass. 01752, U.S.A.), sterile water, scintillation vial, and wax paper. 36.5 mg HH bio oligopeptide was weighed into a scintillation vial and 3.65 mL sterile water added. The HH-Bio oligopeptide sample was placed on the 40° C. incubator shaker for 10 min to dissolve the sample. The HH-Bio oligopeptide sample was checked after incubation 250×rpm for 10 min for residual crystals. If the HH-Bio oligopeptide sample was completely dissolved, the HH-Bio oligopeptide sample was removed from the incubator and set at room temperature until cool. If no crystals formed upon cooling, the HH-Biotin solution sample was ready for use.

The polypropylene sheet having the 3 mil Joncryl® 74 Å films (3 samples total) comprising streptavidin was placed on a flat surface. 1 mL of the HH-Biotin solution was placed on the coating. A section of wax paper was placed on top of the HH-Biotin solution. The HH-Biotin solution was spread under the waxpaper to cover the area of the film that is to contact the biotin. The HH-Biotin solution was incubated for 2 hours at room temperature before removing the wax paper.

It is contemplated that the coating samples will undergo further assays to demonstrate streptavidin and/or labeled hexahistidine incorporation efficiency and/or metal binding (e.g., Ni⁺, Cu⁺, Ag⁺, Co⁺) binding properties. For example, elemental scanning electron microscopy may be conducted quantitate the amount of a metal and the metal's location in a film.

Example 34—This Example demonstrates minimal inhibitory concentration (“MIC”) observed in a 96-well plate assay.

All metals and the Protecoat™ (i.e., comprising SEQ ID no. 40) tested for comparison were prepared as a 10 mg/ml stocks and the highest tested concentration for each is 5 mg/ml (5000 μg/ml). After inoculating each of the test wells with 100 μl of a 2×10⁴ cells/ml inoculum (final well volume=200 μl), the plates are incubated at 37° C. for 24 hours and then are replica plated onto agar to observe the MIC. All tested against E. coli ATCC #8739. The MIC for each coating is shown in the Table below.

TABLE 49
MIC of Protecoat ™ vs Metal Compounds

| Protecoat ™ Standard | MIC (μg/m1) |
|---|---|
| Protecoat ™ in ddH2O | 156 |
| (#12166 C) | |
| Metals | |
| Cobalt(II) chloride hexahydrate | 625 |
| Copper(II) carbonate | 5000 |
| Copper(II) sulfate | 625 |
| Nickel sulfate | 625 |
| Silver nitrate | 20 |
| Copper Powder | Greater than 5000 |

Example 35—This Example describes reversible binding of biomolecular composition(s), ligand(s) for the biomolecular composition(s), and material formulation (e.g., coating) component(s) that may occur to create a “rechargeable” material.

For example, it is contemplated that a proteinaceous molecule comprising a binding sequence that may reversibly bind a component of the material formulation, based on the association equilibrium constant Ka and/or the dissociation equilibrium constant Kdiss (“Kd”), so that the proteinaceous molecule may be released from a material formulation by a mechanism such as elution. The content of the proteinaceous molecule in the material formulation may then be “recharged” by contacting a proteinaceous molecule to the material formulation so it is rebound as part of the material formulation. An example of such a rechargeable system would be a polypeptide comprising a streptavidin sequence and another bioactive proteinaceous sequence (e.g., a metal binding sequence, an anti-fouling sequence, an-anti-biological sequence, an enzyme sequence) and a material formulation comprising an affinity resin comprising biotin as a component, wherein the unbound biotin may be used to bind additional polypeptide molecule(s), which may comprise the same or different bioactive proteinaceous sequence(s), upon contact of the additional polypeptide molecule(s) with the material formulation after a period of time where some of the previously bound polypeptide molecules have been lost due to elution or other environmental conditions(s). For example, the bioactive proteinaceous sequence of such a polypeptide molecule may be binding a metal cation to also “recharge” the anti-fouling metal content of a material formulation such as a marine coating. Of course, other binding sequence(s) and compositions described herein (e.g., an immobilization agent) or as would be know in the art in light of the present disclosures may be used in a rechargeable material formulation/biomolecular composition system.

In some embodiments, it is contemplated that a material formulation comprising a binding sequence and/or an immobilized biomolecular composition may be reprogrammed to bind a like or different ligand for the binding sequence and/or a like or different biomolecular composition bound by the immobilization agent. Reprogramming refers to replacement of a bound (e.g., immobilized) material formulation component (e.g., a metal ion, a proteinaceous molecule) for a different bound component. For example, a material formulation may comprise a plurality of streptavadin molecules to bind a proteinaceous sequence comprising a biotin. As one or more proteinaceous molecule(s) become unbound from the streptavadin, an additional biotin linked proteinaceous molecule, which may comprise the same or different sequences, may bind the free streptavidin. Reprogramming may be conducted by contacting a material formulation with the additional material (e.g., a solution comprising a different biotin linked proteinaceous molecule) to become bound to the immobilization agent. In another example, a material formulation at least one unbound (“free”) biotin molecule may be reprogrammed by being contacted with streptavidin comprising proteinaceous molecule comprising a different sequence that a proteinaceous molecule that previously was bound to the free biotin molecule.

It is also contemplated that numerous proteinaceous molecules, such as those of peptide library(s), whether synthetically or recombinantly produced through genetic modification technique(s), may be screened for variations in binding association and/or dissociation equilibrium constant(s), using the assays and/or techniques described herein or as would be known in the art, to identify and/or optimize the coefficient(s) of binding for a particular application (e.g., binding for a particular metal ligand for use in an anti-fouling coating).

For example, it is contemplated that a metal binding sequence may be selected for having binding coefficient(s) sufficient to accumulate a metal found in a preparation (e.g., a solution) contacted with a material formulation to recharge the metal binding sequence. In another example, the metal binding sequence may be selected for a binding coefficient capable of accumulating a toxic metal at a concentration typically found in the environment the material formulation will be used in. In a specific example, a marine coating comprising a metal binding sequence may accumulate a metal ion from ocean water that contacts the marine coating. In another example, the binding coefficient of the metal binding sequence may be selected to be capable of such accumulation and/or modulating the net loss/rate of release of metal from a material formulation based on the expected concentration of metal ion available in the ocean water, the material formulation, and/or a material used to recharge (e.g., a solution/slurry of metal) the metal content of the material formulation. The table below gives exemplary concentrations of metals dissolved in ocean water.

TABLE 50
Metal Metal Concentrations In Ocean Water

| Metal | Typical Concentration in Ocean Water* |
|---|---|
| Alumina | about 10 to about 20 nmol/L |
| Antimony | about 0.5 to about 2 nmol/L |
| Arsenic | about 15 to about 25 nmol/L |
| Chromium | about 1 to about 4 nmol/L |
| Cobalt | about 20 to about 40 pmol/L |
| Copper | about 1 to about 5 nmol/L |
| Iron | about 70 to about 760 pmol/L |
| Manganese | about 0.25 to about 1 nmol/L |
| Mercury | about 0.7 to about 1.1 pmol/L |
| Silver | about 7 to about 20 pmol/L |
| Zinc | about 0.5 to about 10 nmol/L |

*(see, for example, ″Handbook on the Toxicology of Metals″ pp. 263-278, 2007)

Example 36:—This Example demonstrates the ability of a coating to bind metals by the incorporation of metal binding peptides.

The materials used are shown on the Table below.

TABLE 51
Materials
Nickel sulfate (Sigma Product #656895)
Copper sulfate (Sigma Product #451657)
Cobalt chloride hexahydrate (Sigma Product #255599)
His6 peptide (21st Century Biochemicals Product #HIS6-0400)
Distilled water pH 5 and 10
2 mL microtubes
Pipette
Pipette Tips
Plate Reader
96-well Plate
Oven
Rocker
Glidden Vinyl Latex Paint (Cat #AF 1424)

Films were prepared by adding 93 mg/ml His6 peptide into water and mixing into a Glidden coating at a ratio of 1 part water to 7 parts coating. The coating was mixed well until the peptide was evenly dispersed. A draw down bar was used to make films on a polypropylene block at 8 mils thickness. The films were allowed to cure for 48 hours before use. The control films were made in the same manner without the added peptide.

3 cm² pieces of film were cut from the polypropylene blocks. Individual coupons were placed into labeled 2 mL microtubes. Each of the coupons was tested in triplicate. 1 ml 200 mM nickel sulfate was added into each microtube. The microtubes were placed on a rocker overnight. The liquid was removed from the microtubes and add distilled water (pH 5) into each microtube. The microtubes were placed on a rocker overnight. The liquid was removed (leave the films behind) from the microtubes and place into new microtubes. The microtubes were placed in an oven at 70° C. to remove all of the water. Once the microtubes were dry, 200 ul water (pH 10) was added into each tube, and the water transferred into 96-well plate and read the absorbance at 340 nm. The concentration of nickel was determined using a standard curve. This procedure was repeated 100 mM copper sulfate using an absorbance of 450 nm, and with 42 mM cobalt chloride hexahydrate using an absorbance of 414 nm, to determine the concentration of those metals.

The recorded absorbances were as follows:

TABLE 52
Bound Metal Absorbances

| Metal | Nm | Control Film | H6 Peptide Film |
|---|---|---|---|
| Nickel sulfate | 340 | 0.2648 | 0.4159 |
| Copper sulfate | 450 | 0.1303 | 0.1939 |
| Cobalt chloride hexahydrate | 414 | 0.1382 | 0.1527 |

Using the standard curves for each metal, the following molarities were calculated:

TABLE 53
Standard Curves

| | Control Film (mM) | H6 Peptide Film (mM) |
|---|---|---|
| Nickel sulfate | 77 | 131 |
| Copper sulfate | 60 | 102 |
| Cobalt chloride hexahydrate | 30 | 35 |

Standard curves are shown below.

TABLE 54
Standard Curves (Note: 200 μL well volume used)

| Nickel sulfate | | Copper sulfate | | Cobalt chloride hexahydrate | |
|---|---|---|---|---|---|
| pH 10 | | pH 10 | | pH 10 | |
| mM | Abs 340 nm | mM | Abs 450 nm | mM | Abs 414 nm |
| 200 | 0.6101 | 100 | 0.1944 | 42 | 0.1878 |
| 180 | 0.5405 | 90 | 0.1745 | 38 | 0.1582 |
| 160 | 0.4914 | 80 | 0.1609 | 34 | 0.1473 |
| 140 | 0.4482 | 70 | 0.1462 | 29 | 0.1344 |
| 120 | 0.3897 | 60 | 0.1300 | 25 | 0.1195 |
| 100 | 0.3302 | 50 | 0.1162 | 21 | 0.1061 |
| 80 | 0.2744 | 40 | 0.1019 | 17 | 0.0927 |
| 60 | 0.2213 | 30 | 0.0878 | 13 | 0.0833 |
| 40 | 0.1479 | 20 | 0.0716 | 8 | 0.0671 |
| 20 | 0.1117 | 10 | 0.0593 | 4 | 0.0546 |
| 0 | 0.0423 | 0 | 0.0362 | 0 | 0.0368 |

The Glidden films with the metal binding peptide bound more nickel, copper and cobalt than in the control films. There was stronger nickel and copper binding in the films. The cobalt chloride hexahydrate did not show as much binding as the other metals which may be due to the fact that the cobalt complex is larger than the other metals that were tested. Distilled water at pH 5 was used to wash the films was to optimize the release of metal peptides from the free films back into solution. At lower pH values, there is more competition at the binding sites of the peptide between hydrogen ions and the metals.

Example 37—This Example describes the contemplated ability of a coating to bind silver by the incorporation of a metal binding peptide.

The materials that may be used are shown on the Table below.

TABLE 55
Materials
Silver nitrate
Sodium chloride
Distilled water pH 12
2 mL microtubes
Pipette
Pipette Tips
Plate Reader
96-well Plate
Rocker
Glidden Vinyl Latex Paint (Cat #AF 1424)

Films may be prepared by adding 93 mg/ml His6 peptide into water and mix into a Glidden coating at a ratio of 1 part water to 7 parts coating. The peptide and coating may be mixed well until the peptides will be evenly dispersed. A draw down bar may be used to make films on a polypropylene block at 8 mils thickness. The films may be allowed to cure for 48 hours before use. Control films may be made in the same manner without the added peptide.

3 cm² pieces of film may be cut from the polypropylene blocks. Individual coupons may be placed into labeled 2 mL microtubes. Each of the coupons may be tested in triplicate. 1 ml 20 mM nickel sulfate (pH 12) may be added into each microtube. The microtubes may be placed on a rocker overnight. 100 ul of solution may be taken from each tube and added into 96-well plate. 100 ul 0.2 mg/ml NaCl (pH 12) may be added into each well.

It is contemplated that the addition of NaCl with the presence of silver nitrate will cause a brown precipitate to appear. It is contemplated that the solution that will be exposed to the control free films will show more brown precipitate in solution as opposed to the films containing the metal binding peptide. Thus, there will be more silver nitrate attached to the films containing the peptide as opposed to the control films.

Example 38—This Example describes an assay to detect an incorporated proteinaceous molecule in a coating.

The stain for a proteinaceous molecule used was Coomassie R250. The Coomassie R250 characteristics included the development of intensely colored complexes with proteins, and can determine as little as 0.5 μg/cm² of protein present in a gel matrix. An anion of Coomassie Brilliant Blue formed in the acidic staining medium combines with the protonated amino groups of proteins by electrostatic interaction; though the resulting complex is reversible under the proper conditions.

Films prepared as described above with His peptide or peptide free controls were stained with Coomassie R250 in 40% methanol, 10% acetic acid, 50% water for 1 hr. The films were then washed with 40% methanol, 10% acetic acid, 50% water for ˜40 min. The films were then stored in water. The films prepared with peptide stained darker than the controls, indicating peptide was incorporated into the material.

### REFERENCES

“ASTM Book of Standards, Volume 06.01, Paint—Tests for Chemical, Physical, and Optical Properties; Appearance” (2002) ASTM International, West Conshohocken, Pa., U.S.A.
“ASTM Book of Standards, Volume 06.02, Paint—Products and Applications; Protective Coatings; Pipeline Coatings” (2002) ASTM International, West Conshohocken, Pa., U.S.A.
“ASTM Book of Standards, Volume 06.03, Paint—Pigments, Drying Oils, Polymers, Resins, Naval Stores, Cellulosic Esters, and Ink Vehicles” (2002) ASTM International, West Conshohocken, Pa., U.S.A.
“ASTM Book of Standards, Volume 06.04, Paint—Solvents; Aromatic Hydrocarbons” (2002) ASTM International, West Conshohocken, Pa., U.S.A.
“Directed Enzyme Evolution: Screening and Selection Methods (Methods in Molecular Biology) (Arnold, F. H. and Georgiou, G) Humana Press, Totowa, N.J., 2003.
“High Throughput Protein Expression and Purification Methods and Protocols,” (Doyle, S. A., Ed.) Humana Press, Walnut Creek, Calif., USA, pp. 129-132, 2009.
“Paint and Coating Testing Manual, Fourteenth Edition of the Gardner-Sward Handbook” (Koleske, J. V., Ed.) (1995) American Society for Testing and Materials, Philadelphia, Pa., U.S.A.
“Paints, Coatings and Solvents, Second, Completely Revised Edition” (Stoye, D. and Freitag, W., Eds.) (1998) Wiley-Vch, New York, U.S.A.
Adamitsch, B. F. et al., Lett Appl Microbiol 36:227-229, 2003.
Adams, M. D. et al. Science 287:2185-2195, 2000.
Ahmed, K. et al. J Biosci Bioeng 95:27-34, 2003.
Albizo, J. M. and White, W. E. “The Hydrolysis of GD and VX by Acetone Dried Preparations of Cured and Plasmid-Containing Pseudomonas Diminuta” Chemical Research, Development & Engineering Center Scientific conference on Chemical Defense Research, November 18-21, pp. 643-649, 1986.
Allouch, J., et al. J Biol. Chem. 278:47171-47180, 2003.
Almeida, et al., J. Mol. Biol. 315(4); 749-57, 2002.
Altmann, F. et al. Biochem. Biophys. Res. Commun. 136:329-335, 1986.
Assaf, N. A., and Dick, W. A., Biotechniques, 15:1010-1015, 1993.
Baba, T. and Schneewind, O. EMBO J 15:4789-4797, 1996
Baldridge, G. D. et al, Current Microbiology 51:233-238, 2005
Barbeyron, T. et al. J. Biol. Chem. 275:35499-35505, 2000.
Barras, D. R. and Stone, B. A. Biochim. Biophys. Acta 191:329-341, 1969a.
Barras, D. R. and Stone, B. A. Biochim. Biophys. Acta 191; 342-353, 1969b.
Benning, M. M. et al., Biochem. 33:15001-15007, 1994.
Benning, M. M. et al., Biochem. 34:7973-7978, 1995.
Benning, M. M. et al., J. Biol. Chem. 275:30556-30560, 2000.
Billecke, S. S. et al., Chemico-Biological Interactions 119-120, 251-256, 1999.
Blackburn, S. A. et al. Microbiology 144:73-82, 1998.
Blade, C. C. F. et al. Proc. R. Soc. Lond. B: Biol. Sci. 167:378-388, 1967a.
Blake, C. C. F. et al. Proc. R. Soc. Lond. B: Biol. Sci. 167:365-377, 1967b.
Bobek, et al., Antimicrob Agents Chemother 47(2); 643-52, 2003.
Bosmann, H. B. Biochim. Biophys. Acta. 276:180-191, 1972.
Brockerhoff, H. and Jensen, R. G. “Lipolytic Enzymes.” Academic Press, Inc., New York, N.Y., 1974.
Browder, H. P., et al., Biochem. Biophys. Res. Commun., 19, 383, 1965.
Bugg, C. E. et al., Sci. Am., 269:92-98, 1993.
Bulet, et al., submitted to SWISS-PROT data bank; 2002.
Bussey, H. et al. Proc Natl Acad Sci USA 92:3809-3813, 1995.
Cablo, Ed., Handbook of Coatings Additives, pp. 177-224, 1987.
Calandra, G. B., and Cole, R. M., Infect. Immun., 28:1033-1037, 1980.
Caldwell, S. R. and Raushel, F. M., Biochem. 30:744-7450, 1991c.
Cammue, et al., J. Biol. Chem. 267; 2228-33, 1992.
Canfield, R. E., J. Biol. Chem., 238:2698-2707, 1963.
Chae, M. Y. et al., Bioorg. Med. Chem. Lett. 4:1473-1478, 1994.
Cheng, T.-C. et al., Appl. Environ. Microbiol. 62(5):1636-1641, 1996.
Cheng, T.-C. et al., Applied and Environ. Microbio. 59(9):3138-3140, 1993.
Cheng, T.-C. et al., Chemico-Biological Interactions 119-120:455-462, 1999.
Cheng, T.-C. et al., J. Ind. Microbiol. 18:49-55, 1997.
Chen-Goodspeed, M. et al., Biochemistry, 40:1325-1331, 2001a.
Chen-Goodspeed, M. et al., Biochemistry, 40:1332-1339, 2001b.
Chesters, C. G. C. and Bull, A. T. Biochem. J. 86:31-38, 1963;
Chien, S., et al. Biochem. Biophys. Res. Commun. 76:317-323, 1977.
Chimpanzee Sequencing and Analysis Consortium. Nature 437:69-87, 2005.
Cho, C. M. et al., Applied and Enviro. Microbio., 2026-2030, 2002.
Chohnan et al. FEMS Microbiol Lett 213:13-20, 2002.
Clark, A. G. et al. Nature 450:203-218, 2007.
Clemens, S. et al., EMBO J 18:3325-3333, 1999.
Cobbett, C and Goldsbrough, P. Annu. Rev. Plant Biol. 53:159-182, 2002.
Cohen, A. A. and Shatzmiller, S. E., J. Mol. Graph. 11:166-173, 1993.
Cohen, J. A. and Warring, M. G. Biochim. Biophys. Acta. 26:29-39, 1957.
Coller, B. S. et al., J. Biol Chem. 268:20741-20743, 1993.
Creighton, T. E. Proteins: Structure and Molecular Properties, W. H. Freeman & Co., San Francisco, pp 79-86, 1983.
Cunningham, L. W. and Manners, D. J. Biochem. J 80:42β-43P, 1961.
Cutuli, et al., J Leukoc Biol. 27(2); 233-39, 2000.
Datta, P. K., et al. Can. J. Biochem. Physiol. 41:697-705, 1963
Dave, K. I. et al., Appl. Microbiol. Biotechnol. 41:352-358, 1994b.
Dave, K. I. et al., Biotechnol. Appl. Biochem. 19:271-284, 1994a.
Davies, R. C., et al., Biochim., Biophys. Acta, 178:294-305, 1969.
Dean, P. M., BioEssays, 16:683-687, 1994.
DeFrank, J. J. and Cheng, T.-C., J. Bacteriol 173:1938-1943, 1991.
Destoumieux, et al., Cell. Mol. Life Sci. 57; 1260-71, 2000.
Diago, M. et al. Aliment Pharmacol Ther 26:1131-1138, 2007
diSioudi, B. D. et al., Chemico-Biological Interactions 119-120:211-223, 1999b.
diSioudi, B. et al., Biochemistry 38:2866-2872, 1999a.
Donarski, W. J. et al., Biochemistry 28:4650-4655, 1989.
Duckworth, M. and Turvey, J. R. Biochem. 1 113:687-692, 1969.
Dudev, T. and Lim, C. Annu. Rev. Biophys. 37:97-116, 2008.
Dumas, D. P. et al., Biotech. Appl. Biochem. 11:235-243, 1989a.
Dumas, D. P. et al., Arch. Biochem. Biophys. 277:155-159, 1990.
Dumas, D. P. et al., Experientia, 46:729-731, 1990.
Dumas, D. P. et al., The Journal of Bio. Chem. 264(33):19659-19665, 1989b.
Duvick, et al., J. Biol. Chem. 267; 18814-20, 1992.
Eisenberg, et al.
Elliott, B. W. and Cohen, C. J. Biol. Chem. 261:11259-11265, 1986.
Ellman, G. L. et al., Biochem Pharmacol 7:88-95, 1961.
Elsik, C. G. et al. Science 324:522-528, 2009.
Ezaki, T and Suzuki, S., J. Clin. Microbiol., 16:844-846, 1982.
Farkas, E. et al. J. Chem. Soc., Dalton Trans., 611-614, 1984.
Fernandes, et al., Biochem. 1 368; 611-20, 2002.
Fiedler, et al., J. Chem. Technol. Biotechnol., 32:271-280, 1982.
Fischer, E. H. and Stein, E. A. Cleavage of O- and S-glycosidic bonds (survey), in Boyer, P. D., Lardy, H. and Myrbäck, K. (Eds.), The Enzymes, 2nd edn., vol. 4, Academic Press, New York, pp. 301-312, 1960.
Flick, E. W. “Handbook of Paint Raw Materials, Second Edition” (1989) Noyes Data Corporation/Noyes Publications, Park Ridge, N.J., U.S.A.
Fliss, I., et al., Biotechniques, 11:453-457, 1991.
Fujita, T. et al., Biochim. Biophys. Acta 1308 (1):49-57, 1996.
Fujitani, et al., J. Biol. Chem. 277; 23651, 2002.
Fukui, S. and Tanaka, A. Adv. Biochem. Eng. Biotechnol. 29:33, 1984.
Furka, A., et al. Int. I Pept. Protein Res. 37:487, 1991.
Gaeng, S. et al Appl Environ Microbiol 66:2951-2958, 2000.
Gao, et al., Biochemistry 40 (37); 10973-78, 2001.
Garcia, J. S. et al., Talanta 69:1-15, 2006.
Garden, J. M. et al., Comp. Biochem. Physiol. 52C:95-98, 1975.
Gargouri, Y. et al., Eur J Biochem., 180(2):367-371, 1989.
Gesell, et al., J. Biomol. NMR 9; 127, 1997.
Ghuysen, J.-M. et al. Biochemistry 8:213-222, 1969.
Gibbs, R. A. et al. Nature 428:493-521, 2004.
Gibbs, R. A. et al. Science 316:222-234, 2007.
Gomis-Ruth, F. X. Critical Reviews in Biochemistry and Molecular Biology, 43:5, 319-345, 2008.
Gopal, S. et al., Biochem. and Biophys. Research Comm. 279:516-519, 2000.
Goraya, et al., Eur. J. Biochem. 267; 894-900, 2000.
Grill, E. et al., Proc. Natl. Acad. Sci. USA 86:6838-6842, 1989
Grimsley, J. K. et al., Biochemistry 36(47):14366-14374, 1997.
Guichard, et al., PNAS USA, 91:9765-69, 1994.
Ha, S.-B., et al., Plant Cell 11:1153-1164, 1999.
Halverson, et al., Peptides 21; 469-76, 2000.
Hancock, R. E. W. and Scott, M. G. PNAS 97(16): 8856-8861, 2000.
Hara, et al., Biopolymers 58(4); 437-46, 2001.
Harper, L. et al., Appl. Env. Micro. 54:2586-2589, 1988.
Hartleib, J. and Ruterjans, H Biochim et Biophys Acta 1546:312-324, 2001b.
Hartleib, J. and Ruterjans, Prot. Expression and Purification 21:210-219, 2001a.
Hatfield, R. and Nevins, D. J. Carbohydr. Res. 148:265-278, 1986
He, X.-H. et al., Life Science 64(14):1163-1175, 1999
Herbold, D. R. and Glaser, L. J. Biol. Chem. 250:1676-1682, 1975.
Hill, et al., Science 251; 1481-85, 1991.
Hillier, L. W. et al. Nature 432:695-716, 2004.
Hirata, K. et al. Journal of Bioscience and Bioengineering 100(6): 593-599, 2005.
Hirota, Y. et al. Nucleic Acids Res. 18(21), 6432, 1990.
Hochuli, E. et al. Bio/Technology 6:1321-1325, 1988.
Holler, H., et al., Biochem., 14:2377-2385, 1975.
Holtje, J.-V. et al. J. Bacteriol 124:1067-1076, 1975.
Hong, S.-B. and Raushel, F. M. Biochem. 35:10904-10912, 1996.
Horne, I. et al., Appl. Environ. Microbiol. 68(7):3371-3376, 2002.
Hoskin, F. C. G. et al., Biochemical Pharmacology 34(12):2069-2072, 1985.
Hoskin, F. C. G. et al., Biochemical Pharmacology 46(7):1223-1227, 1993.
Hoskin, F. C. G. et al., Biochemical Pharmacology 49(5):711-715, 1995.
Hoskin, F. C. G. et al., Chemico-Biological Interactions 119-120:399-404, 1999.
Hoskin, F. C. G. et al., Chemico-Biological Interactions 119-120:439-444, 1999.
Hoskin, F. C. G. et al., Fundam. Appl. Toxicol. 4:5165-5172, 1984.
Houghten, R. A., et al. BioTechniques 13:412, 1992.
Houghten, R. A., et al. Nature 354:84, 1991.
Hruby, V. J., Biopolymers, 33:1073-1082, 1993.
Hung, S.-C. and Liao, J. C., Appl. Biochem. Biotechnol. 56(1):37-47, 1996.
Hunter, et al., J. Biol. Chem. 277; 35797, 2002.
Hwang, et al., Biochemistry 37; 4288, 1998.
III, C. R. et al. Biophys. J. 64(3):919-924, 1993
Iijima, et al., Dev. Comp. Immunol. 0; 2002.
In “Engineering of/with Lipases” (F. Xavier Malcata., Ed.) Kluwer Academic Publishers, Dordrecht, The Netherlands, 1996.
In “Handbook on the Toxicology of Metals” (Gunnar Nordberg, G., Fowler, B. A., Nordberg, M. and Friberg, L., Eds.) Academic Press, New York, pp. 263-278, 2007.
In “Paint and Surface Coatings, Theory and Practice, Second Edition,” (Lambourne, R. and Strivens, T. A., Eds.), pp. 193-194, 371-382 and 543-547, 1999
In “PCI Paints & Coatings Industry,” pp. 56, 58, 60, 62, 64, 66-68, 70, 72 and 74, July 2003
Inohue, M. et al. J. Plant Physiol. 154:334-340, 1999.
Isono, et al., J. Chem. Technol. Biotechnol., 32:271-80, 1979.
Jacobs, F. A. et al. Gene 83, 95-10, 1989.
Jam, M. et al. Biochem. 1 385:703-713, 2005.
Jekel, P. A., et al., Anal. Biochem. 134:347-354, 1983.
Jolles, P., Angewandte Chemie, International Edition, 8:227-239, 1969.
Jones, et al., FEBS Lett. 315; 187-92, 1993.
Jones, et al., J. Biol. Chem. 367; 23216-25, 1992.
Josse, D. et al., Chemico-Biological Interactions 119-120:71-78, 1999.
Josse, D. et al., J. Appl. Toxicol. 21:S7-S11, 2001.
Kanehisa, M. and Goto, S. Nucleic Acids Res. 28:27-30, 2000.
Kanehisa, M. et al. Nucleic Acids Res. 34:D354-357, 2006.
Kanehisa, M. et al. Nucleic Acids Res. 36:D480-D484, 2008.
Kaneva, I. et al., Biotechnol. Prog. 14:275-278, 1998.
Karsa, D. R., et al., Eds., Waterborne Coatings and Additives, The Royal Society of Chemistry, 1995.
Karube, I. et al., Appl. Microbiol. Biotechnol. 21:270-272, 1985.
Kim, C. et al., Biotechnol Bioeng 65:108-113, 1999.
Knech, S. et al. J. Mol. Recognit 22:270-279, 2009.
Kobayashi, R et al, J. Ferment Technol. 59:21-26, 1981.
Koepke, J. et al., Acta. Cryst. D 58:1757-1759, 2002.
Koide, N. and Muramatsu, T. J. Biol. Chem. 249:4897-4904, 1974.
Kokryakov, et al., FEBS Lett. 327; 231-36, 1993.
Koo, et al., Biochim. Biophys. Acta 1382; 80-90, 1998.
Kotrba, P. et al. Appl. Environ. Microbiol. 65, 1092-1098, 1999.
Kotrba, P. et al. J. Receptor Signal Transduct. Res. 19, 703-715, 1999.
Kroczynska, B. et al., J. Biol. Chem. 27912:11432-11443, 2004.
Kuo, J. M. and Raushel, F. M., Biochemistry 33:4265-4272, 1994.
Kurt Faber, “Biotransformations in Organic Chemistry, a Textbook, Third Edition.” Springer-verlag Berlin Heidelberg, pp. 114-115, 1997.
Kyte, J. and Doolittle, R. F. J. Mol. Biol., 157:105-132, 1982.
Lalonde, J. J. et al., J. Am Chem Soc 117:6845-6852, 1995.
Lamberty, et al., Biochemistry 40; 11995, 2001.
Law, M. Y. and Halliwell, B. Plant Sci. 43:185-191, 1986; Lederer, C. W. et al., BMC Genomics 8:26, 2007.
Leduc, M. et al., J. Bacteriol. 161:627-635, 1985.
Lee, et al., Biol. Pharm. Bull. 18; 1049-52, 1995.
Lee, et al., Protein Pept Lett 9(5); 395-402, 2002.
Li, S. et al. J Biochem (Tokyo) 124:332-339, 1998.
Li, S. L. et al. J. Bacteriol. 172:6506-6511, 1990.
Li, W.-S. et al., Bioorganic & Medicinal Chemistry, 9:2083-2091, 2001.
Liepinsh, et al., Nat Struct Biol. 4; 793, 1997.
Lindblad-Toh, K. et al. Nature 438:803-819, 2005.
Lineweaver, H. and Burke, D. “J. Am. Chem. Soc. 56:658-666, 1934.
Little, J. S. et al., Biochem Pharmacol 38(1):23-29, 1989.
Liu, C. and Xu, H. Journal of Inorganic Biochemistry 88:77-86, 2002.
Ljungquist, C. et al. Eur. J Biochem. 186,563-569, 1989.
Loessner, M. J. et al., Appl Environ Microbiol 62:3057-3060, 1996.
Lopez, M. et al., Blood 92(12):4602-4611, 1998.
Lopez, R. et al., Res Microbiol 151:437-443, 2000.
Mackinnon, C. M. et al., Eur. J. Biochem. 169:547-553, 1987;
Mackness, M. I. et al., Biochem. 1 245:293-296, 1987.
Macnicol, P. K. Plant Sci. 53:229-235, 1987
Mak, et al., Infect. Immun. 64; 4444-49, 1996.
Mandard, et al., Eur J Biochem. 256; 404, 1998.
Mandard, et al., Eur. J. Biochem. 269; 1190, 2002.
Mandard, et al., J. Biomol. Struct. Dyn. 17; 367, 1999.
Mandeles, S. and Bloch, K. J. Biol. Chem. 214:639-646, 1955
Maret, W. J. Anal. At. Spectrom 19:15-19, 2004.
Martinek, K. et al., Biochim. Biophys. Acta. 485:1-12, 1977.
Martinez, M. B. et al., Biochem 35(4):1179-1186, 1996.
Martinez, M. B. et al., Biochem 40(40):11965-11974, 2001.
Martins, et al., J. Mol. Biol. 258; 322, 1996.
Masaki, T. et al., Biochim. Biophys. Acta 660:51-55, 1981a.
Masaki, T. et al., Biochim. Biophys. Acta. 660:44-50, 1981b.
Matthews, D. J. Current Opinion in Biotechnology, 6:419-424, 1995.
Mauro, J. M. and Pazirandeh, M. Lett. Appl. Microbiol. 30, 161-166, 2000.
McDaniel, S. and Wild, J. Arch. Env. Contam. Toxic. 17:189-194, 1988.
McDaniel, S. et al., J. Bact. 170:2306-2311, 1988a.
McDaniel, S., Ph.D. Dissertation, Texas A&M University, 1985.
Mejáre, M. and Billow, L. Trends in Biotechnology 19(2), 67-73, 2001.
Mejáre, M. et al. Protein Eng. 11, 489-494, 1998.
Melo, E. P. et al., Biochemistry, 34(5):1615-1621, 1995.
Michalowski, et al., submitted to EMBL GenBank DDBJ databases, 1998.
Michel, G. et al. Acta Crystallogr. D Biol. Crystallogr. 55:918-920, 1999.
Michel, G. et al. J. Biol. Chem. 276:40202-40209, 2001.
Michel, G. et al. J. Mol. Biol. 334:421-433, 2003.
Michel, G., et al., Structure 9:513-525, 2001.
Michel, S. L. J., and Berg, J. M. Chemistry & Biology, 9:667-672, 2002.
Mikkelsen, T. S. et al. Nature 447:167-177, 2007.
Miller, C. E. Ph.D. dissertation, Texas A&M University, 1992.
Moore, et al., J. Biol. Chem. 266; 19851-57, 1991.
Moore, G. J., Trends Pharmacol. Sci., 15:124-129, 1994.
Mooreman, et al., Eur. J. Biochem. 269; 4799-810, 2002.
Mor, et al., Eur J Biochem 219(1-2); 145-54, 1994.
Mori, T. et al., Enzymologia, 43:213-226, 1972.
Mulbry, W. and Karns, J., J Bacteriol. 171:6740-6746, 1989.
Myers, F. L. and Northcote, D. H., Biochem. 1 71:749-756, 1959
Nagaoka, et al., DNA Seq. 4; 123-28, 1993.
Nagaoka, et al., FEBS Lett. 280; 287-91, 1991.
Nakamura, Y. et al. DNA Res 10:137-145, 2003.
Nakamura, Y. et al. DNA Res 9:123-130, 2002.
Niwa, t., et al., J. Microbiol. Methods, 61, 251-260, 2005.
O'Flaherty, S. et al., J. Bacteriol 187:7161-7164, 2005.
Ohara, T. et al. J. Biol. Chem. 264:20625-2063, 1989.
Ohbuchi, K. et al., J. Biosci. Bioeng. 91:487, 2001.
Ohta, Y. and Hatada, Y. J. Biochem. (Tokyo) 140:475-481, 2006.
Ohta, Y. et al. Biosci. Biotechnol. Biochem. 68:1073-1081, 2004a.
Ohta, Y. et al. Microbulbifer. Appl. Microbiol. Biotechnol. 64:505-514, 2004b.
Ohta, Y., et al. Curr. Microbiol. 50:212-216, 2005a.
Ohto, T. et al., J Blot Chem. 280(26):24576-24583, 2005b.
Olson, et al., Biochem. Biophys. Res. Commun. 288; 1001-05, 2001.
Omburo, G. A. et al., Biochemistry 32:9148-9155, 1993.
Omburo, G. A. et al., J. Biol. Chem. 267:13278-13283, 1992.
Oppenheim, et al., J. Biol. Chem. 263; 7472-77, 1988.
Orivel, et al., J. Biol. Chem. 276; 17823-29, 2001.
Palenik, B. et al. Nature 424:1037-1042, 2003.
Park, et al., Biochem. Biophys. Res. Commun. 205; 948-54, 1994.
Park, et al., Biochem. Biophys. Res. Commun. 218; 408-13, 1996.
Park, et al., FEBSLett 507(1); 95-100, 2001.
Park, et al., FEBS Lett. 411; 173-78, 1997.
Patton, T. C. “Paint Flow and Pigment Dispersion, A Rheological Approach to Coating and Ink Technology,” 1979
Pazehoski, P. O. Journal of Inorganic Biochemistry 102:522-531, 2008.
Pazirandeh, M. et al. Appl. Environ. Microbiol. 64, 4068-4072, 1998.
Persichetti, R. A. et al., Tetrahedron Lett, 37:6507-6510, 1996.
Petit, J., et al., Trends Genet., 10:4-5, 1994.
Pierce, R. J. et al. Biochem. 1 180:673, 1979.
Pierce, R. J. et al. Biochem. 1 185:261-264, 1980.
Plummer, T. H., Jr. and Tarentino, A. L. J. Biol. Chem. 256:10243-10246, 1981.
Potin, P. et al. Eur. J Biochem. 201; 241-247, 1991.
Potin, P. et al. Eur. J Biochem. 228:971-975, 1995.
Potin, P., et al. Eur. J. Biochem. 214:599-607, 1993.
Powell, M. F. et al., Pharma. Res., 10:1268-1273, 1993.
Primrose, S. et al., “Principles of Gene Manipulation” pp. 301-303, 2001.
Proteins Labfax (Price, N.C., Ed.) BIOS scientific publishers Ltd., Oxford, UK, pp. 52-53, 1996.
Qu, et al., Eur. J. Biochem. 127; 219-24, 1982.
Qureshi, N. et al., Appl. Microbiol. Biotechnol. 21:280-281, 1985.
Raj, et al., Biopolymers 45(1); 51-67; 1998.
Rastogi, V. et al., Biochem. and Biophys. Research Comm. 241:294-296, 1997.
Rauser, W. E. Plant Physiol. 109:1141-1149, 1995.
Raushel, F. M., Microbiology, 5:288-295, 2002.
Raveh, L. et al., Biochemical Pharmacology 44(2):397-400, 1992.
Ravi, et al., J. Biol. Chem. 272; 24480-87, 1997.
Rebuffat, et al., Eur. J. Biochem. 201; 661-74, 1991.
Recsei, P. A., et al. Proc. Natl. Acad. Sci. U.S.A. 84:1127-1131, 1987
Reese, E. T. and Mandels, M. Can. J. Microbiol. 5:173-185, 1959.
Regan, L. and Clarke, N. D., Biochemistry, 29:10878-10883, 1990
Reid, C. W. et al., FEBS Lett 574:73-79, 2004.
Richins, R. D. et al., Biotechnology and Bioengineering 69(6):592-596, 2000.
Richins, R. D. et al., Nature Biotechnology 15:984-987, 1997.
Robinette, et al., Cell. Mol. Life Sci. 54; 467-75, 1998.
Rogers, K. R. et al., Biotech Prog 15:517-521, 1999.
Rowland, S. S. et al., Appl. Microbiol. Biotechnol. 38:94-100, 1992.
Rozek, et al., Biochemistry 39; 15765, 2000.
Ruissen, et al., Peptides 2002 23(8); 1391-99, 2002.
Rupley, J. A., Biochim. Biophys. Acta, 83:245-255, 1964.
Ryle, M. Motor Ship 34, 1999.
Scharff, E. I. et al., Acta Cryst. D 57:148-149, 2001.
Scheurwater, E. et al. Int. J. Biochem. Cell Biol, 2007.
Schibli, et al., Biochemistry 38; 16749, 1999.
Schonwetter, et al., Science 267; 1645-48, 1995.
Scoochi, et al., FEBS Lett. 417; 311-15, 1997.
Scott, J. H. and Schekman, R. et al., J. Bacteriol 142:414-423, 1980.
Segel, I. H. Biochemical Calculations: How to Solve Mathmatical Problems in General Biochemistry 2^{nd} Edition, John Wiley & Sons, Inc., New York, 1976.
Selsted, et al., Proc. Natl. Acad. Sci. USA 85; 592-96, 1988.
Serdar, C. M. et al., Bio/Technology 7:1151-1155, 1989.
Shen, S. H. et al., J Biol Chem 266:1058-1063, 1991.
Shim, H. et al., J. Biol. Chem. 273(28):17445-17450, 1998.
Shimazu, M. et al., Biotech and Bioeng 76(4):318-324, 2001b.
Shimazu, M. et al., Biotechnol. Prog. 17:76-80, 2001a.
Shimazu, M. et al., Biotechnology and Bioengineering 81(1):74-79, 2002.
Skerlavaj, et al., J Biol Chem. 271; 28375-81, 1996.
Skerra, A. et al. BiolTechnol. 9, 273-278, 1991.
Smith, M. C. et al. J Biol. Chem. 263, 7211-7215, 1988.
Smith, M. et al. Inorg. Chem. 26, 1965-1969, 1987.
Smith, T. J. et al., Microbiology 146:249-262, 2000.
Snoke, J. E. et al., J Biol. Chem. 201:573-586, 1953;
Sodergren, E. et al. Science 314:941-952, 2006.
Soedjanaatmadja, et al., Biochim. Biophys. Acta 1209; 144-48, 1994. Tang, et al., Infect. Immun. 67; 6139-44, 1999.
Somara, S. et al., Indian J Exp Biol 40(7):774-779, 2002.
Sousa, C. et al. Bio/Technology 14, 1017-1020, 1996.
Sousa, C. et al. J. Bacteriol. 180, 2280-2284, 1998.
Soya, V. V., Elyakova, L. A. and Vaskovsky, V. E. Biochim. Biophys. Acta 212:111-115, 1970.
Sovago, I. and Osz, K. Dalton Trans., 3841-3854, 2006.
Sugano, Y. et al. Appl. Environ. Microbiol. 59:1549-1554, 1993.
Sugano, Y., et al. J Bacteriol. 176:6812-6818, 1994.
Sugita, C. et al. Photosynth Res 93:55-67, 2007.
Tai, T. et al. J Biol. Chem. 250:8569-8575, 1975.
Takahashi, N. and Nishibe, H. J. Biochem. (Tokyo) 84:1467-1473, 1978.
Takahashi, N. Biochem. Biophys. Res. Commun. 76:1194-1201, 1977.
Tarentino, A. L. et al. Biochemistry 24:4665-4671, 1985.
Tarentino, A. L., et al. J. Biol. Chem. 249:818-824, 1974.
Terras, et al., FEBS Lett. 316; 233-40, 1993.
The C. elegans Sequencing Consortium. Science 282:2012-2018, 1998.
Theil, et al., EMBO J. 2; 1159-63, 1983.
Theologis, A. et al. Nature 408:816-820, 2000.
Thumm, G. and Gotz, F. Mol. Microbiol. 23:1251-1265, 1997.
Thunnissen, A. M. et al. Nature 367:750-753, 1994.
Tinoco, et al., VOL. 277, No. 39; 36351-56, 2002.
Tracey, M. V. Biochem. J. 61:579-586, 1955.
Trayer, H. R., and Buckley, C. E., J. Biol. Chem., 245, 4842-4846, 1970.
Tsuchiya, D., and Taga, M., Phytopathology, 91, 354-360, 2001.
Tsunasawa, S. et al. J. Biol. Chem. 264:3832-3839, 1989.
U.S. application Ser. No. 10/601,207
Ueta, et al., J Pept Res 2001 57(3); 240-49, 2001.
Umemura, I. et al., Appl. Microbiol. Biotechnol. 20:291-295, 1984.
Unger, T. F. The Scientist, 11(17):20, 1997.
U.S. Pat. No. 4,939,090
U.S. Pat. No. 5,885,782
Usha, B. et al., Curr. Sci. 93(9):1215-1219, 2007.
Vails, M. et al., Biochimie, 80:855-861, 1998.
Vanhooke, J. L. et al., Biochemistry 35:6020-6025, 1996.
Vatamaniuk, O. K, et al., Proc. Natl. Acad. Sci. USA 96:7110-7115, 1999.
Ventom, A. M. and Asenjo, J. A. J Biotechnol Tech 4:171-176, 1990.
Vitarius, J. A. and Sultatos, L. G. Life Sciences, 56(2):124-134, 1995.
Vodovar, N. et al., Nat Biotechnol. 246:673-679, 2006.
Vogle, et al., Biochem. Cell Biol. 80; 49-63, 2002.
Walker, A. W. and Keasling, J. D. Biotech. and Bioeng. 78(7):715-721, 2002.
Wang, A. A. et al., Applied and Environ. Microbio. 68(4):1684-1689, 2002.
Wang, et al., Biochem. Biophys. Res. Commun. 279; 407-11, 2000.
Wang, et al., Biochem. Biophys. Res. Commun. 288; 765-70, 2001.
Wang, J. et al., Biomacromolecules 2:700-705, 2001.
Ward, J. B. et al., J. Gen. Microbiol. 128:1171-1178, 1982.
Warren, W. C. et al. Nature 453:175-183, 2008.
Watkins, L. M. et al., J. Biol. Chem. 272(41):25596-25601, 1997a.
Watkins, L. M. et al., Proteins: Struct., Funct., and Gen. 29:553-561, 1997b.
Weigl, J. and Yashe, W. Can. J. Microbiol. 12:939-947, 1966.
Wernersson, R. et al. BMC Genomics 6:70, 2005.
Whitaker, D. R. and Roy, C. Can. J. Biochem. 45:911, 1967.
Whitaker, D. R. et al. Can. J. Biochem. 43:1961-1970, 1965.
Whitaker, D. R. et al. Can. J. Biochem. Physiol. 41:671-696, 1963
Wicks, et al., Organic Coatings, Science and Technology, Volume 1: Film Formation, Components, and Appearance, pp. 318-20, 1992.
Wicks, et al., Organic Coatings, Science and Technology, Volume 2: Applications, Properties and Performance, pp. 145, 309, 319-23, and 340-41, 1992.
Wicks, Jr., Z. W., Jones, F. N., Pappas, S. P. “Organic Coatings, Science and Technology, Volume 1: Film Formation, Components, and Appearance,” pp. 318-320, 1992
Wild, J. R. et al., Proc. U.S. Army Chem. Res. Devel. Eng. Center Sci. Conf. Chem. Defense Res. 18-21 Nov., p. 629-634, 1986.
Wilde, et al., J. Biol. Chem. 264; 11200-03, 1989.
Wiley, R. A. and Rich, D. H. Med. Res. Rev., 13:327-384, 1993.
Wood, V. et al. Nature 415:871-880, 2002.
Wu, C.-F. et al., Appl Microbiol Biotechnol 54:78-83, 2000b.
Wu, C.-F., Biotechnol. Prog. 17:606-611, 2001a.
Wu, C.-F., Biotechnology and Bioengineering 75(1):100-103, 2001b.
Wu, C.-F., Biotechnology and Bioengineering 77(2):212-218, 2002.
Xing, G. and DeRose, V. J. Current Opinion in Chemical Biology 5:196-200, 2001.
Xu, et al., J. Dent. Res 69; 1717-23, 1990.
Yang, Z. et al., Biotechnol Bioeng 45:10-17, 1995.
Yang, Z. et al., Enzyme Microb Technol 18:82-89, 1996.
Yi, et al., FEBS Lett. 398; 87-90, 1996
Yin, et al., Arch Oral Biol. 48(5); 361-68, 2003.
Yokogawa, K., et al., Agric. Biol. Chem., 39:1533-1545, 1975.
Yount, et al., J. Immunol. 155; 4476-84, 1995.
Zasloff, M. Proc. Natl. Acad. Sci. USA 84:5449-5453, 1987.
Zhang, et al., Biochemistry 31; 11348-56, 1992.
Zhao, et al., FEBS Lett. 346; 285-88, 1994.
Zhao, et al., FEBS Lett. 368; 197-202, 1995.
Zhu, et al, Endocrinology 130; 1413-23, 1992.
Zimmermann, et al., Biochemistry 34; 13663, 1995.

1. (canceled)

2. A method of imparting marine anti-fouling properties on a surface of an object, comprising
providing an object having a surface thereof in contact with an aqueous marine environment during use of the object, wherein the aqueous marine environment comprises at least one metal ion that chemically impedes growth of at least one marine-based surface-fouling organism when brought into contact therewith at a sufficient concentration thereof;
prior to the object being subject to an instance of said use, treating the surface of the object with a coating composition comprising a metal binding peptide to which said at least one metal ion of the aqueous marine environment binds when contacted therewith; and
subjecting the object to an instance of said use after performing said treating to thereby cause a quantity of said at least one metal ion from within the aqueous marine environment to bind to the coating composition comprising the metal binding peptide for creating an anti-fouling coating on the surface of the object, wherein the quantity of said at least one metal ion bound to the coating composition comprising the metal binding peptide is at least 60% greater than a quantity of said at least one metal ion bound from within the aqueous marine environment to the coating composition when not comprising the metal binding peptide.

3. The method of claim 2 wherein the metal binding peptide is His6 peptide.

4. The method of claim 2 wherein:
the at least one metal ion is at least one of copper ion and nickel ion; and
the aqueous marine environment is ocean water comprising at least one of copper ion and nickel ion whereby the anti-fouling coating comprises at least one of copper ion and nickel ion.

5. The method of claim 4 wherein the metal binding peptide is His6 peptide.

6. The method of claim 2 wherein the metal binding peptide comprises a His-tag sequence.

7. The method of claim 6 wherein the metal binding peptide is His6 peptide.

8. The method of claim 6 wherein the coating composition comprises a paint and an aqueous solution of the His6 peptide mixed at a ratio of 1 about part of the aqueous solution of the His6 to 7 about parts of the paint.

9. The method of claim 8 wherein:
the at least one metal ion is at least one of copper ion and nickel ion; and
the aqueous marine environment is ocean water comprising at least one of copper ion and nickel ion whereby the anti-fouling coating comprises at least one of copper ion and nickel ion.

10. The method of claim 9 wherein the metal binding peptide is His6 peptide.

11. An anti-fouling bottom surface of an aquatic vessel made by a process comprising:
providing an aquatic vessel having at least a portion of a bottom surface thereof in continuous contact with an aqueous marine environment during use of the vessel, wherein the aqueous marine environment comprises at least one metal ion that chemically impedes growth of at least one marine-based surface-fouling organism when brought into contact therewith at a sufficient concentration thereof, wherein the at least a portion of the bottom surface is exposed to the aqueous marine environment when submersed therein; and
prior to the object being subject to an instance of said use, treating the at least a portion of the bottom surface with a coating composition comprising a metal binding peptide to which said at least one metal ion binds when contacted therewith to cause a quantity of said at least one metal ion from within the aqueous marine environment to bind to the coating composition comprising the metal binding peptide when the at least a portion of the bottom surface is submersed within the aqueous marine environment after performing said treating to thereby cause for creating an anti-fouling coating on the at least a portion of the bottom surface, wherein the quantity of said at least one metal ion bound to the coating composition comprising the metal binding peptide is at least 60% greater than a quantity of said at least one metal ion bound from within the aqueous marine environment to the coating composition when not comprising the metal binding peptide.

12. The anti-fouling bottom surface of the aquatic vessel made by a process of claim 11 wherein the metal binding peptide is His6 peptide.

13. The anti-fouling bottom surface of the aquatic vessel made by a process of claim 11 wherein:
the at least one metal ion is at least one of copper ion and nickel ion; and
the aqueous marine environment is ocean water comprising at least one of copper ion and nickel ion whereby the anti-fouling coating comprises at least one of copper ion and nickel ion.

14. The anti-fouling bottom surface of the aquatic vessel made by a process of claim 13 wherein the metal binding peptide is His6 peptide.

15. The anti-fouling bottom surface of the aquatic vessel made by a process of claim 11 wherein the metal binding peptide comprises a His-tag sequence.

16. The anti-fouling bottom surface of the aquatic vessel made by a process of claim 15 wherein the metal binding peptide is His6 peptide.

17. The anti-fouling bottom surface of the aquatic vessel made by a process of claim 15 wherein the coating composition comprises a paint and an aqueous solution of the His6 peptide mixed at a ratio of 1 about part of the aqueous solution of the His6 to 7 about parts of the paint.

18. The anti-fouling bottom surface of the aquatic vessel made by a process of claim 17 wherein:
the at least one metal ion is at least one of copper ion and nickel ion; and
the aqueous marine environment is ocean water comprising at least one of copper ion and nickel ion whereby the anti-fouling coating comprises at least one of copper ion and nickel ion.
